(19) United States
(12) Patent Application Publication
(10) Pub. No.: US 2023/0220463 A1
(43) Pub. Date: Jul. 13, 2023

(54) MULTIPLEX DETECTION OF NUCLEIC ACIDS USING MIXED REPORTERS

(71) Applicant: SpeeDx Pty Ltd, Eveleigh, New South Wales (AU)

(72) Inventors: Alison Velyian Todd, Glebe, New South Wales (AU); Nicole Jane Hasick, Gymea, New South Wales (AU); Ryung Rae Kim, Rhodes, New South Wales (AU); Andrea Lee Lawrence, Berkeley, New South Wales (AU)

(21) Appl. No.: 17/923,562

(22) PCT Filed: Jun. 30, 2020
(86) PCT No.: PCT/AU2020/050682

§ 371 Date: Dec. 15, 2022

(51) Int. Cl. C12Q 1/6876; C12Q 1/6818; C12Q 1/6844

(52) U.S. Cl. CPC C12Q 1/6876; C12Q 1/6818; C12Q 1/6844; C12Q 2600/16

(57) ABSTRACT

The present invention provides oligonucleotides and methods for their use in the detection and/or differentiation of target nucleic acids. The oligonucleotides and methods find particular application in amplifying, detecting, and/or discriminating multiple targets simultaneously.

### TECHNICAL FIELD

The present invention relates generally to the field of molecular biology. More specifically, the present invention provides oligonucleotides and methods for their use in the detection and/or differentiation of target nucleic acids. The oligonucleotides and methods find particular application in amplifying, detecting, discriminating and/or quantifying multiple targets simultaneously.

### BACKGROUND

Genetic analysis is becoming routine in the clinic for assessing disease risk, diagnosis of disease, predicting a patient's prognosis or response to therapy, and for monitoring a patient's progress. The introduction of such genetic tests depends on the development of simple, inexpensive, and rapid assays for discriminating genetic variations.

Methods of in vitro nucleic acid amplification have wide-spread applications in genetics and disease diagnosis. Such methods include polymerase chain reaction (PCR), reverse transcription polymerase chain reaction (RT-PCR), strand displacement amplification (SDA), nicking enzyme amplification reaction (NEAR), helicase dependent amplification (HDA), Recombinase Polymerase Amplification (RPA), loop-mediated isothermal amplification (LAMP), rolling circle amplification (RCA), transcription-mediated amplification (TMA), self-sustained sequence replication (3SR), nucleic acid sequence based amplification (NASBA), Ligase Chain Reaction (LCR) or Ramification Amplification Method (RAM). Each of these target amplification strategies requires the use of oligonucleotide primer(s). The process of amplification results in the exponential amplification of amplicons which incorporate the oligonucleotide primers at their 5′ termini and which contain newly synthesized copies of the sequences located between the primers.

Commonly used methods for monitoring the accumulation of amplicons in real time, or at the conclusion of amplification, include detection using MNAzymes with universal substrate probes, target-specific Molecular Beacons, Sloppy Beacons, Eclipse probes, TaqMan Probes or Hydrolysis probes, Scorpion Uni-Probes or Bi-Probes, Catcher/Pitcher probes, Dual Hybridization probes and/or the use of intercalating dyes such as SybGreen. High Resolution Melt curve analysis can be performed during or at the conclusion of several of these protocols to obtain additional information since amplicons with different sequences denature at different temperatures, known as the melting temperature or Tm. Such protocols measure melting curves which result from either a) the separation of the two strands of double stranded amplicons in the presence of an intercalating dye, or b) the separation of one strand of the amplicon and a complementary target-specific probe labelled with a fluorophore and quencher or c) separation of non-target related duplexes, for example, Catcher duplexes which are only generated in the presence of target. Melt curve analysis provides information about the dissociation kinetics of two DNA strands during heating. The melting temperature (Tm) is the temperature at which 50% of the DNA is dissociated. The Tm is dependent on the length, sequence composition and G-C content of the paired nucleotides. Elucidation of information about the target DNA from melt curve analysis conventionally involves a series of fluorescence measurements acquired at small intervals, typically over a broad temperature range. Melting temperature does not only depend upon on the base sequence. The melting temperature can be influenced by many factors including the concentrations of oligonucleotides, cations in the buffer (both monovalent (Nat) and divalent (Mg²⁺) salts), and/or the presence or absence of destabilizing agents such as urea or formamide.

In general, the number of available fluorescent channels capable of monitoring discrete wavelengths limits the number of targets which can be detected and specifically identified in a single reaction on a fluorescent reader. Recently, a protocol known as “Tagging Oligonucleotide Cleavage and Extension” (TOCE) expands this capacity allowing multiple targets to be analysed at a single wavelength. TOCE technology uses Pitcher and Catcher oligonucleotides. Pitchers have two regions, the Targeting Portion, which is complementary to the target, and the Tagging portion which is non-complementary and located at the 5′ end. The Capture oligonucleotide is dual labelled and has a region at its 3′ end which is complementary to the tagging portion of the Pitcher. During amplification, the Pitcher binds to the amplicons and when the primers extend the exonuclease activity of the polymerase can cleave the Tagging portion from the Pitcher. The released Tagging portion then binds to the Catcher Oligonucleotide and functions as a primer to synthesise a complementary strand. The melting temperature of the double stranded Catcher molecule (Catcher-Tm) then acts as a surrogate marker for the original template. Since it is possible to incorporate multiple Catchers with different sequences and lengths, all of which melt at different temperatures, it is possible to obtain a series of Catcher-Tm values indicative of a series of targets whilst still measuring at a single wavelength. Limitations with this approach include inherent complexity as it requires the released fragment to initiate and complete a second extension on an artificial target and post amplification analysis of multiple targets requires complex algorithms to differentiate or quantify the proportion of signal related to each specific target.

Hairpin probes or Stem-Loop probes have also proven useful tools for detection of nucleic acids and/or monitoring target amplification. One type of hairpin probe, which is dual labelled with a fluorophore and quencher dye pair, is commonly known in the art as a Molecular Beacon. In general, these molecules have three features; 1) a Stem structure formed by hybridization of complementary 5′ and 3′ ends of the oligonucleotide; 2) a loop region which is complementary to the target, or target amplicon, to be detected; and 3) a fluorophore quencher dye pair attached at the termini of the Molecular Beacon. During PCR, the loop region binds to the amplicons due to complementarity and this causes the stem to open thus separating the fluorophore quencher dye pair. An essential feature of Molecular Beacons is that the loop regions of these molecules remain intact during amplification and are neither degraded or cleaved in the presence of target or target amplicons. The separation of the dye pair attached on the termini of an open Molecular Beacon causes a change in fluorescence which is indicative of the presence of target. The method is commonly used for multiplex analysis of multiple targets in a single PCR test. In general for multiplex analysis, each Molecular Beacon has a different target-specific loop region and a unique fluorophore, such that hybridization of each different Molecular Beacons to each amplicon species can be monitored in a separate channel i.e. at a separate wavelength.

The concept of Molecular Beacons has been extended in a strategy known as Sloppy Beacons. In this protocol the loop region of a single Beacon is long enough such that it can tolerate mismatched bases and hence bind to a number of closely related targets differing by one or more nucleotides. Following amplification, melt curve analysis is performed and different target species can be differentiated based on the temperature at which each of the duplexes formed by hybridization of the target species with the loop region of a Sloppy Beacon separate (melt). In this way multiple closely related species can be detected at a single wavelength and discriminated simultaneously by characterising the melting profile of specific targets with the single Sloppy Beacon. Standard Molecular Beacons and Sloppy Beacons differ from TaqMan and Hydrolysis probes in that they are not intended to be degraded or cleaved during amplification. A disadvantage of DNA hybridisation-based technologies such as sloppy beacons and TOCE is that they may produce false positive results due to non-specific hybridisation between probes and non-target nucleic acid sequences.

Many nucleic acid detection assays utilise melt curve analyses to either identify the presence of specific target sequences in a given sample or to elucidate information about the amplified sequence. Melt curve analysis protocols entail measuring fluorescence at various temperatures over an incrementally increasing temperature range. The change in slope of this curve is then plotted as a function of temperature to obtain the melt curve. This process is often slow and typically takes anywhere between 30-60 mins to complete. Furthermore, melt curve analyses can require interpretation by skilled personnel and/or use of specialised software for results interpretation. Hence, there is a high demand for faster and/or simpler alternatives to melt curve analyses.

Melt Curves are typically analysed post-PCR and therefore only allow for a qualitative determination of the presence or absence of target in a sample. In many instances, a quantitative, or semi-quantitative, determination of the amount of genomic material present in a sample is required. Therefore, there is a high demand for fast alternatives to melt curve analysis that also provide quantitative information about a sample.

A need exists for improved compositions and methods for the simultaneous detection, differentiation, and/or quantification of multiple unique amplicons generated by PCR or by alternative target amplification protocols.

### SUMMARY OF THE INVENTION

The present invention addresses one or more deficiencies existing in current multiplex detection assays.

Provided herein are methods and compositions which extend the capacity to multiplex during amplification protocols. These methods combine “Standard Reporters”, which include Substrates and Probes well known in the art, together with structure(s) referred to herein as LOCS (Loops Connected to Stems). Standard Reporters include, but are not limited to, Probes and Substrates including linear MNAzyme substrates, TaqMan probes or hydrolysis probes, Molecular Beacons, Sloppy Beacons, Eclipse probes, Scorpion Uni-Probes or Bi-Probes, Capture/Pitcher Oligonucleotides, and dual-hybridization probes. The combination of a Standard Reporter system, together with one or more LOCS wherein all species can, for example, be labelled with a single detection moiety (e.g. the same fluorophore and quencher pair) allows multiple targets to be individually discriminated within a single reaction. The approach involves measurement of the signal generated from the “Standard Reporter” and one or more LOCS, at one or more temperatures. The generation of signal from a LOCS can be dependent upon several factors including any one or more of:
the temperature at which the signal is measured;
whether or not the Loop portion of the LOCS has been cleaved or degraded in response to the presence of target;
the melting temperature of the stem portion of the specific LOCS in its “Intact”, or in its cleaved or degraded, “Split” conformation.

The melting temperature of the stem region of a Split LOCS acts as a surrogate marker for the specific target which mediated the target-dependant cleavage or degradation of the Loop of the Intact LOCS. Other methods incorporating stem-loop structures have exploited the change in fluorescent signal following either (a) hybridization of the loop region to target amplicons (e.g. Molecular Beacons & Sloppy Beacons) to increase the distance between dye pairs, or (b) by target-mediated cleavage allowing physical separation of the dyes (e.g. Cleavable Molecular Beacons). Cleavable Molecular Beacons have typically been used to generate a positive or negative signal for a given target at a single wavelength. Multiplex target detection generally requires the detection of different targets via signals emitted at different wavelengths. As such, the incorporation of variant stems into different Cleavable Molecular Beacons labelled with similar or identical detection moieties and designed to detect different targets offer the capacity to discriminate between detectable signals indicative of individual targets based on differences in stem melting temperatures, rather than needing employ distinct detectable signals between targets.

The present invention provides improvements over existing multiplex detection assays which arise, at least in part, through manipulating the melting temperature of the stem portion of stem-loop structures by changing the length and/or sequence composition of the stem such that each stem melts and generates signal at a different temperature.

The present invention can include the use of a Standard Reporters together with a single LOCS reporter or multiple LOCS reporters in a single reaction. Both the Standard and LOCS reporters may be labelled with the same or similar detection moiet(ies) that can be detected in essentially the same manner (e.g. fluorophores that emit in the same region of the visible spectrum, nanoparticles of the same size and/or type for colorimetric or SPR detection, reactive moieties (e.g. alkaline phosphatase or peroxidase enzymes) for chemiluminescent detection, electroactive species (e.g. ferrocene, methylene blue or peroxidase enzymes) for electrochemical detection. When multiple LOCS are present and labelled with, for example, the same detection moiety these may contain (a) different loop sequences which each allow direct or indirect detection of multiple targets simultaneously and/or (b) different stem sequences that melt at discrete temperatures and which can be used to identify the specific target(s) present within the multiple targets under investigation. The methods of the present invention use LOCS which provide one or more advantages over art-known methods such as, for example, the TOCE protocol in that separate catcher molecules are not required, and as such this reduces the number of components in the reaction mix and reduces costs. Furthermore, the methods of the present invention are inherently less complex than the TOCE method which requires the released fragment to initiate and complete a second extension on a synthetic target.

In some embodiments, the LOCS probes may be universal (independent of target sequence) and/or may be combined with a range of detection technologies, thus delivering wide applicability in the field of molecular diagnostics. Additionally, the melting temperature used in other conventional amplification and detection techniques is typically based on hybridisation and melting of a probe with a target nucleic acid. This suffers from the disadvantage of increased false-positives due to non-specific hybridisation between probes and non-target nucleic acid sequences. The methods of the present invention overcome this limitation because those LOCS reporter probes which contain universal substrates do not bind with target sequence. Finally, it is well-known in the art that intramolecular bonds are stronger than intermolecular bonds and thus, the probability that these un-cleaved (intact) LOCS would hybridise with non-specific target to produce false-positive signals is much lower.

As a result of intramolecular bonds being stronger than intermolecular bonds, a dual labelled LOCS will melt at one temperature when intact, and will melt at a lower temperature following target-dependent cleavage or degradation of the loop region which splits the LOCS into two fragments. This property of nucleic acids is exploited in the current invention to extend the capacity of instruments to differentiate multiple targets using a single type of detector such as one fluorescence channel, or a specific mode of colorimetric, surface plasmon resonance (SPR), chemiluminescent, or electrochemical detection.

The temperature dependent fluorescent signals produced by LOCS reporters of the present invention are well-defined and independent of the target DNA. Thus, it is possible to elucidate information about target DNA from measurements of fluorescent signal generated at selected temperatures, rather than a complete temperature gradient, providing an advantage in reduction of the run time on thermal cycling devices (e.g. PCR devices). By way of non-limiting example, on a Bio-Rad CFX96 PCR system, conducting a traditional melt analysis with settings for the temperature between 20° C. and 90° C. with 0.5° C. increments and 5 seconds hold time requires 141 fluorescence measurement cycles and approximately 50 minutes of run time. With the use of LOCS probes, the information about target DNA may be obtained from the same device with 2-6 fluorescence measurements and require approximately 2-5 minutes of run time. Without any specific limitation, the reduction of run time can be advantageous in numerous applications including, for example, diagnostics.

In the present invention, LOCS probes are combined with standard reporters or probes or substrates to simultaneously detect, differentiate, and/or quantify multiple targets. Individual signals indicative of the various targets may be detectable by the same means such as, for example, via signals emitting in a single fluorescent channel or detectable by a specific mode of colorimetric, surface plasmon resonance (SPR), chemiluminescent, or electrochemical detection. In the conventional qPCR, quantification of the target DNA may be determined using the cycle quantification (Cq) value from an amplification curve obtained by measuring fluorescence at a single temperature at each amplification cycle. Cq value is proportional to negative logarithmic value of the concentration of the target DNA, and therefore it is possible to determine the concentration from the experimentally determined Cq value. However, it is challenging to correctly quantify each target where there is more than one target-specific probe in a single channel as it is difficult to identify which probe/s are contributing to the signal. Addressing this problem, LOCS reporters may be used to enable correct and specific quantification of more than one target in a single channel by generating amplification curves obtained by measuring fluorescence at more than one temperature during amplification. This is possible because LOCS reporters may produce a significantly different amount of fluorescence at different temperatures. Furthermore, LOCS reporters can be used to enable correct and specific quantification of a first target, and simultaneous qualitative detection of a second target in a single channel, by acquiring fluorescence at a first temperature in real time (target 1), and at second temperature before and after amplification (target 2). The advantage of the latter scenario is that it does not impact the overall run-time of the amplification protocol and may not require specialised software for analysis. This approach can be useful in scenarios where quantification or Cq determination is only required for one of the targets.

In some embodiments where analysis only requires fluorescent acquisition at a limited number of time points within PCR, for example at or near the start of amplification and following amplification at the endpoint, using LOCS structures eliminates the need for acquisition at each cycle. As such, these embodiments are well suited to very rapid cycling protocols which can reduce the time to result.

As noted above, melt curve analysis protocols entail measuring fluorescence at various temperatures over an incrementally increasing temperature range (e.g. between 30° C. and 90° C.). The change in slope of this curve may then be plotted as a function of temperature to obtain the melt curve. This process is often slow and can take, for example, anywhere between 30-60 mins to complete. Increasing the speed of melt curve analysis requires access to highly specialised instrumentation and cannot be accomplished using standard PCR devices. Thus, there is a high demand for faster alternatives to melt curve analysis that can provide simultaneous detection of multiple targets in a single fluorescence channel using standard instrumentation. The melting temperature (Tm) of the LOCS structures of the present invention are pre-determined and constant for given experimental conditions (i.e. unaffected by target sequence or concentration), and therefore do not require ramping through the entire temperature gradient. Each LOCS structure only requires a single fluorescent measurement at its specific Tm, negating the need to run a full temperature gradient, facilitating a faster time to result and therefore overcoming the above limitations.

Furthermore, melt curve analysis typically requires interpretation by skilled personnel or use of specialised software for results interpretation.

In some embodiments of the present invention, the use of discrete temperature fluorescence measurements following completion of PCR can eliminate the need for subjective interpretation of melt curves and facilitate objective determination of the presence or absence of targets.

In other embodiments of the present invention, analysis may only require fluorescent acquisition at a limited number of time points within PCR, for example pre-PCR and post-PCR, which eliminates the need for acquisition at each cycle. As such, these embodiments are well suited to very rapid cycling protocols which can reduce the time to result.

Several methods have been described which involve fluorescence acquisition at multiple temperatures during PCR, including two temperature acquisition to facilitate distinction between fully matched and mismatched probes. Additionally, some protocols use multiple acquisition temperatures after each PCR cycle to quantify the concentration of each target when two targets are present and detected from a single channel. Other methods for simultaneous quantification of two targets are achieved by performing a complete melt curve at the end of each PCR cycle.

The present invention exploits the advantages of combining LOCS with other types of reporter molecules. LOCS structures may be compatible with most and potentially all existing methods of analysis of real time and endpoint PCR. Whilst it is possible to perform analysis whereby only LOCS probes are used to discriminate multiple targets in a single reaction, it may be advantageous to use multiple types of probes in a single reaction. By way of example, a single LOCS probe can be used in combination with any of the following technologies: a linear MNAzyme substrate, a linear TaqMan probe, probes cleavable with restriction enzymes, an Eclipse probe, a non-cleavable Molecular Beacon probe, a non-cleavable Sloppy Beacon, a Scorpion Uni-Probe, a Scorpion Bi-Probe, a Dual hybridisation probe pair, or probes that utilise Catcher and Pitcher technology (e.g. TOCE probes).

In various embodiments of the present invention, a single LOCS probe and a linear MNAzyme substrate, linear TaqMan probe, or non-cleavable Molecular Beacon probe may be labelled with a same or similar detection moiety. By way of non-limiting example, this could include the same fluorophore for fluorometric detection, the same size and/or type of nanoparticle (e.g. gold or silver) for colorimetric or SPR detection, a reactive moiety (e.g. alkaline phosphatase or peroxidase enzymes) for chemiluminescence detection or an electroactive species (e.g. ferrocene, methylene blue or peroxidase enzymes) for electrochemical detection.

In certain embodiments a linear MNAzyme substrate capable of being cleaved by a first target-specific MNAzyme can be combined with a single LOCS probe capable of being cleaved by a second target-specific MNAzyme. Embodiments wherein one linear MNAzyme substrate and one LOCS probe are used to detect two targets at, or example, one wavelength of the visible spectrum, may be advantageous over embodiments using two LOCS probes, since manufacture of linear probes is simpler and less expensive than manufacture of LOCS probes. This is because linear substrates do not require the additional sequence required for a LOCS probe stem region and hence are shorter. Similarly, manufacture of linear TaqMan probes may be less expensive than for LOCS probes.

Additional advantages relating to use of either a single, or multiple LOCS in combination with other types of Standard Reporters relates to the inherent difference in the background fluorescence of linear probes, in which the temporal/spatial parameters result in greater distance between the fluorophore and quencher and hence higher background fluorescence compared to LOCS probes, where the fluorophore and quencher are held in close proximity by the stem portion. Furthermore, different types of probes generate signal using different mechanisms wherein they exhibit different fluorescence and quenching properties at different temperatures. In various embodiments exemplified below, this difference in fluorescence and quenching capacity provides an additional tool with which an investigator can manipulate the magnitude of the detection signal at specific temperatures to detect, discriminate and/or quantify multiple targets at a single wavelength.

In some embodiments the present invention exploits the fact that LOCS probes and Catcher-Pitcher probes have opposing fluorescence/quenching properties at different temperatures. For example, regardless of the presence or absence of target, Catcher-Pitcher probes would remain quenched at a high temperature (i.e. above the Tm of Catcher-pitcher duplex) due to denaturation of the duplex and a change in conformation of the Catcher strand. Conversely, LOCS probes would remain quenched at a low temperature (i.e. below Tm of split LOCS stem), regardless of the presence or absence of target, because the hybridised stem keeps the fluorophore and quencher in close proximity. Furthermore, in the presence of target, Catcher-Pitcher probes would generate an increase in fluorescence at a low temperature (i.e. below Tm of Catcher-pitcher duplex) whereas LOCS probes would generate an increase in fluorescence at a high temperature (i.e. above Tm of split LOCS stem). These opposing fluorescence/quenching properties at high and low temperatures enable specific detection of two targets allowing one target to be detected at a first, low temperature using a Catcher-Pitcher probe and another target to be detected at a second, higher temperature using a LOCS probe.

In various embodiments of the present invention, the advantages of combining one linear substrate or probe, for example a linear MNAzyme substrate or a TaqMan probe, with a LOCS probe are exploited. For example, an advantage, in comparison to using a pair of LOCS probes with one lower and one higher Tm stem, is that both a cleaved linear MNAzyme substrate, and a degraded TaqMan probe, produce similar fluorescence signals across a broad range of temperatures. Similarly, uncleaved linear MNAzyme substrate, and intact TaqMan probes, produce similar fluorescence signals across a broad range of temperatures. Therefore, for both probe types the signal-to-noise ratio is constant across a wide range of detection temperatures. In comparison, the observed signal to noise ratio arising from Split low-Tm LOCS probes may decrease at higher detection temperatures due to a greater background fluorescence which is generated by denaturation of the Intact LOCS stems. This means that cleavage of a linear MNAzyme substrate, or a TaqMan probe, can be detected across a broader range of detection temperatures compared that of a low-Tm LOCS that has a more restricted detection temperature range. This allows for more flexibility in thermocycling and may be useful for faster and simplified multiplex assay development. A further advantage stems from the ability to combine one or more LOCS probes with existing commercial kits using other technologies such as TaqMan probes and thus expand their multiplexing capacity.

In other embodiments the present invention exploits the advantages conferred by the fact that LOCS probes and Scorpion Uni-Probes or Bi-Probes also behave differently at different temperatures enabling specific detection of two targets at two different detection temperatures. For example, at a high detection temperature, a Scorpion Uni-Probe can always be fluorescent (pre-PCR and post-PCR) regardless of the presence or absence of either target if the stem is open and fluorescing and the loop is unable to bind to the amplicons of the specific target (Target 1). Similarly at a high detection temperature, Scorpion Bi-Probes can always be fluorescent (pre-PCR and post-PCR) regardless of the presence or absence of either target since the complementary quencher sequence may be unable to bind to the probe, and the probe may be unable to bind to the amplicons of the specific target (Target 1). In both cases (uni-probe or bi-probe), at the same high temperature a LOCS probe would only generate fluorescence in the presence of the specific target (Target 2) due to cleavage and dissociation of the stem. Conversely, at a low detection temperature, a LOCS probe would always be quenched (pre-PCR and post-PCR) regardless of the presence or absence of either target since the Tms of the stems of both Intact LOCS and Split LOCS are above this temperature whereas, at this same temperature, a Scorpion Uni-Probe or Bi-Probe would only generate fluorescence in the presence of specific target due to hybridization of the loop or probe regions respectively to Target 1 amplicons. These opposing fluorescence/quenching properties at high and low temperatures enable specific detection of two targets, where Target 1 can be detected at a first, low temperature using either a Scorpion Uni-Probe or a Scorpion Bi-Probe and Target 2 can be detected at a second, higher temperature using a LOCS probe.

Various types of standard reporter substrates and probes will fluorescence over either a wide range of temperatures or only over a restricted range. For example, linear reporter substrates or probes, including but not limited to, linear MNAzyme substrates, Eclipse probes, TaqMan Probes, Hydrolysis probes, and others generally produce fluorescent signal across a broad range of temperatures. Such probes are generally quenched before PCR and fluoresce following PCR if target is present and this fluorescence can be measured over a broad range of temperatures. In contrast, LOCS probes, Molecular Beacons, Scorpion Uni-Probes or, Bi-Probes and Pitcher and Catcher fluorescence systems (e.g. TOCE probes) can be manipulated such that they are fluorescent or quenched within defined temperature ranges.

Molecular Beacons are quenched with the stems hybridised at temperatures which are below that where the Molecular Beacon Loop binds to the target and fluoresces. In contrast, the stem of Intact LOCS probes are hybridised at temperatures which are above that where the Split LOCS melt. Further, while many reporter systems measure an increase in fluorescence in the presence of target, other technologies such as Dual Hybridization probes lead a decrease in fluorescence when the target is present. The present invention provides new methods for combining probes and setting parameters so that increases or decreases in detectable signals at specific temperature with specific probe combinations allow for improved multiplexing scenarios. As such, exploitation of the differing behaviours of different types of Standard Reporter substrates and probes, when combined with LOCS probes labelled with the same or similar detection moiety and present within a single reaction, allows for manipulation of the presence or absence of signal, for example fluorescence or quenching, at multiple temperatures which in turn provide a multitude of advantages for analysis of targets.

The present invention relates at least in part to the following embodiments 1-194:

Embodiment 1. A method for determining the presence or absence of first and second targets in a sample, the method comprising:

(a) preparing a mixture for a reaction by contacting the sample or a derivative thereof putatively comprising the first and second targets with:
a first oligonucleotide for detection of the first target, and comprising a first detection moiety capable of generating a first detectable signal;
an intact stem-loop oligonucleotide for detection of the second target, and comprising a double-stranded stem portion of hybridised nucleotides, opposing strands of which are linked by an unbroken single-stranded loop portion of unhybridised nucleotides,
wherein the stem portion comprises a second detection moiety capable of generating a second detectable signal, wherein the first and second detection moieties are capable of generating detectable signals that cannot be differentiated at a single temperature using a single type of detector; and
a first enzyme capable of digesting one or more of the unhybridised nucleotides of the intact stem-loop oligonucleotide only when the second target is present in the sample;

(b) treating the mixture under conditions suitable for:
the first target to induce a modification to the first oligonucleotide thereby enabling the first detection moiety to generate a first detectable signal,
digestion of one or more of the unhybridised nucleotides of the intact stem-loop oligonucleotide by the first enzyme, only when the second target is present in the sample, to thereby break the single-stranded loop portion and provide a split stem-loop oligonucleotide;

(c) measuring:
a background signal provided by the first and the second detection moieties in the mixture, or, in a control mixture;

(d) determining whether at one or more timepoints during or after said treating:
a first detectable signal arising from said modification is generated at a first temperature which differs from the background signal and is indicative of the presence of the first target in the sample;
a second detectable signal is generated at a second temperature which differs from the background signal and is indicative of the presence of the second target in the sample;
wherein:
at the first temperature the second detectable signal does not differ from the background signal, and at the second temperature:
if present, strands of the double-stranded stem portion of the split stem-loop oligonucleotide are partially or completely dissociated enabling the second detection moiety to provide the second detectable signal; and
if present, strands of the double-stranded stem portion of the intact stem-loop oligonucleotide cannot dissociate thereby preventing the second detectable moiety from providing the second detectable signal.

Embodiment 2. The method of embodiment 1, wherein said determining in part (d) comprises:
using a predetermined threshold value to determine if the first detectable signal arising from said modification differs from any said background signal at the first temperature; and/or
using a predetermined threshold value to determine if the second detectable signal differs from any said background signal at the second temperature.

Embodiment 3. The method of embodiment 1 or embodiment 2, wherein the control mixture does not comprise:
the first target; or
the second target; or
the first and second targets,
   but is otherwise equivalent to the mixture.

Embodiment 4. The method of any one of embodiments 1 to 3, wherein the control mixture comprises a predetermined amount of:
the first target; or
the second target; or
the first and second targets,
   but is otherwise equivalent to the mixture.

Embodiment 5. The method of any one of embodiments 1 to 4, wherein:
the modification to the first oligonucleotide enables the first detection moiety to provide the first detectable signal at or below the first temperature; and
generation of the first detectable signal is reversible.

Embodiment 6. The method of embodiment 5, wherein:
part (c) comprises measuring:
   a first background signal at or within 1° C., 2° C., 3° C., 4° C., or 5° C. of a first temperature, and a second background signal at or within 1° C., 2° C., 3° C., 4° C., or 5° C. of a second temperature;

provided by the first and the second detection moieties in the mixture, or, in the control mixture; and
part (d) comprises determining whether at one or more timepoints during or after said treating:
   a first detectable signal arising from said modification is generated at the first temperature which differs from the first background signal and is indicative of the presence of the first target in the sample;
a second detectable signal is generated at the second temperature which differs from the second background signal and is indicative of the presence of the second target in the sample.

Embodiment 7. The method of embodiment 5 or embodiment 6, wherein:
the first target is a nucleic acid sequence;
the first oligonucleotide is a stem-loop oligonucleotide comprising a double-stranded stem portion of hybridised nucleotides on opposing strands of which are linked by an unbroken single-stranded loop portion of unhybridised nucleotides of which all or a portion is/are complementary to the first target; and
the modification of the first oligonucleotide is a conformational change arising from hybridisation of the target to the single-stranded loop portion of the first oligonucleotide by complementary base pairing.

Embodiment 8. The method of embodiment 7, wherein:
the conformational change is dissociation of strands in the double-stranded stem portion of the first oligonucleotide arising from said hybridisation of the target to the single-stranded loop portion of the first oligonucleotide by complementary base pairing.

Embodiment 9. The method of embodiment 7 or embodiment 8, wherein:
the stem portion of the first oligonucleotide has a melting temperature (Tm) that is: below the Tm of a double-stranded duplex formed from said hybridisation of the target to the single-stranded loop portion of the first oligonucleotide, and above the Tm of the stem portion of the split stem-loop oligonucleotide;
said double-stranded duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: said double-stranded duplex, the stem portion of the first oligonucleotide, the stem portion of the intact stem-loop oligonucleotide, and the stem portion of the split stem-loop oligonucleotide;
the second temperature is below the Tm of: said double-stranded duplex, the stem portion of the first oligonucleotide, and the stem portion of the intact stem-loop oligonucleotide; and is above the Tm of the stem portion of the split stem-loop oligonucleotide; and
the first temperature is below the second temperature.

Embodiment 10. The method of embodiment 7 or embodiment 8, wherein:
the stem portion of the first oligonucleotide has a melting temperature (Tm) that is: below the Tm of a double-stranded duplex formed from said hybridisation of the target to the single-stranded loop portion of the first oligonucleotide, below the Tm of the stem portion of the intact stem-loop oligonucleotide;
said double-stranded duplex has a Tm that is above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: said double-stranded duplex, the stem portion of the first oligonucleotide, the stem portion of the intact stem-loop oligonucleotide, and the stem portion of the split stem-loop oligonucleotide;
the second temperature is above the Tm of: the stem portion of the first oligonucleotide, and the stem portion of the split stem-loop oligonucleotide; and is below the Tm of: said double-stranded duplex, and the stem portion of the intact stem-loop oligonucleotide; and
the first temperature is below the second temperature.

Embodiment 11. The method of embodiment 7 or embodiment 8, wherein:
the stem portion of the first oligonucleotide has a melting temperature (Tm) that is: below the Tm of a double-stranded duplex formed from said hybridisation of the target to the single-stranded loop portion of the first oligonucleotide, below the Tm of the stem portion of the intact stem-loop oligonucleotide, and below the Tm of the stem portion of the split stem-loop oligonucleotide;
said double-stranded duplex has a Tm that is: below the Tm of the stem portion of the intact stem-loop oligonucleotide;
the stem portion of the intact stem-loop oligonucleotide has a Tm that is above the Tm of the stem portion of the split stem-loop oligonucleotide;
the first temperature is below the Tm of: said double-stranded duplex, the stem portion of the first oligonucleotide, the stem portion of the intact stem-loop oligonucleotide, and the stem portion of the split stem-loop oligonucleotide;
the second temperature is above the Tm of: said double-stranded duplex, the stem portion of the first oligonucleotide, and the stem portion of the split stem-loop oligonucleotide; and is below the Tm of: the stem portion of the intact stem-loop oligonucleotide; and
the first temperature is below the second temperature.

Embodiment 12. The method of embodiment 7 or embodiment 8, wherein:
the stem portion of the first oligonucleotide has a melting temperature (Tm) that is: below the Tm of a double-stranded duplex formed from said hybridisation of the target to the single-stranded loop portion of the first oligonucleotide, above the Tm of the stem portion of the intact stem-loop oligonucleotide, and above the Tm of the stem portion of the split stem-loop oligonucleotide;
said double-stranded duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the stem portion of the first oligonucleotide and said double-stranded duplex; and above the Tm of: the stem portion of the intact stem-loop oligonucleotide and the stem portion of the split stem-loop oligonucleotide;
the second temperature is below the Tm of: the stem portion of the first oligonucleotide, said double-stranded duplex, and the stem portion of the intact stem-loop oligonucleotide; and is above the Tm of the stem portion of the split stem-loop oligonucleotide; and
the first temperature is above the second temperature.

Embodiment 13. The method of any one of embodiments 7 to 12, wherein:
the Tm of the stem portion of the first oligonucleotide is between 1° C. and 10° C., 1° C. and 5° C., 5° C. and 10° C., or more than 10° C., below the Tm of said double-stranded duplex; and/or
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of: the stem portion of the first oligonucleotide, and/or said double-stranded duplex; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

Embodiment 14. The method of embodiment 5 or embodiment 6, wherein:
the first target is a nucleic acid sequence;
the first oligonucleotide is a stem-loop oligonucleotide comprising:
   a double-stranded stem portion of hybridised nucleotides, opposing strands of which are linked by a single-stranded loop portion of unhybridised nucleotides, all or a portion of which is/are complementary to the first target, and a second single-stranded portion extending from one of said opposing strands in a 3′ direction and terminating with a sequence that is complementary to a portion of the first target, and
a blocker molecule preceding said sequence that is complementary to the portion of the first target;
the mixture further comprises a polymerase;
said treating the mixture comprises:
   hybridising the second single-stranded portion to the first target by complementary base pairing;
   extending the second single-stranded portion using the polymerase and the first target as a template sequence to provide a double-stranded nucleic acid, wherein said blocker molecule prevents the polymerase extending the first target using the stem portion of the first oligonucleotide as a template; and
   denaturing the double-stranded nucleic acid and hybridising the second single-stranded portion extended by the polymerase to the single-stranded loop portion of the first oligonucleotide by complementary base pairing to produce a signaling duplex and thereby provide said modification to the first oligonucleotide enabling the first detection moiety to provide the first detectable signal.

Embodiment 15. The method of embodiment 14, wherein:
the stem portion of the first oligonucleotide has a melting temperature (Tm) that is: below the Tm of the signaling duplex and above the Tm of the stem portion of the split stem-loop oligonucleotide;
the signaling duplex has a Tm that is: above the Tm of the stem portion of the intact stem-loop oligonucleotide, and above the Tm of the stem portion of the split stem-loop oligo nucleotide;
the stem portion of the intact stem-loop oligonucleotide has a Tm that is above the Tm of the stem portion of the split stem-loop oligonucleotide;
the first temperature is below the Tm of: the signaling duplex, the stem portion of the first oligonucleotide, the stem portion of the intact stem-loop oligonucleotide, and the stem portion of the split stem-loop oligonucleotide;
the second temperature is below the Tm of: the signaling duplex, the stem portion of the first oligonucleotide, and the stem portion of the intact stem-loop oligonucleotide; and is above the Tm of the stem portion of the split stem-loop oligonucleotide; and
the first temperature is below the second temperature.

Embodiment 16. The method of embodiment 14, wherein:
the stem portion of the first oligonucleotide has a melting temperature (Tm) that is: below the Tm of the signaling duplex, below the Tm of the stem portion of the intact stem-loop oligonucleotide;
the signaling duplex has a Tm that is above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the signaling duplex, the stem portion of the first oligonucleotide, the stem portion of the intact stem-loop oligonucleotide, and the stem portion of the split stem-loop oligonucleotide;
the second temperature is above the Tm of: the stem portion of the first oligonucleotide, and the stem portion of the split stem-loop oligonucleotide; and is below the Tm of: the signaling duplex, and the stem portion of the intact stem-loop oligonucleotide; and
the first temperature is below the second temperature.

Embodiment 17. The method of embodiment 14, wherein:
the stem portion of the first oligonucleotide has a melting temperature (Tm) that is: below the Tm of the signaling duplex, below the Tm of the stem portion of the intact stem-loop oligonucleotide, and below the Tm of the stem portion of the split stem-loop oligonucleotide;
the signaling duplex has a Tm that is: below the Tm of the stem portion of the intact stem-loop oligonucleotide,
the stem portion of the intact stem-loop oligonucleotide has a Tm that is above the Tm of the stem portion of the split stem-loop oligonucleotide;
the first temperature is below the Tm of: the signaling duplex, the stem portion of the first oligonucleotide, the stem portion of the intact stem-loop oligonucleotide, and the stem portion of the split stem-loop oligonucleotide;
the second temperature is above the Tm of: the signaling duplex, the stem portion of the first oligonucleotide, and the stem portion of the split stem-loop oligonucleotide; and is below the Tm of: the stem portion of the intact stem-loop oligonucleotide; and
the first temperature is below the second temperature.

Embodiment 18. The method of embodiment 14, wherein:
the stem portion of the first oligonucleotide has a melting temperature (Tm) that is: below the Tm of the signaling duplex, above the Tm of the stem portion of the intact stem-loop oligonucleotide, and above the Tm of the stem portion of the split stem-loop oligonucleotide;
the signaling duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the stem portion of the first oligonucleotide and the signaling duplex; and above the Tm of: the stem portion of the intact stem-loop oligonucleotide and the stem portion of the split stem-loop oligonucleotide;
the second temperature is below the Tm of: the stem portion of the first oligonucleotide, the signaling duplex, and the stem portion of the intact stem-loop oligonucleotide; and is above the Tm of the stem portion of the split stem-loop oligonucleotide; and
the first temperature is above the second temperature.

Embodiment 19. The method of any one of embodiments 14 to 18, wherein:
the Tm of the stem portion of the first oligonucleotide is between 1° C. and 10° C., 1° C. and 5° C., 5° C. and 10° C., or more than 10° C., below the Tm of the signaling duplex; and/or
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of: the stem portion of the first oligonucleotide, and/or the signaling duplex; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

Embodiment 20. The method of any one of embodiments 5 to 19, wherein:
the first detection moiety is a fluorophore and the modification increases its distance from a quencher molecule.

Embodiment 21. The method of embodiment 20, wherein:
the first oligonucleotide comprises the quencher molecule.

Embodiment 22. The method of embodiment 21, wherein:
the fluorophore and the quencher molecule are located on opposing strands of the double-stranded stem portion of the first oligonucleotide.

Embodiment 23. The method of embodiment 5 or embodiment 6, wherein:
the first target is a nucleic acid sequence;
the first oligonucleotide comprises:
   a first double-stranded portion of hybridised nucleotides, a first strand of which extends into a single-stranded portion terminating with a complementary sequence capable of hybridising to a portion of the first target, wherein the first strand comprises a blocker molecule preceding said complementary sequence;
the mixture further comprises a polymerase;
said treating the mixture comprises:
   hybridising said complementary sequence of the single-stranded portion to a portion of the first target by complementary base pairing;
   extending the complementary sequence using the polymerase and the first target as a template sequence to provide a second double-stranded portion, wherein said blocker molecule prevents the polymerase extending the first target using the first strand of the said first double-stranded portion as a template;
   denaturing the first and second double-stranded portions; and
   hybridising the complementary sequence extended by the polymerase to the first strand of the first double-stranded portion by complementary base pairing to produce a signaling duplex and thereby provide said modification to the first oligonucleotide enabling the first detection moiety to provide the first detectable signal.

Embodiment 24. The method of embodiment 23, wherein:
the first double-stranded portion has a melting temperature (Tm) that is: below the Tm of the signaling duplex, and above the Tm of the stem portion of the split stem-loop oligonucleotide;
the signaling duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the signaling duplex, the first double-stranded portion, the stem portion of the intact stem-loop oligonucleotide, and the stem portion of the split stem-loop oligonucleotide;
the second temperature is below the Tm of: the signaling duplex, the first double-stranded portion, and the stem portion of the intact stem-loop oligonucleotide; and is above the Tm of the stem portion of the split stem-loop oligonucleotide; and
the first temperature is below the second temperature.

Embodiment 25. The method of embodiment 23, wherein:
the first double-stranded portion has a melting temperature (Tm) that is: below the Tm of the signaling duplex, below the Tm of the stem portion of the intact stem-loop oligonucleotide;
the signaling duplex has a Tm that is above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the signaling duplex, the first double-stranded portion, the stem portion of the intact stem-loop oligonucleotide, and the stem portion of the split stem-loop oligonucleotide;
the second temperature is above the Tm of: the first double-stranded portion, and the stem portion of the split stem-loop oligonucleotide; and is below the Tm of: the signaling duplex, and the stem portion of the intact stem-loop oligonucleotide; and
the first temperature is below the second temperature.

Embodiment 26. The method of embodiment 23, wherein:
the first double-stranded portion has a melting temperature (Tm) that is: below the Tm of the signaling duplex, below the Tm of the stem portion of the intact stem-loop oligonucleotide, and below the Tm of the stem portion of the split stem-loop oligonucleotide;
the signaling duplex has a Tm that is below the Tm of the stem portion of the intact stem-loop oligonucleotide;
the stem portion of the intact stem-loop oligonucleotide has a Tm that is above the Tm of the stem portion of the split stem-loop oligonucleotide;
the first temperature is below the Tm of: the signaling duplex, the first double-stranded portion, the stem portion of the intact stem-loop oligonucleotide, and the stem portion of the split stem-loop oligonucleotide;
the second temperature is above the Tm of: the signaling duplex, the first double-stranded portion, and the stem portion of the split stem-loop oligonucleotide; and is below the Tm of: the stem portion of the intact stem-loop oligonucleotide; and
the first temperature is below the second temperature.

Embodiment 27. The method of embodiment 23, wherein:
the first double-stranded portion has a melting temperature (Tm) that is: below the Tm of the signaling duplex, above the Tm of the stem portion of the intact stem-loop oligonucleotide, and above the Tm of the stem portion of the split stem-loop oligonucleotide;
the signaling duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the first double-stranded portion and the signaling duplex; and above the Tm of: the stem portion of the intact stem-loop oligonucleotide and the stem portion of the split stem-loop oligonucleotide;
the second temperature is below the Tm of: the first double-stranded portion, the signaling duplex, and the stem portion of the intact stem-loop oligonucleotide;
and is above the Tm of the stem portion of the split stem-loop oligonucleotide; and
the first temperature is above the second temperature.

Embodiment 28. The method of any one of embodiments 23 to 27, wherein:
the Tm of the first double-stranded portion is between 1° C. and 10° C., 1° C. and 5° C., 5° C. and 10° C., or more than 10° C., below the Tm of the signaling duplex; and/or
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of: the first double-stranded portion, and/or the signaling duplex; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

Embodiment 29. The method of any one of embodiments 23 to 28, wherein:
the first detection moiety is a fluorophore and the modification increases its distance from a quencher molecule.

Embodiment 30. The method of embodiment 29, wherein:
the first oligonucleotide comprises the quencher molecule.

Embodiment 31. The method of embodiment 30, wherein:
the fluorophore and the quencher molecule are located on opposing strands of the first double-stranded portion.

Embodiment 32. The method of embodiment 5 or embodiment 6, wherein:
the first target is a nucleic acid sequence;
the mixture further comprises:
   a first primer complementary to a first sequence in the first target,
   a second oligonucleotide comprising a component complementary to a second sequence in the first target that differs from the first sequence, and a tag portion that is not complementary to the first target,
   a first polymerase comprising exonuclease activity, and
   optionally a second polymerase, and
said treating the mixture comprises:
   suitable conditions to hybridise the first primer and the second oligonucleotide to the first target,
   extending the first primer using the first polymerase and the target as a template to thereby cleave off the tag portion,
   hybridising the cleaved tag portion to the first oligonucleotide by complementary base pairing,
   and extending the tag portion using the first or second polymerase and the first oligonucleotide as a template to generate a double-stranded sequence comprising the first oligonucleotide thereby providing said modification to the first oligonucleotide and enabling the first detection moiety to provide the first detectable signal.

Embodiment 33. The method of embodiment 32, wherein:
the double-stranded sequence has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the double-stranded sequence, the stem portion of the intact stem-loop oligonucleotide, and the stem portion of the split stem-loop oligonucleotide;
the second temperature is below the Tm of: the double-stranded sequence, and the stem portion of the intact stem-loop oligonucleotide; and is above the Tm of the stem portion of the split stem-loop oligonucleotide; and
the first temperature is below the second temperature.

Embodiment 34. The method of embodiment 32, wherein:
the double-stranded sequence has a Tm that is: above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the double-stranded sequence, the stem portion of the intact stem-loop oligonucleotide, and the stem portion of the split stem-loop oligonucleotide;
the second temperature is above the Tm of: and the stem portion of the split stem-loop oligonucleotide; and is below the Tm of: the double-stranded sequence, and the stem portion of the intact stem-loop oligonucleotide; and
the first temperature is below the second temperature.

Embodiment 35. The method of embodiment 32, wherein:
the double-stranded sequence has a Tm that is: below the Tm of the stem portion of the intact stem-loop oligonucleotide;
the stem portion of the intact stem-loop oligonucleotide has a Tm that is above the Tm of the stem portion of the split stem-loop oligonucleotide;
the first temperature is below the Tm of: the double-stranded sequence, the stem portion of the intact stem-loop oligonucleotide, and the stem portion of the split stem-loop oligonucleotide;
the second temperature is above the Tm of: the double-stranded sequence, and the stem portion of the split stem-loop oligonucleotide; and is below the Tm of: the stem portion of the intact stem-loop oligonucleotide; and
the first temperature is below the second temperature.

Embodiment 36. The method of embodiment 32, wherein:
the double-stranded sequence has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the double-stranded sequence; and
above the Tm of: the stem portion of the intact stem-loop oligonucleotide and the stem portion of the split stem-loop oligonucleotide;
the second temperature is below the Tm of: the double-stranded sequence and the stem portion of the intact stem-loop oligonucleotide; and is above the Tm of the stem portion of the split stem-loop oligonucleotide; and
the first temperature is above the second temperature.

Embodiment 37. The method of any one of embodiments 32 to 36, wherein:
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of the double-stranded sequence; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

Embodiment 38. The method of any one of embodiments 32 to 37, wherein:
the first oligonucleotide comprises a fluorophore and a quencher molecule, and
said extending the tag portion increases the distance between the fluorophore and the quencher molecule.

Embodiment 39. The method of embodiment 5 or embodiment 6, wherein:
the first target is a nucleic acid sequence;
the first oligonucleotide is complementary to a first portion of the target;
the mixture further comprises a further oligonucleotide complementary to a second portion the first target, wherein the first and second portions of the first target flank one another but do not overlap;
said treating the mixture comprises:
   forming a duplex structure comprising:
      (i) a first double-stranded component by hybridising the first oligonucleotide to the target by complementary base pairing, and
      (ii) a second double-stranded component by hybridising the further oligonucleotide to the target by complementary base pairing,
      thereby bringing the first and further oligonucleotides into proximity, and providing said modification to the first oligonucleotide enabling the first detection moiety to provide the first detectable signal.

Embodiment 40. The method of embodiment 39, wherein:
the duplex structure has a Tm that is below the Tm of the stem portion of the intact stem-loop oligonucleotide;
the stem portion of the intact stem-loop oligonucleotide has a Tm that is above the Tm of the stem portion of the split stem-loop oligonucleotide;
the first temperature is below the Tm of: the duplex structure, the stem portion of the intact stem-loop oligonucleotide, and the stem portion of the split stem-loop oligonucleotide;
the second temperature is above the Tm of: the duplex structure, and the stem portion of the split stem-loop oligonucleotide; and is below the Tm of: the stem portion of the intact stem-loop oligonucleotide; and
the first temperature is below the second temperature.

Embodiment 41. The method of embodiment 39 of embodiment 40, wherein:
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of the duplex structure; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

Embodiment 42. The method of any one of embodiments 39 to 41, wherein:
the first detectable moiety is a fluorophore and the further oligonucleotide comprises a quencher;
said forming of the duplex structure further brings the fluorophore and quencher into proximity; and
said detectable signal is a decrease in fluorescence provided by the first detection moiety.

Embodiment 43. The method of embodiment 5 or embodiment 6, wherein:
the first target is a nucleic acid sequence;
the first detection moiety is: a nanoparticle, a metallic nanoparticle, a noble metal nanoparticle, an alkali metal nanoparticle, a gold nanoparticle, or a silver nanoparticle; to which the first oligonucleotide is bound;
said treating the mixture comprises:
   hybridising the first target to the first oligonucleotide to thereby induce the modification to the first oligonucleotide enabling the first detection moiety to provide a first detectable signal indicative of the presence of the first target in the sample;
   wherein the first detectable signal is:
      (i) a change in refractive index,
      (ii) a change in colour; and/or
      (iii) a change in absorption spectrum,
   arising from the first detection moiety following said modification of the first oligonucleotide.

Embodiment 44. The method of embodiment 5 or embodiment 6, wherein:
the first target is a nucleic acid sequence;
the first detection moiety is an electrochemical agent to which the first oligonucleotide is bound;
said treating the mixture comprises:
   hybridising the first target to the first oligonucleotide to thereby induce or facilitate the modification to the first oligonucleotide enabling the first detection moiety to provide a first detectable signal indicative of the presence of the first target in the sample;

wherein the first detectable signal is a change in electrochemical signal arising from the first detection moiety following said modification of the first oligonucleotide.

Embodiment 45. The method of embodiment 44, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

Embodiment 46. The method of any one of embodiments 5 to 19, 23 to 28, 32 to 37, and 39 to 41 wherein:
the first detection moiety is a nanoparticle, a metallic nanoparticle, a noble metal nanoparticle, an alkali metal nanoparticle, a gold nanoparticle, or a silver nanoparticle; to which the first oligonucleotide is bound; and
the first detectable signal is:
   (i) a change in refractive index,
   (ii) a change in colour; and/or
   (iii) a change in absorption spectrum, arising from the first detection moiety following said modification of the first oligonucleotide.

Embodiment 47. The method of any one of embodiments 5 to 19, 23 to 28, 32 to 37, and 39 to 41 wherein:
the first detection moiety is an electrochemical agent to which the first oligonucleotide is bound; and
the first detectable signal is a change in electrochemical signal arising from the first detection moiety following said modification of the first oligonucleotide.

Embodiment 48. The method of embodiment 47, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

Embodiment 49. The method of any one of embodiments 43 to 48, wherein:
the second detection moiety is a nanoparticle, a metallic nanoparticle, a noble metal nanoparticle, an alkali metal nanoparticle, a gold nanoparticle, or a silver nanoparticle; to which the intact stem-loop oligonucleotide is bound; and
the second detectable signal is:
   (i) a change in refractive index,
   (ii) a change in colour; and/or
   (iii) a change in absorption spectrum,
arising from said strands of the double-stranded stem portion of the split stem-loop oligonucleotide dissociating.

Embodiment 50. The method of any one of any one of embodiments 43 to 48, wherein:
the second detection moiety is an electrochemical agent to which the intact stem-loop oligonucleotide is bound; and
the second detectable signal is a change in electrochemical signal arising from said strands of the double-stranded stem portion of the split stem-loop oligonucleotide dissociating.

Embodiment 51. The method of embodiment 50, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

Embodiment 52. The method of any one of embodiments 20 to 22, 29 to 31, 38, and 42, wherein:
the second detection moiety is a fluorophore, and
the second detectable signal provided by said strands of the double-stranded stem portion of the split stem-loop oligonucleotide dissociating increases the distance of the fluorophore from a quencher molecule.

Embodiment 53. The method of embodiment 52, wherein:
the fluorophore and quencher molecule are located on opposing strands of the double-stranded stem portion of the split stem-loop oligonucleotide.

Embodiment 54. The method of any one of embodiments 1 to 4, wherein:
generation of the first detectable signal is not reversible;
the modification to the first oligonucleotide enables the first detection moiety to provide the first detectable signal at or below the first temperature; and
the first detectable signal provided at or below the first temperature remains detectable at the second temperature.

Embodiment 55. The method of embodiment 54, wherein:
part (c) comprises measuring:
   (i) a first background signal at or within 1° C., 2° C., 3° C., 4° C., or 5° C. of a first temperature, and a second background signal at or within 1° C., 2° C., 3° C., 4° C., or 5° C. of a second temperature, and/or
   (ii) a third background signal at a third temperature;

provided by the first and the second detection moieties in the mixture, or, in a control mixture; and
part (d) comprises determining whether at one or more timepoints during or after said treating:
   (i) a first detectable signal arising from said modification is generated at the first temperature which differs from the first or third background signal, wherein:
      at the first temperature the second detectable signal does not differ from the first or third background signal, and
      detection of a difference between the first detectable signal and the first or third background signal is indicative of said modification of the first oligonucleotide and the presence of the first target in the sample; and
   (ii) a second detectable signal is generated at the second temperature which differs from the second or third background signal and is indicative of the presence of the second target in the sample.

Embodiment 56. The method of embodiment 55, wherein:
when a first target is present in the sample, said determining whether a second detectable signal is generated at the second temperature comprises compensating for the first detectable signal present when measuring the second detectable signal.

Embodiment 57. The method of embodiment 55 or embodiment 56, wherein:
the first signal that differs from the first background signal is generated,
the second signal that differs from the second background signal is generated, and
the second detectable signal differs from the second background signal to a greater extent than the first detectable signal differs from the first background signal, thereby indicating that the second target is present in the sample.

Embodiment 58. The method of embodiment 57, wherein:
the first temperature is below: the second temperature, the Tm of the double-stranded stem portion of the intact stem-loop oligonucleotide, and the Tm of the stem portion of the split stem-loop oligonucleotide.

Embodiment 59. The method of embodiment 57, wherein:
the first temperature is higher than: the second temperature, the Tm of the stem portion of the intact stem-loop oligonucleotide, and the Tm of the stem portion of the split stem-loop oligonucleotide.

Embodiment 60. The method of embodiment 55, wherein:
the first signal that differs from the third background signal is generated,
the second signal that differs from the third background signal is generated, and
the second signal differs from the third background signal to a greater extent than the first signal differs from the third background signal,
thereby indicating that the second target is present in the sample.

Embodiment 61. The method of embodiment 55, wherein:
the second temperature is higher than the first temperature,
the third temperature is lower the Tm of the double-stranded stem portion of the intact stem-loop oligonucleotide,
the first detectable signal that differs from the third background signal is generated,
the second detectable signal that differs from the third background signal is generated, and
the second detectable signal differs from the third background signal to a greater extent than the first signal differs from the third background signal, thereby indicating that the second target is present in the sample.

Embodiment 62. The method of any one of embodiments 55 to 61 wherein:
the Tm of the stem portion of the intact stem-loop oligonucleotide is above the Tm of the stem portion of the split stem-loop oligonucleotide;
the first temperature is below the second temperature, and is below the Tm of the stem portion of the split stem-loop oligonucleotide; and
the second temperature is above the Tm of the stem portion of the split stem-loop oligonucleotide and below the Tm of the stem portion of the intact stem-loop oligonucleotide.

Embodiment 63. The method of embodiment 62, wherein:
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the second temperature; and/or
the first temperature is between 1° C. and 10° C., 1° C. and 5° C., 5° C. and 10° C., or more than 10° C., below the Tm of the stem portion of the split stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide; and/or
the second temperature is between 1° C. and 10° C., 1° C. and 5° C., 5° C. and 10° C., or more than 10° C., below the Tm of the stem portion of the intact stem-loop oligonucleotide.

Embodiment 64. The method of embodiment 62 or embodiment 63, comprising:
measuring said third background signal, wherein the third temperature is below the second temperature.

Embodiment 65. The method of embodiment 64, wherein:
the third temperature is between 1° C. and 10° C., 1° C. and 5° C., 5° C. and 10° C., or more than 10° C., below the second temperature.

Embodiment 66. The method of any one of embodiments 55 to 61 wherein:
the Tm of the stem portion of the intact stem-loop oligonucleotide is above the Tm of the stem portion of the split stem-loop oligonucleotide;
the first temperature is above the second temperature, is above the Tm of the stem portion of the split stem-loop oligonucleotide, and is above the Tm of the stem portion of the intact stem-loop oligonucleotide; and
the second temperature is above the Tm of the stem portion of the split stem-loop oligonucleotide and is below the Tm of the stem portion of the intact stem-loop oligonucleotide.

Embodiment 67. The method of embodiment 66, wherein:
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., above the second temperature; and/or
the first temperature is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., above the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide; and/or
the second temperature is between 1° C. and 10° C., 1° C. and 5° C., 5° C. and 10° C., or more than 10° C., below the Tm of the stem portion of the intact stem-loop oligonucleotide.

Embodiment 68. The method of any one of embodiments 54 to 67, wherein:
the first oligonucleotide is a substrate for a multi-component nucleic acid enzyme (MNAzyme);
the mixture further comprises:
   an MNAzyme capable of cleaving the first oligonucleotide when the first target is present in the sample; and
said treating the mixture further comprises:
   binding of the MNAzyme to the first target and hybridisation of the substrate arms of the MNAzyme to the first oligonucleotide by complementary base pairing to facilitate cleavage of the first oligonucleotide thereby providing said modification to the first oligonucleotide and enabling the first detection moiety to provide the first detectable signal.

Embodiment 69. The method of embodiment 68, wherein:
the first target is a nucleic acid sequence; and
said treating the reaction mixture further comprises:

hybridising the first target to the sensor arms of the MNAzyme by complementary base pairing to thereby facilitate assembly of the MNAzyme.

Embodiment 70. The method of any one of embodiments 54 to 67, wherein:
the first oligonucleotide is a substrate for an aptazyme;
the first target is an analyte, protein, compound or molecule;
the mixture further comprises an aptazyme comprising an aptamer capable of binding to the first target; and
said treating the mixture further comprises:
   binding of the aptazyme to the first target and the first oligonucleotide to facilitate cleavage of the first oligonucleotide thereby providing said modification to the first oligonucleotide and enabling the first detection moiety to generate the first detectable signal.

Embodiment 71. The method of any one of embodiments 54 to 67, wherein:

the first target is a nucleic acid sequence;

the first oligonucleotide comprises a sequence that is complementary to the first target,
the mixture further comprises:
   a primer complementary to a portion of the first target, and
   a polymerase with exonuclease activity;
said treating the mixture comprises:
   hybridising the primer to the first target by complementary base pairing,
   hybridising the first oligonucleotide to the first target by complementary base pairing
   extending the primer using the polymerase and the first target as a template sequence to thereby digest the first oligonucleotide and provide said modification to the first oligonucleotide enabling the first detection moiety to generate the first detectable signal.

Embodiment 72. The method any one of embodiments 54 to 67, wherein:
the first target is a nucleic acid sequence;
the mixture further comprises:
   a restriction endonuclease capable of digesting a double-stranded duplex comprising the first target; and
said treating the mixture comprises:
   hybridising the first oligonucleotide to the first target by complementary base pairing to thereby form a double-stranded duplex,
   digesting the duplex using the restriction endonuclease to thereby provide said modification to the first oligonucleotide and enabling the first detection moiety to provide the first detectable signal.

Embodiment 73. The method of embodiment 72, wherein:
the restriction endonuclease is a nicking endonuclease capable of associating with and cleaving a strand of said double-stranded duplex, and said strand comprises all or a portion of the first oligonucleotide.

Embodiment 74. The method of any one of embodiments 54 to 67, wherein:
the mixture further comprises a DNAzyme or a ribozyme requiring a co-factor for catalytic activity;
said treating of the mixture comprises using conditions suitable for:
   binding of the cofactor to the DNAzyme or ribozyme to render it catalytically active,
   hybridisation of the DNAzyme or ribozyme to the first oligonucleotide by complementary base pairing, and
   catalytic activity of the DNAzyme or ribozyme to thereby digest the first oligonucleotide and thereby provide said modification to the first oligonucleotide enabling the first detection moiety to provide the first detectable signal.
wherein:
the first target is the co-factor.

Embodiment 75. The method of embodiment 74, wherein the co-factor is a metal ion, or a metal ion selected from: Mg²⁺, Mn²⁺, Ca²⁺, Pb²⁺.

Embodiment 76. The method of any one of embodiments 54 to 75, wherein:
the first detection moiety is a fluorophore and the modification to the first oligonucleotide increases the distance of the fluorophore from a quencher molecule.

Embodiment 77. The method of embodiment 76, wherein:
the first oligonucleotide comprises the quencher molecule.

Embodiment 78. The method of embodiment 76 or embodiment 77, wherein:
the second detection moiety is a fluorophore, and
the second detectable signal provided by said strands of the double-stranded stem portion of the split stem-loop oligonucleotide dissociating increases the distance of the fluorophore from a quencher molecule.

Embodiment 79. The method of embodiment 78, wherein:
the fluorophore and quencher molecule are located on opposing strands of the double-stranded stem portion of the split stem-loop oligonucleotide.

Embodiment 80. The method of any one of embodiments 54 to 79, wherein:
the first detection moiety is a nanoparticle, a metallic nanoparticle, a noble metal nanoparticle, an alkali metal nanoparticle, a gold nanoparticle, or a silver nanoparticle; to which the first oligonucleotide is bound; and
the first detectable signal is:
   (i) a change in refractive index,
   (ii) a change in colour; and/or
   (iii) a change in absorption spectrum,
arising from the first detection moiety following said modification of the first oligonucleotide.

Embodiment 81. The method of any one of embodiments 54 to 79, wherein:
the first detection moiety is an electrochemical agent to which the first oligonucleotide is bound; and
the first detectable signal is a change in electrochemical signal arising from the first detection moiety following said modification of the first oligonucleotide.

Embodiment 82. The method of embodiment 81, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

Embodiment 83. The method of any one of embodiments 80 to 82, wherein:
the second detection moiety is a nanoparticle, a metallic nanoparticle, a noble metal nanoparticle, an alkali metal nanoparticle, a gold nanoparticle, or a silver nanoparticle; to which the intact stem-loop oligonucleotide is bound; and
the second detectable signal is:
   (i) a change in refractive index,
   (ii) a change in colour; and/or
   (iii) a change in absorption spectrum, arising from said strands of the double-stranded stem portion of the split stem-loop oligonucleotide dissociating.

Embodiment 84. The method of any one of any one of embodiments 80 to 82, wherein:
the second detection moiety is an electrochemical agent to which the intact stem-loop oligonucleotide is bound; and
the second detectable signal is a change in electrochemical signal arising from said strands of the double-stranded stem portion of the split stem-loop oligonucleotide dissociating.

Embodiment 85. The method of embodiment 84, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

Embodiment 86. The method of any one of embodiments 1 to 85, wherein the intact stem-loop oligonucleotide is not hybridised to the second target during said digestion of the one or more unhybridised nucleotides of the intact stem-loop oligonucleotide by the first enzyme.

Embodiment 87. The method of any one of embodiments 1 to 86, wherein:
the first enzyme is a first MNAzyme, and
said treating the mixture comprises:
   binding of the first MNAzyme to the second target and hybridisation of substrate arms of said first MNAzyme to the loop portion of the intact stem-loop oligonucleotide, to thereby digest the one or more unhybridised nucleotides of the intact stem-loop oligonucleotide and provide the split stem-loop oligonucleotide.

Embodiment 88. The method of embodiment 87, wherein:
the second target is a nucleic acid sequence; and
said treating the mixture further comprises:
   hybridising the second target to the sensor arms of the first MNAzyme by complementary base pairing to thereby facilitate assembly of the first MNAzyme.

Embodiment 89. The method of any one of embodiments 1 to 86, wherein:
the second target is an analyte, protein, compound or molecule;
the first enzyme is an aptazyme comprising an aptamer capable of binding to the second target; and
binding of the second target to the aptamer is capable of rendering the first enzyme catalytically active.

Embodiment 90. The method of embodiment 89, wherein:
the first enzyme is any one of an: apta-DNAzyme, apta-ribozyme, apta-MNAzyme.

Embodiment 91. The method of any one of embodiments 1 to 86, wherein:
the second target is an analyte, protein, compound or molecule;
the first oligonucleotide is a substrate for an aptazyme;
the first enzyme is an aptazyme comprising an aptamer portion capable of binding to the second target, and a nucleic acid enzyme portion capable of digesting the one or more unhybridised nucleotides of the intact stem-loop oligonucleotide
said treating the mixture further comprises:
   binding the second target to the aptamer portion of the aptazyme to facilitate activation of catalytic activity of the nucleic acid enzyme portion, and hybridising the intact stem-loop oligonucleotide to the active nucleic acid enzyme portion to thereby digest the one or more unhybridised nucleotides of the intact stem-loop oligonucleotide.

Embodiment 92. The method of any one of embodiments 1 to 85, wherein:
the second target is a nucleic acid sequence; and
the first enzyme is a first restriction endonuclease, and said treating the mixture comprises:
   using conditions suitable for hybridisation of the second target to the single-stranded loop portion of the intact stem-loop oligonucleotide by complementary base pairing to form a double-stranded sequence for the first restriction endonuclease to associate with and digest the one or more unhybridised nucleotides of the single-stranded loop portion thereby forming the split stem-loop oligonucleotide.

Embodiment 93. The method of embodiment 92, wherein:
the first restriction endonuclease is a first nicking endonuclease capable of associating with and cleaving a strand of said double-stranded sequence for the first restriction endonuclease, and said strand comprises all or a portion of the single-stranded loop portion of the intact stem-loop oligonucleotide.

Embodiment 94. The method of any one of embodiments 1 to 85, wherein:
the first enzyme comprises a polymerase with exonuclease activity,
said treating the mixture comprises using conditions suitable for:
   hybridisation of the second target to the single-stranded loop portion of the intact stem-loop oligonucleotide by complementary base pairing to form a first double-stranded sequence comprising a portion of the second target,
   hybridisation of a first primer oligonucleotide to the second target to form a second double-stranded sequence located upstream relative to the first double-stranded sequence comprising the portion of the second target,
   extending the primer using the polymerase with exonuclease activity and using the second target as a template sequence,
   wherein the first polymerase comprising exonuclease activity digests the single-stranded loop portion of the first double-stranded sequence and thereby forms the split stem-loop oligonucleotide.

Embodiment 95. The method of any one of embodiments 1 to 85, wherein:
the first enzyme is an exonuclease, and
said treating the mixture comprises using conditions suitable for:
   hybridisation of the second target to the single-stranded loop portion of the intact stem-loop oligonucleotide by complementary base pairing to form a first double-stranded sequence comprising a portion of the second target,
   association of the first enzyme comprising exonuclease activity with the double-stranded sequence comprising the second target, and
   catalytic activity of the first enzyme comprising exonuclease activity allowing it to digest the single-stranded loop portion of the first double-stranded sequence comprising the second target and thereby form the split stem-loop oligonucleotide.

Embodiment 96. The method of any one of embodiments 1 to 85, wherein:
the first enzyme is a DNAzyme or a ribozyme requiring a co-factor for catalytic activity, and said treating the mixture comprises using conditions suitable for:
binding of the cofactor to the first enzyme to render it catalytically active,
hybridisation of the DNAzyme or ribozyme to the single-stranded loop portion of the intact stem-loop oligonucleotide by complementary base pairing,
catalytic activity of the DNAzyme or ribozyme to digest the one or more unhybridised nucleotides of the single-stranded loop portion of the intact stem-loop oligonucleotide and thereby form the split stem-loop oligonucleotide,
wherein:
the second target is the co-factor.

Embodiment 97. The method of embodiment 96, wherein the co-factor is a metal ion, or a metal ion selected from: Mg²⁺, Mn²⁺, Ca²⁺, Pb²⁺.

Embodiment 98. The method of any one of embodiments 1 to 97, wherein:
the first target differs from the second target; and/or
the first oligonucleotide comprises or consists of a sequence that is not within the single-stranded loop portion of the intact stem-loop oligonucleotide.

Embodiment 99. The method of any one of embodiments 1 to 98, wherein:
the first enzyme does not digest the second target.

Embodiment 100. The method of any one of embodiments 1 to 71, 74 to 91, or 94 to 99, wherein:
any said enzyme does not digest the first target and/or the second target.

Embodiment 101. The method of any one of embodiment 1 to 100, wherein:
the first temperature differs from the second temperature by more than: 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 25° C., 30° C., 35° C., 40° C., 45° C., 50° C., 55° C., or 60° C.

Embodiment 102. The method of any one of embodiments 1 to 101, wherein said determining comprises detection of the first detectable signal and/or any said background signal(s):
at one or more timepoints during said treating; or
at one or more timepoints during said treating and at one or more timepoints after said treating.

Embodiment 103. The method of any one of embodiments 1 to 101, wherein said determining comprises detection of the first detectable signal and/or any said background signal(s):
at one or more timepoints after said treating.

Embodiment 104. The method of any one of embodiments 1 to 101, wherein said determining comprises detection of the second detectable signal and/or any said background signal(s):

at one or more timepoints during said treating; or
at one or more timepoints during said treating and at one or more timepoints after said treating.

Embodiment 105. The method of any one of embodiments 1 to 101, wherein said determining comprises detection of the second detectable signal and/or any said background signal(s):
one or more timepoints after said treating.

Embodiment 106. The method of any one of embodiments 1 to 105, wherein:
said determining the presence or absence of the first and second targets comprises a melt curve analysis.

Embodiment 107. The method of embodiment 6, wherein:
said determining the presence or absence of the first and second targets comprises a melt curve analysis comprising the first and second detectable signals and the optionally the first and second background signals.

Embodiment 108. The method of embodiment 55, wherein:
said determining the presence or absence of the first and second targets comprises a melt curve analysis comprising the first and second detectable signals and the optionally the first and second background signals; or
the first and second detectable signals and optionally the third background signal.

Embodiment 109. The method of any one of embodiments 1 to 108, wherein:
the first target and/or the second target is an amplicon of a nucleic acid.

Embodiment 110. The method of any one of embodiments 1 to 109, wherein:
the first target is a nucleic acid and/or the second target is a nucleic acid, and
the mixture further comprises reagents for amplification of said first and/or second target,
said treating the mixture further comprises conditions suitable for conducting amplification of the first and/or second targets.

Embodiment 111. The method of embodiment 110, wherein:
the amplification is any one or more of polymerase chain reaction (PCR), strand displacement amplification (SDA), nicking enzyme amplification reaction (NEAR), helicase dependent amplification (HDA), Recombinase Polymerase Amplification (RPA), loop-mediated isothermal amplification (LAMP), rolling circle amplification (RCA), transcription-mediated amplification (TMA), self-sustained sequence replication (3SR), nucleic acid sequence based amplification (NASBA), Ligase Chain Reaction (LCR) or Ramification Amplification Method (RAM), and/or reverse transcription polymerase chain reaction (RT-PCR).

Embodiment 112. The method of embodiment 110 or embodiment 111, wherein said determining:
occurs prior to said amplification or within 1, 2, 3, 4, or 5 cycles of said amplification commencing; and/or
occurs after completion of said amplification.

Embodiment 113. The method of any one of embodiments 110 to 112, wherein said determining:
occurs prior to said amplification or within 1, 2, 3, 4, or 5 minutes of said amplification commencing; and/or
occurs after completion of said amplification.

Embodiment 114. The method of any one of embodiments 110 to 113, wherein said determining occurs:
at a first timepoint prior to said amplification; and
at a second timepoint after completion of said amplification.

Embodiment 115. The method of any one of embodiments 110 to 114, wherein:
the amplification method is polymerase chain reaction (PCR); and
said determining occurs at multiple cycles optionally at each cycle.

Embodiment 116. The method of embodiment 110 or embodiment 111, further comprising normalising:
the first detectable signal at the first temperature measured at a timepoint during or after said amplification using a positive control signal generated at the first temperature prior to said amplification and/or prior to said treating the reaction; and/or
the second detectable signal at the second temperature measured at a timepoint during or after said amplification using a positive control signal generated at the second temperature prior to said amplification and/or prior to said treating the reaction.

Embodiment 117. The method of embodiment 110 or embodiment 111, further comprising normalising:
the first detectable signal using a detectable signal generated by the intact stem-loop oligonucleotide at the first temperature prior to said amplification and/or prior to said treating the reaction; and/or
the second detectable signal using a detectable signal generated by the intact stem-loop oligonucleotide at an additional temperature prior to said amplification and/or prior to said treating the reaction;
wherein the additional temperature is above the Tm of the intact stem-loop oligonucleotide.

Embodiment 118. The method of any one of embodiments 1 to 117 further comprising:
generating a first target positive control signal using a known concentration of the first target and/or a known concentration of the first oligonucleotide after said modification.

Embodiment 119. The method of any one of embodiments 1 to 118:
further comprising generating a first target positive control signal by repeating the method on a separate control sample comprising said first target.

Embodiment 120. The method of embodiment 119, wherein:
the separate control sample comprising the first target comprises a known concentration of the first target.

Embodiment 121. The method of embodiment 119 or embodiment 120, wherein:
the separate control sample comprising the first target further comprises the second target.

Embodiment 122. The method of any one of embodiments 1 to 121, further comprising:
generating a second target positive control signal using a known concentration of the second target and/or a known concentration of the stem-loop oligonucleotide after said modification.

Embodiment 123. The method of any one of embodiments 1 to 122, further comprising:
generating a second target positive control signal by repeating said method on a separate control sample comprising the second target.

Embodiment 124. The method of embodiment 123, wherein:
the control sample comprising the second target comprises a known concentration of the second target.

Embodiment 125. The method of embodiment 123 or embodiment 124, wherein:
said control sample comprising the second target further comprises said first target.

Embodiment 126. The method of any one of embodiments 1 to 125, further comprising:
generating a combined positive control signal by repeating said method on a separate control sample comprising the first target and the second target.

Embodiment 127. The method of embodiment 126, wherein:
the combined control sample comprises a known concentration of the first target and/or a known concentration of the second target.

Embodiment 128. The method of any one of embodiments 116 to 127, further comprising:
normalising the first detectable signal and/or the second detectable signal using any said positive control signal.

Embodiment 129. The method of any one of embodiments 116 to 128, further comprising:
assessing levels of a negative control signal by repeating the method of any one of embodiments 1 to 115 on a separate negative control sample that does not contain: (i) said first target; or
   (ii) said second target; or
   (iii) said first target or said second target.

Embodiment 130. The method of embodiment 129, further comprising:
normalising the first detectable signal and/or the second detectable signal using said negative control signal.

Embodiment 131. The method of any one of embodiments 116 to 130, wherein:
any said control signal is a fluorescent control signal.

Embodiment 132. The method of any one of embodiments 1 to 131, further comprising comparing the first and/or second detectable signals to a threshold value wherein:
the threshold value is generated using detectable signals derived from a series of samples or derivatives thereof tested according to the method of any one of embodiments 1 to 115, and comprising any one or more of:
   (i) a no template control and the first target
   (ii) a no template control and the second target
   (iii) a no template control, the first target, and the second target to thereby determine said presence or absence of the first and second targets in the sample.

Embodiment 133. The method of embodiment 132, wherein:
the series of samples or derivatives thereof is tested using a known concentration of the first oligonucleotide and/or a known concentration of the intact stem-loop oligonucleotide.

Embodiment 134. The method of any one of embodiments 1 to 133, wherein:
the sample is a biological sample obtained from a subject.

Embodiment 135. The method of any one of embodiments 1 to 133:
wherein the method is performed in vitro.

Embodiment 136. The method of any one of embodiments 1 to 133:
wherein the method is performed ex vivo.

Embodiment 137. The method of any one of embodiments 1 to 136, wherein:
the first and second detectable moieties emit in the same colour region of the visible spectrum.

Embodiment 138. A composition comprising:
a first oligonucleotide for detection of a first target, wherein the first target is a nucleic acid and complementary to at least a portion of the first oligonucleotide, and
a first detection moiety, wherein:
   the first detection moiety is capable of generating a first detectable signal upon modification of the first oligonucleotide, and the modification is induced by hybridisation of the first target to the first oligonucleotide by complementary base pairing;
an intact stem-loop oligonucleotide for detection of the second target, and comprising a double-stranded stem portion of hybridised nucleotides opposing strands of which are linked by an unbroken single-stranded loop portion of unhybridised nucleotides, wherein at least one strand of the double-stranded stem portion comprises a second detection moiety; and
a first enzyme capable of digesting one or more of the unhybridised nucleotides of the intact stem-loop oligonucleotide only when the second target is present in the sample, to thereby break the single-stranded loop portion and provide a split stem-loop oligonucleotide;
wherein:
the second detection moiety is capable of generating a second detectable signal upon dissociation of the double-stranded stem portion of the split stem-loop oligonucleotide, and
the first and second detection moieties are capable of generating detectable signals that cannot be differentiated at a single temperature using a single type of detector.

Embodiment 139. The composition of embodiment 138, wherein:
the region of the first oligonucleotide which is complementary to the first target has a different melting temperature (Tm) to each strand of the double-stranded stem portion of the intact stem-loop oligonucleotide.

Embodiment 140. The composition of embodiment 138 or embodiment 139, wherein the first oligonucleotide differs in sequence from:
each strand of the double-stranded stem portion of the intact stem-loop oligonucleotide; and
the single-stranded loop portion of the intact stem-loop oligonucleotide.

Embodiment 141. The composition of any one of embodiments 138 to 140, wherein:
the first oligonucleotide is a stem-loop oligonucleotide comprising a double-stranded stem portion of hybridised nucleotides on opposing strands of which are linked by an unbroken single-stranded loop portion of unhybridised nucleotides of which all or a portion, is/are complementary to the first target.

Embodiment 142. The composition of embodiment 141, wherein:
the first target is hybridised to the first oligonucleotide by complementary base pairing causing dissociation of strands in the double-stranded stem portion of the first oligonucleotide thereby enabling the first detection moiety to provide the first detectable signal.

Embodiment 143. The composition of any one of embodiments 138 to 140, wherein:
the first oligonucleotide is a stem-loop oligonucleotide comprising:
   a double-stranded stem portion of hybridised nucleotides, opposing strands of which are linked by a single-stranded loop portion of unhybridised nucleotides, all or a portion of which is/are complementary to the first target, and
   a second single-stranded portion extending from one of said opposing strands in a 3′ direction and terminating with a sequence that is complementary to a portion of the first target, and
   a blocker molecule preceding said sequence that is complementary to the portion of the first target.

Embodiment 144. The composition of embodiment 143, wherein:
the first target is hybridised to the second single-stranded portion thereof by complementary base pairing;
   the composition further comprises a polymerase capable of extending the second single-stranded portion using the first target as a template sequence to provide a double-stranded nucleic acid, wherein said blocker molecule is capable of preventing the polymerase extending the first target using said one opposing strand as a template, and
   upon denaturing the double-stranded nucleic acid, the second single-stranded portion extended by the polymerase is capable of hybridising to the single-stranded loop portion of the first oligonucleotide by complementary base pairing to produce a signaling duplex and thereby enable the first detection moiety to provide a first detectable signal.

Embodiment 145. The composition of any one of embodiments 141 to 144, wherein:
the first detection moiety is a fluorophore.

Embodiment 146. The composition of embodiment 145, wherein:
the first oligonucleotide comprises a quencher molecule, and the fluorophore and the quencher molecule are located on opposing strands of the double-stranded stem portion of the first oligonucleotide.

Embodiment 147. The composition of any one of embodiments 138 to 140, wherein:
the first oligonucleotide comprises:
   a first double-stranded portion of hybridised nucleotides, a first strand of which extends into a single-stranded portion terminating with a complementary sequence capable of hybridising to a portion of the first target, wherein the first strand comprises a blocker molecule preceding said complementary sequence.
the composition further comprises a polymerase.

Embodiment 148. The composition of embodiment 147, wherein:
a portion of the first target is hybridised to said complementary sequence of the single-stranded portion by complementary base pairing; and
the composition further comprises a polymerase capable of extending the complementary sequence using the first target as a template sequence to provide a second double-stranded portion, wherein said blocker molecule prevents the polymerase extending the first target using the single-stranded portion as a template; and
when the first and second double-stranded portions are denatured, the complementary sequence extended by the polymerase is capable of hybridising to the first strand of the first double-stranded portion by complementary base pairing to produce a signaling duplex and thereby enable the first detection moiety to provide the first detectable signal.

Embodiment 149. The composition of embodiment 147 or embodiment 148, wherein:
the first detection moiety is a fluorophore and the modification increases its distance from a quencher molecule;

Embodiment 150. The composition of embodiment 149, wherein:
the first oligonucleotide comprises a quencher molecule, and the fluorophore and the quencher molecule are located on opposing strands of the first double-stranded portion.

Embodiment 151. The composition of any one of embodiments 138 to 140, wherein:
the first oligonucleotide is complementary to a first portion of the target;
the composition further comprises an additional oligonucleotide complementary to a second portion the first target, wherein the first and second portions of the first target flank one another but do not overlap, and are each capable of hybridising to the first target to form a duplex structure comprising:
   (i) a first double-stranded component by hybridising the first oligonucleotide to the target or by complementary base pairing, and
   (ii) a second double-stranded component by hybridising the additional oligonucleotide to the target by complementary base pairing, thereby bringing the first and additional oligonucleotides into proximity, and enabling the first detection moiety to provide the first detectable signal.

Embodiment 152. The composition of embodiment 151, wherein:
the first detectable moiety is a fluorophore and the additional oligonucleotide comprises a quencher;
said forming of the duplex structure further brings the fluorophore and quencher into proximity; and
said detectable signal is a decrease in fluorescence provided by the first detection moiety.

Embodiment 153. The method of any one of embodiments 138 to 140, wherein:
the first oligonucleotide is hybridised to the first target by complementary base pairing,
the composition further comprises:
   a primer hybridised to a portion of the first target by complementary base pairing, and
   a polymerase with exonuclease activity capable of extending the primer using the first target as a template sequence to thereby digest the first oligonucleotide and modify the first oligonucleotide enabling the first detection moiety to provide the first detectable signal.

Embodiment 154. The composition of any one of embodiments 138 to 140, wherein:
the first target is hybridised to the first oligonucleotide by complementary base pairing to thereby form a double-stranded duplex,
the composition further comprises a restriction endonuclease capable of digesting a double-stranded duplex comprising the first target thereby modifying the first oligonucleotide and enable the first detection moiety to provide the first detectable signal.

Embodiment 155. The composition of embodiment 154, wherein:
the restriction endonuclease is a nicking endonuclease capable of associating with and cleaving a strand of said double-stranded duplex, and said strand comprises the first oligonucleotide.

Embodiment 156. The composition of any one of embodiments 153 to 155, wherein:
the first detection moiety is a fluorophore and said modifying of the first oligonucleotide increases the distance of the fluorophore from a quencher molecule.

Embodiment 157. The composition of embodiment 156, wherein:
the first oligonucleotide comprises the quencher molecule.

Embodiment 158. A composition comprising:
a first oligonucleotide for detection of a first target comprising a first detection moiety, wherein:
   the first detection moiety is capable of generating a first detectable signal upon modification of the first oligonucleotide, and
   the modification is induced by the first target;
an intact stem-loop oligonucleotide for detection of the second target, and comprising a double-stranded stem portion of hybridised nucleotides opposing strands of which are linked by an unbroken single-stranded loop portion of unhybridised nucleotides, wherein at least one strand of the double-stranded stem portion comprises a second detection moiety; and
a first enzyme capable of digesting one or more of the unhybridised nucleotides of the intact stem-loop oligonucleotide only when the second target is present in the sample, to thereby break the single-stranded loop portion and provide a split stem-loop oligonucleotide;
wherein:
the second detection moiety is capable of generating a second detectable signal upon dissociation of the double-stranded stem portion of the split stem-loop oligonucleotide, and
the first and second detection moieties are capable of generating detectable signals that cannot be differentiated at a single temperature using a single type of detector.

Embodiment 159. The composition of embodiment 158, wherein the first oligonucleotide differs in sequence from:

each strand of the double-stranded stem portion of the intact stem-loop oligonucleotide; and

the single-stranded loop portion of the intact stem-loop oligonucleotide.

Embodiment 160. The composition of embodiment 158 or embodiment 159 wherein:
the first target is a nucleic acid sequence;
the composition further comprises:
   a first primer complementary to a first sequence in the first target,
   a second oligonucleotide comprising a component complementary to a second sequence in the first target that differs from the first sequence, and a tag portion that is not complementary to the first target,
   a first polymerase comprising exonuclease activity, and
   optionally a second polymerase.

Embodiment 161. The composition of embodiment 160, wherein:
the first primer and the second oligonucleotide are each hybridised to the first target by complementary base pairing,
   the first polymerase is capable of extending the first primer using the target as a template to thereby cleave off the tag portion, allowing the cleaved tag portion to hybridise to the first oligonucleotide by complementary base pairing, and
   the first polymerase or the optional second polymerase is/are capable of extending the tag portion using the first oligonucleotide as a template to generate a double-stranded sequence comprising the first oligonucleotide thereby modify the first oligonucleotide and enabling the first detection moiety to provide the first detectable signal.

Embodiment 162. The composition of embodiment 160 or embodiment 161, wherein:
the first oligonucleotide comprises a fluorophore and a quencher molecule.

Embodiment 163. The composition of embodiment 162, wherein:
the first oligonucleotide comprises a fluorophore and a quencher molecule, and
said extending the tag portion increases the distance between the fluorophore and the quencher molecule.

Embodiment 164. The composition of embodiment 158 or embodiment 159, wherein:
the first target is a co-factor for enzyme catalytic activity;
the composition further comprises a DNAzyme or a ribozyme requiring the co-factor for catalytic activity, and
DNAzyme or ribozyme is capable of binding to the first target and hybridising to the first oligonucleotide by complementary base pairing, thereby digesting and modifying the first oligonucleotide enabling the first detection moiety to generate the first detectable signal.

Embodiment 165. The composition of embodiment 164, wherein the co-factor is a metal ion, or a metal ion selected from: Mg²⁺, Mn²⁺, Ca²⁺, Pb²⁺.

Embodiment 166. The method of embodiment 158 or embodiment 159, wherein:
the first oligonucleotide is a substrate for a multi-component nucleic acid enzyme (MNAzyme);
the composition further comprises an MNAzyme capable of cleaving the first oligonucleotide when the first target is present in the sample; and
wherein the MNAzyme is capable of binding to the first target and hybridising to the first oligonucleotide by complementary base pairing via its substrate arms, and said hybridisation facilitates cleavage of the first oligonucleotide thereby modifying it and enabling the first detection moiety to provide the first detectable signal.

Embodiment 167. The composition of embodiment 166, wherein:
the first target is a nucleic acid sequence; and
the first target is hybridised to the sensor arms of the MNAzyme by complementary base pairing to thereby facilitate assembly of the MNAzyme.

Embodiment 168. The composition of embodiment 158 or embodiment 159, wherein:
the first target is an analyte, protein, compound or molecule;
the first oligonucleotide is a substrate for an aptazyme; and
the composition further comprises an aptazyme comprising an aptamer portion capable of binding to the first target, and a nucleic acid enzyme portion capable of digesting the first oligonucleotide and thereby modifying it enabling the first detection moiety to provide the first detectable signal.

Embodiment 169. The composition of embodiment 168, wherein:
the first target is bound to the aptamer portion of the aptazyme and the first oligonucleotide is hybridised to the active nucleic acid enzyme portion by complementary base pairing facilitating digestion of the first oligonucleotide and thereby modifying it enabling the first detection moiety to provide the first detectable signal.

Embodiment 170. The composition of any one of embodiments 166 to 169, wherein:
the first detection moiety is a fluorophore and said modifying the first oligonucleotide increases the distance of the fluorophore from a quencher molecule.

Embodiment 171. The composition of embodiment 170, wherein:
the first oligonucleotide comprises the quencher molecule.

Embodiment 172. The composition of any one of embodiments 138 to 144, 147, 148, 151, 153 to 155, 158 to 161, and 164 to 169, wherein:
the first detection moiety is: a nanoparticle, a metallic nanoparticle, a noble metal nanoparticle, an alkali metal nanoparticle, a gold nanoparticle, or a silver nanoparticle; to which the first oligonucleotide is bound; and
the first detectable signal is:
   (i) a change in refractive index,
   (ii) a change in colour; and/or
   (iii) a change in absorption spectrum, arising from the first detection moiety following said modification of the first oligonucleotide.

Embodiment 173. The composition of embodiment 172, wherein:
the first detection moiety is an electrochemical agent to which the first oligonucleotide is bound;
the first detectable signal is a change in electrochemical signal.

Embodiment 174. The composition of embodiment 173, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

Embodiment 175. The composition of any one of embodiments 172 to 174, wherein:
the second detection moiety is: a nanoparticle, a metallic nanoparticle, a noble metal nanoparticle, an alkali metal nanoparticle, a gold nanoparticle, or a silver nanoparticle; to which at least one strand of the double-stranded stem portion of the second oligonucleotide is bound and
the second detectable signal is:
   (i) a change in refractive index,
   (ii) a change in colour; and/or
   (iii) a change in absorption spectrum, arising upon said dissociation of the double-stranded stem portion of the split stem-loop oligonucleotide.

Embodiment 176. The composition any one of embodiments 172 to 174, wherein:
the second detection moiety is an electrochemical agent to which the second oligonucleotide is bound; and
the second detectable signal is a change in electrochemical signal arising upon said dissociation of the double-stranded stem portion of the split stem-loop oligonucleotide.

Embodiment 177. The composition of embodiment 176, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

Embodiment 178. The composition of any one of embodiments 145, 146, 149, 150, 152, 156, 157, 162, 163, 170, and 171 wherein:
the second detection moiety is a fluorophore, and
the second detectable signal provided by said second detection moiety upon dissociation of the double-stranded stem portion of the split stem-loop oligonucleotide increases the distance of the fluorophore from a quencher molecule.

Embodiment 179. The composition of embodiment 178, wherein:
the fluorophore and quencher molecule are located on opposing strands of the double-stranded stem portion of the stem-loop oligonucleotide.

Embodiment 180. The composition of any one of embodiments 138 to 179, wherein:
the first enzyme is a first MNAzyme,
the first MNAzyme is bound to the second target,
the substrate arms of said first MNAzyme are hybridised by complementary base pairing to the single loop portion of the intact stem-loop oligonucleotide, thereby facilitating digestion of the one or more unhybridised nucleotides of the intact stem-loop oligonucleotide and providing the split stem-loop oligonucleotide.

Embodiment 181. The composition of embodiment 180, wherein:
the second target or is a nucleic acid sequence; and
the second target is hybridised to the sensor arms of the first MNAzyme by complementary base pairing to thereby facilitate assembly of the first MNAzyme.

Embodiment 182. The composition of any one of any one of embodiments 138 to 179, wherein:
the second target is an analyte, protein, compound or molecule;
the first enzyme is an aptazyme comprising an aptamer capable of binding to the second target; and
the aptamer is bound to the second target thereby rendering the first enzyme catalytically active.

Embodiment 183. The composition of embodiment 182, wherein:
the first enzyme is any one of an: apta-DNAzyme, apta-ribozyme, apta-MNAzyme.

Embodiment 184. The composition of any one of embodiments 138 to 179, wherein:
the second target is an analyte, protein, compound or molecule;
the single-stranded loop portion of the intact stem-loop oligonucleotide is a substrate for an aptazyme; and
the composition further comprises an aptazyme comprising an aptamer portion capable of binding to the second target, and a nucleic acid enzyme portion capable of digesting the one or more unhybridised nucleotides of the intact stem-loop oligonucleotide to thereby form the split stem-loop oligonucleotide.

Embodiment 185. The composition of embodiment 184, wherein:
the second target is bound to the aptamer portion of the aptazyme and the single-stranded loop portion of the intact stem-loop oligonucleotide is hybridised to the active nucleic acid enzyme portion by complementary base pairing, facilitating digestion of the one or more unhybridised nucleotides of the intact stem-loop oligonucleotide to thereby form the split stem-loop oligonucleotide.

Embodiment 186. The composition of any one of embodiments 138 to 179, wherein:
the second target is a nucleic acid sequence; and
the first enzyme is a first restriction endonuclease, and
the second target is hybridised to the single-stranded loop portion of the intact stem-loop oligonucleotide by complementary base pairing to form a double-stranded sequence for the first restriction endonuclease to associate with and digest the one or more unhybridised nucleotides of the intact stem-loop oligonucleotide to thereby form the split stem-loop oligonucleotide.

Embodiment 187. The composition of embodiment 186, wherein:
the first restriction endonuclease is a first nicking endonuclease capable of associating with and cleaving a strand of said double-stranded sequence for the first restriction endonuclease, and said strand comprises the intact stem-loop oligonucleotide.

Embodiment 188. The composition of any one of embodiments 138 to 179, wherein:
the first enzyme comprises a polymerase with exonuclease activity,
the second target is hybridised to the single-stranded loop portion of the intact stem-loop oligonucleotide by complementary base pairing to form a first double-stranded sequence comprising a portion of the second target,
the composition further comprises a first primer oligonucleotide hybridised by complementary base pairing to the second target to form a second double-stranded sequence located upstream relative to the first double-stranded sequence comprising the portion of the second target, and
the primer can be extended using the polymerase with exonuclease activity and the second target as a template sequence, digesting the single-stranded loop portion of the first double stranded sequence and thereby forming a split stem-loop oligonucleotide.

Embodiment 189. The composition of any one of embodiments 138 to 179, wherein:
the first enzyme is an exonuclease, and
the second target is hybridised by complementary base pairing to the single-stranded loop portion of the intact stem-loop oligonucleotide forming a first double-stranded sequence comprising a portion of the second target, to which the first enzyme comprising exonuclease activity can associate and thereby digest the single-stranded loop portion of the first double stranded sequence comprising the second target to form the split stem-loop oligonucleotide.

Embodiment 190. The composition of any one of embodiments 138 to 179, wherein:
the first enzyme is a DNAzyme or a ribozyme requiring a co-factor for catalytic activity, and
the second target is the co-factor and is bound to the DNAzyme or ribozyme,
the DNAzyme or ribozyme is hybridised to the single-stranded loop portion of the intact stem-loop oligonucleotide by complementary base pairing, allowing it to digest the one or more unhybridised nucleotides of the single-stranded loop portion of the intact stem-loop oligonucleotide and thereby form the split stem-loop oligonucleotide.

Embodiment 191. The composition of embodiment 190, wherein the co-factor is a metal ion, or a metal ion selected from: Mg²⁺, Mn²⁺, Ca²⁺, Pb²⁺.

Embodiment 192. The composition of any one of embodiments 138 to 150, 153, 156 to 158,166 or 167, wherein:
the first oligonucleotide is selected from any one or more of: a Molecular Beacon®, a Scorpions® primer, a TaqMan® primer, or an MNAzyme substrate.

Embodiment 193. The composition of any one of embodiments 138 to 192 wherein:
the first target and/or the second target is an amplicon of a nucleic acid.

Embodiment 194. A method for determining the presence or absence of first and second targets in a sample, the method comprising:

(a) preparing a mixture for a reaction by contacting the sample or a derivative thereof putatively comprising the first and second targets or amplicons thereof with:
a first oligonucleotide for detection of the first target or amplicon thereof, and comprising a first detection moiety capable of generating a first detectable signal;
an intact stem-loop oligonucleotide for detection of the second target or amplicon thereof, and comprising a double-stranded stem portion of hybridised nucleotides, opposing strands of which are linked by an unbroken single-stranded loop portion of unhybridised nucleotides, wherein the stem portion comprises a second detection moiety capable of generating a second detectable signal,
wherein the first and second detectable signals cannot be differentiated at a single temperature using a single type of detector; and
a first enzyme capable of digesting one or more of the unhybridised nucleotides of the intact stem-loop oligonucleotide only when the second target or amplicon thereof is present in the sample;
(b) treating the mixture under conditions suitable for:
   the first target or amplicon thereof to induce a modification to the first oligonucleotide thereby enabling the first detection moiety to provide a first detectable signal,
   digestion of one or more of the unhybridised nucleotides of the intact stem-loop oligonucleotide by the first enzyme, only when the second target or amplicon thereof is present in the sample, to thereby break the single-stranded loop portion and provide a split stem-loop oligonucleotide;
(c) measuring:
   a first background signal at or within 5° C. of a first temperature, and a second background signal at or within 5° C. of a second temperature, or
   a third background signal at a third temperature;
provided by the first and the second detection moieties in the mixture, or, in a control mixture;
(d) determining at one or more timepoints during or after said treating:
   if a first detectable signal is generated at the first temperature that differs from the first or third background signal, wherein:
      the second detectable signal generated at the first temperature does not differ from the first or third background signal, and
      detection of a difference between the first detectable signal and the first or third background signal is indicative of said modification of the first oligonucleotide and the presence of the first target or amplicon thereof in the sample; and
   if a second detectable signal is generated at the second temperature that differs from the second or third background signal, wherein at the second temperature:
      strands of the double-stranded stem portion of the split stem-loop oligonucleotide dissociate enabling the second detection moiety to provide a second detectable signal indicative of the presence of the second target or amplicon thereof in the sample; and
      strands of the double-stranded stem portion of the intact stem-loop oligonucleotide cannot dissociate thereby ensuring suppression of the second detectable moiety and an absence of the second detectable signal indicative of the absence of the second target in the sample.

### BRIEF DESCRIPTION OF THE DRAWINGS

Preferred embodiments of the present invention will now be described, by way of example only, with reference to the accompanying Figures as set out below.

FIG. 1 An Exemplary LOCS reporter and its melting temperatures (Tm) in the Intact and Split conformations are illustrated. A LOCS reporter as exemplified can be used in combination with various standard reporter probes and substrates well known in the art for detection of nucleic acids. Exemplary Intact LOCS reporters (FIG. 1A, LHS; top and bottom) have a Loop region which can be cleaved or degraded, a Stem region and detection moiety, for example a fluorophore (F) quencher (Q) dye pair. Cleavage or degradation of the Loop region in the presence of target can produce Split LOCS reporter structures (FIG. 1B RHS; top and bottom). The melting temperatures of the stem regions of the Intact LOCS (Tm A) is higher than the Tm of the stem regions in Split LOCS (Tm B). As such the Stem of the Intact LOCS will melt and separate at temperatures at or above Tm A. In contrast, the stem holding the two fragments of the Split LOCS will melt and separate at temperatures at or above Tm B resulting in increased fluorescence.

FIG. 2 illustrates an exemplary strategy for detection of a target using LOCS oligonucleotides which are universal and can be used to detect any target. In this scheme the LOCS oligonucleotide contains a stem region, a fluorophore quencher dye pair and a Loop region. The loop region comprises a universal substrate for a catalytic nucleic acid such as an MNAzyme, also known in the art as a PlexZyme. MNAzymes form when target sensor arms of component partzymes align adjacently on a target. The Loop region of the LOCS oligonucleotide binds to the substrate binding arms of the assembled MNAzyme and the substrate within the LOCS Loop is cleaved by the MNAzyme to generate a Split LOCS structure. Both the Intact LOCS and the Split LOCS will be either quenched, or will generate fluorescence, depending upon whether the temperature of the reaction milieu is above or below the melting temperature of their stems, namely Tm A and Tm B respectively. The presence of fluorescence at temperatures between Tm B and Tm A is indicative of the presence of the target which facilitates the cleavage. The target can be directly detected, or target amplicons produced by target amplification protocols, can be detected.

FIG. 3 illustrates an exemplary strategy for a preferred embodiment of the present invention where a Linear MNAzyme substrate is used in conjunction with a single LOCS probe comprising an MNAzyme substrate within its Loop. The Linear MNAzyme substrate and the single LOCS probe are both labelled with the same detection moieties, for example a specific fluorophore (F)/quencher (Q) dye pair. The linear substrate contains a first substrate sequence which is cleavable by a first MNAzyme which assembles in the presence of a first target 1 (FIG. 3A). In the presence of target 1 the linear substrate is cleaved, resulting in an increase in fluorescence which can be detected at all temperatures. The LOCS probe contains a second substrate sequence within its Loop which is cleavable by a second MNAzyme which assembles in the presence of a second target 2 (FIG. 3B). In the presence of target 2 the LOCS substrate is cleaved to generate a Split LOCS which melts at Tm B which is lower than the melting temperature of the Intact LOCS (Tm A). At temperatures below the Tm B, the stem portions of the Split LOCS remain hybridized (closed) and hence remain quenched. At temperatures above Tm B, the stem portions of the Split LOCS dissociate (separate) and fluorescence increases. When both targets are present, and fluorescence is measured at temperatures below Tm B, the increase in fluorescence is associated with target 1 only; when fluorescence is measured at temperatures above Tm B, but below Tm A, the increase in fluorescence may be associated with targets 1 and/or target 2.

FIG. 4 illustrates exemplary strategies for detection of a target using LOCS oligonucleotides which are specific for a target, which can be used in combination with other types of reporter probes or substrates such standard TaqMan, Molecular Beacons, Scorpion Uni-Probe, Scorpion Bi-Probes or linear MNAzyme substrate probes. The Intact LOCS oligonucleotides may contain a stem region, a fluorophore quencher dye pair and a Loop region which comprises a region complementary to the target amplicon. In the scheme illustrated in FIG. 4A, the Loop region of the LOCS oligonucleotides is complementary to and binds to target amplicons during amplification. During extension of primers, the exonuclease activity of the polymerase degrades the Loop region but leaves the stem region intact. The stem regions are complementary to each other but not to their target. Degradation generates a Split LOCS, wherein the stem remains hybridized and quenched at temperature below the Tm of the stem. When the temperature is raised to above the Tm of the stem, the strands separate, and fluorescence may be emitted. In the scheme illustrated in FIG. 4B, the Loop region of the LOCS oligonucleotide comprises a region complementary to the target amplicon and further contains a recognition site for a restriction enzyme, for example a nicking enzyme. The Loop region of the LOCS oligonucleotide binds to the target and the nicking enzyme cleaves the Loop region, leaving the target molecule intact. This splits the LOCS and fluorescent signal is emitted at temperature above the melting temperature of the stem. At lower temperatures the stem regions of the Split LOCS structure can anneal and quench fluorescence. The strategy may be used to directly detect target sequences or may detect target amplicons when combined with a target amplification method.

FIG. 5 illustrates embodiments wherein a non-cleavable Molecular Beacon may be combined with a LOCS probe which is cleavable by an MNAzyme. Both the Molecular Beacon and the LOCS probe may be labelled with the same fluorophore. The Molecular Beacon may have a stem region with a Tm A and a Loop region which can specifically hybridize with a first target 1 with a Tm B; where Tm B is greater than Tm A. This may be combined with an Intact LOCS probe which may have a stem region with a Tm C and a Loop region which can be cleaved by an MNAzyme in the presence a second target 2 thus generating a Split LOCS with a Tm D; where Tm D is less than Tm C. The presence of target 1 and/or target 2 can be discriminated by measuring the fluorescence at two temperatures either in real time; or using discrete measurements acquired at, or near, the beginning of amplification and following amplification.

FIG. 6 illustrates exemplary PCR amplification curves for quantitative analysis of a first target 1 (CTcry) at a first temperature in the presence or absence of varying concentrations of a second target 2 (NGopa). The protocol combined one linear MNAzyme substrate (for target 1) with one LOCS reporter (for target 2). Results were obtained in the HEX channel for quantitative detection of CTcry (target 1) at the acquisition temperature of 52° C. for reactions containing 20,000 (black dot), 4,000 (black dash), 800 (black square), 160 (grey solid) or 32 (grey dot) copies of CTcry template either alone (FIG. 6A) or in a background of either 20,000 (FIG. 6B) or 32 (FIG. 6C) copies of NGopa (target 2). Fluorescent data at 52° C. was also collected for reactions lacking CTcry but containing either 20,000 (black line) or 32 copies (grey line) of NGopa template (FIG. 6D). The no target controls (nuclease free H₂O) are shown in FIG. 6A-6C (black solid line). The amplification curves are the averages of the fluorescence level from triplicate reactions.

FIG. 7 illustrates simultaneous qualitative detection of a target 1 (CTcry) and/or a target 2 (NGopa) at two temperatures (D₁ and D₂) using endpoint analysis method 1 in the HEX channel. Results presented are the averages from triplicate reactions and the errors bars represent the standard deviation between these replicates. Specifically, the data shows the change in fluorescent signal wherein one linear substrate and one LOCS probe allow detection and discrimination of CTcry (CT copy number 20K, 4, 800, 160, 32 or 0) and/or NGopa (NG copy number 20K, 32 or 0) targets respectively which were present in a background of human genomic DNA. Further, the presence of the human TFRC gene in genomic DNA was measured in the Texas Red Channel for use as a calibrator. FIG. 7A shows the change in signal (ΔS; where ΔS=S_{D-post-PCR}−S_{D-pre-PCR}). The ΔS at temperature 1 (ΔS_{D1}; 52° C.) is shown as black and white pattern and the ΔS at temperature 2 (ΔS_{D2}; 70° C.) is shown in grey. The results show that signal at temperature 1 (ΔS_{D1}; black bars) crosses threshold 1 (X₁) when CTcry is present within the sample, but does not cross this threshold when only NGopa is present. Therefore, an ΔS_{D1} greater than Threshold 1 at Temperature 1 is indicative of the presence of a first target, (CTcry). The results in FIG. 7A also show that when the change in signal at temperature 2 (ΔS_{D2}; grey bars) is greater than that at Temperature 1 (ΔS_{D2}>ΔS_{D1}), and greater than Threshold X₁ (ΔS_{D2}>X₁), then NGopa is present within the sample. Results in FIGS. 7B and 7C show use of calibrator signal for calibrating ΔS_{D1} and ΔS_{D2}. FIG. 7B illustrates the change in TFRC calibrator signal (ΔC) measured in the Texas Red channel, wherein the values exceeding threshold C indicates a positive signal for ΔC while the values below threshold C indicates a negative signal for ΔC (NTC). The calibrator template (TFRC) was present in the human genomic DNA in the background of all samples, excluding the no target control (NF H₂O). FIG. 7C shows the change in signal at each temperature calibrated against ΔC (ΔS/AC). The change in signal at temperature 1 (ΔS_{D1}/AC; 52° C.) is shown as black and white pattern and the change in signal at temperature 2 (ΔS_{D2}/AC; 70° C.) is shown in grey where results were obtained for reactions positive for ΔC in FIG. 7B, but not for those negative for ΔC (denoted as Not Applicable (N/A)). Further, the data in FIG. 7C shows that the calibration of the signals does not alter the results obtained using Endpoint Analysis Method 1 (FIG. 7A) since the pattern is consistent.

FIG. 8 illustrates simultaneous qualitative detection of a first target (CTcry) and/or a second target (NGopa) at two temperatures using analysis method 2. CTcry (CT copy number 20K, 4, 800, 160, 32 or 0) and/or NGopa (NG copy number 20K, 32 or 0) targets were present in a background of human genomic DNA. The data shows the difference in the change in fluorescent signal obtained in the HEX channel at Temperature 2 (D₂; 70° C.) and Temperature 1 (D₁; 52° C.) using analysis method 2 (ΔS_{D2} minus ΔS_{D1}=ΔΔS_{D2}ΔS_{D1}). The results show that the ΔΔS_{D2}ΔS_{D1} crosses Threshold 2 (X₂) when NGopa is present within the sample, but does not cross this threshold when CTcry only is present, and/or when NGopa is absent (NTC) from the sample. Therefore, a difference in changes in fluorescent signal greater than Threshold 2 (ΔΔS_{D2}ΔS_{D1}>X₂) is indicative of the presence of NGopa.

FIG. 9 illustrates the change in fluorescent signal (ΔS) obtained during PCR in the HEX channel at two different temperatures (52° C. and 70° C.) in presence of a first target (CTcry) and/or a second target (NGopa) using endpoint analysis method 3. CTcry (CT copy number 20K, 4, 800, 160, 32 or 0) and/or NGopa (NG copy number 20K, 32 or 0) targets were present in a background of human genomic DNA. In FIG. 9A shows ΔS at temperature 2 (ΔS_{D2}). When ΔS_{D2} is larger than Threshold X₁, this indicates CTcry and/or NGopa is present in the sample. When ΔS_{D2} is lower than Threshold X₁, this indicates neither CTcry nor NGopa are present in the reaction. FIG. 9B shows the ratio ΔS_{D1}: ΔS_{D2} which is used to indicate which targets are present in the reaction. When the ratio is higher than Threshold R₁, this indicates CTcry is present but not NGopa. When the ratio is lower than Threshold R₂, it indicates NGopa is present but not CTcry. When the ratio is between Thresholds R₁ and R₂, it indicates both CTcry and NGopa are present. When neither CTcry or NGopa are present in the reaction (FIG. 9A), the need for calculation of the ratio is negated and indicated as N/A, as shown in FIG. 9B.

FIG. 10 illustrates PCR amplification curves acquired at 52° C. in HEX (A-D) and FAM (E-H) channels for various targets. PCR curves shown with a dashed line represent the presence of a single gene target per reaction whereas those shown with a solid line represent the presence of both gene targets per reaction with 20,000 copies (black line) and 32 copies (grey line) of target. In the HEX channel, results are shown for CTcry only (A), CTcry and NGopa (B), NGopa only (C), and all remaining off-target controls including 10,000 copies TFRC (genomic DNA endogenous control), TVbtub, and MgPa (D). In the FAM channel, results are shown for TVbtub only (E), TVbtub and MgPa (F), MgPa only (G), and all remaining off-target controls including 10,000 copies TFRC (genomic DNA endogenous control) or 20,000 copies and 32 copies of CTcry and NGopa (H). In all figures, the black dotted line represents the no-template controls (NF H₂O). The amplification curves are the averages of the fluorescence level from triplicate reactions.

FIG. 11 shows the change in signal (ΔS; where ΔS=S_{D-post-PCR}−S_{D-pre-PCR}) for detection of (A) CTcry and NGopa in HEX and (B) TVbtub and MgPa in FAM using endpoint analysis method 1. Results are represented in black and white pattern for ΔS_{D1} (52° C.) and grey for ΔS_{D2} (70° C.). The values for each sample are the average of three replicates and the error bars represent the standard deviation between these replicates.

FIG. 12 illustrates qualitative endpoint detection of NGopa in the HEX channel (A) and MgPa in the FAM channel (B) using endpoint analysis method 2 (ΔS_{D2}−ΔS_{D1}=ΔΔS_{D2}ΔS_{D1}). The values for each sample are averages of three replicates and the error bars represent the standard deviation between these replicates.

FIG. 13 illustrates changes in fluorescent signal obtained in the HEX (FIGS. 13A & 13B) and FAM (FIGS. 13C & 13D) channels at two different temperatures using endpoint analysis method 3. (FIG. 13A) Detection of CTcry (CT; 20,000 (20K) or 32 copies) and/or NGopa (NG; 20,000 (20K) or 32 copies) using ΔS_{D2} in the HEX channel (FIG. 13B) Differentiation of CTcry and NGopa in HEX using ratio ΔS_{D1}:ΔS_{D2}. Detection of CTcry alone is determined when ΔS_{D1}:ΔS_{D2}>Threshold R₁. Detection of NGopa alone is determined when ΔS_{D1}:ΔS_{D2}<Threshold R₂. Detection of coinfection containing both targets is determined when ΔS_{D1}:ΔS_{D2}>Threshold R₂ and <Threshold R₁. (FIG. 13C) Detection of TVbtub and/or MgPa using ΔS_{D2} in the FAM channel (FIG. 13D) Differentiation of TVbtub and MgPa in FAM using ratio ΔS_{D1}:ΔS_{D2}. Detection of TVbtub alone is determined when ΔS_{D1}:ΔS_{D2}>Threshold R₁. Detection of MgPa alone is determined when ΔS_{D1}:ΔS_{D2}<Threshold R₂. Detection of coinfection containing both targets is determined when ΔS_{D1}:ΔS_{D2}>Threshold R₂ and <Threshold R₁. The values for each sample are averages of three replicates and the error bars represent the standard deviation between these replicates.

FIG. 14 illustrates detection of TFRC at Temperature 1 (D₁) (FIG. 14A) and TPApolA at temperature 2 (D₂) (FIG. 14B) in Cy5.5. channel using endpoint analysis method 2. Detection of TFRC (FIG. 14A) is achieved by the subtraction of pre-PCR fluorescence at Temperature 1 from the post-PCR fluorescence a Temperature 1 (S_{D1}). Detection of TPApolA (FIG. 14B) is achieved by the subtraction of pre-PCR fluorescence at Temperature 2 from the post-PCR fluorescence a Temperature 2 (ΔS_{D2}), followed by the subtraction of ΔS_{D1} (ΔΔS_{D2}ΔS_{D1}). The values for each sample are averages of two replicates for NF H₂O, 10,000 cps TPApolA, 40 cps TPApolA, 10,000 cps TPApolA and 10,000 cps TFRC, 40 cps TPApolA and 10,000 cps TFRC. The values for 10,000 cps TFRC are the averages of 48 replicates because TFRC was used as an endogenous control and is present in genomic DNA at 10,000 cps in each reaction well, except for the TPApolA-only samples. Error bars represent the standard deviation between each replicate for each sample.

FIG. 15 (FIG. 15A) Melt signature produced by the cleavage of Substrate 4 in the presence of 10,000 copies of TFRC (solid black line). (FIG. 15B) Melt signature produced by the cleavage of LOCS-3 in the presence of 10,000 copies of TPApolA (solid black line) and 40 copies of TPApolA (solid grey line). (C) Melt signature produced by the cleavage of Substrate 4 and LOCS-3 in the presence of 10,000 copies of TPApolA plus 10,000 copies of TFRC (solid black line), and 40 copies of TPApolA plus 10,000 copies of TFRC (solid grey line). The melt signature resulting from the absence of both targets (NF H₂O) is represented in (FIGS. 15A-C), with a dashed line. Results represent the rate of change in fluorescence with temperature (−d(RFU)/dT).

FIG. 16 illustrates PCR amplification plots obtained from reactions containing either 20,000 copies of CTcry (solid black line), 20,000 copies of NGopa (dashed black line), 20,000 copies of both targets (solid grey line) or neither target (NTC; grey dashed line) at 39° C. (A) and 72° C. (B). Threshold values Threshold X and Threshold Y are indicated for amplification plots obtained at 39° C. and 72° C. respectively. Endpoint fluorescence values, designated E_{X1}, E_{X2}, E_{Y1} and E_{Y2}, are indicated.

FIG. 17 illustrates PCR amplification plots obtained from reactions containing copies of target X/CTcry; namely either 0 copies (solid black line), 32 copies (grey line) or 20,000 copies of CTcry (dotted black line) in a background of target Y/NGopa at varying copy numbers as indicated. Plots A, D and G show fluorescence at 39° C. for reactions containing NGopa at 20,000 copies (A), 32 copies (D) or no copies (G). Plots B, E and H show fluorescence at 74° C. for reaction containing NGopa at 20,000 copies (B), 32 copies (E) or no copies (H). Plots C, F and I show fluorescence at 74° C. after normalisation with FAF for reactions containing NGopa at 20,000 copies (C), 32 copies (F) or no copies (I).

FIG. 18 PCR amplification curves for the quantitative detection of human GAPDH at (D₁) 52° C. in the FAM channel. (FIG. 18A) Curves represent signal produced from 10,000 copies (grey solid line) and 100 copies (black dashed line) of GAPDH target alone (FIG. 18B) Results produced by 10,000 copies (solid grey line) and 100 copies (black dashed line) of MgPa target alone. (FIG. 18C) Curves represent signal produced from target mixtures containing 10,000 copies (grey solid line) and 100 copies (black dashed line) each of GAPDH and MgPa. The no template control (NF H₂O) is represented in (FIGS. 18A-C) as a black solid line. The amplification curves are the averages of the fluorescence level from triplicate reactions.

FIG. 19 illustrates qualitative detection of GAPDH and MgPa using one TaqMan probe and one LOCS probe, respectively, at two temperatures in the FAM channel. Results were obtained using endpoint analysis methods 1-3. The values for each sample are the average of three replicates and the error bars represent the standard deviation between these replicates. (FIG. 19A) Results obtained with endpoint analysis method 1 show the change in signal (ΔS) between Post-PCR and Pre-PCR fluorescent measurements. Results are represented in black and white pattern for ΔS_{D1} (52° C.) and grey for ΔS_{D2} (70° C.). (FIG. 19B) Detection of MgPa alone with results obtained from endpoint analysis method 2 (ΔΔS_{D2}ΔS_{D1}). (FIGS. 19C and 19D) Results obtained from endpoint analysis method 3 (ΔS_{D1}:ΔS_{D2}). (FIG. 19C) Detection of GAPDH (Human 10,000 or 100 copies) and/or MgPa (MG 10,000 or 100 copies) using ΔS_{D2} in the FAM channel (FIG. 19D) Differentiation of GAPDH and MgPa in FAM using ΔS_{D1}:ΔS_{D2} ratio. Detection of GAPDH alone is determined when ΔS_{D1}:ΔS_{D2}>Threshold R₁. Detection of MgPa alone is determined when ΔS_{D1}:ΔS_{D2}<Threshold R₂. Detection of coinfection containing both targets is determined when ΔS_{D1}:ΔS_{D2}>Threshold R₂ and <Threshold R₁. Results are measured in Ratio Units.

FIG. 20 illustrates PCR amplification curves obtained in the FAM channel from reactions containing either 25,600 copies of TVbtub (black dotted line), 25,600 copies of MgPa (black dashed line), a mixture containing 25,600 copies of both targets (grey solid line) or no target (NF H₂O; black solid line) at 52° C. (FIG. 20A) and 74° C. (FIG. 20B). An increase in fluorescence at 52° C. (D₁) indicates the presence of TVbtub detected by a Molecular Beacon and an increase in fluorescence at 74° C. (D₂) indicates the presence of MgPa detected by the LOCS probe. The Cq values determined at D₁ and D₂ were used to quantify the amount of TVbtub and MgPa, respectively, in a sample without the need for special analysis methods. Curves represent the average fluorescence level from triplicate reactions.

FIG. 21 illustrates the standard curve obtained at 52° C. (D₁) that was used for quantification of TVbtub (FIG. 20A) and the standard curve obtained at 74° C. (D₂) that was used for quantification of MgPa (FIG. 20B). Triplicates of 25600, 6400, 1600, 400 and 100 copies of synthetic TVbtub and MgPa G-Block templates were used to generate the standard curves.

FIG. 22 illustrates embodiments wherein a Dual Hybridization Probe may be combined with a LOCS probe which may be cleavable by an MNAzyme. Both the Dual Hybridization Probe and the LOCS probe may be labelled with the same fluorophore. The two Dual Hybridization Probes may be capable of binding to target 1 with a Tm A and a Tm B respectively. These may be combined with an Intact LOCS probe which may have a stem region with a Tm C and a Loop region which can be cleaved by an MNAzyme in the presence a second target 2 thus generating a Split LOCS with a Tm D; where Tm D is less than Tm C. The presence of target 1 and/or target 2 can be determined by measuring the fluorescence at two temperatures either in real time; or using discrete measurements acquired at, or near, the beginning of amplification and following amplification.

FIG. 23 illustrates the simultaneous endpoint detection of two targets (TVbtub and MgPa) in the FAM channel using one non-cleavable molecular beacon and one LOCS probe by measuring the change in fluorescence during PCR at 52° C. (ΔS_{D1}, FIG. 23A) and 74° C. (ΔS_{D2}, FIG. 23B). Values plotted are the average of triplicate reactions containing varying amounts of TVbtub and/or MgPa, as specified in the graph. In FIG. 23A, ΔS_{D1} above the specified threshold indicates the presence of TVbtub in the reaction or the absence if below. In FIG. 23B, ΔS_{D2} above the specified threshold indicates the presence of MgPa in the reaction or the absence if below. The y-axis is the determined increase in fluorescence at 52° C. (ΔS_{D1}, FIG. 23A) or 74° C. (ΔS_{D2}, FIG. 23B).

FIG. 24 illustrates the simultaneous endpoint detection of two targets (TVbtub and MgPa) in the FAM channel using one non-cleavable molecular beacon and one LOCS probe by measuring the change in calibrated fluorescence signal during PCR at 52° C. (ΔS_{D1}/C, FIG. 24A) and 74° C. (ΔS_{D2}/C, FIG. 24B). Values plotted are the mean of replicates containing varying amounts of TVbtub and/or MgPa templates, as specified in the graph, and the errors bars represent the standard deviation between these replicates. In FIG. 24A, ΔS_{D1}/C above Threshold 1 indicates the presence of TVbtub in the reaction or the absence if below. In FIG. 24B, ΔS_{D2}/C above Threshold 2 indicates the presence of MgPa in the reaction or the absence if below.

FIG. 25 illustrates simultaneous endpoint detection of two targets (CTcry and NGopa) in the HEX channel using one linear MNAzyme substrate and one LOCS probe by measuring the change in fluorescence signal during PCR at 52° C. (ΔS_{D1}, FIG. 25A) and 70° C. (ΔS_{D2}, FIG. 25B), and the calibrated signals at 52° C. (ΔS_{D1}/C, FIG. 25C) and 70° C. (ΔS_{D2}/C, FIG. 25D) across the three Bio-Rad CFX96 machines tested (Machine 1 in black stripes, Machine 2 in grey and Machine 3 in white). Values plotted are the mean of triplicates containing varying amounts of CTcry and/or NGopa templates, as specified in the graph. and the error bars represent the standard deviation between these replicates. The signal in FIG. 25A or the calibrated signal in FIG. 25C above Threshold C₁ indicates the presence of CTcry in the reaction or the absence if below. The calibrated signal in FIG. 25D indicates the absence of CTcry and NGopa when below Threshold C₂, the presence of both CTcry and NGopa when above Threshold C₃, and the presence of only one of CTcry and NGopa when between Thresholds C₂ and C₃. FIG. 25B shows that the value of Threshold C₃ varies between the machines without calibration unlike that shown in FIG. 25D (Threshold C₂ not shown in FIG. 25B).

FIG. 26 illustrates simultaneous endpoint detection of target 1 (CTcry) and/or target 2 (NGopa) in the HEX channel using one linear MNAzyme substrate and one LOCS probe by using endpoint analysis method 2. Graph illustrating NS_{D1} (LHS) and ΔNS_{D2}NS_{D1} (RHS) were determined for CTcry detection and NGopa detection, respectively, by taking the post-PCR signals acquired at D₁ (52° C.) and D₂ (70° C.) from experimental samples and determining the background signals as the pre-PCR fluorescence measurements (S_{D3}) from the same reaction well at 40° C. (FIG. 26A-B); 52° C. (FIG. 26C-D) and 62° C. (FIG. 26E-F). In FIG. 26A, 26C and 26E, where the NS_{D1} is above threshold X₁, it indicates the presence of CTcry in the reaction or the absence if below. In FIG. 26B, 26D and 26F, where the ΔNS_{D2}NS_{D1} is above Threshold X₂, this indicates the presence of NGopa in the reaction or the absence if below.

FIG. 27 illustrates simultaneous endpoint detection of two targets (CTcry and NGopa) in the HEX channel using one linear MNAzyme substrate and one LOCS probe by using endpoint analysis method 2. NS_{D1} (LHS) and ΔNS_{D2}NS_{D1} (RHS) were determined for CTcry detection and NGopa detection, respectively, by taking the post-PCR signals acquired at D₁ (52° C.) and D₂ (70° C.) and determining the background signals as the pre-PCR fluorescence measurements (S_{D3}) as the mean of no template control signals measured at D₁/D_{3B} prior to PCR (FIGS. 27A-B) and at D₁ and D₂ prior to PCR (FIG. 27C-D) and following PCR (FIG. 27E-F). In FIGS. 27A, 27C and 27E, where the NS_{D1} is above threshold X₁, it indicates the presence of CTcry in the reaction or the absence if below. In FIG. 26B, 26D and 26F, where the ΔNS_{D2}NS_{D1} is above Threshold X₂, in indicates the presence of NGopa in the reaction or the absence if below.

### DEFINITIONS

As used in this application, the singular form “a”, “an” and “the” include plural references unless the context clearly dictates otherwise. For example, the phrase “polynucleotide” also includes a plurality of polynucleotides.

As used herein, the term “comprising” means “including”. Variations of the word “comprising”, such as “comprise” and “comprises,” have correspondingly varied meanings. Thus, for example, a polynucleotide “comprising” a sequence of nucleotides may consist exclusively of that sequence of nucleotides or may include one or more additional nucleotides.

As used herein the term “plurality” means more than one. In certain specific aspects or embodiments, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any integer derivable therein, and any range derivable therein.

As used herein, the term “subject” includes any animal of economic, social or research importance including bovine, equine, ovine, primate, avian and rodent species. Hence, a “subject” may be a mammal such as, for example, a human or a non-human mammal. Also encompassed are microorganism subjects including, but not limited to, bacteria, viruses, fungi/yeasts, protists and nematodes. A “subject” in accordance with the presence invention also includes infectious agents such as prions.

As used herein, the terms “polynucleotide” and “nucleic acid” may be used interchangeably and refer to a single- or double-stranded polymer of deoxyribonucleotide or ribonucleotide bases, or analogues, derivatives, variants, fragments or combinations thereof, including but not limited to DNA, methylated DNA, alkylated DNA, RNA, methylated RNA, microRNA, siRNA, shRNA, mRNA, tRNA, snoRNA, stRNA, smRNA, pre- and pri-microRNA, other non-coding RNAs, ribosomal RNA, derivatives thereof, amplicons thereof or any combination thereof. By way of non-limiting example, the source of a nucleic acid may be selected from the group comprising synthetic, mammalian, human, animal, plant, fungal, bacterial, viral, archael or any combination thereof.

As used herein, the term “oligonucleotide” refers to a segment of DNA or a DNA-containing nucleic acid molecule, or RNA or RNA-containing molecule, or a combination thereof. Examples of oligonucleotides include nucleic acid targets; substrates, for example, those which can be modified by an MNAzyme; primers such as those used for in vitro target amplification by methods such as PCR; components of MNAzymes; and various other types of reporter probes, including but not limited to, TaqMan or Hydrolysis probes; Molecular Beacons; Sloppy Beacons; Eclipse probes; Scorpion Uni-Probe, Scorpion Bi-Probes primer/probes, Capture/Pitchers and dual-hybridization probes. The term “oligonucleotide” includes reference to any specified sequence as well as to the sequence complementary thereto, unless otherwise indicated. Oligonucleotides may comprise at least one addition or substitution, including but not limited to the group comprising 4-acetylcytidine, 5-(carboxyhydroxylmethyl)uridine, 2′-O-methylcytidine, 5-carboxymethylaminomethyl thiouridine, dihydrouridine, 2′-O-methylpseudouridine, beta D-galactosylqueosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, beta D-mannosylmethyluridine, 5-methoxycarbonylmethyluridine, 5-methoxyuridine, 2-methylthio-N6-isopentenyladenosine, N-((9-beta-ribofuranosyl-2-methylthiopurine-6-yl)carbamoyl)threonine, N-((9-beta-ribofuranosylpurine-6-yl)N-methyl-carbamoyl)threonine, uridine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-beta-D-ribofuranosylpurine-6-yl)carbamoyl)threonine, 2′-O-methyl-5-methyluridine, 2′-O-methyluridine, wybutosine, 3-(3-amino carboxypropyl)uridine, beta D-arabinosyl uridine, beta D-arabinosyl thymidine.

The terms “polynucleotide” and “nucleic acid” “oligonucleotide” include reference to any specified sequence as well as to the sequence complementary thereto, unless otherwise indicated.

As used herein, the terms “complementary”, “complementarity”, “match” and “matched” refer to the capacity of nucleotides (e.g. deoxyribonucleotides, ribonucleotides or combinations thereof) to hybridise to each other via Watson-Crick base-pairing, noncanonical base-pairing including wobble base-pairing and Hoogsteen base-pairing (e.g. LNA, PNA or BNA) or unnatural base pairing (UBP). Bonds can be formed via Watson-Crick base-pairing between adenine (A) bases and uracil (U) bases, between adenine (A) bases and thymine (T) bases, between cytosine (C) bases and guanine (G) bases. A wobble base pair is a noncanonical base pairing between two nucleotides in a polynucleotide duplex (e.g. guanine-uracil, inosine-uracil, inosine-adenine, and inosine-cytosine). Hoogsteen base pairs are pairings that, like Watson-Crick base pairs, occur between adenine (A) and thymine (T) bases, and cytosine (C) and guanine (G) bases, but with differing conformation of the purine in relation to the pyrimidine compared to in Watson-Crick base pairings. An unnatural base pair is a manufactured subunit synthesized in the laboratory and not occurring in nature. Nucleotides referred to as “complementary” or that are the “complement” of each other are nucleotides which have the capacity to hybridise together by either Watson-Crick base pairing or by noncanonical base pairing (wobble base pairing, Hoogsteen base pairing) or by unnatural base pairing (UBP) between their respective bases. A sequence of nucleotides that is “complementary” to another sequence of nucleotides herein may mean that a first sequence is 100% identical to the complement of a second sequence over a region of 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides. Reference to a sequence of nucleotides that is “substantially complementary” to another sequence of nucleotides herein may mean that a first sequence is at least 60%, 65%, 70%, 75%, 80%, 85%, 90%, 95%, 97%, 98% or 99% identical to the complement of a second sequence over a region of 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides.

As used herein, the terms “non-complementary”, “not complementary”, “mismatch” and “mismatched” refer to nucleotides (e.g. deoxyribonucleotides, ribonucleotides, and combinations thereof) that lack the capacity to hybridize together by either Watson-Crick base pairing or by wobble base pairing between their respective bases. A sequence of nucleotides that is “non-complementary” to another sequence of nucleotides herein may mean that a first sequence is 0% identical to the complement of a second sequence over a region of 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides.

Reference to a sequence of nucleotides that is “substantially non-complementary” to another sequence of nucleotides herein may mean that a first sequence is less than 1%, 2%, 3%, 4%, 5%, 10%, 15%, 20%, 25%, 30%, 35%, or 40% identical to the complement of a second sequence over a region of 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides.

As used herein, the term “target” refers to any molecule or analyte present in a sample that the methods of the present invention may be used to detect. The term “target” will be understood to include nucleic acid targets, and non-nucleic acid targets such as, for example proteins, peptides, analytes, ligands, and ions (e.g. metal ions).

As used herein, an “enzyme” refers to any molecule which can catalyze a chemical reaction (e.g. amplification of a polynucleotide, cleavage of a polynucleotide etc.). Non-limiting examples of enzymes suitable for use in the present invention include nucleic acid enzymes and protein enzymes. Non-limiting examples of suitable nucleic acid enzymes include ribozymes, MNAzymes DNAzymes and aptazymes. Non-limiting examples of suitable protein enzymes include exonucleases and endonucleases. The enzymes will generally provide catalytic activity that assists in carrying out one or more of the methods described herein. By way of non-limiting example, the exonuclease activity may be an inherent catalytic activity of, for example, a polymerase. By way of non-limiting example, the endonuclease activity may be an inherent catalytic activity of, for example, a restriction enzyme including a Nicking endonuclease, a riboendonuclease or a duplex specific nuclease (DSN).

As used herein, an “amplicon” refers to nucleic acid (e.g. DNA or RNA, or a combination thereof) that is a product of natural or artificial nucleic acid amplification or replication events including, but not limited to PCR, RT-PCR, SDA, NEAR, HDA, RPA, LAMP, RCA, TMA, LCR, RAM, 3SR, NASBA, and any combination thereof.

As used herein, the term “stem-loop oligonucleotide” will be understood to mean a DNA or DNA-containing molecule, or an RNA or RNA-containing molecule, or a combination thereof (i.e. DNA-RNA hybrid molecule or complex), comprising or consisting of a double-stranded stem component joined to a single-stranded loop component. The double-stranded stem component comprises a forward strand hybridised by complementary base pairing to a complementary reverse strand, with the 3′ nucleotide of the forward strand joined to the 5′ nucleotide of the single-stranded loop component, and the 5′ nucleotide of the reverse strand joined to the 3′ nucleotide of the single-stranded loop component. The double-stranded stem component may further comprise one or more detection moieties, including but not limited to, a fluorophore on one strand (e.g. the forward strand), and one or more quenchers on the opposing strand (e.g. the reverse strand). Other non-limiting examples include a gold or silver nanoparticle on both strands for colorimetric detection, immobilization of one strand to a gold surface (e.g. the forward strand) and a gold nanoparticle on the opposing strand (e.g. the reverse strand) for SPR detection, and immobilization of one strand to an electrode surface (e.g. the forward strand) and a methylene blue molecule on the opposing strand (e.g. reverse strand) for electrochemical detection.

As used herein, the term “stem-loop oligonucleotide” will be understood to include “LOCS”, also referred to herein as a “LOCS oligonucleotide”, “LOCS structure” “LOCS reporter”, “Intact LOCS”, “Closed LOCS” and “LOCS probes. The single-stranded loop component of a LOCS may comprise a region capable of serving as a substrate for a catalytic nucleic acid such as, for example, an MNAzyme, a DNAzyme, a ribozyme, an apta-MNAzyme, or an aptazyme. Additionally or alternatively, the single-stranded loop component may comprise a region which is complementary to a target nucleic acid (e.g. a target for detection, quantification and the like), and/or amplicons derived therefrom, and which may further be capable of serving as a substrate for an exonuclease enzyme. By way of non-limiting example, the exonuclease may be an inherent activity of a polymerase enzyme. Additionally or alternatively, the single-stranded loop component region may comprise a region which may: (i) be complementary to the target being detected, (ii) comprise one strand of a double stranded restriction enzyme recognition site; and (iii) be capable of serving as a substrate for a restriction enzyme, for example a nicking endonuclease. As used herein, the terms “split stem-loop oligonucleotide”, “split LOCS”, “split LOCS oligonucleotide”, “split LOCS structure” “split LOCS reporters”, “split LOCS probes”, “cleaved LOCS” and “degraded LOCS” are used herein interchangeably and will be understood to be a reference to a “LOCS” in which the single-stranded loop component has been cleaved, digested, and/or degraded (e.g. by an enzyme as described herein) such that at least one bond between adjacent nucleotides within the loop is removed, thereby providing an non-contiguous section in the loop region. In split LOCS, the forward and reverse strands of the double-stranded stem portion may retain the ability to hybridize to each other to form a stem in a temperature-dependent manner.

LOCS are designed to include a cleavable loop region enabling target-dependent cleavage of the loop region by an enzyme generating a split LOCS. This in turn may facilitate detection of the target from a detectable signal generated at specific temperature(s) following association (hybridisation) or dissociation of the stem portion of intact or split LOCS. In contrast, a Molecular Beacon as used herein refers to a stem loop oligonucleotide designed to include a loop region that is not cleavable during the methods described herein. Molecular Beacons may mediate target detection by generating detectable signal at specific temperatures following association (hybridization) or dissociation (separation) of the loop portion of the probe with the target to be detected. As such, a primary difference between these two types of stem loop structures in the context of the present invention is that LOCS are monitored by measuring changes in signals due to hybridization or dissociation of the stem region of intact or split LOCS, whereas Molecular Beacons are monitored by measuring changes in signal due to hybridization or dissociation of the loop region and the target.

As used herein, the term “universal stem” refers to a double stranded sequence which can be incorporated into any LOCS structure. The same “universal stem” may be used in LOCS which contain Loops which comprise either catalytic nucleic acid substrates or sequence which is complementary to a target of interest. A single universal stem can be used as a surrogate marker for any target which is capable of facilitating the splitting of a specific LOCS. A series of universal stems can be incorporated into a series of LOCS designed for analysis of any set of targets.

As used herein, the term “universal LOCS” refers to a LOCS structure which contains a “universal stem”, and a “universal Loop” which comprises a universal catalytic nucleic acid substrate which can be cleaved by any MNAzyme with complementary substrate binding arms regardless of the sequences of the MNAzyme target sensing arms. A single universal LOCS can be used as a surrogate marker for any target which is capable of facilitating the splitting of a specific LOCS. A series of universal LOCS can be incorporated into any multiplex assay designed to analyse any set of targets.

As used herein, the terms “nucleic acid enzyme”, “catalytic nucleic acid”, “nucleic acid with catalytic activity”, and “catalytic nucleic acid enzyme” are used herein interchangeably and shall mean a DNA or DNA-containing molecule or complex, or an RNA or RNA-containing molecule or complex, or a combination thereof (i.e. DNA-RNA hybrid molecule or complex), which may recognize at least one substrate and catalyse a modification (such as cleavage) of the at least one substrate. The nucleotide residues in the catalytic nucleic acids may include the bases A, C, G, T, and U, as well as derivatives and analogues thereof. The terms above include uni-molecular nucleic acid enzymes which may comprise a single DNA or DNA-containing molecule (also known in the art as a “DNA enzyme”, “deoxyribozyme” or “DNAzyme”) or an RNA or RNA-containing molecule (also known in the art as a “ribozyme”) or a combination thereof, being a DNA-RNA hybrid molecule which may recognize at least one substrate and catalyse a modification (such as cleavage) of the at least one substrate. The terms above include nucleic acid enzymes which comprise a DNA or DNA-containing complex or an RNA or RNA-containing complex or a combination thereof, being a DNA-RNA hybrid complex which may recognize at least one substrate and catalyse a modification (such as cleavage) of the at least one substrate. The terms “nucleic acid enzyme”, “catalytic nucleic acid”, “nucleic acid with catalytic activity”, and “catalytic nucleic acid enzyme” include within their meaning MNAzymes.

As used herein, the terms “MNAzyme” and “multi-component nucleic acid enzyme” as used herein have the same meaning and refer to two or more oligonucleotide sequences (e.g. partzymes) which, only in the presence of an MNAzyme assembly facilitator (for example, a target), form an active nucleic acid enzyme that is capable of catalytically modifying a substrate. An “MNAzyme” is also known in the art as a “PlexZyme”. MNAzymes can catalyse a range of reactions including cleavage of a substrate, and other enzymatic modifications of a substrate or substrates. MNAzymes with endonuclease or cleavage activity are also known as “MNAzyme cleavers”. Component partzymes, partzymes A and B each of bind to an assembly facilitator (e.g. a target DNA or RNA sequence) through base pairing. The MNAzyme only forms when the sensor arms of partzymes A and B hybridize adjacent to each other on the assembly facilitator. The substrate arms of the MNAzyme engage the substrate, the modification (e.g. cleavage) of which is catalyzed by the catalytic core of the MNAzyme, formed by the interaction of the partial catalytic domains of partzymes A and B. MNAzymes may cleave DNA/RNA chimeric reporter substrates. MNAzyme cleavage of a substrate between a fluorophore and a quencher dye pair may generate a fluorescent signal. The terms “multi-component nucleic acid enzyme” and “MNAzyme” comprise bipartite structures, composed of two molecules, or tripartite structures, composed of three nucleic acid molecules, or other multipartite structures, for example those formed by four or more nucleic acid molecules.

It will be understood that the terms “MNAzyme” and “multi-component nucleic acid enzyme” as used herein encompass all known MNAzymes and modified MNAzymes including those disclosed in any one or more of PCT patent publication numbers WO/2007/041774, WO/2008/040095, WO2008/122084, and related US patent publication numbers 2007-0231810, 2010-0136536, and 2011-0143338 (the contents of each of these documents are incorporated herein by reference in their entirety). Non-limiting examples of MNAzymes and modified MNAzymes encompassed by the terms “MNAzyme” and “multi-component nucleic acid enzyme” include MNAzymes with cleavage catalytic activity (as exemplified herein), disassembled or partially assembled MNAzymes comprising one or more assembly inhibitors, MNAzymes comprising one or more aptamers (“aptaMNAzymes”), MNAzymes comprising one or more truncated sensor arms and optionally one or more stabilizing oligonucleotides, MNAzymes comprising one or more activity inhibitors, multi-component nucleic acid inactive proenzymes (MNAi), each of which is described in detail in one or more of WO/2007/041774, WO/2008/040095, US 2007-0231810, US 2010-0136536, and/or US 2011-0143338.

As used herein, the terms “partzyme”, “component partzyme” and “partzyme component” refer to a DNA-containing or RNA-containing or DNA-RNA-containing oligonucleotide, two or more of which, only in the presence of an MNAzyme assembly facilitator as herein defined, can together form an “MNAzyme.” In certain preferred embodiments, one or more component partzymes, and preferably at least two, may comprise three regions or domains: a “catalytic” domain, which forms part of the catalytic core that catalyzes a modification; a “sensor arm” domain, which may associate with and/or bind to an assembly facilitator; and a “substrate arm” domain, which may associate with and/or bind to a substrate. The terms “sensor arm”, “target sensor arm” or “target sensing arm” or “target arm” may be used interchangeably to describe the domain of the partzymes which binds to the assembly facilitator, for example the target. Partzymes may comprise at least one additional component including but not limited to an aptamer, referred to herein as an “apta-partzyme.” A partzyme may comprise multiple components, including but not limited to, a partzyme component with a truncated sensor arm and a stabilizing arm component which stabilises the MNAzyme structure by interacting with either an assembly facilitator or a substrate.

The terms “assembly facilitator molecule”, “assembly facilitator”, “MNAzyme assembly facilitator molecule”, and “MNAzyme assembly facilitator” as used herein refer to entities that can facilitate the self-assembly of component partzymes to form a catalytically active MNAzyme by interaction with the sensor arms of the MNAzyme. As used herein, assembly facilitators may facilitate the assembly of MNAzymes which have cleavage or other enzymatic activities. In preferred embodiments an assembly facilitator is required for the self-assembly of an MNAzyme. An assembly facilitator may be comprised of one molecule, or may be comprised of two or more “assembly facilitator components” that may pair with, or bind to, the sensor arms of one or more oligonucleotide “partzymes”. The assembly facilitator may comprise one or more nucleotide component/s which do not share sequence complementarity with sensor arm/s of the MNAzyme. The assembly facilitator may be a target. The target may be a nucleic acid selected from the group consisting of DNA, methylated DNA, alkylated DNA, RNA, methylated RNA, microRNA, siRNA, shRNA, tRNA, mRNA, snoRNA, stRNA, smRNA, pre- and pri-microRNA, other non-coding RNAs, ribosomal RNA, derivatives thereof, amplicons, or any combination thereof. The nucleic acid may be amplified. The amplification may comprise one or more of: PCR, RT-PCR, SDA, NEAR, HDA, RPA, LAMP, RCA, TMA, RAM, LCR, 3SR, or NASBA.

MNAzymes are capable of cleaving linear substrates and/or substrates which are present within the Loop region of a stem-loop LOCS reporter probe structures. Cleavage of a linear substrate may separate a fluorophore and quencher allowing detection of a target. Cleavage of the Loop region of a LOCS by an MNAzyme may generate a Split LOCS structure composed of two fragment which may remain hybridized and associated at temperatures below the melting temperature of the stem and which may separate and dissociate at temperature above the melting temperature of the stem of the split LOCS.

The terms “detectable effect” and “detectable signal” are used interchangeably herein and will be understood to have the same meaning. The terms refer to a signal or an effect generated from a detection moiety that is attached to or otherwise associated with an oligonucleotide of the present invention (e.g. a probe, reporter or substrate), typically upon modification of the oligonucleotide to alter its conformation, structure, orientation, position relative to other entit(ies), and the like. The modification may, for example, be induced by the presence of a target that the oligonucleotide is designed to detect. Non-limiting examples of such modifications (e.g. those induced by the presence of the target) include the opening of the stem-loop portion of a Molecular Beacon, the opening of double-stranded portion of Scorpion Uniprobes and Biprobes, the binding of Dual Hybridisation Probes to a target sequence, the production of a Catcher Duplex, and cleavage/digestion of a linear MNAzyme substrate or a TaqMan probe, and the like. The detectable effect may be detected by a variety of methods, including fluorescence spectroscopy, surface plasmon resonance (SPR), mass spectroscopy, NMR, electron spin resonance, polarization fluorescence spectroscopy, circular dichroism, immunoassay, chromatography, radiometry, photometry, scintigraphy, electronic methods, electrochemical methods, UV, visible light or infra-red spectroscopy, enzymatic methods or any combination thereof. The detectable signal/effect can be detected or quantified, and its magnitude may be indicative of the presence and/or quantity of an input such as the amount of a target molecule present in a sample. Further, the magnitude of the detectable signal/effect provided by the detection moiety may be modulated byaltering the conditions of a reaction in which an oligonucleotide comprising the detectable moiety is utilised, including but not limited to, the reaction temperature. The capacity of the detectable moieties attached to or otherwise associated with the oligonucleotides to generate target-dependent signal, and/or target-independent background signal, can thus be modulated.

As used herein the terms “background signal” and “baseline signal” are used interchangeably and will be understood to have the same meaning. The terms refer to signal generated by a detectable moiety attached to or otherwise associated with an oligonucleotide of the present invention, that is independent of the presence or absence of the specific target which the oligonucleotide is designed to measure or detect under the specific conditions of measurement.

The terms “polynucleotide substrate”, “oligonucleotide substrate” and “substrate” as used herein include any single- or double-stranded polymer of deoxyribonucleotide or ribonucleotide bases, or analogues, derivatives, variants, fragments or combinations thereof, which is capable of being recognized, acted upon or modified by an enzyme including a catalytic nucleic acid enzyme. A “polynucleotide substrate” or “oligonucleotide substrate” or “substrate” may be modified by various enzymatic activities including but not limited to cleavage. Cleavage or degradation of a “polynucleotide substrate” or “oligonucleotide substrate” or “substrate” may provide a “detectable effect” for monitoring the catalytic activity of an enzyme. The “polynucleotide substrate” or “substrate” may be capable of cleavage or degradation by one or more enzymes including, but not limited to, catalytic nucleic acid enzymes such as MNAzymes, AptaMNAzymes, DNAzymes, Aptazymes, ribozymes and/or protein enzymes such as exonucleases or endonucleases.

A “reporter substrate” as used herein is a substrate that is particularly adapted to facilitate measurement of either cleavage or degradation of a substrate or the appearance of a cleaved product in connection with a catalyzed reaction. Reporter substrates can be free in solution or bound (or “tethered”), for example, to a surface, or to another molecule. A reporter substrate can be labelled by any of a large variety of means including, for example, fluorophores (with or without one or more additional components, such as quenchers), radioactive labels, biotin (e.g. biotinylation) or chemiluminescent labels.

As used herein, a “linear MNAzyme substrate” is a substrate, for example, a reporter substrate, that is recognized by and acted on catalytically by a plurality of MNAzymes. A “linear MNAzyme substrate” does not contain sequences at its 5′ or 3′ ends which are capable of hybridizing to form a stem. Alternatively, MNAzyme substrates may be present within the Loop region of a LOCS probe.

As used herein, a “universal substrate” is a substrate, for example, a reporter substrate, that is recognized by and acted on catalytically by a plurality of MNAzymes, each of which can recognize a different assembly facilitator. The use of such substrates facilitates development of separate assays for detection, identification, or quantification of a wide variety of assembly facilitators using structurally related MNAzymes all of which recognize a universal substrate. These universal substrates can each be independently labelled with one or more labels. In preferred embodiments, independently detectable labels are used to label one or more universal substrates to allow the creation of a convenient system for independently or simultaneously detecting a variety of assembly facilitators using MNAzymes. In some embodiments the substrates may be capable of catalytic modification by DNAzymes which are catalytically active in the presence of a cofactor, for example a metal ion co-factor such as lead or mercury. In some embodiments the substrates may be amenable to catalytic modification by aptazymes which may become catalytically active in the presence of an analyte, protein, compound or molecule capable of binding to the aptamer portion of the aptazyme thereby activating the catalytic potential of the nucleic acid enzyme portion.

The terms “probe” and “reporter” as used herein refer to an oligonucleotide that is used for detection of a target molecule (e.g. a nucleic acid or an analyte). Non-limiting examples of Standard Probes or Reporters, which are well known in the art include, but are not limited to, linear MNAzyme substrates, TaqMan probes or hydrolysis probes, Molecular Beacons, Sloppy Beacons, Eclipse probes, Scorpion Uni-Probe, Scorpion Bi-Probes primer/probes, capture/pitcher oligonucleotides, and dual-hybridization probes. Embodiments of the present invention combine standard probes with LOCS probes. Some LOCS probes comprise nucleic acid enzyme substrates within the loop regions which may be universal, and which are capable of catalytic cleavage by nucleic acid enzymes such as MNAzymes, DNAzymes and aptazymes. Other LOCS probes comprise target specific sequences within the loop region which are capable of catalytic cleavage by protein enzymes including endonucleases and exonucleases.

The term “product” refers to the new molecule or molecules that are produced as a result of enzymatic modification of a substrate. As used herein the term “cleavage product” refers to a new molecule produced as a result of cleavage or endonuclease activity by an enzyme. In some embodiments, the products of enzymatic cleavage or degradation of an intact, LOCS structure comprise two oligonucleotide fragments, collectively referred to as a Split LOCS, wherein the two oligonucleotide fragments may be capable of either hybridization or dissociation/separation depending upon the temperature of the reaction.

As used herein, use of the terms “melting temperature” and “Tm” in the context of a polynucleotide will be understood to be a reference to the melting temperature (Tm) as calculated using the Wallace rule, whereby Tm=2° C. (A+T)+4° C. (G+C) (see Wallace et al., (1979) Nucleic Acids Res. 6, 3543), unless specifically indicated otherwise. The effects of sequence composition on the melting temperature can be understood using the nearest neighbour method, which is governed by the following formula: Tm (° C.)=ΔH°/(ΔS°+R In[oligo])−273.15. In addition to stem length and sequence composition, other factors that are known to impact the melting temperature include ionic strength and oligonucleotide concentration. A higher oligonucleotide and/or ion concentration increases the chance of duplex formation which leads to an increase in melting temperature. In contrast, a lower oligonucleotide and/or ion concentration favours dissociation of the stem which leads to a decrease in melting temperature.

As used herein the term “quencher” includes any molecule that when in close proximity to a fluorophore, takes up emission energy generated by the fluorophore and either dissipates the energy as heat or emits light of a longer wavelength than the emission wavelength of the fluorophore. Non-limiting examples of quenchers include Dabcyl, TAMRA, graphene, FRET fluorophores, ZEN quenchers, ATTO quenchers, Black Hole Quenchers (BHQ) and Black Berry Quenchers (BBQ).

As used herein, the term “base” when used in the context of a nucleic acid will be understood to have the same meaning as the term “nucleotide”.

As used herein the term “blocker” or “blocker molecule” refers to any molecule or functional group which can be incorporated into an oligonucleotide to prevent a polymerase using a portion of the oligonucleotide as a template for the synthesis of a complementary strand. By way of a non-limiting example, a hexathylene glycol blocker can be incorporated into, for example, a Scorpion probe to link its 5′ probing sequence to its 3′ priming sequence, wherein the blocker functions to prevent a polymerase using the probing sequence as a template.

As used herein the terms “normalise”, “normalising” and “normalised”, refer to the conversion of a measured signal (e.g. a detectable signal generated by a detection moiety) to a scale relative to a known and repeatable value or to a control value.

As used herein, the term “kit” refers to any delivery system for delivering materials. Such delivery systems include systems that allow for the storage, transport, or delivery of reaction reagents (for example labels, reference samples, supporting material, etc. in the appropriate containers) and/or supporting materials (for example, buffers, written instructions for performing an assay etc.) from one location to another. For example, kits may include one or more enclosures, such as boxes, containing the relevant reaction reagents and/or supporting materials. The term “kit” includes both fragmented and combined kits.

As used herein, the term “fragmented kit” refers to a delivery system comprising two or more separate containers that each contains a subportion of the total kit components. The containers may be delivered to the intended recipient together or separately. Any delivery system comprising two or more separate containers that each contains a subportion of the total kit components are included within the meaning of the term “fragmented kit”.

As used herein, a “combined kit” refers to a delivery system containing all of the components of a reaction assay in a single container (e.g. in a single box housing each of the desired components).

It will be understood that use the term “about” herein in reference to a recited numerical value includes the recited numerical value and numerical values within plus or minus ten percent of the recited value.

It will be understood that use of the term “between” herein when referring to a range of numerical values encompasses the numerical values at each endpoint of the range. For example, a polypeptide of between 10 residues and 20 residues in length is inclusive of a polypeptide of 10 residues in length and a polypeptide of 20 residues in length.

Any description of prior art documents herein, or statements herein derived from or based on those documents, is not an admission that the documents or derived statements are part of the common general knowledge of the relevant art.

For the purposes of description all documents referred to herein are hereby incorporated by reference in their entirety unless otherwise stated.

### Abbreviations

The following abbreviations are used herein and throughout the specification:

LOCS: loop connected to stems
MNAzyme: multi-component nucleic acid enzyme;
Partzyme: Partial enzyme containing oligonucleotide;
PCR: polymerase chain reaction;
gDNA: genomic DNA
NTC: No template control
qPCR: Real-time quantitative PCR
Ct; Threshold cycle
Cq; Quantification cycle
R²; Correlation coefficient

### nM; Nanomolar

### mM; Millimolar

### μL; Microlitre

### μM; Micromolar

dNTP; Deoxyribonucleotide triphosphate
NF—H₂O: nuclease-free water;
LNA: locked nucleic acid;
F: fluorophore;
Q: quencher;
N=A, C, T, G, or any analogue thereof;
N′=any nucleotide complementary to N, or able to base pair with N;
(N)ₓ: any number of N;
(N)ₓ: any number of N′;

### W: A or T;

### R: A, G, or AA;

rN: any ribonucleotide base;
(rN)ₓ: any number of rN;

### rR: A or G;

### rY: C or U;

### M: A or C;

### H: A, C, or T;

### D: G, A, or T;

JOE or 6-JOE: 6-carboxy-4′,5′-dichloro-2,7′-dimethoxyfluorescein;

### FAM or 6-FAM: 6-Carboxyfluorescein.

### BHQ1: Black Hole Quencher 1

### BHQ2: Black Hole Quencher 2

RT-PCR: reverse transcription polymerase chain reaction
SDA: strand displacement amplification

### NEAR: Nicking Enzyme Amplification Reaction

HDA: helicase dependent amplification

### RPA: Recombinase Polymerase Amplification

LAMP: loop-mediated isothermal amplification
RCA: rolling circle amplification
TMA: transcription-mediated amplification
3SR: self-sustained sequence replication
NASBA: nucleic acid sequence based amplification

### LCR: Ligase Chain Reaction

### RAM: Ramification Amplification Method

### IB: Iowa Black® FQ

### IBR: Iowa Black® RQ

shRNA: short hairpin RNA
stRNA: short interfering RNA
mRNA: messenger RNA
tRNA: transfer RNA
snoRNA: small nucleolar RNA
stRNA: small temporal RNA
smRNA: small modulatory RNA
pre-microRNA: precursor microRNA
pri-microRNA: primary microRNA
LHS: Left hand side
RHS: Right hand side
DSO: double stranded oligonucleotide

### Tm: Melting Temperature

### RFU: Relative Fluorescence Units

CT: Chlamydia trachomatis
NG: Neisseria gonorrhoeae
SPR: surface plasmon resonance
GNP: gold nanoparticles

### DETAILED DESCRIPTION

The following detailed description conveys exemplary embodiments of the present invention in sufficient detail to enable those of ordinary skill in the art to practice the present invention. Features or limitations of the various embodiments described do not necessarily limit other embodiments of the present invention or the present invention as a whole. Hence, the following detailed description does not limit the scope of the present invention, which is defined only by the claims.

The present invention relates to methods and compositions for the improved multiplexed detection of targets (e.g. nucleic acids, proteins, analytes, compounds, molecules and the like). The methods and compositions each employ a combination of a LOCS oligonucleotide together with other oligonucleotide reporters, probes or substrates, which may be used in combination with various other agent/s.

Reporters, Probes and Substrates

According to the present invention, multiplex detection of target molecules is facilitated using LOCS in combination with another oligonucleotide suitable for use as a probe in a multiplex detection assay.

Many oligonucleotides for detection of nucleic acid targets have been described and are well known in the art. Suitable oligonucleotides that can be used in combination with LOCS include, but are not limited to, MNAzyme substrates, TaqMan or Hydrolysis probes, Molecular Beacons, Sloppy Beacons, Eclipse probes, Scorpion Uni-Probe, Scorpion Bi-Probes, dual-hybridization probes and Capture/Pitcher probes.

In some embodiments, these oligonucleotides bind directly to the target or target amplicon to facilitate their detection, however, MNAzyme substrates and Capture/Pitcher oligonucleotides provide an exception as they may be universal and suitable for detection of any target.

In some embodiments, the oligonucleotides generate fluorescence in the presence of target due to enzymatically mediated cleavage or degradation, for example, MNAzyme substrates and TaqMan or Hydrolysis probes.

In other embodiments, the oligonucleotides provide different levels of fluorescent signal as a result of a conformation change induced by binding to a target or target amplicon (e.g. Molecular Beacons, Sloppy Beacons, Eclipse probes, Scorpion Uni-Probe, Scorpion Bi-Probes and dual-hybridization probes).

In the TOCE system, the Catcher changes fluorescence as a result of conformation changes induced by binding and extension of the Pitcher which is only activated and released in the presence of target.

Any, or all, of these types of reporter oligonucleotides are suitable for use in conjunction with LOCS probes to mediate detection of multiple targets by measurement of changes related to a single detection moiety, including but not limited to, a change in fluorescence measured at a single wavelength.

Oligonucleotides for combination with LOCS can be synthesised according to standard protocols. For example, they may be synthesised by phosphoramidite chemistry, using nucleoside and non-nucleoside phosphoramidites in sequential synthetic cycles that involves removal of the protective group, coupling the phosphoramidites, capping and oxidation, either in solid-phase or solution-phase and optionally in an automated synthesiser device. Alternatively, they may be purchased from commercial sources. Non-limiting examples of commercial sources from which MNAzyme substrates, TaqMan or Hydrolysis probes, Molecular Beacons, Sloppy Beacons, Eclipse probes, Scorpion Uni-Probe, Scorpion Bi-Probes, dual-hybridization probes and Capture/Pitcher probes include, can be purchased or otherwise obtained include: MNAzyme substrates can be purchased from SpeeDx (plexper.com); TaqMan and hydrolysis probes can be purchased from Thermo Fisher Scientific (www.thermofisher.com), Sigma Aldrich (www.sigmaaldrich.com), Promega (www.promega.com), Generi Biotech (www.generi-biotech.com); Molecular Beacons and Sloppy beacons may be purchased from Integrated DNA Technologies (www.idtdna.com), Eurofins (www.eurofinsgenomics.com), Sigma Aldrich (www.sigmaaldrich.com) and TriLink BioTechnologies (www.trilinkbiotech.com); Eclipse probes can be purchased from Integrated DNA Technologies (www.idtdna.com); Scorpion Uni-Probes can be purchased from Sigma Aldrich (www.sigmaaldrich.com) and Bio-Synthesis (https://www.biosyn.com); Scorpion bi-probes can be purchased from Bio-Synthesis (https://www.biosyn.com); Dual-hybridisation probes can be purchased from Bio-Synthesis (https://www.biosyn.com), Sigma Aldrich (www.sigmaaldrich.com) and Eurofins (www.eurofinsgenomics.com) and Catcher Pitcher assays may be purchased from Seegene (www.seegene.com).

LOCS Oligonucleotides

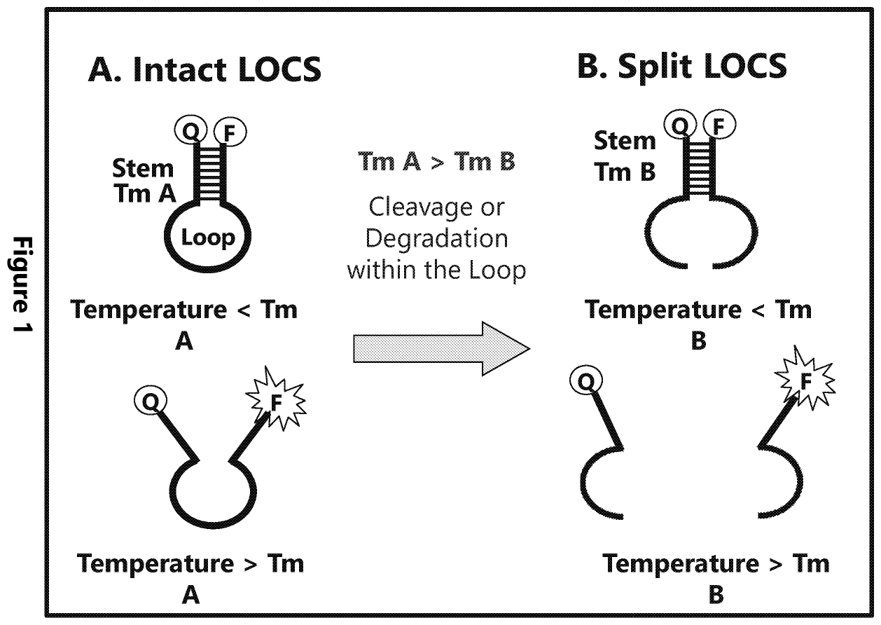

Exemplary LOCS oligonucleotides for use in the present invention are illustrated in FIG. 1. The exemplary Intact LOCS oligonucleotide shown (FIG. 1A, LHS) has a Loop region, a Stem region and a fluorophore (F)/quencher (Q) dye pair. Although exemplified with a fluorophore/quencher pair, the skilled addressee will recognise that any other suitable detection moiet(ies) may be used for the same purpose. The Loop region contains a substrate region which is amenable to enzymatic cleavage or degradation in the presence of target or target amplicons. Cleavage or degradation of the Loop within an Intact LOCS, generates the Split LOCS duplex (FIG. 1B, RHS).

In some embodiments, the melting temperature (“Tm”) of the Intact LOCS oligonucleotide is higher than the Tm of the Split LOCS structure.

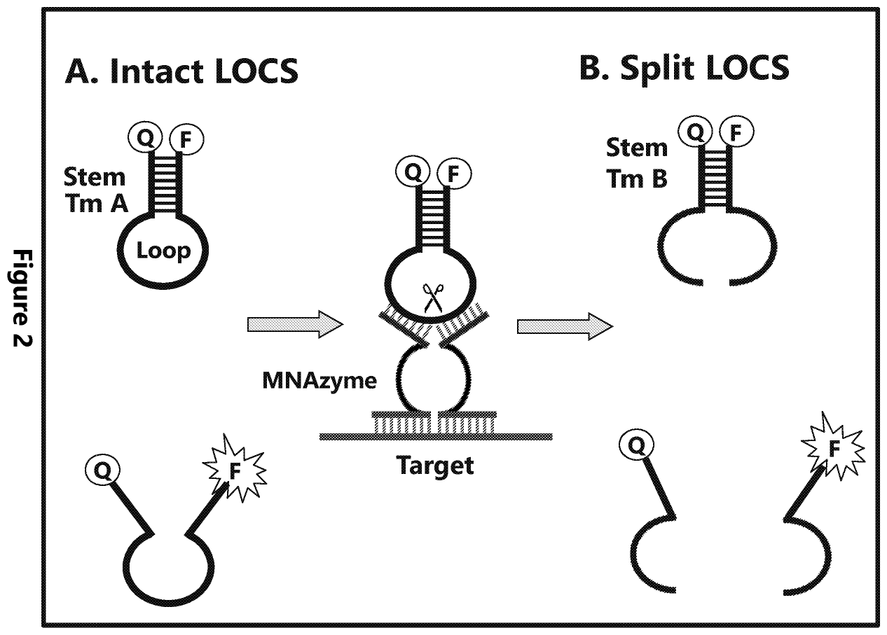

Since intramolecular bonds are stronger than intermolecular bonds, the stem regions of the intact LOCS structures will generally melt at a higher temperature than the stems of the Split, cleaved or degraded LOCS oligonucleotide structures. For example, the Stem of intact LOCS A will melt at Tm A which is higher than Tm B which is the temperature at which Split LOCS stem melts (FIG. 1B). The presence of fluorescence at a temperature which allows melting of Split LOCS but not Intact LOCS is indicative of the presence of target, or target amplicons. In the exemplary LOCS depicted in FIG. 2, the sequence of the Loop region of a LOCS oligonucleotide may be, for example, a substrate for a MNAzyme or other catalytic nucleic acid/s.

An exemplary LOCS suitable for use in the invention, may contain a Loop region comprising a substrate for a catalytic nucleic acid is illustrated in FIG. 2. In these embodiments, LOCS oligonucleotides comprise universal substrates which can be used to detect any target. The LOCS oligonucleotide contains a stem region, a fluorophore quencher/dye pair (alternative detection moiet(ies) as described herein may be employed) and an intervening Loop region which comprises a universal substrate for a catalytic nucleic acid such as an MNAzyme. The MNAzyme may detect a target directly or may be used to detect amplicons generated during target amplification. The MNAzyme forms when the target sensor arms of the partzymes each hybridise to a target, or to target amplicons, by complementary base pairing to form the active catalytic core of the MNAzyme. The Loop region of the LOCS oligonucleotide hybridises to the substrate binding arms of the MNAzyme by complementary base pairing and the substrate within the Loop is cleaved by the MNAzyme. This generates a Split LOCS structure which has a stem with a Tm B that is lower than the Tm A of the Intact LOCS. Measurement of a fluorescent signal at temperatures above Tm B but below Tm A is indicative of the presence of target in the reaction. Persons skilled in the art will recognize that the targets can be detected in real time or at the end of the reaction.

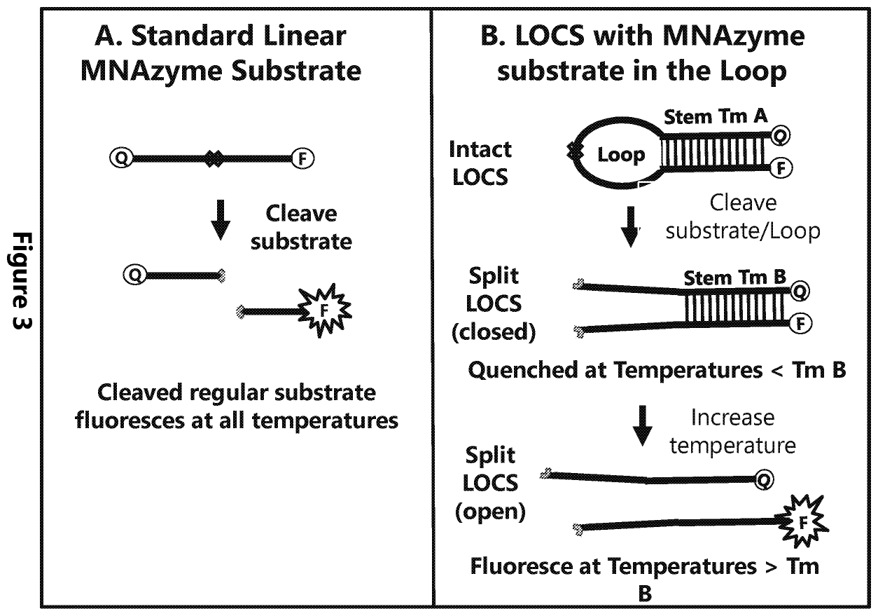

Referring to the exemplary embodiment illustrated in FIG. 3, a Standard Linear MNAzyme substrate is shown and used in conjunction with a single LOCS probe comprising an MNAzyme substrate within its Loop. The Linear MNAzyme substrate and the single LOCS probe may both be labelled with the same (or similar) detection moieties, for example a specific fluorophore(F)/quencher(Q) dye pair. Alternative detection moiet(ies) as described herein may be employed. The linear substrate comprises a first substrate sequence which is cleavable by a first MNAzyme that assembles in the presence of a first target (FIG. 3A). In the presence of the first target, the linear substrate is cleaved by the first MNAzyme, resulting in an increase in fluorescence which can be detected across a broad range of temperatures. The LOCS contains a second substrate sequence within its Loop which is cleavable by a second MNAzyme which assembles in the presence of a second target (FIG. 3B). In the presence of the second target the LOCS is cleaved to generate a Split LOCS that melts at Tm B which is lower than the melting temperature of the Intact LOCS (Tm A). At temperatures below Tm B, the stem portions of the Split LOCS remains hybridized and hence the fluorophore is quenched due to the proximity to the quencher molecule. At temperatures above Tm B, the stem portions of the Split LOCS dissociate and separate the fluorophore from the quencher molecule resulting in a fluorescence increase. When both targets are present, and fluorescence is measured at a first temperature below Tm B, the increase in fluorescence is associated with the first target 1 only. When fluorescence is measured at second temperatures above Tm B, but below TmA, the increase in fluorescence is associated with the first target and/or second target. When both target 1 and target 2 are present, the observed change in fluoresence during amplification at the second temperatures is greater than the change at the first temperature thus allowing determination of whether target 1, or target 2, or targets 1 and 2, or no neither target, are present in the reaction.

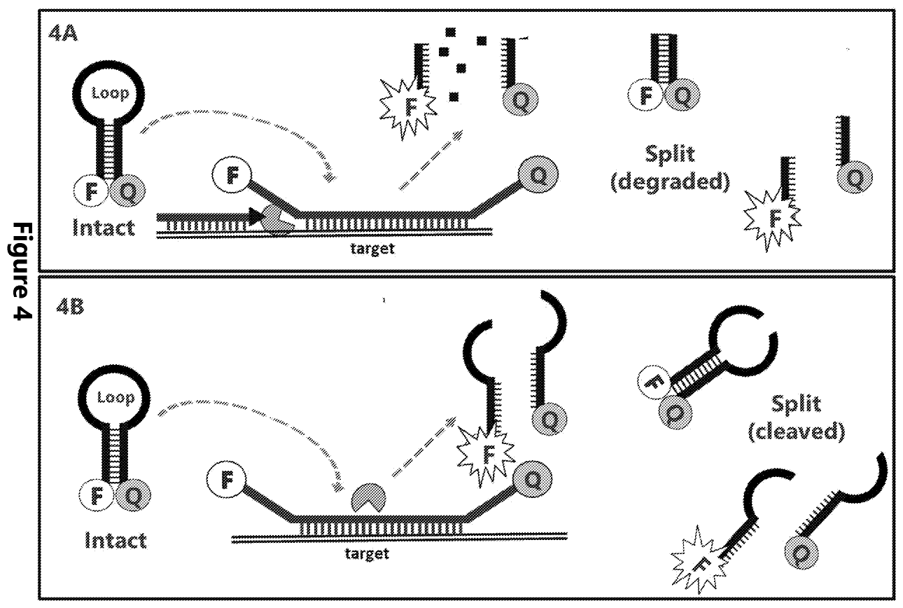

In other embodiments of the present invention, alternative LOCS structures useful for combination with Standard Reporter probes can be used. As exemplified in FIG. 4A and FIG. 4B, the Loop region of a LOCS oligonucleotide may comprise a target-specific sequence which is fully or partially complementary to the target to be detected, and which, when double-stranded, may serve as substrate for degradation by an exonuclease, for example, by exonuclease activity inherent to a polymerase (FIG. 3A). In yet a further embodiment, illustrated in FIG. 3B, the target specific sequence within the Loop may further comprise one strand of a double-stranded restriction enzyme recognition site. Hybridisation of the Loop sequence to the target sequence can result in a functional, cleavable restriction site. In preferred embodiments the restriction enzyme is a nicking enzyme which is capable of cleaving the Loop strand of the LOCS oligonucleotide while leaving the target intact.

In various embodiments of the present invention, different combinations of well-known reporter probes or substrates can be combined with a LOCS probe in a single reaction. By way of non-limiting example, a reaction for detection of two targets may comprise any combination of a first probe selected from group 1 including, but not limited to, linear MNAzyme substrates, TaqMan or Hydrolysis probes, Molecular Beacons, Sloppy Beacons, Eclipse probes, Scorpion Uni-Probes, Scorpion Bi-Probes, Capture/Pitcher oligonucleotides, and dual-hybridization probes, together with a second probe selected from group 2 including, but not limited to, a LOCS probe comprising a universal MNAzyme substrate, a LOCS probe comprising a target-specific substrate for an exonuclease, and a LOCS probe comprising a target-specific substrate for an endonuclease such as a nicking enzyme.

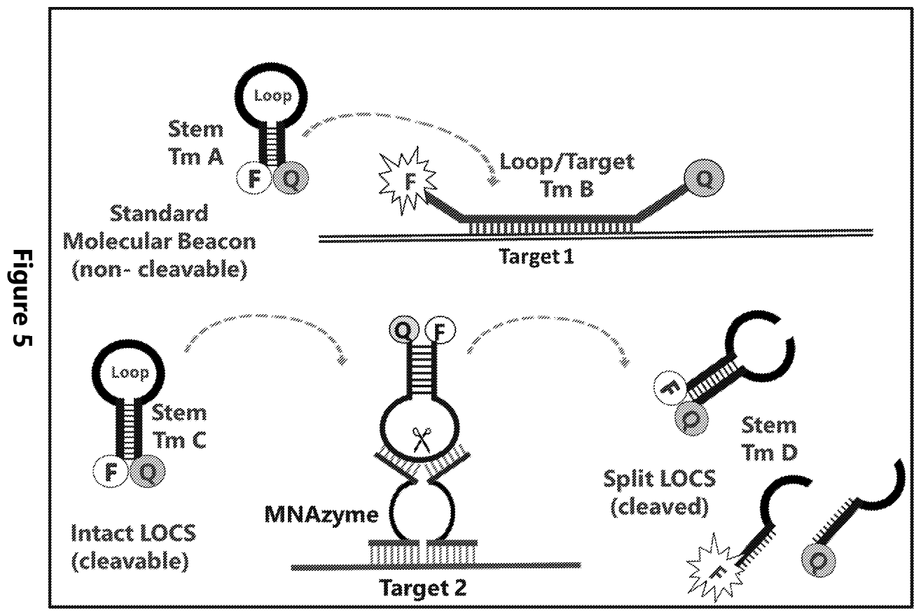

Any combination of a group 1 probe with a group 2 probe can be used to measure multiple targets in a single reaction according to the methods of the present invention. The embodiment illustrated in FIG. 3 illustrates the exemplary combination of a linear MNAzyme substrate cleavable by a first MNAzyme combined with a LOCS probe which is cleavable by a second MNAzyme. Other non-limiting embodiments of the present invention are illustrated in FIG. 5 in which a non-cleavable Molecular Beacon may be combined with a LOCS probe which is cleavable by an MNAzyme. Both the Molecular Beacon and the LOCS probe may be labelled with the same (or similar) detection moiety, for example the same fluorophore or fluorophores that emit at similar wavelengths. The Molecular Beacon may have a stem region with a Tm A and a Loop region which can specifically hybridize with a first target 1 with a Tm B; where Tm B is greater than Tm A. This may be combined with an Intact LOCS probe which may have a stem region with a Tm C and a Loop region which can be cleaved by an MNAzyme in the presence a second target 2 thus generating a Split LOCS with a Tm D, where Tm D is less than Tm C. The presence of target 1 and/or target 2 can be discriminated by measuring the fluorescence at two temperatures either in real-time, or using discrete measurements acquired at, or near, the beginning of amplification and following amplification.
LOCS Combinations Comprising Reversible Probes

In some embodiments the compositions and methods of the present invention comprise a combination of LOCS and an oligonucleotide probe capable of generating target-dependent detectable signals which can be reversibly modulated by temperature. Further, the LOCS and the oligonucleotide probe may be amenable to modulation of target-independent signal generation by temperature thus allowing manipulation of background noise or baseline levels.

For example, the oligonucleotide probe may adopt a first conformation or arrangement in the absence of the target in which the emission of a detectable signal is suppressed, and a second conformation or arrangement in the presence of the target that facilitates the emission of a detectable signal indicative of the presence of the target. Non-limiting examples of oligonucleotide probes in this category include Molecular Beacons, Sloppy Beacons, Scorpion Uniprobes, Scorpion Bi-Probes, and Capture/Pitcher Oligonucleotides.

Alternatively, in the case of Dual Hybridisation probes, two additional oligonucleotides in addition to the LOCS may adopt an arrangement in which a detectable signal is suppressed in the presence of the target and in which the detectable signal is generated when the target is absent.

In the embodiments above, the Intact LOCS undergoes a target-dependent cleavage event to provide a Split LOCS. The double-stranded stem portion of the Split LOCS can be designed to dissociate at a temperature that differs from the temperature at which the target-dependent change in conformation or arrangement of the first oligonucleotide(s) and associated detectable signal is generated.

In some embodiments, the oligonucleotide is a Molecular Beacon. The following scenarios provide non-limiting examples of multiplex detection assays according to embodiments of the present invention. In these scenarios, a Molecular Beacon may be used in combination with a LOCS for detection of targets 1 and 2, respectively. The Molecular Beacon may comprise a Tm A being the melting temperature of its double-stranded stem portion, and a Tm B being the melting temperature of a duplex formed between its single-stranded loop duplex and target 1. The LOCS may comprise a Tm C being the melting temperature of its double-stranded stem portion when Intact, and a Tm D being the melting temperature of its double-stranded stem portion when Split. Opposing strands of the double-stranded stem portion of the Molecular Beacon may be labelled with a fluorophore and quencher, as may those of the LOCS. The fluorophore of the Molecular Beacon may be the same, or emit in the same region of the visible spectrum, as the fluorophore of the LOCS. In alternative embodiments, different detection moieties may be utilised including, for example, nanoparticles of the same or similar size and/or type for colorimetric or SPR detection, reactive moieties (e.g. alkaline phosphatase or peroxidase enzymes) for chemiluminescent detection, electroactive species (e.g. ferrocene, methylene blue or peroxidase enzymes) for electrochemical detection. The skilled person will readily understand that the Molecular Beacon in these cases may be substituted with a Sloppy Beacon, Scorpion Uniprobe, or a Scorpion Bi-Probe.

Various relationship may exist between the temperatures at which reaction are measured and the melting temperatures of various regions of the reporter probes. Four scenarios are described in detail below within the context of reactions comprising one Molecular Beacon and one LOCS probe and non-limiting exemplary temperatures for such scenarios are outlined in Table 1 below.

TABLE 1
Non-limiting Exemplary Scenarios for interpretation of fluorescence changes following amplification in the
presence (+T) or absence (−T) of Target 1 (T1 detected by Molecular Beacon) and/or Target 2 (T2 detected by LOCS
probe), when the melting temperatures (Tms) the stem and loop of a Beacon, and the stems of Intact and Split
LOCS probes, are varied and measured at two different temperatures (D₁ and D₂ where D2 > D1);
Fluorescence (F), Quenched (Q), Background (Bgd); (Bgd F or Bgd Q levels Pre and Post PCR independent
of +/− any T; Pre PCR Bgd present at initiation); ΔS_{D1} = Change in F signal at D₁; ΔS_{D2} =
Change in F signal at D₂. All Scenarios: Tm B > Tm A; Tm C > Tm D.

| | Molecular Beacon: Bgd F > Tm A; Signal T1 at < Tm B | | | | | | | | |
|---|---|---|---|---|---|---|---|---|---|
| | LOCS: Bgd F at > Tm D Signal T2 at > Tm D < Tm C | | | | | | | | |
| | Scenario 1 | | | Scenario 2 | | | Scenario 3 | | |
| | D₁ < Tm A; D₂ < Tm C; D₂ > Tm D | | | | | | | | |
| | | D₁ | D₂ | | D₁ | D₂ | | D₁ | D₂ |
| | Tm | 50° C. | 60° C. | Tm | 50° C. | 65° C. | Tm | 50° C. | 75° C. |
| Beacon stem | 65 | Q −T₁, +/−T₂ | | 60 | Q −T₁, +/−T₂ | Bgd | 60 | Q −T₁, +/−T₂ | Bgd |
| Tm A | | | | | | F +/− | | | F +/− |
| | | | | | | any T | | | any T |
| Beacon loop/ | 70 | F −T₁, +/−T₂ | | 70 | F −T₁, +/−T₂ | | 70 | F −T₁, +/−T₂ | |
| T1 Tm B | | | | | | | | | |
| Intact LOCS | 65 | Bgd | Q −T₂, +/−T₁ | 70 | Bgd | Q −T₂, +/−T₁ | 80 | Bgd | Q −T₂, +/−T₁ |
| stem Tm C | | Q +/− | | | Q +/− | | | Q +/− | |
| Split LOCS | 55 | any T | F +T₂, +/−T₁ | 60 | any T | F +T₂, +/−T₁ | 70 | any T | F +T₂, +/−T₁ |
| stem Tm D | | | | | | | | | |

| | | | |
|---|---|---|---|
| Pre-PCR Bgd | Q at D₁; Q at D₂ | Q at D₁; F at D₂ | Q at D₁; F at D₂ |
| Positive ΔS_{D1} | T₁ present | T₁ present | T₁ present |
| Positive ΔS_{D2} | T₁ or T₂ or T₁ + T₂ present | T₂ present | T₂ present |
| | ΔS_{D2} > ΔS_{D1} → T₂ present | | |

| | Molecular Beacon: Bgd F > Tm A; Signal T1 at < Tm B | | |
|---|---|---|---|
| | LOCS: Bgd F at > Tm D Signal T2 at > Tm D < Tm C | | |
| | Scenario 4 | | |
| | D₁ > Tm C; D₂ > Tm D; | | |
| | D₂ < Tm C | | |
| | | D₁ | D₂ |
| | Tm | 70° C. | 60° C. |
| Beacon stem | 75 | Q −T₁, +/−T₂ | Q +/− |
| Tm A | | | any T |
| Beacon loop/ | 80 | F +T₁, +/−T₂ | |
| T1 Tm B | | | |
| Intact LOCS | 65 | Bgd | Q −T₂, +/−T₁ |
| stem Tm C | | F +/− | |
| Split LOCS | 55 | any T | F +T₂, +/−T₁ |
| stem Tm D | | | |

| | |
|---|---|
| Pre-PCR Bgd | F at D₁; Q at D₂ |
| Positive ΔS_{D1} | T₁ present |
| Positive ΔS_{D2} | T₁ or T₂ or T₁ + T₂ present |
| | ΔS_{D2} > ΔS_{D1} → T₂ present |

### Scenario 1

In a first non-limiting example, the Tm A may be greater than Tm D, and Tm B may be greater than Tm D. The presence of target 1 and/or target 2 can be discriminated by measuring the fluorescence at two temperatures acquired either at, or near, the beginning of amplification and following amplification. In the presence of target 1 and/or target 2 measurement of the fluorescence at the first temperature 1, which may be less than Tm A, Tm B and Tm D, may generate a signal indicative of the presence of target 1 only. At this temperature, the Molecular Beacon will be hybridized to target 1 (if present) and fluorescing; but in the absence of target 1, its stem will remain internally hybridized and hence quenched. At temperature 1 both intact and/or split LOCS species will be quenched due to hybridization of their respective stems at this temperature. Additionally, in the presence of target 1 and/or target 2, measurement of fluorescence at the second temperature 2, which is greater than both temperature 1 and Tm D, but less than both Tm B and Tm C, can be indicative of the presence of target 1 and/or target 2. At this second temperature the Molecular Beacon will be hybridized to the target 1 (if present) and fluoresce, but in the absence of target 1 its stem will remain internally hybridized and hence quenched. Additionally, at this second temperature, if target 2 is absent, the LOCS probe will remain intact and quenched, but will be split by an MNAzyme specific for target 2 (if present) and its stem will dissociated to generate fluorescence. In this scenario if fluorescence at temperature 1 increases during amplification, this indicates target 1 is present. Further, if the increase in fluorescence observed at temperature 2 during the course of amplification is greater than that observed at temperature 1, this indicates target 2 is present.

### Scenario 2

In a second non-limiting example, Tm A may be similar to Tm D, Tm B may be similar to Tm C, and Tm B may be greater than Tm D. The presence of target 1 and/or target 2 can be discriminated by measuring the fluorescence at two temperatures either in real time; or using single measurements acquired either at, or near, the beginning of amplification and following amplification. In the presence of target 1 and/or target 2, measurement of fluorescence at the first temperature 1 which may be less than Tm A, and less than Tm B, and less than Tm D, may generate signal indicative of the presence of target 1 only. At this temperature, the Molecular Beacon will be hybridized to target 1 (if present) and fluorescing, but its stem will remain internally hybridized in the absence of target 1 and will be quenched. At this first temperature 1, both intact and/or split LOCS species will be quenched due to hybridization of the stem region at this temperature. Additionally, in the presence of target 1 and/or target 2, measurement of fluorescence at the second temperature 2 which is greater than both temperature 1 and Tm A and Tm D, but less than both Tm B and Tm C, can be indicative of the presence of target 2. At this second temperature the Molecular Beacon will be hybridized to the target 1 (if present) and fluoresce; or will fluoresce in a target-independent manner due to dissociation and opening of its stem at this temperature. As such the Molecular Beacon will fluoresce regardless of the presence or absence of either target, giving a background fluorescence level at this temperature which may remain unchanged during amplification. Additionally, at this second temperature, if target 2 is absent the LOCS probe will remain intact and quenched; but will be split by an MNAzyme specific for target 2 (if present) and its stem will dissociate and thus generate fluorescence. In this scenario, if fluorescence at temperature 1 increases during amplification, this indicates target 1 is present and detected by the Molecular Beacon, whilst an increase in fluorescence at temperature 2 during amplification indicates target 2 is present and detected by the LOCS probe. Qualitative data can be obtained using discrete temperature measurements at/near the start and at the end of the PCR; and/or quantitative data can be read directly from the two amplification curves generated at each temperature during PCR. Alternatively, where Tm A and Tm D are not similar, the same relationship between the fluorescence level at the two temperatures and the presence or absence of target/s holds if both Tm A and Tm D are greater than Temperature 1 and less than Temperature 2.

The approach above may provide major advantages over other method(s) known in the art which exploit measurement at multiple temperatures to distinguish multiple targets at a single wavelength such as, for example, TOCE. TOCE measures fluorescence from a first target at a first temperature, and measures fluorescence from two targets at a second temperature (the first target plus a second target). This data is analyzed so as to mathematically subtract the amount of fluorescence related to the first target at the second temperature to quantify the second target in complex analysis which additionally requires adjustment to account for inherent difference in fluorescence which relate to temperature per se. The embodiments of the current invention described here exploits a Molecular Beacon and a LOCS probe in a method which negates the need for complex post PCR analysis since it allows direct quantification of a first target from a first amplification curve generated at a first temperature and direct quantification of a second target from a second amplification curve generated at a second temperature. These embodiments measure each target individually and further there is no requirement for adjustment to account for difference in fluorescence output of the same molecules since each target will only generate a signal that is detectable above background at one of the two temperatures selected for data acquisition.

### Scenario 3

In a third non-limiting example, Tm A may be less than Tm D, Tm B may be similar to Tm D and Tm C may be greater than Tm B. The presence of target 1 and/or target 2 can be discriminated by measuring the fluorescence at two temperatures either in real time; or using single measurements acquired either at, or near, the beginning of amplification and following amplification. In the presence of target 1 and/or target 2, measurement of fluorescence at a first temperature 1, which may be less than Tm A, and less than Tm B, and less than Tm D, may generate signal indicative of the presence of target 1 only. At this temperature, the Molecular Beacon will be hybridized to target 1 (if present) and fluorescing; but its stem will remain internally hybridized in the absence of target 1 and will be quenched. At this temperature 1, both intact and/or split LOCS species will be quenched due to hybridization of their stems at this temperature. Additionally, in the presence of target 1 and/or target 2, measurement of fluorescence at a second temperature 2, which is greater than both temperature 1 and Tm D and Tm A and Tm B, but is less Tm C, can be indicative of the presence of target 2. At this second temperature the Molecular Beacon cannot hybridize to target 1, and will always have an open dissociated stem and hence will fluoresce regardless of the presence or absence of either target, giving a background fluorescence level at this temperature which may remain unchanged during amplification. Additionally, at this second temperature, if target 2 is absent, the LOCS probe will remain intact and quenched, but will be split by MNAzymes specific for target 2 (if present) and its stem will be dissociated thus generating fluorescence. In this scenario, if fluorescence at temperature 1 increases during amplification, this indicates target 1 is present and detected by the Molecular Beacon, whilst an increase in fluorescence at temperature 2 indicates target 2 is present and detected by the LOCS probe. Qualitative data can be obtained using discrete temperature measurements at/near the start and at the end of the PCR; and/or quantitative data can be read directly from the two amplification curves generated at each temperature during PCR. Alternatively, where Tm A and Tm D are similar or Tm A is greater than Tm D, the same relationship between the fluorescence level at the two temperatures and the presence or absence of target/s holds if both Tm A and Tm D are greater than Temperature 1 and less than Temperature 2.

### Scenario 4

In a fourth non-limiting example, both Tm A and Tm B may be greater than Tm C and Tm D. The presence of target 1 and/or target 2 can be discriminated by measuring the fluorescence at two temperatures acquired either at, or near, the beginning of amplification and following amplification. In the presence of target 1 and/or target 2, measurement of fluorescence at a first temperature 1, which may be less than Tm A and Tm B but greater than Tm C and Tm D, may generate signal indicative of the presence of target 1. At this temperature, the Molecular Beacon will be hybridized to target 1 (if present) and fluorescing; but its stem will remain internally hybridized in the absence of target 1 and it will be quenched. At this temperature, the LOCS will fluoresce regardless of the presence or absence of target 2 and hence will only contribute to background which will remain unchanged during amplification. In the absence of target 2 the stem of the intact LOCS will dissociate and fluoresce, and similarly in the presence of target 2 the stem of the Split LOCS will dissociate and fluoresce. Additionally, an increase in fluorescence during the course of amplification at temperature 2 which is less than temperature 1, and less than Tm C and Tm A and Tm B, but greater than Tm D, can be indicative of the presence of target 1 and/or target 2. At this second temperature the Molecular Beacon will be hybridized to the target 1 (if present) and hence fluoresce, or it will remain quenched with a hybridized stem in the absence of target 1. Additionally, at this second temperature, if target 2 is absent the Intact LOCS probe stem will remain hybridized and quenched, or if target 2 is present the LOCS will be split by an MNAzyme specific for target 2 and the stem of the Split LOCS will dissociate and fluoresce. In this scenario if fluorescence at temperature 2 increases during amplification, this indicates that target 1 and/or target 2 are present. Further, if the increase in fluorescence observed during the course of amplification at temperature 2 is greater than that observed at temperature 1 this indicates target 2 is present.

LOCS Combinations Comprising Catcher-Pitcher Probes

In some embodiments the compositions and methods of the present invention may comprise a combination of a LOCS and a first oligonucleotide that functions as a Catcher component of a TOCE assay. This combination may, for example, allow simultaneous detection and quantification of two targets in a single fluorescent channel by acquiring fluorescence readings at two temperatures in real-time during PCR. Alternatively, in absence of real-time monitoring the approach could be applied to fluorescent data collected at discrete time points, for example near or at the beginning of amplification and following amplification.

In one embodiment, a first oligonucleotide comprising a Catcher can be combined with a LOCS probe, both of which may be labelled with the same fluorophore and quencher moieties for simultaneous detection in the same fluorescence channel. The reaction may also contain a Pitcher comprising a single-stranded oligonucleotide that includes a 5′ tag region which is complementary to the Catcher and a 3′ sensor region which is complementary to a first target 1. The Catcher may comprise a single-stranded oligonucleotide labelled with a quencher at the 5′ end and a fluorophore downstream to the quencher and a 3′ region that is complementary to the tag portion of the Pitcher. When the Catcher is in a single-stranded conformation, the fluorophore comes into close proximity with the quencher and the signal is quenched.

Taking the non-limiting example of an amplification reaction such as PCR, the primers and the 3′ sensor region of the Pitcher may hybridize to target 1. During primer extension using target 1 or amplicons thereof as template, the Pitcher may be degraded by the exonuclease activity of the DNA polymerase resulting in release of the tag portion. The released tag may then hybridize to the complementary 3′ portion of the Catcher, and be extended by the DNA polymerase, thus generating a double-stranded Catcher duplex with a Tm A wherein the fluorophore and quencher are separated resulting in increased fluorescence indicative the presence of target 1. Additionally, the reaction could contain an intact LOCS probe with a stem region with a Tm C and a Loop region which can be cleaved by an MNAzyme in the presence a second target 2, to generate a Split LOCS with a Tm D which is lower than Tm A. Various relationships may exist between the temperatures at which reactions are measured and the melting temperatures of the Catcher duplex and LOCS reporters. Three scenarios are described in detail below within the context of reactions comprising one Catcher probe and one LOCS probe and non-limiting exemplary temperatures for such scenarios are outlined in Table 2 below.

TABLE 2
Non-limiting Exemplary Scenarios for interpretation of fluorescence changes following amplification in the
presence (+T) or absence (−T) of Target 1 (T1 detected by Catcher-Pitcher probes) and/or Target 2 (T₂
detected by LOCS probe), when the melting temperatures (Tms) the Catcher Duplex, and the stems of Intact and Split
LOCS probes, are varied and measured at two different temperatures (D₁ and D₂ where D2 > D1);
Fluorescence (F), Quenched (Q), Background (Bgd); (Bgd F or Bgd Q levels Pre and Post PCR independent of +/−
any T; Pre PCR Bgd present at initiation); ΔS_{D1} = Change in F signal at D₁;
ΔS_{D2} = Change in F signal at D₂.

| | Catcher Duplex: Bgd Q > Tm B: Signal T1 at < Tm B | | | | | | | | |
|---|---|---|---|---|---|---|---|---|---|
| | LOCS: Bgd F at > Tm D Signal T2 at > Tm D < Tm C | | | | | | | | |
| | Scenario 1 | | | Scenario 2 | | | Scenario 3 | | |
| | D₁ < Tm A, D₁ < Tm C, D₁ < Tm D; | | | | | | D₁ > Tm C; D₂ > Tm D; | | |
| | D₂ < Tm C; D₂ > Tm D | | | | | | D₂ < Tm C | | |
| | | D₁ | D₂ | | D₁ | D₂ | | D₁ | D₂ |
| | Tm | 50° C. | 60° C. | Tm | 50° C. | 75° C. | Tm | 70° C. | 60° C. |
| Catcher Duplex | 70 | Q −T₁, +/−T₂ | | 70 | Q −T₁, +/−T₂ | Q +/− any T | 80 | Q −T₁,+/−T₂ | |
| Tm A | | F +T₁, +/−T₂ | | | F +T₁, +/−T₂ | | | F +T₁, +/−T₂ | |
| Intact LOCS | 65 | Bgd | Q −T₂, +/−T₁ | 80 | Bgd | Q −T₂, +/−T₁ | 65 | Bgd | Q −T₂, +/−T₁ |
| stem Tm C | | Q +/− | | | Q +/− | | | F +/− | |
| Split LOCS | 55 | any T | F +T₂, +/−T₁ | 70 | any T | F +T₂, +/−T₁ | 55 | any T | F +T₂, +/−T₁ |
| stem Tm D | | | | | | | | | |

| | | | |
|---|---|---|---|
| Pre-PCR Bgd | Q at D₁; Q at D₂ | Q at D₁; Q at D₂ | F at D₁; F at D₂ |
| Positive ΔS_{D1} | T₁ present | T₁ present | T₁ present |
| Positive ΔS_{D2} | T₁ or T₂ or T₁ + T₂ present | T₂ present | T₁ or T₂ or T₁ + T₂ present |
| | ΔS_{D2} > ΔS_{D1} → T₂ present | | ΔS_{D2} > ΔS_{D1} → T₂ present |

In a first non-limiting scenario, (see Table 2) when signal is measured at a first detection temperature, which is lower than the Tm A, Tm C and Tm D, then in the presence of target 1, the Catcher duplex can form and fluoresce, whilst in the absence of target 1, the single stranded Catcher will remain quenched. At this temperature, both the stems of the Intact and the Split LOCS will be hybridized and hence will be quenched regardless of the presence or absence of target 2. As such, an increase in fluorescence during amplification is indicative of target 1. Additionally, fluorescence measurement at a second detection temperature, which is higher than the Tm D but lower than Tm A and Tm C can be indicative of the presence of target 1 and/or target 2. At this second temperature, the Catcher remains single-stranded and quenched in the absence of target and forms a duplex and fluoresces in the presence of target. Additionally, at this second temperature, if target 2 is absent, the LOCS probe will remain intact and quenched, but will be cleaved in the presence of target 2 and its stem will dissociate to generate fluorescence. In this scenario if fluorescence at temperature 1 increases during amplification, this indicates target 1 is present. Further, if the increase in fluorescence observed at temperature 2 during the course of amplification is greater than that observed at temperature 1, this indicates target 2 is present. An increase the fluorescence at the second temperature, but not at the first, indicate target 2 only is present.

In a second non-limiting scenario (see Table 2), specific detection of a first target at a first detection temperature can be achieved according to scenario 1 above. Additionally, at a second detection temperature, which is higher than the Tm A and Tm D but lower than Tm C, the Catcher duplex is dissociated (i.e. single-stranded) and quenched regardless of the presence or absence of target 1 and/or target 2 since the temperature is above that where the Catcher duplex can form. In the absence of target 2, all LOCS will be intact and quenched; however, when target 2 is present Split LOCS will be generated, their stems will dissociate and an increase in fluorescence can be observed. Therefore, an increase in fluorescence during PCR at the first temperature is indicative of the presence of target 1 regardless of the presence or absence of target 2; and conversely, an increase in fluorescence during PCR at the second temperature is indicative of the presence of target 2 regardless of the presence or absence of target 1. As such, the combination of LOCS and Catcher-Pitcher probes may allow detection of target 1 only using Catcher-Pitcher probes, as monitored by an increase in fluorescence above background at a first temperature; and detection of target 2 only using LOCS probes, as monitored by an increase in fluorescence above background at a second temperature.

In a third non-limiting scenario (see Table 2), when signal is measured at a first detection temperature, which is lower than the Tm A but higher than the Tm C and Tm D, then in the presence of target 1 only, the Catcher duplex can form and fluoresce, whilst in the absence of target 1, the single stranded Catcher will remain quenched. At this temperature, both the stems of the Intact and the Split LOCS will be dissociated and generating a high level of background fluorescence, regardless of the presence or absence of target 2. As such, an increase in fluorescence above the background fluorescence during amplification is indicative of target 1 only. Additionally, fluorescence measurement at a second detection temperature, which is lower than the Tm A and Tm C but higher than the Tm D can be indicative of the presence of target 2 only. At this second temperature, the Catcher remains single-stranded and quenched in the absence of target and forms a duplex and fluoresces in the presence of target. Additionally, at this second temperature, if target 2 is absent, the LOCS probe will remain intact and quenched, but will be cleaved in the presence of target 2 and its stem will dissociate to generate fluorescence. In this scenario if fluorescence at temperature 1 increases during amplification, this indicates the presence of target 1 only and if fluorescence at temperature 2 increases during amplification, this indicates the presence of target 2 only.

In another non-limiting format, for example detection by SPR, the Catcher can be attached to a gold nanoparticle (GNP) and free in solution, whilst the Pitcher may be attached to a gold surface. In the presence of target 1, and at temperatures below Tm A, the catcher duplex may form and bring the GNP in close proximity to the gold surface which would produce a measurable shift in SPR signal. However, in the absence of target 1, the Catcher would be single-stranded and free in solution (i.e. not in close proximity to the gold surface) and would therefore not produce any measurable shift in SPR signal above that of the baseline SPR signal. Therefore, any measurable shift in SPR signal at this temperature would be indicative of the presence of target 1 in a sample. Further the LOCS may be attached at one end to a GNP and at the other end attached to the gold surface. In the absence of target 2, the GNP would always be attached, whereas in the presence of target 2 a Split LOCS would be generated and as such in this case the GNP would only be in proximity to the gold surface when the detection temperature is below that of the split LOCS. As before, in a scenario similar to scenario 2 above, if the first detection temperature is below Tm B, Tm C and Tm D then a change in signal would indicate the presence of target 1 since the GNP on the Catcher would be close to the gold surface. If the second detection temperature is below Tm C, but above Tm A and Tm D then a change in signal would indicate the presence of target 2 since the GNP on the Split LOCS would move away from the gold surface.

In yet another format, for example colorimetric detection, both the Catcher and the LOCS probe may be labelled on both ends with GNPs. At a first temperature, below Tm A, Tm C and Tm D, then the presence of target 1 would result in a measurable colour change from purple (GNP aggregated) to red (GNP dispersed), regardless of the presence or absence of target 2. At a second temperature above Tm A and Tm D but below Tm C then the presence of target 2 would result in a measurable colour change from purple (GNP aggregated) to red (GNP dispersed), regardless of the presence or absence of target 1.

In yet another format, the catcher can be labelled with an electroactive moiety such as methylene blue or ferrocene and the pitcher could be attached to an electrode surface. At the first temperature (below Tm A), the Catcher duplex would form on the electrode surface (if target 1 present), bringing the electroactive moiety in close proximity to the electrode surface which would produce a measurable shift in electrochemical signal (i.e. oxidation or reduction current). However, in the absence of target 1, the catcher would be free in solution and not in close proximity with the electrode surface and would therefore not produce any measurable shift in electrochemical signal (i.e. oxidation or reduction current) above that of the baseline signal. Therefore, any measurable shift in electrochemical signal at this temperature would be indicative of the presence of target 1 in a sample.

LOCS Combined with Dual Hybridization Probes

In some embodiments the compositions and methods of the present invention comprise a combination of LOCS and an oligonucleotide probe that comprises two target specific components.

Dual Hybridization Probes may contain a first oligonucleotide with a Tm A and second oligonucleotide with a Tm B, wherein Tm A and Tm B may be equal, or Tm A and Tm B may be different. The first oligonucleotide can be labelled at its 3′ terminus with a fluorophore and the second oligonucleotide could be labelled at its 5′ terminus with a quencher. (Alternatively, one oligonucleotide could be labelled at its 3′ terminus with a quencher and the other can be labelled at its 5′ terminus with a fluorophore.) The two oligonucleotide probes hybridize adjacently or substantially adjacently (e.g. less than 2, 3, 4, or 5 nucleotides gap) on target 1 and form a duplex structure with a Tm equal to whichever is the lowest of Tm A and Tm B. In this scenario, a suitable first temperature for data acquisition can be below Tm A and Tm B, and below Tm C and Tm D; namely the Tms of the stems of the Intact and Split LOCS respectively. At this first temperature, the first and second oligonucleotide are free in solution and fluorescent prior to amplification, or in the absence of target 1, but hybridization to target 1 if present brings the fluorophore and quencher into close proximity causing quenching. Further at the first temperature, both the stem of intact and split LOCS would be quenched. As such, an observed decrease in fluorescence at a first temperature is indicative of the presence of target 1, regardless of the presence or absence of target 2.

The LOCS probe may be cleaved by an MNAzyme in the presence a second target 2, Data acquisition can be performed at a second temperature which is above Tm A and/or Tm B, above Tm D but below Tm C. In this scenario, the Intact LOCS remains quenched prior to amplification and in the absence of target, but in the presence of target 2 the split LOCS stem will dissociate resulting in increases fluorescence. At this temperature, the first and second oligonucleotide would fluoresce regardless of the presence or absence of target 1 since they cannot hybridize to target 1 at this temperature. This fluorescence contributes to background signal at this temperature. As such an increase in fluorescence above background following amplification is indicative of the presence of Target 2 regardless of the presence or absence of target 1.

As such, the combination allows detection of target 1 using Dual Hybridization probes, determined as a decrease in fluorescence at a first temperature; and detection of target 2 only using LOCS probes, determined by an increase in fluorescence at a second temperature.

In another format, for example detection by SPR, the first oligonucleotide may be attached to a gold nanoparticle (GNP) and the second oligonucleotide may be attached to a gold surface. At the first temperature, the two oligonucleotide probes can hybridize adjacently on target 1 (if present), forming a duplex structure and bringing the GNP in close proximity to the gold surface which may produce a measurable shift in SPR signal. However, in the absence of target 1, the first oligonucleotide can be free in solution and not in close proximity to the gold surface and therefore not produce any measurable shift in SPR signal above that of the baseline SPR signal. Therefore, any measurable shift in SPR signal at this temperature can be indicative of the presence of target 1 in a sample.

In yet another format, for example colorimetric detection, both the first and second oligonucleotides can be labelled with gold nanoparticles. At the first temperature, the first and second oligonucleotides may be free in solution and exhibiting a red colour in the absence of target 1. However, in the presence of target 1, the two oligonucleotide probes can hybridize adjacently on the target, forming a duplex structure and bringing the GNPs in close proximity to each other to produce a measurable colour change from red to purple. Therefore, a measurable colour change from red to purple can be indicative of the presence of target 1 in a sample.

In yet another format, the first oligonucleotide can be labelled with an electroactive moiety such as methylene blue or ferrocene and the second oligonucleotide can be attached to an electrode surface. At the first temperature, the two oligonucleotide probes may hybridize adjacently on target 1 (if present), forming a duplex structure on the electrode surface and bringing the electroactive moiety in close proximity to the electrode surface which would produce a measurable shift in electrochemical signal (i.e. oxidation or reduction current). However, in the absence of target 1, the first oligonucleotide can be free in solution and not in close proximity with the electrode surface and would therefore not produce any measurable shift in electrochemical signal (i.e. oxidation or reduction current) above that of the baseline signal. Therefore, any measurable shift in electrochemical signal at this temperature can be indicative of the presence of target 1 in a sample.

LOCS Combinations Comprising Digestable Probes

In some embodiments the compositions and methods of the present invention comprise a combination of LOCS and an oligonucleotide probe that generates target-dependent detectable signals which cannot be reversibly modulated by temperature. However, the LOCS remains amenable to modulation of signal generation by temperature in a target-independent manner, thus allowing manipulation of background noise or baseline levels.

For example, the first oligonucleotide probe for a first target 1 may undergo a target-dependent modification that provided a detectable signal, which cannot be suppressed upon changing the temperature of detection. The target-dependent modification may be cleavage or digestion of the first oligonucleotide to thereby trigger the detectable signal indicative of the presence of the target. Non-limiting examples of oligonucleotide probes in this category include TaqMan probes, MNAzyme substrates and probes which are cleavable by restriction enzymes in a target dependent manner. Following modification, the signal generated by these probes can be measured over a wide range of temperatures.

In these embodiments, the Intact LOCS designed to detect a second target 2 can undergo cleavage to generate a Split LOCS only in the presence of this target. Measurement of target 2 requires detection at a temperature below the Tm of the stem of the intact LOCS but above the Tm of the stem of the Split LOCS. In contrast the temperature at which the first target is detected may be either below the Tms of the stems of both the Intact and Split LOCS, or above the Tms of the stems of both the Intact and Split LOCS. When the first detection temperature is below the Tms of the stems, the background signal will be suppressed as both Intact and/or Split stem will remain associated and quenched, whereas when the first detection temperature is above the Tms of the stems, the background signal will be higher due to dissociation of both Intact and/or Split stems. As such, detection of signal at the first temperature can be specific for target 1, regardless of the presence or absence of target 2. Detection of a signal at the second temperature indicates the presence of target 1 and/or target 2; however, if the increase in signal observed at the second temperature is greater than that observed at the first temperature, then this indicates target 2 is present. If a change in signal is only observed at the second temperature and not at the first, this would indicate the presence of the second target only.

The following scenarios provide non-limiting examples of multiplex detection assays according to embodiments of the present invention and are summarized in Table 3 below. In these scenarios, the LOCS may comprise a Tm A being the melting temperature of its double-stranded stem portion when Intact, and a Tm B being the melting temperature of its double-stranded stem portion when Split. The oligonucleotide probe (e.g. MNAzyme substrate) may be labelled with a fluorophore and quencher, as may opposing strands of the double-stranded stem portion of the LOCS. The fluorophore of the oligonucleotide probe may be the same, or emit in the same region of the visible spectrum, as the fluorophore of the LOCS. In alternative embodiments, different detection moieties may be utilised including, for example, nanoparticles of the same or similar size and/or type for colorimetric or SPR detection, reactive moieties (e.g. alkaline phosphatase or peroxidase enzymes) for chemiluminescent detection or electroreactive species (e.g. ferrocene, methylene blue or peroxidase enzymes) for electrochemical detection. The skilled person will readily understand that the oligonucleotide probe may, for example, be an MNAzyme substrate, a TaqMan probe, a hydrolysis probe, or a probe cleavable by a restriction enzyme in a target-dependent manner (RE probe).

The change in signal due to the presence or absence of a first target can be obtained by measuring the fluorescence following amplification (Post PCR) at a first temperature normalised against the background fluorescence acquired either at, or near, the beginning of amplification to PCR (pre-PCR) at this same first temperature; whilst the change in signals due to the presence or absence of a second target can be obtained by acquiring total fluorescence post-PCR at a second temperature normalised against the background fluorescence measured pre-PCR at this second temperature. Alternatively, the change in signal due to the presence or absence of a first and second target respectively can be obtained by measuring total fluorescence following amplification at the first and second temperature both of which can be normalised against the background fluorescence at the third temperature acquired either pre-PCR in the same reaction or at any time in a control reaction which is equivalent but know to lack target. Measurement of background may be performed at a third temperature provided the first temperature is less than the second temperature, and the third temperature is less than the Tm of the intact stem.

In all scenarios the presence or absence of a signal indicative of the first target is measured at the first temperature using a first probe which may, for example, be an MNAzyme substrate, a TaqMan probe or a RE probe. Prior to amplification this probe will be quenched; however, if target 1 is present, the probe will be cleaved during amplification resulting in an increase in fluorescence that is detectable at both the first and second temperatures. The presence or absence of the second target is determined at the second temperature using a LOCS probe where the second temperature is always above the Tm of the split LOCS (Tm B) but below the Tm of the Intact LOCS (Tm A).

TABLE 3
Non-limiting Exemplary Scenarios for interpretation of fluorescence changes following amplification in the
presence (+T) or absence (−T) of Target 1 (T1 detected by First Oligo which may be either an MNAzyme
Substrate, a TaqMan Probe, a linear probe cleavable by a RE) and/or Target 2 (T2 detected by LOCS probe), when
the Tms of the stems of Intact and Split LOCS probes arc measured at two different temperatures following
amplification (D₁ and D₂); Fluorescence (F), Quenched (Q)Temperature D₁ = First temperature; Temperature D₂ =
Second temperature < intact LOCS but > split LOCS; Optional Temperature D₃ = Measured pre-PCR (prior
amplification of T1 or T2) or measured in a control reaction lacking T1 and T2 where D3 < Tm Intact LOCS;
Background - Bkg; Number of Signals = Number of detectable moieties/probes that are fluorescing.
ΔS_{D1} = S_{D1post} − S_{D1 pre (Background)} or ΔS_{D1} = S_{D1post} − S_{D3 (Background)};
ΔS_{D2} = S_{D2post} − S_{D2 pre (Background)} or ΔS_{D2} = S_{D2post} − S_{D3 (Background)}

| | Scenario 1; SD₃ < SD₁ < SD₂ | | | Scenario 2; SD₁ > SD₂ | | | Scenario 3; SD₁ < SD₃ < SD₂ | | |
|---|---|---|---|---|---|---|---|---|---|
| | S_{D3} | S_{D1post} | S_{D2post} | | S_{D1post} | S_{D2post} | S_{D3} | S_{D1post} | S_{D2post} |
| | 40° C. | 50° C. | 60° C. | S_{D3} | 70° C. | 60° C. | 55° C. | 50° C. | 60° C. |
| First Oligo | Q | Q minus T₁ +/−T₂ | | N/A | Q minus T₁ +/−T₂ | | Q | Q minus T₁ +/−T₂ | |
| (Uncleaved) | | | | | | | | | |
| First Oligo | Not | F plus T₁ +/−T₂ | | | F plus T₁ +/−T₂ | | Not | F plus T₁ +/−T₂ | |
| (Cleaved) | present | | | | | | present | | |
| Intact LOCS | Q | Q +/− | Q minus | | F +/− | Q minus | Q | Q +/− | Q minus |
| stem | | any T | T₂ +/−T₁ | | any T | T₂ +/−T₁ | | any T | T₂ +/−T₁ |
| Tm = 65° C. | | | | | | | | | |
| Split LOCS | Not | | F plus | | | F plus T₂ | Not | | F plus |
| stem | present | | T₂ +/−T₁ | | | (+/−T₁) | present | | T₂ +/−T₁ |
| Tm = 55° C. | | | | | | | | | |
| Number of | 0 | 0 to 1 | 0 to 1 | | N/A | | 0 | 0 to 1 | 0 to 1 |
| signals | | (T1 | (T1 or T2) | | | | | (T1 | (T1 or T2) |
| SD₃ = Bkg | | present) | 0 to 2 | | | | | present) | 0 to 2 |
| | | | (T1 + T2) | | | | | | (T1 + T2) |
| Number of | N/A | 0 to 1 | 0 to 1 | N/A | 1 to 2 | 0 to 1 | N/A | 0 to 1 | 0 to 1 |
| signals | | (T1 | (T1 or T2) | | (T1 | (T1 or T2) | | (T1 | (T1 or (T2) |
| D1 & D2 pre- | | present) | 0 to 2 | | present) | 0 to 2 | | present) | 0 to 2 |
| PCR = Bkg | | | (T1 + T2) | | | (T1 + T2) | | | (T1 + T2) |

| | | | |
|---|---|---|---|
| Positive ΔS_{D1} | T₁ present | T₁ present | T₁ present |

| | | | |
|---|---|---|---|
| Positive ΔS_{D2} | T₁ or T₂ or T₁ + T₂ present | T₁ or T₂ or T₁ + T₂ present | T₁ or T₂ or T₁ + T₂ present |
| | ΔS_{D2} > ΔS_{D1} → T₂ present | ΔS_{D2} > ΔS_{D1} → T₁ + T₂ present | ΔS_{D2} > ΔS_{D1} → T₂ present |

### Scenario 1.

In a first non-limiting example, as described in Table 3, the first temperature is lower than the second temperature, and lower than the Tm A and Tm B. An increase in fluorescence at the first temperature will indicate the presence of target 1 and background signal will reflect the presence of quenched LOCS where both intact and/or split stems remain associated. The background at the first temperature and the second temperature may be measured prior to PCR at each temperature respectively. Alternatively, background measurement may be measured at a third temperature, where the third temperature is below both the first and second temperatures.

### Scenario 2

In a second non-limiting example, as described in Table 3, the first temperature is higher than the second temperature, and also higher than the Tm A and Tm B. An increase in fluorescence at the first temperature will indicate the presence of target 1 and background signal will reflect fluorescence from Intact and/or Split LOCS with dissociated stems. The background at the first temperature and the second temperature may be measured prior to PCR at each temperature respectively.

Scenario 3

In a third non-limiting example, as described in Table 3, the first temperature is lower than the second temperature, and lower than the Tm A and Tm B. An increase in fluorescence at the first temperature will indicate the presence of target 1 and background signal will reflect the presence of quenched LOCS where both intact and/or split stems remaining associated. The background at the first temperature and the second temperature may be measured prior to PCR at each temperature respectively. Alternatively, the background measurement may be measured at a third temperature, where the third temperature is below the second temperature but above the first temperature.

In other formats, for example detection by SPR, the first oligonucleotide and the LOCS probe can be attached to a gold nanoparticle (GNP) at one end and attached to a gold surface at the other end. In the absence of target, the first oligonucleotide can remain intact and the GNP would be in close proximity to the gold surface, producing a baseline level of SPR signal. In the presence of target 1, the first oligonucleotide can be cleaved, separating the GNP from the gold surface and producing a measurable shift in SPR signal, indicating the presence of the first target in the sample. This can be measured at a first temperature below the Tm A and Tm B such that GNP attached or associated with either intact or Split LOCS can remain on the surface. Measurement of a change in the SPR signal at a second temperature above the Tm of Split LOCS can indicate the presence of the first and/or the second target(s). Further, if a change in signal is observed at the first temperature and a greater change was observed at the second temperature this would indicate the presence of target 2. Alternatively if a signal is detected at the second temperature but not at the first, this would also be indicative of the second target only.

In another format, for example colorimetric detection, the first oligonucleotide probe and the LOCS can both be labelled at both ends with gold nanoparticles. At the first temperature below Tm A and Tm B, the first oligonucleotide can remain intact in the absence of target 1 wherein the GNP can be in an aggregated state in close proximity to each other, exhibiting a purple colour. However, the first oligonucleotide may be cleaved in the presence of target 1, separating the GNP and exhibiting a measurable colour change from purple to red. Therefore, a measurable colour change from purple to red can be indicative of the presence of target 1 in a sample. At a second temperature, which is above the first temperature and above Tm B but below Tm A, in the presence of the second target only, the LOCS can be split and the GNPs would in a dispersed state, and the colour will change from purple to red. If both targets are present, the shift in colour can be more intense.

In yet another format, the first oligonucleotide can be labelled with an electroactive moiety such as methylene blue or ferrocene at one end attached to an electrode surface at the other end. At the first temperature and in the absence of target 1, the first oligonucleotide can remain intact and the electroactive species in close proximity to the electrode surface, producing a baseline level of electrochemical signal (i.e. oxidation or reduction current). However, in the presence of target 1, the first oligonucleotide can be cleaved and the electroactive moiety can be released into solution, producing a measurable shift in electrochemical signal (i.e. oxidation or reduction current) above that of the baseline signal. Therefore, any measurable shift in electrochemical signal at this temperature can be indicative of the presence of target 1 in a sample.

### FURTHER EXEMPLARY EMBODIMENTS

In certain embodiments, reporter oligonucleotides including LOCS oligonucleotides of the present invention may be used to detect target directly. In other exemplary embodiments reporter probes or substrates may be used to detect target amplicons generated by target amplification technologies including, but not limited to, PCR, RT-PCR, SDA, NEAR, HDA, RPA, LAMP, RCA, TMA, 3SR, LCR, RAM or NASBA. Cleavage or degradation resulting in splitting of LOCS may occur in real time during target amplification or may be performed following amplification, at the end point of the reaction. The Loop region may be split by target-dependent cleavage or degradation mediated by the enzymatic activity of a catalytic nucleic acid including, but not limited to an MNAzyme, a DNAzyme, a ribozyme, or by the enzymatic activity of a protein enzyme including an exonuclease or an endonuclease. By way of non-limiting example, the exonuclease activity may be an inherent catalytic activity of, for example, a polymerase. By way of non-limiting example, the endonuclease activity may be an inherent catalytic activity of, for example, a restriction enzyme including a Nicking endonuclease, a riboendonuclease or a duplex specific nuclease (DSN).

Reactions of the present invention are designed to detect multiple targets simultaneously using a single LOCS oligonucleotide in combination with other type(s) of reporter probes. As would be evident to persons skilled in the art, standard reporter probes can further be combined with additional LOCS, for example, wherein each LOCS may comprise a different universal substrate within its Loop, and a different stem region capable of melting at a different temperature following cleavage of the substrate/Loop by different MNAzymes.

The reaction mix may further comprise additional reporter probes or substrates combined with a LOCS labelled with different fluorophore and quencher pairs. By way of non-limiting example, a Reporter oligonucleotide 1 and Intact LOCS oligonucleotide 2 may be labelled with fluorophore A, and Reporter oligonucleotide 3 and Intact LOCS oligonucleotide 4 may be labelled with fluorophore B. In an embodiment wherein, the Reporter oligonucleotides 1 and 3 are linear MNAzyme substrates, an MNAzyme 1 may form in the presence of target 1 and cleave the Reporter oligonucleotide 1 to generate fluorescence at all temperatures; and MNAzyme 2 may form in the presence of target 2 and cleave substrate 2 within LOCS oligonucleotide 2 resulting in a cleaved, split LOCS structure 2 containing Stem 2 which melts at temperature X. MNAzyme 3 may form in the presence of target 3 and cleave the Reporter oligonucleotide 3 to generate fluorescence at all temperatures. MNAzyme 4 may form in the presence of target 4 and cleave substrate 4 within LOCS oligonucleotide 4 resulting in a Split LOCS structure 4 containing Stem 4 which melts at temperature Y.

When fluorescence is analysed at a temperature below both X and Y, then excitation at the wavelength of Fluorophore A is indicative of target 1 and excitation at the wavelength of Fluorophore B is indicative of presence of target 3. When fluorescence is analysed at a temperature above both X and Y but below the melting temperatures of both Intact LOCS oligonucleotides 2 and 4, then excitation at the wavelength of Fluorophore A is indicative of target 1 and/or target 2 and excitation at the wavelength of Fluorophore B is indicative of the presence of target 3 and/or target 4.

When the melting profile of the reaction is analysed at the excitation wavelength of Fluorophore A, the presence of a peak at a first temperature X and the absence of a peak at second temperature 2, which is higher than temperature X, indicates the LOCS reporter 2 has been Split/Cleaved by an MNAzyme in the presence of a target 2. Alternatively, the absence of a peak at temperature X and the presence of a peak at temperature 2, indicates the LOCS reporter 2 remains intact and has not been cleaved by an MNAzyme due to the absence of target 2 in the sample. When the melting profile of the reaction is analysed at the excitation wavelength of Fluorophore B, the presence of a peak at a third temperature Y and the absence of a peak at fourth temperature 4, which is higher than temperature Y, indicates cleavage of a LOCS reporter 4 by an MNAzyme in the presence of target 4, whereas the absence of a peak at temperature Y and the presence of a peak at temperature 4, indicates the LOCS reporter 4 remains intact and has not been cleaved by an MNAzyme due to the absence of target 4 in the sample. As such analysis at two wavelengths, read in two channels on an instrument, can be used as a confirmatory tool to detect and differentiate targets, provided that the presence of the remaining two targets is determined using other means of detection such as real-time detection. The skilled person will recognise that the strategy can be extended to monitor cleavage of more than two targets at one specific wavelength and further the number of fluorophores analysed can be increased to that determined by the maximum capacity of the available instrument to discriminate individual wavelengths.

In a further exemplary embodiment, a linear MNAzyme substrate and a LOCS oligonucleotide may be combined wherein both contain the same fluorophore/quencher dye pair and the substrate regions are specific for a DNAzyme or a ribozyme, for example, a DNAzyme or ribozyme which can only be catalytically active in the presence of a specific metal ion. Specific DNAzymes and ribozymes are known in the art to require a metal cation cofactor to enable catalytic activity. For example, some DNAzymes and ribozymes can only be catalytically active in the presence of, for example, lead or mercury. Such metals may be present in, for example, an environmental sample. A reaction could include one linear MNAzyme substrate for a DNAzyme, which is, for example, mercury dependent, wherein the presence of mercury in a sample could result in cleavage of the linear MNAzyme substrate and generation of a fluorescent signal. The same reaction could also include a LOCS reporter which contains a loop comprising a substrate for a DNAzyme, which is, for example, lead dependent, wherein the presence of lead in a sample could result in cleavage of the LOCS and generation of a fluorescent signal at a temperature higher than the Tm of the split LOCS. An increase in fluorescence at a first temperature, which is below the Tm of the Split LOCS, would indicate the presence of mercury. An increase in fluorescence at a second temperature, which is above the Tm of the Split LOCS but below the Tm of the Inatact LOCS, would indicate the presence of mercury and/or lead. If mercury were detected at the first temperature, then a further increase in fluorescence at the second temperature would indicate the presence of lead. This could be confirmed by allowing the reaction to be cooled so that the stem of the cleaved, Split LOCS structure re-anneals, and then performing melt curve analysis to determine the presence or absence of peaks indicative of the presence of split LOCS structures. One skilled in the art would readily recognize that multiple probes cleavable in the presence of specific metal cofactors, could be combined in a single reaction and detected either in real time or at the end of the reaction.

Non-limiting examples of target nucleic acids (i.e. polynucleotides), which may be detected using LOCS oligonucleotides in combination with other well-known probes types could include DNA, methylated DNA, alkylated DNA, complementary DNA (cDNA), RNA, methylated RNA, microRNA, siRNA, shRNA, mRNA, tRNA, snoRNA, stRNA, smRNA, pre- and pri-microRNA, other non-coding RNAs, ribosomal RNA, derivatives thereof, amplicons thereof or any combination thereof (including mixed polymers of deoxyribonucleotide and ribonucleotide bases).

### Generation of Detectable Signals

The methods and compositions of the present invention utilise detection moieties to provide detectable signals. The nature of the detectable signal that the moieties are capable of producing will depend on the type of detection moiety and/or the conformation of the oligonucleotide to which it is associated.

Any suitable detectable moiety can be utilised that is capable of providing a detectable signal upon the modification of an oligonucleotide to which it is associated. Non-limiting examples of suitable detectable moieties include fluorophores for fluorescent signal generation, nanoparticles for colorimetric or SPR signal generation, reactive moieties (e.g. alkaline phosphatase or peroxidase enzymes) for chemiluminescent signal generation, electroactive species for electrochemical signal generation, and any combination thereof. By way of non-limiting example, suitable electroactive species include Methylene blue, Toluene Blue, Oracet Blue, ferrocene, Hoechst 33258, [Ru(phen)3]2+ or Daunomycin and the most common electrode materials include gold, glassy carbon, pencil graphite or carbon ionic liquid, Methods for the detection and measurement of fluorescent, chemiluminescent, colorimetric, surface plasmon resonance (SPR) and electrochemical signals are well known to persons skilled in the art.

By way of non-limiting example, oligonucleotides of the present invention, including LOCS, may have one or more fluorophores attached. The detectable signal inherently generated by the fluorophore may be quenched due to proximity to one or more quencher molecules. For example and without limitation, the fluorophore(s) may be attached to a single strand of a double-stranded stem portion (e.g. at the 5′ or 3′ terminus) of a Molecular Beacon or a LOCS, and the quencher(s) may be attached to an opposing strand of the double-stranded stem portion (e.g. at the 5′ or 3′ terminus). Alternatively, the quencher(s) may be attached to another entity (e.g. a surface or another oligonucleotide) to which the oligonucleotide is bound such that the detectable signal inherently generated by the fluorophore may be quenched. In the presence of a target, the oligonucleotide may undergo a modification that distances the fluorophore(s) from the quencher molecule(s) thus generating a detectable signal.

Additionally or alternatively, the oligonucleotides (including LOCS) may be attached to GNP for colorimetric detection. When gold nanoparticles are aggregated in close proximity to each other they exhibit a purple colour (i.e. absorbance at a longer wavelength) and when gold nanoparticles are separated they exhibit a red colour (i.e. absorbance at a shorter wavelength) wherein, a measurable colour change from purple to red (e.g. LOCS, linear MNAzyme substrates, Catcher-Pitcher probes, TaqMan probes and restriction enzyme probes) or alternatively from red to purple (e.g. dual hybridisation probes) is indicative of the presence of a specific target in a sample.

Additionally or alternatively, the oligonucleotides (including LOCS) and/or oligonucleotide components may be attached to a GNP and/or a gold surface for SPR detection of a target in a sample. When GNPs move into close proximity, or alternatively when they move away from a gold surface, they can generate a change in measurable SPR signal where a decrease in SPR signal using some approaches (e.g. LOCS, linear MNAzyme substrates, TaqMan probes and restriction enzyme probes) can be indicative of the presence of a specific target in a sample or alternatively wherein an increase in SPR signal using other approaches (e.g. Catcher-Pitcher probes and dual hybridisation probes) can be indicative of the presence of a specific target in a sample.

Additionally, or alternatively, the oligonucleotide reporter and probes (including LOCS) and/or oligonucleotide components may be attached to electroactive species and/or on an electrode surface for electrochemical detection. When the oligonucleotides attached to electroactive species move into close proximity with, or alternatively when they move away from, an electrode surface they can generate a measurable change in oxidation or reduction current. In some embodiments (e.g. LOCS, linear MNAzyme substrates, TaqMan probes and restriction enzyme probes), the resulting measurable signal arising from an electroactive species moving away from the electrode surface is indicative of the presence of a specific target in a sample Alternatively, in other embodiments (e.g. Catcher-Pitcher probes and dual hybridisation probes), the resulting measurable signal arising from an electroactive species moving into close proximity to the electrode surface is indicative of the presence of a specific target in a sample.

In some embodiments, the compositions and methods of the present invention utilise LOCS attached to a specific detection moiety in combination with another oligonucleotide probe that is attached to the same detection moiety, or a similar detection moiety that generates a detectable signal capable of being detected simultaneously with signal generated by the detectable moiety of the LOCS (e.g. using a single type of detector such as one fluorescence channel, or a specific mode of colorimetric, surface plasmon resonance (SPR), chemiluminescent, or electrochemical detection).

### Analyses of Fluorescent Signals

Without limitation and by way of example only, detectable moieties used in accordance with the present invention include fluorescent signals generated by these detectable moieties upon modification, cleavage or digestion of oligonucleotide probes to which they are attached, coupled, or otherwise associated, including dissociation of split LOCS structures, can be analysed in any suitable manner to detect, differentiate, and/or quantify target molecules in accordance with the methods of the present invention.

While standard melting curve analyses can be used, various other approaches are disclosed and exemplified herein (see Examples) which can be readily adopted to the analysis of various assay formats. associated

By way of non-limiting example, measurements of fluorescent signal at a single temperature, or at multiple temperatures, may be obtained at various time points within a reaction suitable for detecting cleavage or degradation of the loop regions of LOCS oligonucleotides. By way of non-limiting examples, these time points may comprise (i) a time point at, or near, the initiation of a reaction, and/or (i) a single time point, or multiple time points, during the course of the reaction; and/or (iii) a time point at the conclusion or endpoint of the reaction.

In some embodiments, measurement of fluorescent signal may be obtained at two or more temperatures at each cycle during an amplification reaction, such as PCR amplification. Analysis may be performed by comparing levels of fluorescence obtained at a first and/or second temperature and/or at a further temperature.

In several embodiments, measurement of fluorescent signal may be obtained at two temperatures in reactions which are tailored to measure two targets at the same wavelength.

In some embodiments a first target may be detected using a first oligonucleotide reporter probe or substrate where fluorescent signals can be measured at multiple time points, or at multiple cycles, for example, at each cycle during PCR. In the same reaction a second target may be detected using a LOCS probes by comparing pre-PCR and post PCR fluorescence levels. In such embodiments, quantitative data may be determined for the first target, whilst qualitative data may be generated for the second target. By way of non-limiting example, the first oligonucleotide probe may be an MNAzyme substrate cleaved by a first MNAzyme in the presence of a first target and monitored in real time; whereas a LOCS probe may be cleaved by a second MNAzyme in the presence of a second and monitored using endpoint detection analysis. Examples of combining real time quantitative analysis and endpoint qualitative analysis include Example 1, which utilizes one linear MNAzyme Substrate and one LOCS probe, and Example 6 which utilizes one TaqMan probe and one LOCS probe.

In some embodiments an increase in fluorescence at the first temperature is indictive of the presence of the first target and an increase in fluorescence at the second temperature is indictive of the presence of the first and/or second targets. In other embodiments an increase in fluorescence at the first temperature is indictive of the presence of the first target and an increase in fluorescence at the second temperature is indictive of the presence of the second target. In yet other embodiments a decrease in fluorescence at the first temperature is indictive of the presence of the first target and an increase in fluorescence at the second temperature is indictive of the presence of the second target. In other embodiments, measurement of fluorescent signal may be obtained at two or more temperatures at each cycle during PCR, and amplification curves may be plotted for each series of measurement obtained at each temperature. Threshold fluorescence values can be assigned to each amplification plot for each specific temperature and Cq values may be measured as the cycle number where the amplification plots cross the threshold values. In embodiments wherein a first probe is a standard linear MNAzyme substrate for detection of target 1 and the second probe is a LOCS reporter for detection of target 2; and wherein measurement of fluorescent signal is obtained at two temperatures at each cycle during PCR, the Cq measured using fluorescent signal from the linear MNAzyme substrate at the lower temperature, which is below that of the Split LOCS Tm, may allow direct quantification of the starting concentration of a first target; and the Cq measured using the total fluorescent signal from both the linear MNAzyme substrate and the LOCS reporter at the higher temperature, which is above the Tm of the Split LOCS but below the Tm of the Intact LOCS, may be analysed as exemplified in Example 4, thus allowing quantification of the starting concentration of a second target.

In other embodiments, measurement of fluorescent signal may be obtained at two or more temperatures at each cycle during PCR, and amplification curves may be plotted for each series of measurement obtained at each temperature. Threshold fluorescence values can be assigned to each amplification plot for each specific temperature and Cq values may be measured as the cycle number where the amplification plots cross the threshold values. In embodiments wherein a first probe for detection of a first target, being either a standard Molecular Beacon, or a Catcher/Pitcher probe, or a Scorpion Uni-Probe, or a Scorpion Bi-Probe, is combined with a second probe for detection of a second target, which may be a LOCS reporter; and wherein measurement of fluorescent signal is obtained at two temperatures at each cycle during PCR, the Cq measured using fluorescent signal from the first probe at the lower temperature, which is below the Tm of a Split LOCS, may allow direct quantification of the starting concentration of a first target; and the Cq measured using fluorescent signal from the LOCS reporter at the higher temperature, which is above the Tm of the Split LOCS but below the Tm of the Intact LOCS, may allow direct quantification of the starting concentration of a second target. Specific combinations of a first standard probe with a second LOCS probe may have additional requirements for the Tm of specific regions of the first probe, and for the two temperatures at which data is acquired, as outlined above and as exemplified for Molecular Beacons in Examples 5 and 7; for Scorpion Probes in Example 9, and for Catcher Pitcher oligonucleotides in Example 11.

By way of non-limiting example, baseline fluorescence signal can be obtained by measuring fluorescence at selected temperatures, for example a first and second temperature, at a time point which is either at, or near, the initiation of a reaction, for example pre-PCR. Prior to PCR and at a first temperature, a standard reporter probe, for example a linear MNAzyme substrate or a TaqMan probe or a Molecular Beacon, and the Intact LOCS would be quenched and not producing significant fluorescence signal, providing this temperature is below the Tm of the stem of the Intact LOCS (and Molecular Beacon if present). Analysis may be performed by comparing levels of fluorescence obtained at the first and second temperature at a time point at the initiation of a reaction (e.g. pre-PCR) and levels of fluorescence obtained at the first and second temperatures at a time point during and/or after the reaction (e.g. during PCR or post-PCR).

As demonstrated in Example 1, cleavage of a linear MNAzyme substrate, measured at a first temperature post-PCR time point, produces significant fluorescence signal relative to signal of an intact, linear MNAzyme substrate measured at a first temperature pre-PCR timepoint. This relative signal (ΔS_{D1}) crosses a first pre-determined threshold which indicates the presence of a first target in a sample. At this first temperature, an intact, LOCS and/or a cleaved, split LOCS do not contribute to production of significant fluorescence signal relative to signal obtained pre-PCR due to the Tm of the stem of both LOCS structures. At a second temperature, which is higher than the first temperature, and at a post-PCR time-point, cleavage of the LOCS produces significant fluorescence signal, relative to signal obtained at a second temperature at a pre-PCR time-point. This relative signal (ΔS_{D2}) crosses a second pre-determined threshold and is greater than the relative signal at the first temperature (ΔS_{D1}), as demonstrated in Example 1 (Endpoint Analysis Method 1), regardless of whether the linear MNAzyme substrate is cleaved or not and thus indicates detection of the split LOCS and hence the presence of a second target. At this second temperature and at a time-point post-PCR, an intact LOCS does not contribute to production of significant fluorescence signal relative to a pre-PCR time-point at the same second temperature and does not cross a pre-determined threshold. Alternatively, the difference between the relative signal obtained pre- and post-PCR at a second temperature and the relative signal obtained pre- and post-PCR at a first temperature (ΔS_{D2}−ΔS_{D1}) can be compared to a pre-determined threshold to determine the presence of a cleaved, split LOCS. As demonstrated in Example 1 (Endpoint Analysis Method 2), the presence of a cleaved, split LOCS, indicating the presence of a second target in a sample, can be determined when the difference is greater than a pre-determined threshold (ΔS_{D2}−ΔS_{D1}>threshold). The absence of a second target in a sample can be determined when the difference value is lower than a pre-determined threshold (ΔS_{D2}−ΔS_{D1}<threshold). Additionally, when the difference between the relative signal obtained pre- and post-PCR at a second temperature and the relative signal obtained pre- and post-PCR at a first temperature (ΔS_{D2}−ΔS_{D1}) crosses the pre-determined threshold, the unique ratio of the fluorescence changes at temperature 1 to temperature 2 (ΔS_{D1}:ΔS_{D2}), or the inverse ratio (ΔS_{D2}: ΔS_{D1}), can be used to determine whether target 1, target 2 or both targets 1 and 2 are present within the sample. As demonstrated in Example 1 (Endpoint Analysis Method 3), if ΔS_{D1}:ΔS_{D2} is greater than a threshold R₁, this indicates that target 1 only is present; if ΔS_{D1}:ΔS_{D2} is less than threshold R₂, this indicates that target 2 only is present; and if ΔS_{D1}:ΔS_{D2} is less than threshold R₁ but greater than threshold R₂, this indicates both target 1 and target 2 are present.

Exemplary Applications of LOCS Oligonucleotides when Combined with Other Standard reporter/probes

Detection of Targets During or Following Target Amplification

LOCS oligonucleotides of the present invention may be used determine the presence of amplified target nucleic acid sequences. No particular limitation exists in relation to amplification techniques to which the LOCS reporters may be applied. Amplicons generated by various reactions may be detected by LOCS reporters, provided the presence of target amplicons can promote the cleavage or degradation of LOCS reporter to produce Split LOCS structures. Non-limiting examples of methods useful in cleaving or degrading Loop regions contained within LOCS structures include cleavage by MNAzymes, DNAzymes, ribozymes, restriction enzymes, endonucleases or degradation by exonucleases including but not limited to the exonuclease activity of a polymerase.

In general, nucleic acid amplification techniques utilise enzymes (e.g. polymerases) to generate copies of a target nucleic acid that is bound specifically by one or more oligonucleotide primers. Non-limiting examples of amplification techniques in which LOCS oligonucleotides may be used include one or more of the polymerase chain reaction (PCR), the reverse transcription polymerase chain reaction (RT-PCR), strand displacement amplification (SDA), helicase dependent amplification (HDA), Recombinase Polymerase Amplification (RPA), loop-mediated isothermal amplification (LAMP), rolling circle amplification (RCA), transcription-mediated amplification (TMA), self-sustained sequence replication (3SR), nucleic acid sequence based amplification (NASBA), Ligase Chain Reaction (LCR) or Ramification Amplification Method (RAM).

The skilled addressee will readily understand that the applications of LOCS oligonucleotides described above are provided for the purpose of non-limiting exemplification only. The LOCS oligonucleotides disclosed may be used in any primer-based nucleic acid amplification technique and the invention is not so limited to those embodiments specifically described.

Detection of Amplicons Generated Using LOCS Reporters

As discussed above, LOCS reporters of the present invention may be utilised in any polynucleotide amplification technique, non-limiting examples of which include the PCR, RT-PCR, SDA, HDA, RPA, LAMP, RCA, TMA, RAM, LCR, 3SR, or NASBA.

Amplicons generated by these techniques may be detected utilizing LOCS reporters which may be may cleaved or degraded using any suitable method known in the art. Non-limiting examples include the use of catalytic nucleic acids, exonucleases, endonucleases and the like.

An MNAzyme may be utilised to generate Split LOCS reporters by detecting amplicons generated through methods such as PCR, RT-PCR, SDA, HDA, RPA, TMA, LAMP, RCA, LCR, RAM, 3SR, and NASBA. The MNAzyme may comprise one or more partzyme(s). MNAzymes are multi-component nucleic acid enzymes which are assembled and are only catalytically active in the presence of an assembly facilitator which may be, for example, a target to be detected such as an amplicon generated from a polynucleotide sequence using primers. MNAzymes are composed of multiple part-enzymes, or partzymes, which self-assemble in the presence of one or more assembly facilitators and form active MNAzymes which catalytically modify substrates. The substrate and assembly facilitators (target) are separate nucleic acid molecules. The partzymes have multiple domains including (i) sensor arms which bind to the assembly facilitator (such as a target nucleic acid); (ii) substrate arms which bind the substrate, and (iii) partial catalytic core sequences which, upon assembly, combine to provide a complete catalytic core. MNAzymes can be designed to recognize a broad range of assembly facilitators including, for example, different target nucleic acid sequences. In response to the presence of the assembly facilitator, MNAzymes modify their substrates. This substrate modification can be linked to signal generation and thus MNAzymes can generate an enzymatically amplified output signal. The assembly facilitator may be a target nucleic acid present in a biological or environmental sample (e.g. an amplicon generated from a polynucleotide target using primers). In such cases, the detection of the modification of the substrate by the MNAzyme activity is indicative of the presence of the target. Several MNAzymes capable of cleaving nucleic acid substrates are known in the art. MNAzymes and modified forms thereof are known in the art and disclosed in PCT patent publication numbers WO/2007/041774, WO/2008/040095, WO2008/122084, and related US patent publication numbers 2007-0231810, 2010-0136536, and 2011-0143338 (the contents of each of these documents are incorporated herein by reference in their entirety).

Use of LOCS Reporters as Internal Calibrator for Machine-to-Machine Variation or Well-to-Well Variation

The calibrator method demonstrated in Example 12 has several advantages including that it does not require the use of additional reagents to be added to the reaction nor does it require the use of data obtained from other wells. This method functions to calibrate and correct for well-to-well variations that may be present. Furthermore, the calibration is processed using the data acquired in the same channel and therefore is not affected by any channel-to-channel variations that may be present between the instruments. Where multiple channels are utilised for a multiplex reaction, each channel can be independently calibrated against the LOCS calibration signal in each channel. This is favorable to a scenario where the signals are calibrated against signals in a different channel, such as that from the internal control or endogenous control, as the calibration is adversely affected if the ratio of the expected signal intensity between the channels differs significantly between the instruments, causing channel-to-channel variations.

Diagnostic Applications

Methods using standard report probes in combination with LOCS oligonucleotides may be used for diagnostic and/or prognostic purposes in accordance with the methods described herein. The diagnostic and/or prognostic methods may be performed ex vivo or in vitro. However, the methods of the present invention need not necessarily be used for diagnostic and/or prognostic purposes, and hence applications that are not diagnostic or prognostic are also contemplated.

In some embodiments, the methods described herein may be used to diagnose infection in a subject. For example, the methods may be used to diagnose infection by bacteria, viruses, fungi/yeast, protists and/or nematodes in the subject. In one embodiment, the virus may be an enterovirus. The subject may be a bovine, equine, ovine, primate, avian or rodent species. For example, the subject may be a mammal, such as a human, dog, cat, horse, sheep, goat, or cow. The subject may be afflicted with a disease arising from the infection. For example, the subject may have meningitis arising from an enterovirus infection. Accordingly, methods of the present invention may in certain embodiments be used to diagnose meningitis.

The methods of the present invention may be performed on a sample. The sample may be derived from any source. For example, the sample may be obtained from an environmental source, an industrial source, or by chemical synthesis.

It will be understood that a “sample” as contemplated herein includes a sample that is modified from its original state, for example, by purification, dilution or the addition of any other component or components.

The methods of the present invention including, but not limited to diagnostic and/or prognostic methods, may be performed on a biological sample. The biological sample may be taken from a subject. Stored biological samples may also be used. Non-limiting examples of suitable biological samples include whole blood or a component thereof (e.g. blood cells, plasma, serum), urine, stool, saliva, lymph, bile fluid, sputum, tears, cerebrospinal fluid, bronchioalveolar lavage fluid, synovial fluid, semen, ascitic tumour fluid, breast milk and pus.

### Kits

The present invention provides kits comprising one or more agents for performing methods of the present invention. Typically, kits for carrying out the methods of the present invention contain all the necessary reagents to carry out the method.

In some embodiments the kits may comprise oligonucleotide components capable of forming an MNAzyme in the presence of an appropriate assembly facilitator (e.g. an amplicon as described herein). For example, the kit may comprise at least a first and second oligonucleotide component comprising a first and second partzyme, and a second container comprising a substrate, wherein self-assembly of the first and second partzymes, and the substrate, into an MNAzyme requires association of an assembly facilitator (e.g. an amplicon) present in a test sample. Accordingly, in such embodiment, the first and second partzymes, and a LOCS oligonucleotide comprising a substrate within the Loop region, may be applied to the test sample in order to determine the presence of one or more target amplicons. In general, the kits comprise at least one LOCS oligonucleotide provided herein.

Typically, the kits of the present invention will also comprise other reagents, wash reagents, enzymes and/or other reagents as required in the performance of the methods of the invention such as PCR or other nucleic acid amplification techniques.

The kits may be fragmented kits or combined kits as defined herein.

Fragmented kits comprise reagents that are housed in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion.

Such kits may also include a container which will accept the test sample, a container which contains the reagents used in the assay, containers which contain wash reagents, and containers which contain a detection reagent.

Combined kits comprise all of the components of a reaction assay in a single container (e.g. in a single box housing each of the desired components).

A kit of the present invention may also include instructions for using the kit components to conduct the appropriate methods. Kits and methods of the invention may be used in conjunction with automated analysis equipment and systems, for example, including but not limited to, real time PCR machines.

For application to amplification, detection, identification or quantitation of different targets, a single kit of the invention may be applicable, or alternatively different kits, for example containing reagents specific for each target, may be required. Methods and kits of the present invention find application in any circumstance in which it is desirable to detect, identify or quantitate any entity.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be further described in greater detail by reference to the following specific examples, which should not be construed as in any way limiting the scope of the invention.

### Example 1

Method for analysis of multiple targets at single wavelength using one linear MNAzyme substrate and one LOCS probe in a format allowing either simultaneous real-time quantification of one target combined with qualitative endpoint detection of a second target, or simultaneous qualitative endpoint analysis of two targets per channel.

The following example demonstrates an approach where one linear substrate and one LOCS reporter are used in combination for detection and differentiation of two targets (CTcry and NGopa) and for quantification of one target (CTcry) in a single fluorescent channel without the need for melt curve analysis. The assay is designed such that CTcry can be detected and quantified using a linear MNAzyme substrate, and NGopa can be detected and differentiated at endpoint using a LOCS reporter which comprises a different MNAzyme substrate within its Loop.

During PCR, MNAzyme 1 can cleave linear substrate 1 in the presence of CTcry to separate a fluorophore and quencher to produce an increase in signal that can be detected across a broad range of temperatures. In this example, real-time detection and quantification of CTcry is achieved by acquiring fluorescence at each cycle during PCR at temperature 1 (D₁) (52° C.). The stem of LOCS-1 in both the intact and split configurations has a melting temperature (Tm) that is higher than temperature 1 (52° C.) and therefore LOCS-1 does not contribute signal at temperature 1 regardless of the presence or absence of NGopa in the sample.

In the presence of NGopa, the MNAzyme 2 can cleave LOCS-1 during PCR. Endpoint detection of NGopa can be achieved by comparing fluorescence signal at a higher temperature 2 (70° C.) prior to and following amplification (ΔS_{D2}). Since the Tm of the intact LOCS-1 stem is higher than temperature 2 (Tm>70° C.), and the Tm of the split LOCS-1 stem is lower than temperature 2 (Tm<70° C.), then the increase in fluorescence during PCR, above that which is related to cleaved linear substrate 1 at this temperature (if present), is associated with split LOCS-1 with dissociated stems and is indicative of the presence of NGopa.

In the present example, real-time quantification of target 1 (CTcry) coupled with endpoint detection of Target 2 (NGopa) is demonstrated. Also, in this example endpoint detection of both target 1 (CTcry) and target 2 (NGopa) is demonstrated along with multiple alternative methods of analysis of these data.

The following Endpoint Analysis Methods (1 to 3) require measurement of fluorescence at discrete time points only, namely; at, or near, the initiation of the amplification reaction (pre-PCR fluorescence) and following amplification (endpoint or post-PCR fluorescence). Readings are taken at these time points at multiple temperatures (D₁ and D₂) and analysis allows elucidation of the presence of target 1, or target 2, or targets 1 and 2, or neither target 1 nor target 2.

### Endpoint Analysis Method 1

At temperature 1 (D₁), target-mediated cleavage of substrate 1 produces a significant increase in fluorescence signal during PCR (ΔS_{D1}), which is measured as the difference between the post-PCR fluorescence at D₁ (S_{D1-post-PCR}) and pre-PCR fluorescence at D₁ (S_{D1-pre-PCR}), and which exceeds a first threshold (X₁) such that S_{D1-post-PCR}−S_{D1-pre-PCR}=ΔS_{D1}>X₁. In contrast at this temperature, if cleavage of LOCS-1 has occurred, this does not produce a significant increase in fluorescence signal and thus does not exceed the threshold (X₁). This occurs because the Tm of the split stem is higher than D₁ and the structure remains quenched. Therefore, the comparison of pre-PCR and post-PCR measurements of fluorescence at temperature 1 allows for a specific detection of the cleaved substrate 1 and hence target 1.

At temperature 2 (D₂), which is higher than temperature 1 (D₁), target-mediated cleavage of LOCS-1 produces a significant increase in fluorescence signal during amplification. In the presence of split LOCS-1, the increase in signal at D₂ (ΔS_{D2}) is greater than threshold X₁. Further, the increase in signal at D₂ (ΔS_{D2}) is greater than that at D₁ (ΔS_{D1}), regardless of whether substrate 1 is cleaved or not. Therefore, when the magnitude of the increase in fluorescence at D₂ (ΔS_{D2}), measured as a difference between the post-PCR fluorescence at D₂ (S_{D2-post-PCR}) and pre-PCR fluorescence at D₂ (S_{D2-pre-PCR}) such that S_{D2-post-PCR}−S_{D2-pre-PCR}=ΔS_{D2}, exceeds both that at D₁ (ΔS_{D1}) and threshold X₁, such that ΔS_{D2}>ΔS_{D1} and ΔS_{D2}>threshold X₁, this indicates detection of the split LOCS-1 and hence the presence of target 2.

### Endpoint Analysis Method 2

At temperature 1 (D₁), cleavage of substrate 1 produces a significant increase in fluorescence signal (ΔS_{D1}) during PCR that exceeds a first threshold (X₁) such ΔS_{D1}>X₁; however, cleavage of LOCS-1 does not produce significant increase in fluorescence signal at D₁ and does not exceed a threshold (X₁) due to the high Tm of the stem which exceeds D₁ when either intact or split. Therefore, comparison of pre-PCR and post-PCR fluorescence measurements at D₁ (ΔS_{D1}) allows for a specific detection of the cleaved substrate 1 and hence target 1.

At temperature 2 (D₂), which is higher than temperature 1 (D₁), cleavage of LOCS-1 throughout PCR produces a change in fluorescence signal (ΔS_{D2}) which is greater than the change observed at temperature 1 (ΔS_{D1}) wherein the difference between ΔS_{D2} and ΔS_{D1} (ΔS_{D2}−ΔS_{D1}) crosses a second threshold (X₂); such that ΔS_{D2}−ΔS_{D1}=ΔΔS_{D2}ΔS_{D1}>X₂. In contrast, cleavage of substrate 1 alone produces similar ΔS_{D2} and ΔS_{D1} values, wherein the difference between these two values (ΔΔS_{D2} ΔS_{D1}) is not significant and does not cross a second threshold (X₂). Therefore, the analysis of fluorescence at temperature 2 in this manner allows for a specific detection of the cleaved, split LOCS-1 which is indicative of the presence of target 2.

### Endpoint Analysis Method 3

When either target 1 and/or target 2 is present within a sample, the increase in signal during amplification at temperature 2 (ΔS_{D2}) is significant and crosses a threshold X₁. Therefore, when ΔS_{D2}>X₁, this is indicative that either CTcry and/or NGopa are present within the sample. Furthermore, when ΔS_{D2}<X₁ this is indicative that neither CTcry or NGopa are present in the sample.

Further, when ΔS_{D2}>X₁, the unique ratio of the fluorescence changes at temperature 1 to temperature 2 (ΔS_{D1}:ΔS_{D2}), or the inverse ratio (ΔS_{D2}:ΔS_{D1}), can be used to determine whether target 1, target 2 or both targets 1 and 2 are present within the sample.

In this case, if ΔS_{D1}:ΔS_{D2} is greater than threshold R₁, this indicates that target 1 only is present; if ΔS_{D1}:ΔS_{D2} is less than threshold R₂, this indicates the target 2 only is present; and if ΔS_{D1}:ΔS_{D2} is less than threshold R₁ but greater than threshold R₂, this indicates both target 1 and target 2 are present.

### Endpoint Analysis Methods 1, 2 and 3

Furthermore, in the Endpoint Analysis Methods 1-3 above, the changes in signals at temperatures 1 and 2 (ΔS_{D1} and ΔS_{D2}) can also be calibrated against signal from a calibrator (C) or the difference between the post-PCR and pre-PCR signal from a calibrator (ΔC). By means of non-limiting example, a calibrator could include an endogenous control, an internal control or designated calibrator oligos, which may be measured in the same channel or in a different channel at a predefined temperature or temperatures. The changes in signals at temperatures 1 and 2 (ΔS_{D1} and ΔS_{D2}) can be expressed as a ratio to a change in signal from a calibrator (E.g. ΔS_{D2}/AC, ΔS_{D1}/AC) or alternatively as a reciprocal ratio (E.g. ΔC/ΔS_{D2}, ΔC/ΔS_{D1}), where ΔC is determined as a positive signal, such that ΔC is greater than threshold C (ΔC>threshold C).

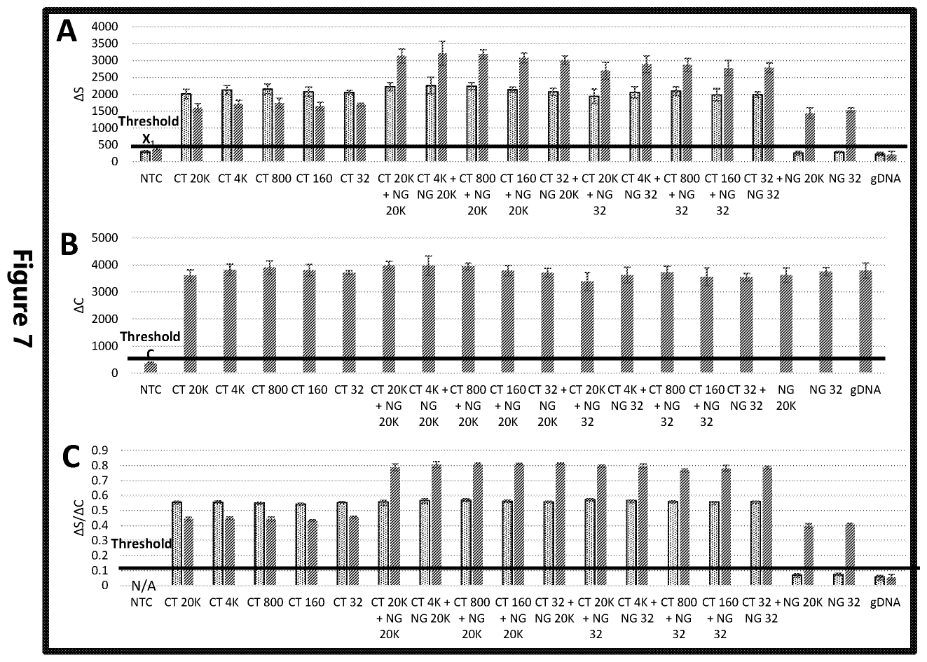

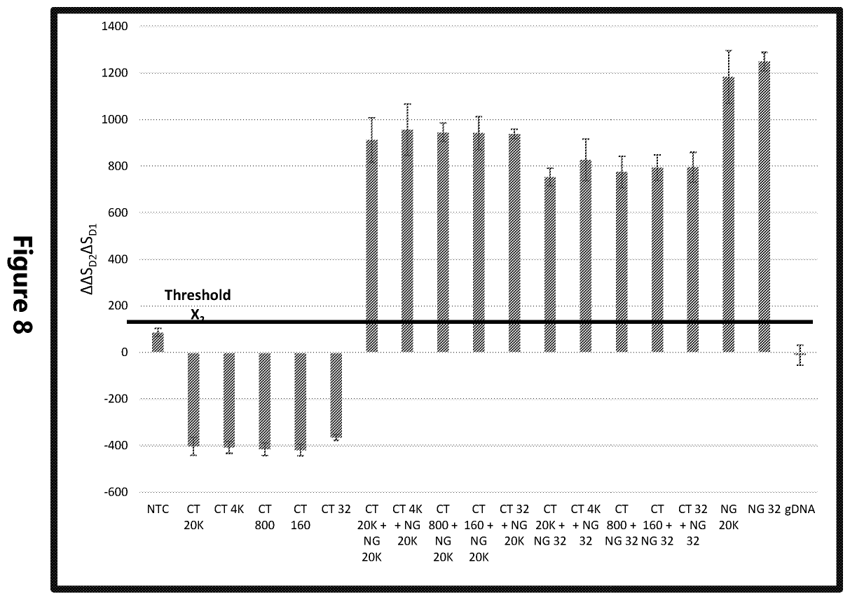

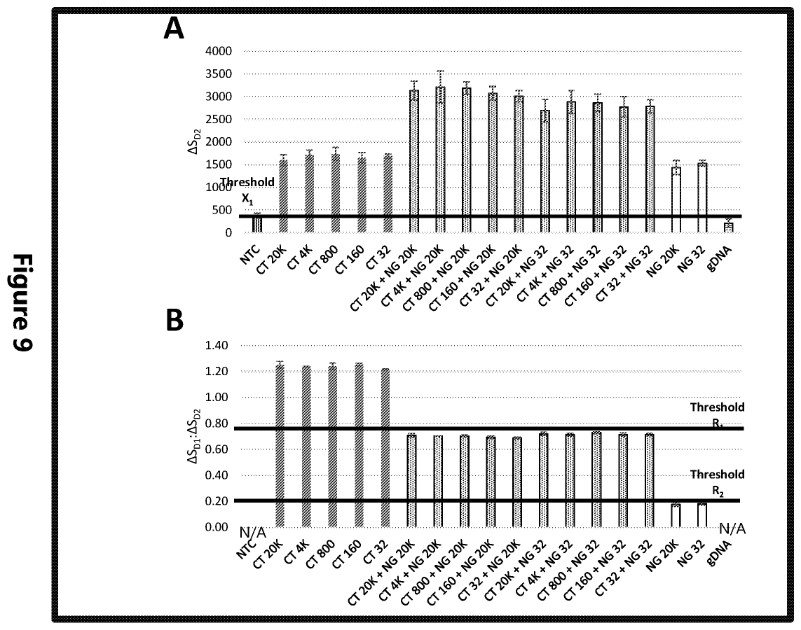

TABLE 4
Summary of analytical protocols allowing specific detection
of cleaved linear substrate and/or split LOCS reporters
in a single channel using measurements of Relative
Fluorescence Units (RFU) collected at specific temperature-points.

| | Criteria for determining | | |
|---|---|---|---|
| | the presence of cleaved | | |
| | linear substrate 1 and/or | Target(s) | Illustrated |
| Method | split LOCS in the sample | present | in FIG. # |
| 1 | Cleaved Substrate 1: | target 1 | FIG. 7 |
| | S_{D1-post-PCR} − S_{D1-pre-PCR} = | | |
| | ΔS_{D1} > threshold X₁ | | |
| | (or ΔS_{D1}/ΔC > threshold | | |
| | X₁ and ΔC > threshold C) | | |
| | Cleaved LOCS-1: | target 2 | |
| | S_{D2-post-PCR} − S_{D2-pre-PCR} = | | |
| | ΔS_{D2} > ΔS_{D1} | | |
| | (or ΔS_{D2}/ΔC > S_{D1}/ΔC and | | |
| | ΔC > threshold C); and | | |
| | ΔS_{D2} > threshold X₁ (or | | |
| | ΔS_{D2}/ΔC > threshold X₁ and | | |
| | ΔC > threshold C) | | |
| 2 | Cleaved Substrate 1: | target 1 | FIG. 7 |
| | ΔS_{D1} > threshold X₁ | | |
| | (or ΔS_{D1}/ΔC > threshold X₁ | | |
| | and ΔC > threshold C) | | |
| | Cleaved LOCS-1: | target 2 | FIG. 8 |
| | (ΔS_{D2} − ΔS_{D1}) = | | |
| | ΔΔS_{D2}ΔS_{D1} > threshold X₂ | | |
| | (or ΔS_{D2}/ΔC − ΔS_{D1}/ΔC > | | |
| | threshold X₂ and ΔC > | | |
| | threshold C) | | |
| 3 | ΔS_{D2} > threshold X₁ | target 1 | FIG. 9 |
| | (or ΔS_{D2}/ΔC > threshold X₁ | and/or | |
| | and ΔC > threshold C) | target 2 | |
| | ΔS_{D1}:ΔS_{D2} > threshold R₁ | target 1 | FIG. 9 |
| | ΔS_{D1}:ΔS_{D2} < threshold R₂ | target 2 | |
| | threshold R₁ > ΔS_{D1}: ΔS_{D2} > | Both | |
| | threshold R₂ | targets 1 | |
| | | and 2 | |

### Oligonucleotides

The oligonucleotides specific to this experiment include; Forward primer 1 (SEQ ID NO: 1), Reverse primer 1 (SEQ ID NO: 2), Partzyme A1 (SEQ ID NO: 3), Partzyme B1 (SEQ ID NO: 4), Forward primer 2 (SEQ ID NO: 5), Reverse primer 2 (SEQ ID NO: 6), Partzyme A2 (SEQ ID NO: 7), Partzyme B2 (SEQ ID NO: 8), Forward primer 3 (SEQ ID NO: 9), Reverse primer 3 (SEQ ID NO: 10), Partzyme A3 (SEQ ID NO: 11), Partzyme B3 (SEQ ID NO: 12), linear MNAzyme Substrate 1 (SEQ ID NO: 13), LOCS-1 (SEQ ID NO: 14) and linear MNAzyme Substrate 2 (SEQ ID NO: 15). The sequences are listed in the sequence listing.

The oligonucleotides specific for target 1 (CTcry) amplification and detection are Substrate 1, Partzyme A1, Partzyme B1 (MNAzyme 1), Forward Primer 1 and Reverse Primer 1. The oligonucleotides specific for target 2 (NGopa) amplification and detection are LOCS-1, Partzyme A2, Partzyme B2 (MNAzyme 2), Forward Primer 2 and Reverse Primer 2. The oligonucleotides specific for calibrator gene (TFRC) amplification and detection are Substrate 2, Partzyme A3, Partzyme B3 (MNAzyme 3), Forward Primer 3 and Reverse Primer 3.

### Reaction Conditions

Real-time amplification and detection were performed in a total reaction volume of 20 μL using a BioRad® CFX96 thermocycler. The cycling parameters, and fluorescent data acquisition (DA) points, were: 1 cycle of 95° C. for 2 minutes, 52° C. for 15 seconds (DA), 70° C. for 15 seconds (DA); 10 cycles of 95° C. for 5 second and 61° C. for 30 seconds (0.5° C. decrement per cycle); 40 cycles of 95° C. for 5 second and 52° C. for 40 seconds (DA at each cycle); and 1 cycle of 70° C. for 15 seconds (DA). All reactions were run in triplicate and contained 40 nM of each forward primer, 200 nM of each reverse primer, 200 nM of each partzyme A, 200 nM of each partzyme B, 200 nM of each substrate, 200 nM LOCS-1 and 1× SensiFast Buffer (Bioline). The reactions contained either no target (NF H₂O), or synthetic G-Block of CTcry (20,000, 4,000, 800, 160 or 32 copies); or NGopa gene (20,000 or 32 copies); or various concentrations of CTcry gene (20,000, 4,000, 800, 160 or 32 copies) in a background of NGopa gene (20,000 and 32 copies). All reactions except for the no target control (NF H₂O) further contained a background of 34.5 ng (10,000 copies) of human genomic DNA. Finally, an additional control contained genomic DNA only without any CTcry or NGopa G block.

### Results

In this example, three MNAzymes (MNAzymes 1-3) were used in a single PCR to simultaneously detect and differentiate three target nucleic acids (CTcry, NGopa and human TFRC, respectively) using only two fluorescent channels (HEX and Texas red). The presence or absence of CTcry and/or NGopa were detected and differentiated in the HEX channel and the presence of TFRC gene was detected in the Texas Red channel. The presence of CTcry and TFRC genes were detected in real time by an increase in fluorescence signal at 52° C. (D₁) in the HEX and Texas Red channels respectively, and were also detected at endpoint at 52° C. The presence of NGopa was detected using discrete measurements at 70° C. (D₂) in the HEX channel which monitors cleavage of LOCS-1.

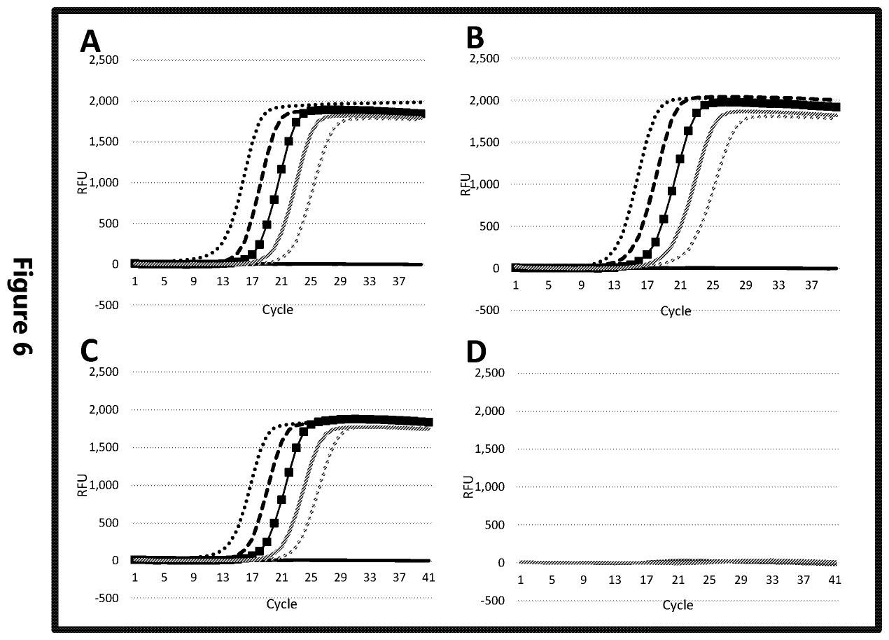

The results shown in FIG. 6 illustrate the PCR amplification curves obtained in the HEX channel at the acquisition temperature of 52° C. from reactions containing 20,000 (black dot), 4,000 (black dash), 800 (black square), 160 (grey solid) or 32 (grey dot) copies of CTcry template either alone (FIG. 6A) or in a background of either 20,000 (FIG. 6B) or 32 (FIG. 6C) copies of NGopa template. Fluorescent data at of 52° C. was also collected for reactions lacking CTcry but containing either 20,000 (black line) or 32 copies (grey line) of NGopa template (FIG. 6D). The no target controls (NF H₂O) are shown in FIG. 6A-6C (black solid line). The amplification curves are the averages of the fluorescence level from triplicate reactions. The calculated copy numbers of CTcry for each of the above reactions are shown in Table 5 where Not Applicable (N/A) refers to where there is no Cq value determined at 52° C. consistent with the absence of CTcry in those reactions. Cq values were determined using regression mode on BioRad software (Baseline Subtracted Curve Fit).

The results in Table 5 show that CTcry standard curves had R² values greater than 0.99, and that PCR efficiencies were high (100-106%). The presence of 20,000 or 32 copies of NGopa in the reaction did not change the calculated gene copy numbers of CTcry significantly (paired Student's t-test, p-value 0.144 and 0.315 respectively), thus demonstrating that real time detection at 52° C. can be used for direct quantitative analysis of CTcry in a sample.

TABLE 5
Copy number determination of CTcry for samples containing
varying gene copies of CTcry and NGopa

| | CTcry copy number calculated from standard curve in | | |
|---|---|---|---|
| | the presence of varying NGopa copy numbers | | |
| Copy number | 0 copies of | 20000 copies of | 32 copies of |
| of CTcry | NGopa | NGopa | NGopa |
| 20,000 | 19740 | 19738 | 21100 |
| 4000 | 4191 | 4534 | 4329 |
| 800 | 755 | 1000 | 775 |
| 160 | 166 | 209 | 162 |
| 32 | 33 | 41 | 40 |
| 0 | N/A | N/A | N/A |
| p-value (paired | — | 0.144 | 0.315 |
| Student's t-test to | | | |
| 0 copies of | | | |
| NGopa group) | | | |
| Efficiency (%) | 100.3 | 106.1 | 102.7 |
| R² | 0.997 | 0.999 | 0.999 |

The results in FIG. 7 illustrate the change in fluorescent signal during PCR measured in the HEX channel at two different temperatures using endpoint Analysis Method 1. In FIG. 7A, the change in signal at temperature 1 (ΔS_{D1}; 52° C.) is shown in black and white pattern and the change signal at temperature 2 (ΔS_{D2}; 70° C.) is shown in grey. The results show that the change in signal at temperature 1 (ΔS_{D1}; black and white pattern bars) crosses threshold 1 (X₁) when CTcry is present within the sample regardless of whether NGopa is present or absent, but does not cross this threshold in the presence NGopa only and/or when CTcry is absent from the sample. Therefore, an endpoint signal change greater than threshold 1 at temperature 1 is indicative of the presence of CTcry. The results in FIG. 7A also show that when the change in signal at temperature 2 (ΔS_{D2}; grey bars) is greater than the change in signal at temperature 1 (ΔS_{D2}>ΔS_{D1}), and is also greater than threshold X₁ (ΔS_{D2}>X₁), then NGopa is present within the sample.

Alternatively, the change in signal at temperatures 1 and 2 can be calibrated against the change in the calibrator signal (ΔC). FIG. 7B illustrates the change in TFRC calibrator signal (ΔC) measured in the Texas Red channel, wherein the values exceeding threshold C indicates a positive signal for ΔC while the values below threshold C indicates a negative signal for ΔC. In FIG. 7C, the change in signal at temperature 1 calibrated against ΔC (ΔS_{D1}/ΔC; 52° C.) is shown in black and white pattern and the change signal at temperature 2 calibrated against ΔC (ΔS_{D2}/ΔC; 70° C.) is shown in grey, for the reactions deemed positive for ΔC in FIG. 7B, where Not Applicable (N/A) refers to the reactions deemed negative for ΔC in FIG. 7B. The results in FIG. 7C can be analysed in the same way as FIG. 7A, which elucidates the same conclusion on the detection of targets 1 and 2.

The results in FIG. 8 illustrate the difference in the change in fluorescent signal obtained in the HEX channel at temperature 2 (ΔS_{D2}) and temperature 1 (ΔS_{D1}) using endpoint Analysis Method 2 (ΔΔS_{D2}ΔS_{D1}). The results show that the ΔΔS_{D2} ΔS_{D1} crosses threshold 2 (X₂) when NGopa is present within the sample, but does not cross this threshold when CTcry only is present within the sample and/or when NGopa is absent from the sample (NTC and genomic DNA only). Therefore, a difference in endpoint fluorescent signal greater than threshold 2 (ΔΔS_{D2} ΔS_{D1}>X₂) is indicative of the presence of NGopa.

The results in FIG. 9 illustrate the change in fluorescent signal obtained in the HEX channel at two different temperatures using Endpoint Analysis Method 3. In FIG. 9A, where ΔS_{D2} is larger than threshold X₁, this indicates CTcry and/or NGopa is present in the sample. Where ΔS_{D2} is lower than threshold X₁, it indicates neither CTcry nor NGopa are present in the reaction (NTC and genomic DNA only). FIG. 9B shows the ratio ΔS_{D1}:ΔS_{D2} can be used to indicate which targets are present in the reaction. When the ratio is higher than threshold R₁, this indicates CTcry is present but not NGopa. Where the ratio is lower than threshold R₂, this indicates NGopa is present but not CTcry. When the ratio is between thresholds R₁ and R₂, this indicates both CTcry and NGopa are present. When neither CTcry or NGopa are present in the reaction (FIG. 9A), the need for calculation of the ratio is negated and indicated as N/A, as shown in FIG. 9B.

Overall, this example demonstrates that two targets can be detected in a single fluorescent channel at two different temperatures by monitoring fluorescence in real time and at discrete time points (pre-PCR and post-PCR). With this method one target can be quantified using a linear MNAzyme substrate, and the other target detected using a LOCS probe. The simple method does not require post-PCR melt curve analysis. The example also demonstrates that qualitative data can be obtained for multiple targets at a single wavelength by comparison of pre-PCR and post-PCR fluorescence values at multiple temperatures. Furthermore, several analysis methods can be applied for analysis of this data.

### Example 2

Method for simultaneous real-time quantification of two targets and qualitative detection of two targets across each of two fluorescent channels using two linear MNAzyme substrates and two LOCS reporters.

The following example demonstrates use of two fluorescent channels, HEX and FAM, for simultaneous detection and differentiation of four targets, wherein Cq determination can be made for two of these targets in a single reaction. In the HEX channel, real-time detection and Cq determination of one target (CTcry), as well as endpoint detection of a second target (NGopa), were achieved using one linear MNAzyme substrate and one LOCS reporter, respectively. Similarly, in the FAM channel, real-time detection and Cq determination of a third target (TVK), as well as endpoint detection of a fourth target (MgPa), were achieved using a second linear MNAzyme substrate and a second LOCS reporter, respectively. Finally, a fifth target, the human TFRC gene was detected in background human genomic DNA using a third linear MNAzyme substrate read in the Texas Red channel.

Target-mediated cleavage of linear substrates by their respective MNAzymes causes separation of a fluorophore and quencher to produce an increase in signal across a broad range of temperatures. In this example, the signal from the linear substrates can be detected in real-time by acquiring fluorescence at each cycle during PCR at Temperature 1 (D₁₌₅₂° C.) or at endpoint by measuring the increase in fluorescence that occurs during PCR when target is present. At this temperature, both the split and the intact LOCS reporters for NGopa and MgPa do not produce any detectable signal, since the Tms of their stem regions are higher than Temperature 1. Therefore, the Cq values obtained at Temperature 1 at each wavelength during PCR reflects the starting quantity of targets Ctcry, TVK and TFRC (regardless of the presence or absence of the other targets) and thus can be used for quantitation.

Target-mediated cleavage of LOCS reporters by their respective MNAzymes causes an increase in signal across a specific range of temperatures. In this example, this fluorescence signal is measured at Temperature 2 (D₂₌₇₀° C.), which is higher than the Tms of both of the split LOCS reporters, but lower than the Tms of both intact LOCS reporters (Tm intact LOCS reporters>Temperature 2>Tm split LOCS reporter) and therefore the intact LOCS reporters do not contribute significant signal at Temperature 2. Therefore, an increase in signal at Temperature 2 reflects the presence of the split LOCS reporter, which confirms the presence of the specific targets NGopa and MgPa in the HEX and FAM channels, respectively.

The example demonstrates concurrent generation of mixed quantitative and qualitative data in a single reaction with real-time monitoring and Cq determination for three targets, combined with simple detection of two other targets elucidated by comparison of pre and post PCR fluorescence readings. Additionally, the example demonstrates qualitative detection of the four targets in two fluorescent channels coupled with the Endpoint Analysis Methods 1-3 (as outlined in Example 1).

### Oligonucleotides

The oligonucleotides specific to this experiment include; Forward primer 1 (SEQ ID NO: 1), Reverse primer 1 (SEQ ID NO: 2), Partzyme A1 (SEQ ID NO: 3), Partzyme B1 (SEQ ID NO: 4), Forward primer 2 (SEQ ID NO: 5), Reverse primer 2 (SEQ ID NO: 6), Partzyme A2 (SEQ ID NO: 7), Partzyme B2 (SEQ ID NO: 8), Forward primer 3 (SEQ ID NO: 9), Reverse primer 3 (SEQ ID NO: 10), Partzyme A3 (SEQ ID NO: 11), Partzyme B3 (SEQ ID NO: 12), linear MNAzyme Substrate 1 (SEQ ID NO: 13), LOCS-1 (SEQ ID NO: 14), linear MNAzyme Substrate 2 (SEQ ID NO: 15), Forward primer 4 (SEQ ID NO: 16), Reverse primer 4 (SEQ ID NO: 17), Partzyme A4 (SEQ ID NO: 18), Partzyme B4 (SEQ ID NO: 19), Forward primer 5 (SEQ ID NO: 20), Reverse primer 5 (SEQ ID NO: 21), Partzyme A5 (SEQ ID NO: 22), Partzyme B5 (SEQ ID NO: 23), linear MNAzyme Substrate 3 (SEQ ID NO: 24), LOCS-2 (SEQ ID NO: 25). The sequences are listed in the Sequence Listing. The oligonucleotides specific for CTcry amplification and detection are Substrate 1, Partzyme A1, Partzyme B1 (MNAzyme 1), Forward Primer 1 and Reverse Primer 1. The oligonucleotides specific for NGopa amplification and detection are LOCS-1, Partzyme A2, Partzyme B2 (MNAzyme 2), Forward Primer 2 and Reverse Primer 2. The oligonucleotides specific for TFRC amplification and detection are Substrate 2, Partzyme A3, Partzyme B3 (MNAzyme 3), Forward Primer 3 and Reverse Primer 3. The oligonucleotides specific for TVK amplification and detection are Substrate 3, Partzyme A4, Partzyme B4 (MNAzyme 4), Forward Primer 4 and Reverse Primer 4. The oligonucleotides specific for MgPa amplification and detection are LOCS-2, Partzyme A5, Partzyme B5 (MNAzyme 5), Forward Primer 5 and Reverse Primer 5.

### Reaction Conditions

Real-time amplification and detection were performed in a total reaction volume of 20 μL using a BioRad® CFX96 thermocycler. The cycling parameters, and fluorescent data acquisition (DA) points, were: 1 cycle of 95° C. for 2 minutes, 52° C. for 15 seconds (DA), 70° C. for 15 seconds (DA), 10 cycles of 95° C. for 5 second and 61° C. for 30 seconds (0.5° C. decrement per cycle), 40 cycles of 95° C. for 5 second and 52° C. for 40 seconds (DA at each cycle) and 70° C. for 15 seconds (DA). All reactions were run in duplicate and contained 40 nM of each forward primer, 200 nM of each reverse primer, 200 nM of each partzyme A, 200 nM of each partzyme B, 200 nM of each substrate, 200 nM LOCS-1, 300 nM LOCS-2 and 1× SensiFast Buffer (Bioline). The reactions contained either no target (NF H₂O), synthetic G-Block of CTcry (20,000 or 32 copies), NGopa gene (20,000 or 32 copies), both CTcry and NGopa (20,000 or 32 copies each), TVK (20,000 or 32 copies), MgPa gene (20,000 or 32 copies) or both TVK and MgPa (20,000 or 32 copies each). All reactions except for the no target control (NF H₂O) contained a background of 10,000 copies of human genomic DNA. An additional control contained genomic DNA only.

### Results

In this example, five MNAzymes (MNAzymes 1-5) were used in a single PCR reaction to simultaneously detect and differentiate five target nucleic acids (CTcry, NGopa, TFRC, TVK and MgPa respectively) using only three fluorescent channels (HEX, Texas red and FAM). The presence or absence of CTcry and/or NGopa were detected and differentiated in the HEX channel, the presence of TFRC gene in genomic DNA was detected in the Texas Red channel and the presence or absence of TVK and/or MgPa were detected and differentiated in the FAM channel. The presence of CTcry, TFRC and TVK genes were detected in real-time by an increase in fluorescence signal at 52° C. in the HEX, Texas Red and FAM channels respectively, and were also detected at endpoint at 52° C. The presence of NGopa and MgPa were detected at endpoint at 70° C. in the HEX and FAM channels respectively, which monitor cleavage of LOCS-1 and LOCS-2 respectively.

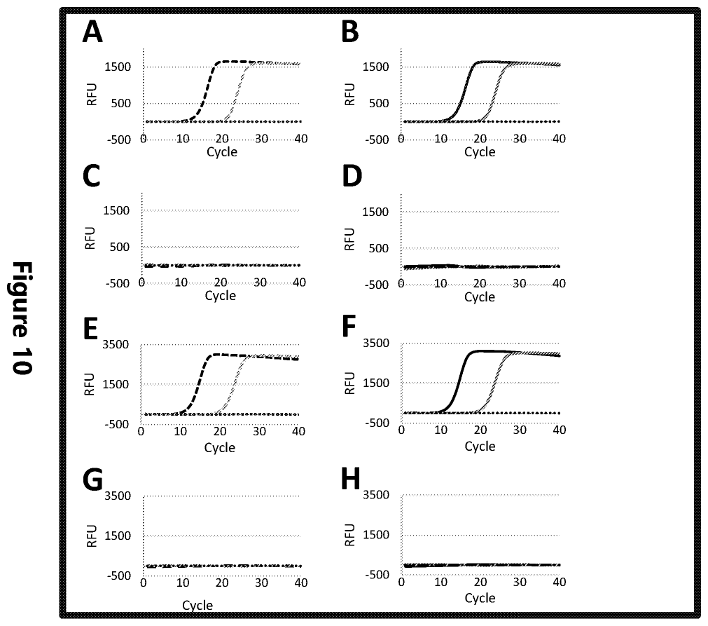

The results shown in FIG. 10 illustrate the PCR amplification curves obtained in the HEX (FIG. 10A-D) and FAM (FIG. 10E-H) channels at the acquisition temperature of 52° C. The amplification curves produced represent 20,000 copies and 32copies of CTcry template alone obtained in the HEX channel (FIG. 10A) or TVK alone (FIG. 10E) obtained in the FAM channel. Amplification curves were also produced for template mixtures containing either 20,000 copies and 32 copies each of both the CTcry and NGopa templates (FIG. 10B) obtained in the HEX channel, or 20,000 copies and 32 copies each of TVK and MgPa templates (FIG. 10F) obtained in the FAM channel. No amplification was seen in the HEX channel for samples containing 20,000 copies and 32 copies of NGopa alone (FIG. 10C), nor was there any amplification recorded for any concentration of the remaining targets including TVK, MgPa and TFRC (endogenous control) (FIG. 10D). Likewise, in the FAM channel, no amplification was recorded for samples containing 20,000 copies and 32 copies of MgPa template alone (FIG. 10G), nor was there any amplification recorded for any concentration of the remaining targets including CTcry, NGopa and TFRC (FIG. 10H). There was no increase in signal for all no target control (NF H₂O) reactions and results are shows as a black dotted line (FIG. 10A-H). The amplification curves are the averages from triplicate reactions and were plotted using Microsoft Excel (Version 14). Cq values were determined using single threshold method set at 100 RFU (Baseline Subtracted Curve Fit).

The Cq values for each of the above reactions are shown in Table 6, where not applicable (N/A) refers to where there is no Cq value determined at 52° C., consistent with the absence of CTcry and TVK in those reactions. Results indicate that Cq values for CTcry and TVK are unaffected by the presence of NGopa templates in the FAM channel or MgPa template in the HEX channel, respectively. Also, each of the non-target reactions did not produce detectable amplification curves or Cq values. Therefore, the Cq values obtained from the HEX and FAM channels can be used for direct quantitative analysis of CTcry and TVK in a sample, respectively.

TABLE 6
Cq values obtained in HEX and FAM
channels during PCR at 52° C. (D₁)

| | Cq values | Standard | Cq values | Standard |
|---|---|---|---|---|
| Target present in | obtained in | deviation | obtained in | deviation |
| sample | HEX | (HEX) | FAM | (FAM) |
| TVK 20 K | N/A | N/A | 10.20 | 0.02 |
| TVK 32 | N/A | N/A | 19.32 | 0.16 |
| MgPa 20K | N/A | N/A | N/A | N/A |
| MgPa 32 | N/A | N/A | N/A | N/A |
| TVK + MgPa 20K | N/A | N/A | 10.22 | 0.08 |
| TVK + MgPa 32 | N/A | N/A | 19.34 | 0.26 |
| CTcry 20K | 12.49 | 0.03 | N/A | N/A |
| CTcry 32 | 21.13 | 0.11 | N/A | N/A |
| NGopa 20K | N/A | N/A | N/A | N/A |
| NGopa 32 | N/A | N/A | N/A | N/A |
| CTcry + NGopa 20K | 12.42 | 0.06 | N/A | N/A |
| CTcry + NGopa 32 | 21.05 | 0.21 | N/A | N/A |
| Genomic DNA only | N/A | N/A | N/A | N/A |

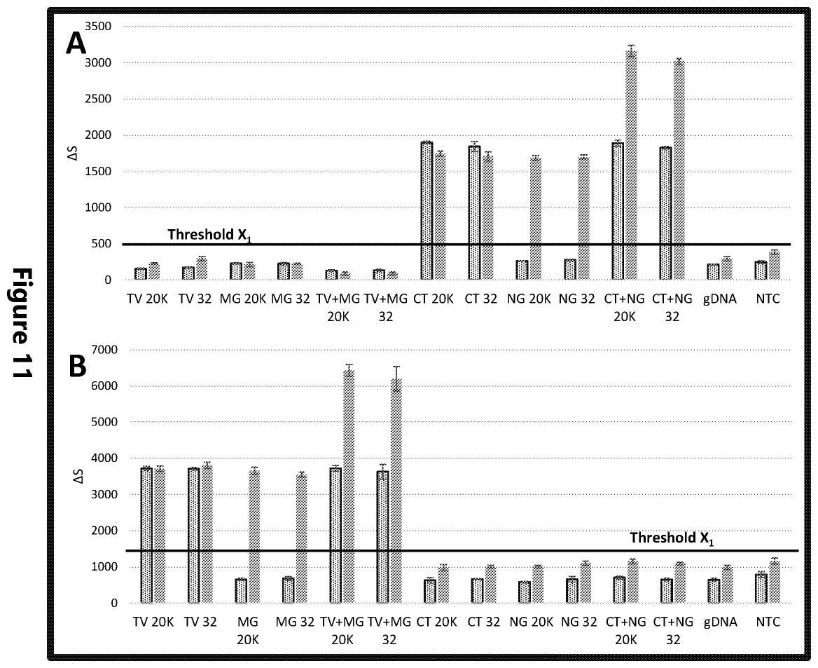

The results in FIG. 11 illustrate the changes in fluorescent signal during amplification (post-PCR minus pre-PCR) obtained in the HEX (FIG. 11A) and FAM channels (FIG. 11B) at two different temperatures using endpoint Analysis Method 1. The change in signal at Temperature 1 (ΔS_{D1}; 52° C.) is shown as black and white pattern and the change in signal at Temperature 2 (ΔS_{D2}; 70° C.) is shown in grey. FIG. 11A shows that ΔS_{D1} crosses Threshold X₁ when CTcry is present within the sample but does not cross this threshold when CTcry is absent from the sample. Therefore, ΔS_{D1}>X₁ is indicative of the presence of CTcry. The results in FIG. 11A also show that when the change in signal at Temperature 2 (ΔS_{D2}) is greater than the change at Temperature 1 (ΔS_{D2}>ΔS_{D1}), and is also greater than Threshold X₁ (ΔS_{D2}>X₁), then NGopa is present within the sample. When ΔS_{D2} crosses Threshold X₁ but ΔS_{D1} does not, this indicates the presence of NGopa alone. Similarly, FIG. 11B shows that change in signal at Temperature 1 (ΔS_{D1}) crosses Threshold X₁ when TVK is present within the sample but does not cross this threshold when TVK is absent from the sample. Therefore, ΔS_{D1}>X₁ is indicative of the presence of TVK. The results in FIG. 11B also show that when the change in signal at Temperature 2 (ΔS_{D2}) is significantly greater than the change in signal at Temperature 1 (ΔS_{D2}>ΔS_{D1}), and is also greater than Threshold X₁ (ΔS_{D2}>X₁), then MgPa is present within the sample. When S_{D2} crosses Threshold X₁ but S_{D1} does not, this indicates the presence of MgPa alone.

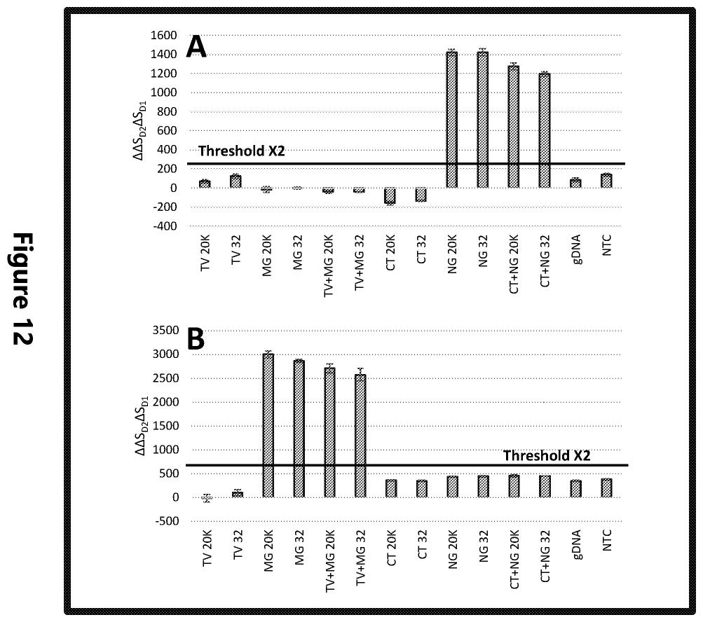

The results in FIG. 12 illustrate the differences in endpoint fluorescent signal increases obtained in the HEX (FIG. 12A) and FAM (FIG. 12B) channels at Temperature 2 and Temperature 1 using endpoint Analysis Method 2 (ΔΔS_{D2}ΔS_{D1}). The results in FIG. 12A show that ΔΔS_{D2} ΔS_{D1} crosses Threshold X₂ when NGopa is present within the sample but does not cross this threshold when NGopa is absent from the sample. Similarly, the results in FIG. 12B show that the ΔΔS_{D2}ΔS_{D1} crosses Threshold X₂ when MgPa is present within the sample but does not cross this threshold when MgPa is absent from the sample. Therefore, when ΔΔS_{D2} ΔS_{D1}>X₂ in the HEX and FAM channels this indicates the presence of NGopa and MgPa respectively.

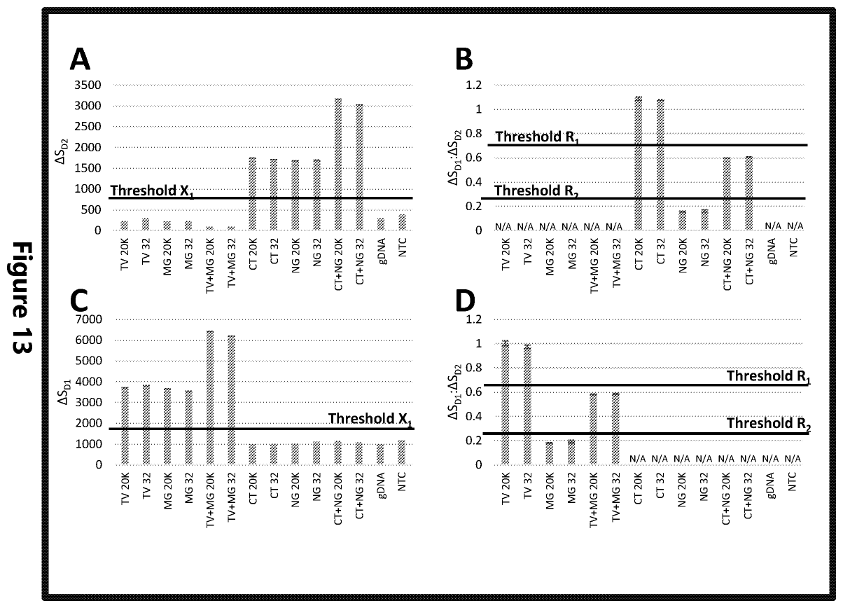

The results in FIG. 13 illustrate the change in fluorescent signal obtained in the HEX (FIG. 13A, B) and FAM (FIG. 13C, D) channels at two different temperatures using Endpoint Analysis Method 3. In FIG. 13A, where ΔS_{D2} in the HEX channel is larger than Threshold X₁, it indicates CTcry and/or NGopa is present in the sample. Where ΔS_{D2} in the HEX channel is lower than Threshold X₁, it indicates neither CTcry nor NGopa are present in the reaction. FIG. 13B shows the ratio ΔS_{D2}: ΔS_{D1} in the HEX channel, which indicates which targets are present in the reaction. Where the ratio is higher than Threshold R₁, it indicates CTcry is present but not NGopa. Where the ratio is lower than Threshold R₂, it indicates NGopa is present but not CTcry. Where the ratio is between Thresholds R₁ and R₂, it indicates both CTcry and NGopa are present. FIG. 13A shows when neither CTcry nor NGopa are present in the reaction, therefore the need for ratio calculation for FIG. 13B is negated and indicated as N/A. In FIG. 13C, ΔS_{D2} in the FAM channel is larger than Threshold X₁, indicating that TVK and/or MgPa is present in the sample. Where ΔS_{D2} in the FAM channel is lower than Threshold X₁, it indicates neither TVK nor MgPa is present in the reaction. FIG. 13D shows the ratio ΔS_{D2}:ΔS_{D1} in the FAM channel which indicates which targets are present in the reaction. Where the ratio is higher than Threshold R₁, it indicates TVK is present but not MgPa. Where the ratio is lower than Threshold R₂, it indicates MgPa is present but not TVK. Where the ratio is between Thresholds R₁ and R₂, it indicates both MgPa and TVK are present. FIG. 13C shows when neither TVK nor MgPa are present in the reaction, therefore the need for ratio calculation for FIG. 13D is negated and indicated as N/A.

### Example 3

Endpoint detection and differentiation of two targets in a single channel using one linear MNAzyme substrate and one LOCS reporter with additional confirmation using melt curve analysis.

The following example demonstrates an approach where one linear MNAzyme substrate and one LOCS reporter are used in combination for detection and differentiation of two targets (TPApolA and TFRC) in a single fluorescent channel using endpoint analysis and melt curve analysis. The assay is designed such that TFRC can be detected and differentiated at endpoint using a linear MNAzyme substrate and TPApolA can be detected and differentiated at endpoint using a LOCS reporter. Confirmatory detection of TPApolA can also be achieved using melt curve analysis in tandem.

In this example, TFRC is included as an endogenous control at a single concentration of 10,000 copies per reaction. TPApolA was tested at two different concentrations: 10,000 copies per reaction and 40 copies per reaction. The simultaneous detection of both TPApolA and TFRC were tested at the following concentrations: 10,000 copies of TFRC together with 10,000 copies of TPApolA per reaction, and 10,000 copies of TFRC together with 40 copies of TPApolA per reaction. This assay is a 10-target multiplex that also contained the primers, partzymes and LOCS for the amplification and detection of eight other targets including: CTcry, LGV, NGopa, NGporA, MgPa, TVbtub, HSV-1 and HSV-2.

During PCR, MNAzyme 6 can cleave linear Substrate 4 in the presence of TFRC to separate a fluorophore and quencher to produce an increase in signal that can be detected across a broad range of temperatures. In this example, endpoint detection of TFRC is achieved by acquiring fluorescence before and after PCR cycling at Temperature 1 (48° C.).

During PCR, MNAzyme 7 can cleave LOCS-3 in the presence of TPApolA. The assay is designed such that the stem of LOCS-3, for both intact and split configurations, has a melting temperature (Tm) that is higher than temperature 1 (48° C.) and therefore does not contribute signal at Temperature 1 regardless of the presence or absence of TPApolA in the sample. Further, the assay is designed such that an intact LOCS-3 has greater Tm than temperature 2 (Tm>68° C.) and the split configuration has a Tm equal to or lower than temperature 2 (Tm<68° C.). Therefore, endpoint detection and differentiation of TPApolA is achieved by acquiring fluorescence before and after PCR cycling at Temperature 2 (68° C.).

In this example, the detection and differentiation of TFRC and TPApolA is achieved by taking fluorescence measurements before and after PCR cycling at Temperature 1 (48° C.) and Temperature 2 (68° C.), respectively. The fluorescence values acquired before PCR cycling are subtracted from the fluorescence values acquired post PCR cycling at each temperature to remove background fluorescence that is unrelated to the target-initiated cleavage of MNAzyme 6 or MNAzyme 7. Further analysis is then performed, as follows:

As previously described in analysis method 2 (Example 1), at temperature 1 (D₁), cleavage of Substrate 4 produces a significant increase in fluorescence signal (ΔS_{D1}) during PCR that exceeds a first threshold (X₁) such that ΔS_{D1}>X₁; however, cleavage of LOCS-3 does not produce significant increase in fluorescence signal at D₁ and does not exceed a threshold (X₁) due to the high Tm of the stem which exceeds D₁ when either intact or split. Therefore, comparison of pre-PCR and post-PCR fluorescence measurements at D₁ (ΔS_{D1}) allows for a specific detection of the cleaved substrate 4 and hence TFRC target.

At temperature 2 (D₂), which is higher than temperature 1 (D₁), cleavage of LOCS-3 throughout PCR produces a change in fluorescence signal (ΔS_{D2}) greater than the change observed at temperature 1 (ΔS_{D1}) wherein the difference between ΔS_{D2} and ΔS_{D1} (ΔS_{D2}−ΔS_{D1}) crosses a second threshold (X₂); such that ΔS_{D2}−ΔS_{D1}=ΔΔS_{D2}ΔS_{D1}>X₂. In contrast, cleavage of substrate 4 alone produces similar ΔS_{D2} and ΔS_{D1} values, wherein the difference between these two values (ΔΔS_{D2}ΔS_{D1}) is not significant and does not cross a second threshold (X₂). Therefore, the analysis of fluorescence at D₂ in this manner allows for a specific detection of the cleaved, split LOCS-3 which is indicative of the presence of TPApolA.

The detection and differentiation of TPApolA can also be confirmed using melt curve analysis. A unique melt curve signature is produced when TPApolA is present in the sample which is distinct from reactions where TPApolA is absent from a sample. This allows visual confirmation of whether TPApolA is present in a sample or not.

### Oligonucleotides

The oligonucleotides specific to this experiment include: Forward primer 3 (SEQ ID NO: 9), Reverse primer 3 (SEQ ID NO: 10), Partzyme A6 (SEQ ID NO: 26), Partzyme B6 (SEQ ID NO: 27), Substrate 4 (SEQ ID NO: 28), Forward Primer 6 (SEQ ID NO: 29), Reverse Primer 6 (SEQ ID NO: 30), Partzyme A7 (SEQ ID NO: 31), Partzyme B7 (SEQ ID NO: 32), LOCS-3 (SEQ ID NO: 33), Forward primer 1 (SEQ ID NO: 1), Forward primer 2 (SEQ ID NO: 2), Partzyme A1 (SEQ ID NO: 3), Partzyme B1 (SEQ ID NO: 4), LOCS-4 (SEQ ID NO: 34), Forward primer 2 (SEQ ID NO: 5), Forward primer 3 (SEQ ID NO: 6), Partzyme A8 (SEQ ID NO: 35), Partzyme B8 (SEQ ID NO: 36), LOCS-5 (SEQ ID NO: 37), Forward Primer 7 (SEQ ID NO: 38), Reverse Primer 7 (SEQ ID NO: 39), Partzyme A9 (SEQ ID NO: 40), Partzyme B9 (SEQ ID NO: 41), LOCS-6 (SEQ ID NO: 42), Forward Primer 8 (SEQ ID NO: 43), Reverse Primer 8 (SEQ ID NO: 44), Partzyme A10 (SEQ ID NO: 45), Partzyme B10 (SEQ ID NO: 46), LOCS-7 (SEQ ID NO: 47), Forward Primer 9 (SEQ ID NO: 48), Reverse Primer 9 (SEQ ID NO: 49), Partzyme A11 (SEQ ID NO: 50), Partzyme B11 (SEQ ID NO: 51), LOCS-8 (SEQ ID NO: 52), Forward Primer 10 (SEQ ID NO: 53), Reverse Primer 10 (SEQ ID NO: 54), Partzyme A12 (SEQ ID NO: 55), Partzyme B12 (SEQ ID NO: 56), LOCS-9 (SEQ ID NO: 57), Forward Primer 11 (SEQ ID NO: 58), Reverse Primer 11 (SEQ ID NO: 59), Partzyme A13 (SEQ ID NO: 60), Partzyme B13 (SEQ ID NO: 61), LOCS-10 (SEQ ID NO: 62), Forward Primer 12 (SEQ ID NO: 63), Reverse Primer 12 (SEQ ID NO: 56), Partzyme A14 (SEQ ID NO: 65), Partzyme B14 (SEQ ID NO: 66), LOCS-11 (SEQ ID NO: 67). The sequences are listed in the Sequence Listing.

The oligonucleotides specific for TFRC amplification and detection are Substrate 4, Partzyme A6, Partzyme B6 (MNAzyme 6), Forward Primer 3 and Reverse Primer 3. The oligonucleotides specific for TPApolA amplification and detection are LOCS-3, Partzyme A7, Partzyme B7 (MNAzyme 7), Forward Primer 6 and Reverse Primer 6. The oligonucleotides specific for CTcry amplification and detection are LOCS-4, Forward Primer 1, Reverse Primer 1, Partzyme A1 and Partzyme B1 (MNAzyme 1). The oligonucleotides specific for LGV amplification and detection are LOCS-6, Forward Primer 7, Reverse Primer 7, Partzyme A9 and Partzyme B9 (MNAzyme 9). The oligonucleotides specific for NGopa amplification and detection are LOCS-5, Forward Primer 2, Reverse Primer 2, Partzyme A8 and Partzyme B8 (MNAzyme 8). The oligonucleotides specific for NGporA amplification and detection are LOCS-7, Forward Primer 8, Reverse Primer 8, Partzyme A10 and Partzyme B10 (MNAzyme 10). The oligonucleotides specific for MgPa amplification and detection are LOCS-10, Forward Primer 11, Reverse Primer 11, Partzyme A13 and Partzyme B13 (MNAzyme 13). The oligonucleotides specific for TVbtub amplification and detection are LOCS-11, Forward Primer 12, Reverse Primer 12, Partzyme A14 and Partzyme B14 (MNAzyme 14). The oligonucleotides specific for HSV-1 amplification and detection are LOCS-8, Forward Primer 9, Reverse Primer 9, Partzyme A11 and Partzyme B11 (MNAzyme 11). The oligonucleotides specific for HSV-2 amplification and detection are LOCS-9, Forward Primer 10, Reverse Primer 10, Partzyme A12 and Partzyme B12 (MNAzyme 12).

### Reaction Conditions

Real-time amplification and detection were performed in a total reaction volume of 20 μL using a BioRad® CFX96 thermocycler. The cycling parameters, and fluorescent data acquisition (DA) points, were: I cycle each of 42° C. for 20 seconds (DA), 48° C. for 20 seconds (DA), 65° C. for 20 seconds (DA), 68° C. for 20 seconds (DA) and 95° C. for 2 minutes; 10 cycles of 95° C. for 5 second and 61° C. for 30 seconds (0.5° C. decrement per cycle); 40 cycles of 95° C. for 5 second, 52° C. for 40 seconds and 65° C. for 5 seconds (DA at each cycle); and 1 cycle each of 30° C. for 20 seconds (DA), 42° C. for 20 seconds (DA), 48° C. for 20 seconds (DA), 65° C. for 20 seconds (DA), 68° C. for 20 seconds (DA). Melt curve parameters were 0.5° C. increments from 20° C. to 95° C. with a 5 sec hold (DA on hold). All reactions were run in duplicate and contained 40 nM of each forward primer, 200 nM of each reverse primer, 200 nM of each partzyme A, 200 nM of each partzyme B, 200 nM of Substrate 4, 300 nM each of LOCS-3, LOCS-4, LOCS-7, LOCS-8, 200 nM each of LOCS-5, LOCS-6, LOCS-9, 240 nM of LOCS-10, 120 nM of LOCS-11, 8 mM MgCl2 (Bioline), 0.2 mM dNTPs (Bioline), 2 units MyTaq polymerase (Bioline) and 1× NH4 Buffer (Bioline). The reactions contained either synthetic G-Block template (10,000 or 40 copies) homologous to the TPApolA, NGopa and/or porA, gpd and/or gpd3, TV-Btub and/or MgPa, CTcry and/or LGV genes or no target (NF H₂O). All reactions except the TPApolA-only reactions contained a background of 10,000 copies of human genomic DNA which harbours the TFRC gene target which serves as an endogenous control. The detection of TFRC gene was monitored in every reaction (except the TPApolA-only reactions) as an internal control by an increase in fluorescence in the Cy5.5 channel.

### Results

In this example, two MNAzymes (MNAzymes 6 and 7) were used in a single PCR reaction to simultaneously detect and differentiate two target nucleic acids (TFRC and TPApolA respectively) using one fluorescent channel (Cy5.5). The presence or absence of TPApolA and/or TFRC was detected and differentiated in the Cy5.5 channel using endpoint analysis. TPApolA was detected at 68° C. (D₂) via an increase in fluorescence caused by the cleavage and melting of LOCS-3 and TFRC was detected at 48° C. (D₁) via cleavage of Substrate 4. The presence of TPApolA in a sample was confirmed in melt curve analysis by the presence of a melt peak at 68° C. (D₂), representing fluorescence produced by cleaved and split LOCS-3. The absence of TPApolA in a sample was confirmed in the melt curves by the presence of a melt peak at 85° C., representing fluorescence produced by uncleaved LOCS-3.

In this example, eight additional targets (two targets per channel) were successfully amplified, detected and differentiated using melt curve analysis (data not shown). While TFRC and TPApolA were detected and differentiated in channel Cy5.5, CTcry and LGV were detected and differentiated in the Cy5 channel, NGopa and NGporA were detected and differentiated in the FAM channel, MgPa and TVbtub were detected and differentiated in the Texas Red channel, and HSV-1 and HSV-2 were detected and differentiated in the JOE channel (data not shown).

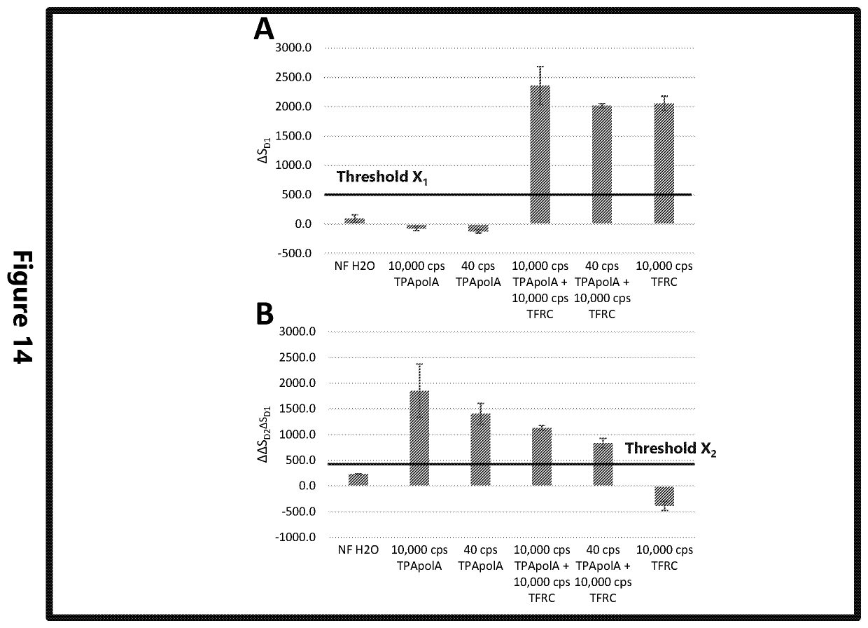

The results in FIG. 14 illustrate the difference in endpoint fluorescent signal obtained in the Cy5.5 channel at Temperature 1 (D₁) and Temperature 2 (D₂) using endpoint analysis (ΔS_{D1} and ΔΔS_{D2} ΔS_{D1}, respectively). The results are averages of duplicate reactions and were plotted using Microsoft Excel (Version 14). Results show that ΔS_{D1} crosses Threshold 1 (X₁) when TFRC is present in the sample but does not cross the threshold when TPApolA only is present and/or when TFRC (and TPApolA) are absent (FIG. 14A). Likewise, ΔΔS_{D2} ΔS_{D1} crosses Threshold 2 (X₂) when TPApolA is present within the sample but does not cross this threshold when TFRC only is present within the sample and/or when TPApolA (and TFRC) are absent (FIG. 14B). Therefore, an endpoint fluorescent signal greater than Threshold 1 (ΔS_{D1}>X₁) indicates the presence of TFRC and a difference in endpoint fluorescence signal between ΔS_{D2} and ΔS_{D1} that is greater than Threshold 2 (ΔΔS_{D2} ΔS_{D1}>X₂) is indicative of the presence of TPApolA regardless of whether TPApolA is present alone or together with TFRC in the reaction.

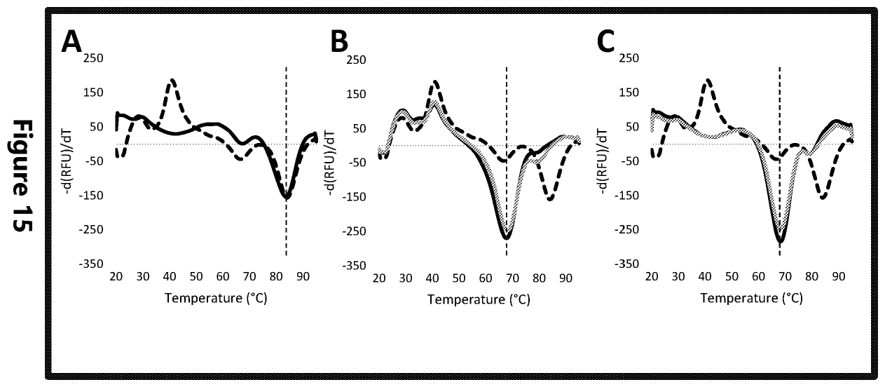

In this example, PCR amplification and endpoint analysis were followed by a post-PCR melt cycle whereby the presence or absence of TPApolA, was confirmed based on LOCS melt peaks at either 68° C. (Split LOCS) or 85° C. (Intact LOCS) respectively. The results shown in FIG. 15 illustrate the respective melt curve signatures obtained post amplification from reactions containing 10,000 copies of TFRC (FIG. 15A), 10,000 copies and 40 copies of TPApolA (FIG. 15B) and coinfections containing 10,000 copies of TPApolA with 10,000 copies of TFRC and 40 copies of TPApolA with 10,000 copies of TFRC (FIG. 15C). The results are the averages from duplicate reactions for all samples containing TPApolA and for reactions that did not contain target (NF H₂O) and the results for TFRC alone are averages from 48 replicates, where 10,000 copies of TFRC was used as an endogenous control. Curves were plotted using Microsoft Excel (Version 14). The results demonstrate that the melt signature produced by Substrate 4 in the presence of TFRC gene target, or with no target, (peak at Tm 85° C.) is distinct from the melt signature produced by LOCS-3 in the presence of TPApolA or in the presence of both TFRC and TPApolA (peak at Tm 68° C.). The results are summarised in Table 7.

TABLE 7
Summary of Melting Temperatures (Tms)
of LOCS-3 in the presence of one or
two targets in channel Cy5.5 as illustrated in FIG. 15.

| | | Melt Curve Tms |
|---|---|---|
| FIG. | Targets Present | (LOCS structure) |
| FIG. 15A, 15B, 15C | NF H₂O (no target) | 85° C. (Intact LOCS) |
| FIG. 15A | TFRC only | 85° C. (Intact LOCS) |
| FIG. 15B | TPApolA only | 68° C. (Split LOCS) |
| FIG. 15C | TFRC and TPApolA | 68° C. (Split LOCS) |

Although for this example the melt curve was performed across a large temperature range from 20° C. to 90° C. and measurements were taken at every half degree, this is not necessary when using LOCS reporters. Since the Tm of the uncleaved and the cleaved, split LOCS do not change with differing target concentrations, smaller temperature ranges could be employed to produce the confirmatory melt curves. The Tms of the cleaved, split LOCS-3 and the uncleaved Intact LOCS-3 are 68° C. and 85° C., respectively, therefore melt curve analysis can be run from ˜50° C. to 90° C. to capture both peaks and reduce time to result. Likewise, data collection points can be limited to acquisition every 1° C. for example, rather than every 0.5° C. to simplify melt curve analysis and theoretically halve the time needed to produce the melt curve. This is advantageous when melt curve analysis is being used as a confirmatory tool to support endpoint analysis.

This example demonstrates that two targets can be detected in a single fluorescent channel at two different temperatures using endpoint analysis with optional melt curve analysis wherein one target is detected using a linear MNAzyme substrate and the other is detected using a LOCS reporter. All target scenarios were able to be detected using endpoint analysis and the presence or absence of TPApolA could be further confirmed by the melt peak signatures. This example provides two methods for detecting multiple targets in a single fluorescent channel, wherein the endpoint fluorescence measurement method can be used independently or in tandem with melt curve analysis for results confirmation.

### Example 4

Method for simultaneous detection and quantification of two targets in a single fluorescent channel using one linear MNAzyme substrate and one LOCS reporter.

The following example demonstrates an approach where the combination of one linear MNAzyme substrate and one LOCS reporter allows simultaneous detection and quantification of two targets in a single fluorescent channel by acquiring fluorescence readings at two temperatures in real time during PCR. Further, this example describes a method for quantifying the amount of either and/or both targets, if present in the sample.

In this example, one linear MNAzyme substrate and one LOCS reporter were used to simultaneously detect, differentiate and quantify two targets (target X and target Y) in the JOE channel by measuring fluorescence in real time at two different temperatures (D₁ and D₂) at each PCR cycle. The assay is designed such that target X is monitored using a linear substrate X (cleavable by MNAzyme X only in the presence of target X), and target Y is monitored using LOCS-Y (cleavable by MNAzyme Y only in the presence of Target Y). Further, the assay is designed such that a split LOCS-Y has a Tm that is higher than the lower detection temperature (D₁).

The lower detection temperature (D₁) is selected such that the cleaved linear Substrate X will fluoresce; whereas an un-cleaved linear Substrate X, a split LOCS-Y and an intact LOCS⁻Y will all remain quenched. The higher temperature (D₂) is selected such that the stem of the split LOCS-Y will melt (dissociate) resulting in increased fluorescence whilst the stem of an intact LOCS-Y will remain associated and quenched.

Two PCR amplification curves can be plotted using fluorescence measurements taken at D₁ and at D₂ temperatures. Threshold values for determination of the presence of targets X and/or Y are set for both the D₁ plot (Threshold X) and for the D₂ plot (Threshold Y). The thresholds (Threshold X and Threshold Y), and various endpoints where reactions are known to plateau, may be pre-determined based on prior experiments, where reactions containing only target X plateau at endpoint X₁ (E_{X1}) at D₁ or at endpoint X₂ (E_{X2}) at D₂; reactions containing only target Y plateau at endpoint Y₁ (E_{Y1}) at D₂, and reactions containing both Target X and Target Y plateau at endpoint Y₂ (E_{Y2}) at D₂. Optionally, endpoints E_{X1}, E_{X2}, E_{Y1} and E_{Y2} may be derived from positive controls run in parallel with experimental samples.

With respect to the D₁ amplification plot, if PCR produces an amplification curve that crosses Threshold X and plateaus at an endpoint E_{X1}, which exceeds the Threshold X, this result would indicate the presence of cleaved linear substrate X associated with target X. If target Y were also present, the amplification curve should not be affected since cleaved LOCS-Y does not produce fluorescence at D₁. Therefore, the Cq values obtained from an amplification curve crossing Threshold X allow quantification of target X in the sample.

With respect to the D₂ amplification plot, if PCR produces an amplification curve that crosses Threshold Y and plateaus at either E_{Y1} or E_{Y2}, this indicates the presence of target Y. Threshold Y is set above the value Ext, so that the amplification curve from a reaction containing only target X does not cross this threshold. When both targets X and Y are present, cleavage of linear substrate X and LOCS-Y will produce amplification curve which crosses Threshold Y and plateaus at endpoint E_{Y2} which is greater than endpoint E_{Y1}, which is associated with cleaved LOCS-Y in the presence of target Y only. Since Ext<Threshold Y<E_{Y1}<E_{Y2} and E_{X2}≠Threshold Y≠E_{Y1}≠E_{Y2}, the endpoints at which amplification curves plateau can indicate the presence of target X, Y or both. However, the Cq values obtained from D₂ amplification curves crossing Threshold Y will be affected by the amount of both target X and Y, and therefore without further data manipulation the results are only semi-quantitative for target Y. An analytical method for calculating the amount of target Y present in a sample where target X is also present is described below.

Target Y quantification method

The two amplification curves arising from cleaved linear MNAzyme substrate X alone at D₁ (39° C.) and D₂ (72° C.) have the same efficiency and Cq but plateau at different endpoints, E_{X1} and Ext respectively. Therefore, the fluorescence signal arising from cleaved substrate X (S_{X}) at 72° C. (S_{X}D₂) can be extrapolated using the signal arising from cleaved substrate X at 39° C. (S_{X}D₁) by applying a fluorescence adjustment factor (FAF). The FAF is the ratio of endpoints E_{X1} and Ex (FAF=E_{X2}/E_{X1}). The total fluorescence signal in the amplification curve at 72° C. (S_{XY}D₂) includes signal arising from both cleaved linear substrate X (S_{X}D₂), if present, and split LOCS-Y (S_{Y}D₂), if present. From this, the signal at 72° C. arising from LOCS-Y alone (S_{Y}D₂) can be extrapolated by the following:

S_{XY}D₂=S_{X}D₂+S_{Y}D₂=(S_{X}D₁*FAF)+S_{Y}D₂

S_{Y}D₂=S_{XY}D₂−(S_{X}D₁*FAF)

The Cq values obtained from the amplification curve constructed from the calculated S_{Y}D₂ values at each cycle may thus provide quantitative data for target Y. The formula can be applied whether the sample contains target X, Y, both X and Y or no target to correctly determine the amount of target Y in a sample.

### Oligonucleotides

The oligonucleotides specific to this experiment include; linear MNAzyme Substrate 1 (SEQ ID: 13), LOCS-1 (SEQ ID: 14), Partzyme A1 (SEQ ID: 3), Partzyme B1 (SEQ ID: 4), Partzyme A2 (SEQ ID: 7), Partzyme B2 (SEQ ID: 8), Forward Primer 1 (SEQ ID: 1), Reverse Primer 1 (SEQ ID: 2), Forward Primer 2 (SEQ ID: 5) and Reverse Primer 2 (SEQ ID: 6). The sequences are listed in the Sequence Listing. The oligonucleotides specific for target X (CTcry) amplification and quantification are Substrate 1, Partzymes A1 and B1 (MNAzyme 1; MNAzyme X) Forward Primer 1 and Reverse Primer 1. The oligonucleotides specific for target Y (NGopa) amplification and quantification are LOCS-1, Partzymes A2 and B2 (MNAzyme 2; MNAzyme Y), Forward Primer 2 and Reverse Primer 2.

### Reaction Conditions

Real-time detection of the target sequence was performed in a total reaction volume of 20 μL using a BioRad® CFX96 thermocycler. The cycling parameters were 95° C. for 2 minutes followed by 10 touchdown cycles of 95° C. for 5 seconds and 61° C. for 30 seconds (0.5° C. decrement per cycle) and 40 cycles of 95° C. for 5 seconds, 52° C. for 40 seconds, 39° C. for 5 sec and 72° C. for 5 sec (data collected at both the 39° C. and 72° C. steps). All reactions were run in duplicate and contained 40 nM of each forward primer, 200 nM of each reverse primer, 200 nM of each Partzyme, 100 nM of linear MNAzyme Substrate 1, 200 nM of LOCS-1 and 1× PlexMastermix (Bioline). The reactions contained either no target (NF H₂O), synthetic G-Block CTcry (20,000, 4,000, 800, 160 or 32 copies), synthetic G-Block of NGopa gene (20,000, 4,000, 800, 160 or 32 copies), various concentrations of synthetic G-Block of CTcry gene (20,000, 4,000, 800, 160 or 32 copies) in a background of synthetic G-Block of NGopa gene (20,000, 4,000, 800, 160 or 32 copies) or various concentrations of synthetic G-Block of NGopa gene (20,000, 4,000, 800, 160 or 32 copies) in a background of synthetic G-Block of CTcry gene (20,000, 4,000, 800, 160 or 32 copies).

### Results

During PCR, two MNAzymes (MNAzyme 1 and MNAzyme 12 are used to monitor amplification of target nucleic acids in real-time via cleavage of linear MNAzyme Substrate 1 and LOCS 1, respectively. MNAzyme 1 was designed to detect sequences homologous to CTcry (Target X) for detection of Chlamydia trachomatis and to cleave Substrate 1. MNAzyme 2 was designed to detect sequences homologous to NGopa (Target Y) for detection of Neisseria gonorrhoeae and to cleave and split LOCS-1.

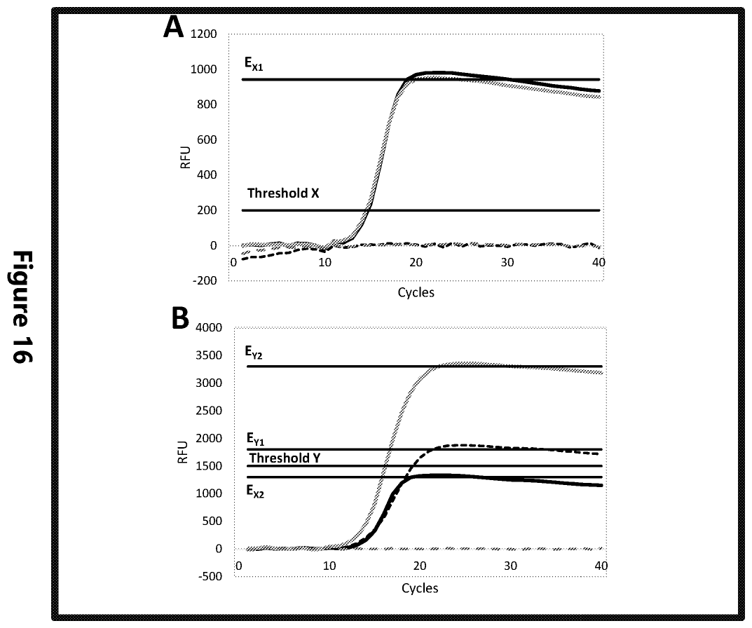

The results shown in FIG. 16 illustrate the comparative amplification curves obtained in the JOE channel from reactions containing either 20,000 copies of CTcry or NGopa or 20,000 copies of both gene targets, measured at 39° C. (D₁) (FIG. 16A) and 72° C. (D₂) (FIG. 16B), respectively. The amplification curves are the averages from duplicate reactions and were plotted using Microsoft Excel (Version 14). Cq values were determined using single threshold method set at 400 RFU and 500 RFU for D₁ (39° C.) and D₂ (68° C.) respectively (Baseline Subtracted Curve Fit). At 39° C., samples containing 20,000 copies of CTcry template (solid black line) show amplification curves with fluorescence levels that exceed Threshold X (black horizontal line) and reach an endpoint (E_{X1}) (black horizontal line denoted as E_{X1}). At 39° C., samples containing 20,000 copies of both CTcry and NGopa templates (solid grey line) also show amplification curves with fluorescence levels that exceed Threshold X and reach E_{X1}. At 39° C., samples that contain NGopa template only (dashed black line) or that do not contain CTcry template (NTC; grey dashed line) do not exceed Threshold X and do not reach E_{X1}. Therefore, the amplification curve at 39° C. plateauing at E_{X1} confirms the presence of CTcry in the sample. The graph shows the samples containing 20,000 copies of CTcry only (solid black line) and 20,000 copies of both CTcry and NGopa (solid grey line) have comparable Cq values. Reactions containing NGopa only (dashed black line), or no target (dashed grey line) did not show an increase in fluorescence during PCR.

Since the Cq values at 39° C. are unaffected by the presence of NGopa, it can be used for quantification of the amount of CTcry in the sample. The results for quantification of CTcry in the samples of all combinations of 0, 32, 160, 800, 4,000 and 20,000 copies of CTcry together with 0, 32, 160, 800, 4,000 and 20,000 copies of NGopa gene targets are summarised in Table 8 where N/A refers to where there is no Cq value determined at 39° C. and therefore there is no CTcry present in the sample.

TABLE 8
Copy number determination of CTcry for samples containing
varying copy numbers of CTcry and NGopa

| | CTcry copy number calculated from standard curve | | | | | |
|---|---|---|---|---|---|---|
| | in the presence of varying NGopa copy numbers | | | | | |
| Copies of | 20000 cps | 4000 cps | 800 cps | 160 cps | 32 cps | |
| NGopa | CTcry | CTcry | CTcry | CTcry | CTcry | 0 |
| 20000 | 20500 | 4260 | 848 | 170 | 32 | N/A |
| 4000 | 21050 | 4175 | 894 | 202 | 29 | N/A |
| 800 | 22400 | 4550 | 958 | 177 | 36 | N/A |
| 160 | 22200 | 4410 | 898 | 179 | 29 | N/A |
| 32 | 23200 | 4285 | 973 | 195 | 35 | N/A |
| 0 | 20500 | 4260 | 848 | 170 | 32 | N/A |

FIG. 16B shows amplification curves at 72° C. for samples containing 20,000 copies of NGopa template (dotted black line) with fluorescence levels that exceed a Threshold Y (black horizontal line) and plateau at a first endpoint Y (E_{Y1}) (black horizontal line denoted as E_{Y1}). At 72° C., samples containing 20,000 copies of both CTcry and NGopa (solid grey line) show amplification curves with fluorescence levels that exceed Threshold Y and plateau at a second Endpoint Y (E_{Y2}) (black horizontal line denoted as E_{Y2}). At 72° C., samples that do not contain NGopa template (solid black line) but contain CTcry do not exceed Threshold Y and do not reach E_{Y1} and E_{Y2}, but instead plateaus at endpoint Ext (black horizontal line denoted as endpoint E_{X2}). At 72° C., samples that do not contain either NGopa or CTcry templates (NTC; grey dashed line) do not show any amplification curve and therefore do not exceed Threshold Y and do not reach E_{Y1} and E_{Y2}. Therefore, the amplification curve at 72° C. plateauing at Ext confirms the presence of target CTcry, but not NGopa in the sample; plateauing at E_{Y1} confirms the presence of NGopa, but not CTcry in the sample; plateauing at E_{Y2} confirms the presence of both CTcry and NGopa in the sample.

At 72° C., the Cq value of samples containing 20,000 copies of both CTcry and NGopa (solid grey line) is not the same as the Cq value of the samples containing 20,000 copies of NGopa template only (dashed black line). Therefore, use of Cq values at 72° C. without normalisation cannot be used to accurately determine the concentration of NGopa in a sample when CTcry is also present.

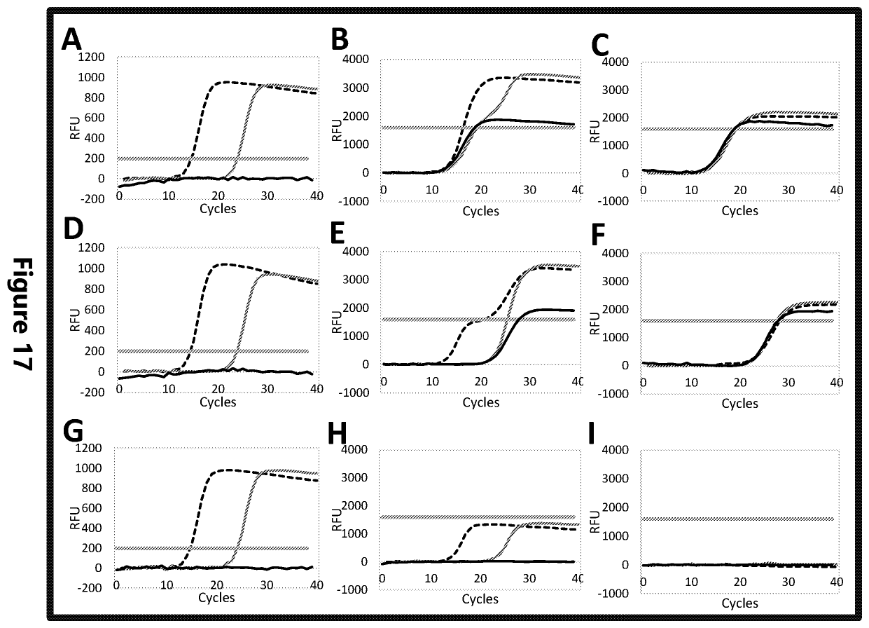

The results shown in FIG. 17 illustrate the comparative amplification curves obtained in the JOE channel from reactions containing all possible combinations of 0, 32 and 20,000 copies of CTcry and 0, 32 and 20,000 copies of NGopa gene targets, measured at 39° C. (FIGS. 17A, 17D and 17G) and 72° C. (FIGS. 17B, 17E and 17H) respectively. The results are plotted as the averages from duplicate reactions and were plotted using Microsoft Excel (Version 14). Cq values were determined using single threshold method set at 400 RFU and 500 RFU for D₁ (39° C.) and D₂ (72° C.) respectively (Baseline Subtracted Curve Fit). The samples that contain 20,000 copies of CTcry are presented in dashed black line, the samples that contain 32 copies of CTcry are presented in solid grey line and the samples that contain 0 copies of CTcry are presented in solid black line.

FIGS. 17A, 17D and 17G show fluorescence at 39° C. (D₁) for reactions containing NGopa (solid black line) at 20,000 copies (FIG. 17A), 32 copies (FIG. 17D) or no copies (FIG. 17G). Results show that there is no increase in signal at 39° C. in the presence of NGopa (FIGS. 17A & 17D) and the signal is comparable to reactions that do not contain NGopa or CTry (NTC; FIG. 17G), thus demonstrating that real time detection at 39° C. (D₁) can be used for determining the presence of CTcry in a sample.

FIGS. 17B, 17E and 17H show fluorescence at 74° C. (D₂) for reactions containing NGopa (solid black line) at 20,000 copies (FIG. 17B), 32 copies (FIG. 17E) or no copies (FIG. 17H). Results show that the non-normalised NGopa amplification curves (FIGS. 17B and 17E) are shifted when 20,00 copies (dashed black line) or 32 copies (grey line) of CTcry is present within the sample.

FIGS. 17C, 17F and 17I show fluorescence at 74° C. (D₂) after normalisation with FAF for reactions containing NGopa (black solid line) at 20,000 copies (FIG. 17C), 32 copies (FIG. 17F) or no copies (FIG. 17I). Results show that the normalised amplification curves at 72° C. (D₂) display similar Cq values where the same number of NGopa templates are present regardless of the amount of CTcry in the sample (FIGS. 17C and 17F). FIG. 17I demonstrates that the extrapolated amplification curves at 72° C. do not show any significant amplification where samples do not contain any NGopa templates. The effect of the aforementioned FAF normalisation method is further demonstrated in Table 9 (S_{Y}D₂ Cq values where S_{Y}D₂=S_{XY}D₂−S_{X}D₂FAF) and Table 10 (quantification of NGopa after S_{Y}D₂=S_{XY}D₂−S_{X}D₂FAF normalisation), which are the analysis of the amplification at 72° C. (D₂) of the samples of all combinations of 0, 32, 160, 800, 4,000 and 20,000 copies of CTcry and 0, 32, 160, 800, 4,000 and 20,000 copies of NGopa gene targets after normalising by subtraction with S_{Y}D₂=S_{XY}D₂ −S_{X}D₂FAF. Not Applicable (N/A) is where there is no Cq value determined at 72° C. and therefore there is no NGopa present in the sample.

TABLE 9
Determination of Cq values of amplification curves
at 72° C. for samples containing varying copy
numbers of CTcry and NGopa after normalisation with FAF

| Copies of | Copies of CTcry | | | | | |
|---|---|---|---|---|---|---|
| NGopa | 20000 | 4000 | 800 | 160 | 32 | 0 |
| 20000 | 15.14 | 15.41 | 15.45 | 15.37 | 15.51 | 15.75 |
| 4000 | 17.31 | 17.34 | 17.60 | 17.60 | 17.98 | 18.10 |
| 800 | 19.31 | 19.37 | 19.53 | 19.95 | 20.09 | 20.21 |
| 160 | 22.07 | 21.83 | 21.65 | 22.07 | 22.39 | 22.55 |
| 32 | 24.27 | 23.96 | 23.48 | 23.83 | 24.19 | 25.00 |
| 0 | N/A | N/A | N/A | N/A | N/A | N/A |

TABLE 10
Copy number determination for NGopa for samples
containing varying copy numbers of CTcry
and NGopa after normalisation with FAF

| | NGopa copy number calculated from standard curve | | | | | |
|---|---|---|---|---|---|---|
| | in the presence of varying CTcry copy numbers | | | | | |
| Copies | 20000 | 4000 | 800 | 160 | 32 | |
| of | cps | cps | cps | cps | cps | 0 cps |
| CTcry | NGopa | NGopa | NGopa | NGopa | NGopa | NGopa |
| 20000 | 29484 | 6388 | 1554 | 223 | 47 | N/A |
| 4000 | 24493 | 6244 | 1492 | 263 | 59 | N/A |
| 800 | 23733 | 5195 | 1334 | 298 | 82 | N/A |
| 160 | 25103 | 5195 | 993 | 222 | 64 | N/A |
| 32 | 22801 | 3980 | 896 | 177 | 50 | N/A |
| 0 | 19244 | 3650 | 827 | 158 | 28 | N/A |

### Prophetic Example 5

Detection and discrimination of multiple targets at a single wavelength using a combination of Molecular Beacons and LOCS.

A non-cleavable Molecular Beacon could be combined with a LOCS probe, which is cleavable by an MNAzyme, to detect and discriminate multiple targets at a single wavelength. As illustrated in FIG. 5, both the Molecular Beacon and the LOCS probe may be labelled with the same detection moiety, for example the same fluorophore. The Molecular Beacon may have a stem region with a Tm A and a Loop region which can specifically hybridize with a first target 1 with a Tm B; where Tm B is greater than Tm A (Tm B>Tm A). This may be combined with an Intact LOCS probe which may have a stem region with a Tm C and a Loop region which can be cleaved by an MNAzyme in the presence a second target 2, thus generating a Split LOCS with a Tm D; where Tm D is less than Tm C (Tm D<Tm C). The presence of target 1 and/or target 2 can be discriminated by measuring the fluorescence at two temperatures (D₁ and D₂) either in real-time; or using single measurements acquired either at, or near, the beginning of amplification and following amplification. The options for endpoint, or real-time measurement of fluorescent output, and the strategy for interpretation of results, is dependent upon the relative temperatures of Tm A, Tm B, Tm C, Tm D in relation to each other, and with respect to D₁ and D₂.

The following scenarios are summarized in Table 1 which provides exemplary Tms for Molecular Beacons and LOCS, and acquisition temperatures for Scenarios 1-4, together with anticipated outcomes.

### Scenario 1

In a first protocol, the Tm A could be 65° C., Tm B could be 70° C., Tm C could be 65° C. and Tm D could be 55° C. (Tm A>Tm D and Tm B>Tm C, Tm B>Tm D). PCR amplification could be performed to amplify target 1 and/or target 2, if present, and fluorescence measurement could be taken at two temperatures at or near the beginning of amplification and again following amplification. In the presence of target 1 and/or target 2, an increase in fluorescence at a first temperature (D₁ 50° C.), which is less than Tm A, Tm B and Tm D (D₁<Tm A, D₁<Tm B, D₁<Tm D), would indicate the presence of target 1 only. At this temperature, the Molecular Beacon would fluoresce in response to hybridization to target 1 or would be quenched in its absence and maintain an internally hybridized stem. At the same temperature D₁, both intact and/or split LOCS species would be quenched due to hybridization of their respective stems at this temperature, regardless of the presence or absence of either target 1 or target 2 in the reaction. Further, an increase in fluorescence at a second temperature (D₂ 60° C.) which is greater than both the first temperature D₁ and Tm D, but less than both Tm B and Tm C (D₂>D₁, D₂>Tm D, D₂<Tm B, D₂<Tm C), would indicate the presence of target 1 and/or target 2. At this second temperature the Molecular Beacon would fluoresce in response to hybridization to target 1 or would be quenched in the absence of target 1 and maintain an internally hybridized stem. The Molecular Beacon would not be affected by the presence or absence of target 2. Additionally, at D₂ if target 2 was absent, the LOCS probe would remain intact and quenched. If target 2 was present, fluorescence would increase since cleavage by MNAzymes specific for target 2 would generate Split LOCS with stem regions which dissociate at this temperature. As such, an increase in fluorescence at temperature 2 during PCR that is greater than the increase in fluorescence observed at temperature 1, would indicate presence of target 2 (ΔF D₂>ΔF D₁).

### Scenario 2

In a second protocol, the Tm A could be 60° C., Tm B could be 70° C., Tm C could be 70° C. and Tm D could be 60° C. (Tm A≈Tm D, Tm B≈Tm C, and Tm B>Tm D). PCR amplification could be performed to simultaneously amplify target 1 and/or target 2 if present and fluorescence measurement could be taken at two temperatures either in real-time, or at/near the beginning of amplification and again following amplification. In the presence of target 1 and/or target 2, an increase in fluorescence at a first temperature (D₁ 50° C.), which is less than Tm A, Tm B and Tm D (D₁<Tm A, D₁<Tm B, D₁>Tm D), would indicate the presence of target 1 only. At this temperature, the Molecular Beacon would fluoresce in response to hybridization to target 1 or would be quenched in its absence and maintain an internally hybridized stem. At the same temperature D₁, both intact and/or split LOCS species would be quenched due to hybridization of their respective stems at this temperature, regardless of the presence or absence of either target 1 or target 2 in the reaction. Further, an increase in fluorescence at a second temperature (D₂ 65° C.) which is greater than both the first temperature D₁ and Tm A and Tm D, but less than Tm B and Tm C (D₂>D₁, D₂>Tm A, D₂>Tm B, D₂>Tm C, D₂<Tm D), would indicate the presence of target 1 and/or target 2. At this second temperature the Molecular Beacon would fluoresce in response to hybridization to target 1 or would be fluorescent due to dissociation of its stem at this temperature regardless of the presence or absence of target 2 in the reaction. As such, fluorescence of the Molecular Beacon will provide the background fluorescence level at this temperature, before, during and following amplification. Additionally, at D₂ if target 2 was absent, the LOCS probe would remain intact and quenched. If target 2 was present, fluorescence would increase above background levels during PCR since cleavage by MNAzymes specific for target 2 would generate Split LOCS with stem regions dissociated at this temperature. As such, an increase in fluorescence at temperature 2 during PCR would indicate presence of target 2. Overall, increase of fluorescence at D₁ during PCR indicates the presence of target 1 detected by the Molecular Beacon and an increase of fluorescence during PCR at D₂ indicates the presence of target 2 detected by the LOCS probe.

### Scenario 3

In a third protocol, the Tm A could be 60° C., Tm B could be 70° C., Tm C could be 80° C. and Tm D could be 70° C. (Tm A<Tm D, Tm A<Tm C and Tm B—Tm D). PCR amplification could be performed to amplify target 1 and/or target 2 if present and fluorescence measurement could be taken at two temperatures either in real time; or at/near the beginning of amplification and again following amplification. In the presence of target 1 and/or target 2, an increase in fluorescence at a first temperature (D₁ 50° C.), which is less than Tm A and Tm B and Tm C and Tm D (D₁<Tm A, D₁<Tm B, D₁<Tm C, D₁<Tm D), would indicate the presence of target 1 only. At this temperature, the Molecular Beacon would fluoresce in response to hybridization to target 1 or would be quenched in its absence and maintain an internally hybridized stem. At the same temperature D₁, both intact and/or split LOCS species would be quenched due to hybridization of their respective stems at this temperature, regardless of the presence or absence of either target 1 or target 2 in the reaction. Further, an increase in fluorescence at a second temperature (D₂ 75° C.) which is greater than both the first temperature D₁ and Tm A, and Tm B and Tm D but less than Tm C (D₂>D₁, D₂>Tm A, D₂>Tm B, D₂>Tm D, D₂<Tm C), would indicate the presence of target 2. At this second temperature the Molecular Beacon would be unable to hybridize to target 1 but would fluoresce due to dissociation of its stem at this temperature regardless of the presence or absence of either target 1 or target 2 in the reaction. As such, fluorescence of the Molecular Beacon will provide the background fluorescence level at this temperature, before, during and following amplification. Additionally, at D₂ if target 2 was absent, the LOCS probe would remain intact and quenched. If target 2 was present, fluorescence would increase above background levels during PCR since cleavage by MNAzymes specific for target 2 would generate Split LOCS with stem regions dissociated at this temperature. As such, an increase in fluorescence at temperature 2 during PCR would indicate presence of target 2. Overall, increase of fluorescence at D₁ during PCR indicates the presence of target 1 detected by the Molecular Beacon and an increase of fluorescence during PCR at D₂ indicates the presence of target 2 detected by the LOCS probe.

Like scenario 2 this embodiment provides a major advantage over other methods known in the art which exploit measurement at multiple temperatures. This specific embodiment combines a Molecular Beacons and a LOCS probe, in a method which negates the need for complex post PCR analysis. The format allows direct quantification of a first target from a first amplification curve generated at a first temperature and direct quantification of a second target from a second amplification curve generated at a second temperature.

### Scenario 4

In a fourth protocol, the Tm A could be 75° C., Tm B could be 80° C., Tm C could be 65° C. and Tm D could be 55° C. (Tm A>Tm C, Tm A>Tm D and Tm B>Tm C). PCR amplification could be performed to amplify target 1 and/or target 2 if present and fluorescence measurement could be taken at two temperatures at/near the beginning of amplification and again following amplification. In the presence of target 1 and/or target 2, an increase in fluorescence at a first temperature (D₁ 70° C.), which is less than Tm A and Tm B but greater than Tm C and Tm D (D₁<Tm A, D₁<Tm B, D₁>Tm C, D₁>Tm D), would indicate the presence of target 1. At this temperature, the Molecular Beacon would fluoresce in response to hybridization of target 1 or would be quenched in the absence of target 1 and maintain an internally hybridized stem. At the same temperature D₁, both the Intact and/or Split LOCS would fluoresce due to dissociation of their respective stem regions at this temperature. Further, an increase in fluorescence at a second temperature (D₂ 60° C.) which is greater than Tm D, but less than the first temperature, Tm A, Tm B and Tm C (D₂<D₁, D₂<Tm A D₂<Tm B, D₂<Tm C, D₂>Tm D), would indicate the presence of target 1 and/or target 2. At this second temperature, the intact LOCS species would be quenched due to hybridization of its stem at this temperature. If target 2 was present, fluorescence would increase during PCR since cleavage by MNAzymes specific for target 2 would generate Split LOCS with stem regions dissociated at this temperature. The Molecular Beacon would fluoresce at this temperature in response to hybridization of target 1 in the presence of target 1 or would be quenched in the absence of target 1 and maintain an internally hybridized stem. The Molecular Beacon would not be affected by the presence or absence of target 2. As such, an observed increase in fluorescence at temperature 2 during PCR that is greater than the observed increase in fluorescence at temperature 1 (ΔS_{D2}>ΔS_{D1}), would indicate presence of target 2.

### Example 6

Method for analysis of multiple targets at a single wavelength using one TaqMan probe and one LOCS probe in a format allowing either simultaneous real-time quantification of one target and qualitative endpoint detection of a second target, or simultaneous qualitative endpoint analysis of two targets per channel.

The following example demonstrates an approach where one TaqMan probe and one LOCS reporter are used together for detection and differentiation of two gene targets (GAPDH and MgPa) with Cq determination of one target (GAPDH) in a single fluorescent channel without the need for melt curve analysis. The assay is designed such that the GAPDH gene can be detected in real-time using a TaqMan probe, and the MgPa gene can be detected and differentiated at endpoint using a LOCS reporter.

During PCR, the TaqMan probe is cleaved in the presence of GAPDH to separate a fluorophore and quencher to produce an increase in signal that can be detected across a broad range of temperatures. In this example, real-time detection and Cq determination of GAPDH is achieved by acquiring fluorescence at each cycle during PCR at temperature 1 (52° C.). The stem of the LOCS probe in both the intact and split configurations have a melting temperature (Tm) that is higher than temperature 1 (52° C.) and therefore the LOCS reporter does not contribute signal at Temperature 1 regardless of the presence or absence of MgPa in the sample.

In the presence of MgPa, MNAzyme 5 can cleave the LOCS probe during PCR. Qualitative detection of MgPa is achieved by measuring the fluorescence at a higher temperature 2 (70° C.) both before and following amplification. Since the Tm of the intact LOCS stem is higher than temperature 2 (Tm>70° C.) and the Tm of the stem of split LOCS is lower than temperature 2 (Tm<70° C.), then the increase in fluorescence is associated with the split and dissociated LOCS and is indicative of the presence of MgPa. This increase in fluorescence must be above any background fluorescence caused by degraded TaqMan probe at this temperature to be considered indicative of the presence of MgPa target.

This example demonstrates mixed quantitative real-time measurement of a first target with qualitative discrete temperature detection of a second target in a single fluorescent channel (FAM). Further, the example demonstrates qualitative analysis of the same two targets by measurement of discrete fluorescence measurements using the Endpoint Analysis Methods 1-3 as explained in Example 1.

### Oligonucleotides

The oligonucleotides specific to this experiment include Forward primer 5 (SEQ ID NO: 20), Reverse primer 5 (SEQ ID NO: 21), Partzyme A5 (SEQ ID NO: 22), Partzyme B5 (SEQ ID NO: 23), LOCS-2 (SEQ ID NO: 25), which are specific for amplification and detection of MgPa. The oligonucleotides specific for amplification and detection of GAPDH are included within the proprietary TaqMan™ Gene Expression Assay (FAM) Human GAPDH (Applied Biosystems).

Reaction Conditions Real-time amplification and detection were performed in a total reaction volume of 20 μL using a BioRad® CFX96 thermocycler. The cycling parameters, and fluorescent data acquisition (DA) points, were: 1 cycle of 95° C. for 2 minutes, 52° C. for 15 seconds (DA), 70° C. for 15 seconds (DA); 10 cycles of 95° C. for 5 second and 61° C. for 30 seconds (0.5° C. decrement per cycle); 40 cycles of 95° C. for 5 second and 52° C. for 40 seconds (DA at each cycle at 52° C.); and 1 cycle of 70° C. for 15 seconds (DA). All reactions were run in triplicate and contained 40 nM of Forward primer 5, 200 nM of Reverse primer 5, 200 nM of Partzyme A5, 200 nM of Partzyme B5, 300 nM LOCS-2, 1× TaqMan™ Gene Expression Assay (FAM) Human GAPDH (Applied Biosystems), and 1× SensiFast Buffer (Bioline). The reactions contained either no target (NF H₂O), or human genomic DNA (10,000 or 100 copies), or synthetic G-Block containing a region of the MgPa gene (10,000 or 100 copies), or both human genomic DNA and synthetic MgPa G-Block (10,000 or 100 copies each).

### Results

In this example, one TaqMan probe, and one LOCS probe cleavable by MNAzyme 5, were combined in a single PCR reaction to simultaneously detect and differentiate two targets GAPDH and MgPa respectively using only a single fluorescent channel (FAM). The presence of GAPDH gene was detected in real-time by an increase in fluorescence signal at 52° C. in the FAM channel and was also detected by analysis of fluorescence at discrete time points at 52° C. before and after PCR cycling. The presence of MgPa was detected by monitoring the cleavage of LOCS-2 through analysis of fluorescence at discrete time points at 70° C. before and after PCR cycling in the same channel.

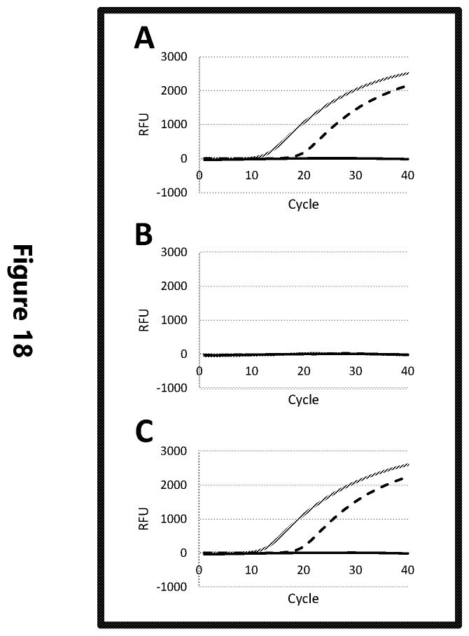

The results shown in FIG. 18A illustrate the PCR amplification curves obtained in the FAM channel at the acquisition temperature of 52° C. from reactions mixtures containing 10,000 (grey line) and 100 (dashed line) copies of human GAPDH templates alone (FIG. 18A); 10,000 (grey line) and 100 (dashed line) copies of MgPa alone (FIG. 18B); and 10,000 (grey line) and 100 (dashed line) copies each of human GAPDH and MgPa templates (FIG. 18C). The no target controls (NF H₂O) are shown in FIG. 18A-C(black solid line).

The Cq values obtained from the above reactions are shown in Table 11, where Not applicable (N/A) refers to where there is no Cq value determined at 52° C., consistent with the absence of human GAPDH. The Cq values were determined using a single threshold value at 200 RFU. Results indicate that Cq values for human GAPDH are comparable regardless of the presence or absence of MgPa. Therefore, the Cq values obtained in the FAM channel can be used for direct quantitative analysis of human GAPDH in a sample.

TABLE 11
Cq determination of GAPDH for samples containing varying
copy numbers of GAPDH and MgPa gene targets

| | Without MgPa | | With MgPa | |
|---|---|---|---|---|
| | Mean | Standard | Mean | Standard |
| | Cq | deviation | Cq | deviation |
| 10000 copies of Human GAPDH | 13.59 | 0.066 | 13.30 | 0.132 |
| 100 copies of Human GAPDH | 20.21 | 0.064 | 20.09 | 0.035 |

| | |
|---|---|
| 10000 copies of MgPa only | N/A |
| 100 copies of MgPa only | N/A |
| No template control | N/A |

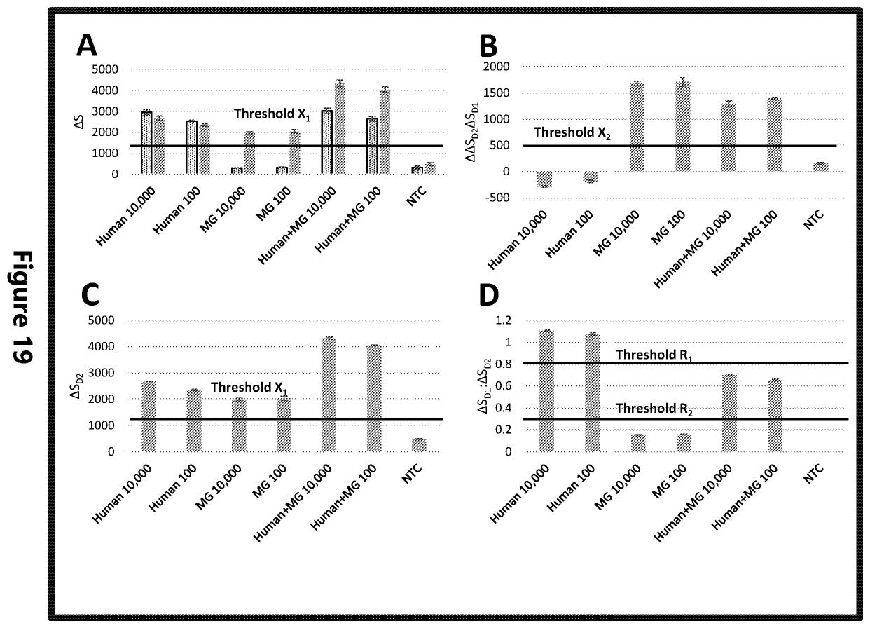

The results in FIG. 19 illustrate the results of qualitative detection of the GAPDH and MgPa genes in the FAM channel. The results for Endpoint Analysis Method 1 are in FIG. 19A, which illustrates the change in fluorescent signal obtained at two different temperatures. ΔS_{D1} (measured at 52° C.) is shown in black and white pattern and ΔS_{D2} (measured at 70° C.) is shown in grey. Results indicate ΔS_{D1} crosses Threshold X₁ when GAPDH is present in the reaction but does not cross the threshold when GAPDH is absent. Therefore, the ΔS_{D1} greater than Threshold X₁ is indicative of the presence of GAPDH. The results also show that when MgPa is present, the ΔS_{D2} crosses Threshold X₁, and is greater than ΔS_{D1} in the reaction (ΔS_{D2}>ΔS_{D1}).

The results in FIG. 19B illustrate the difference in fluorescent signal obtained in the FAM channel at Temperature 2 and Temperature 1 using Endpoint Analysis Method 2 (ΔΔS_{D2}ΔS_{D1}) for the detection of MgPa only. The results show that the ΔΔS_{D2}ΔS_{D1} crosses Threshold X₂ when MgPa is present within the reaction but does not cross this threshold when MgPa is absent from the sample. Therefore, ΔΔS_{D2} ΔS_{D1} greater than Threshold X₂ is indicative of the presence of MgPa.

The results in FIGS. 19C and 19D illustrate the change in fluorescent signal obtained in the FAM channel at two different temperatures using Endpoint Analysis Method 3. In FIG. 19C, where ΔS_{D2} is larger than Threshold X₁, it indicates GAPDH and/or MgPa are present in the reaction. Where ΔS_{D2} is lower than Threshold X₁, it indicates neither GAPDH nor MgPa are present in the reaction. FIG. 19D shows the ratio ΔS_{D1}:ΔS_{D2} can be used to indicate which targets are present in the reaction. Where the ratio is higher than Threshold R₁, it indicates GAPDH is present but not MgPa. Where the ratio is lower than Threshold R₂, it indicates MgPa is present but not GAPDH. Where the ratio is between Thresholds R₁ and R₂, it indicates both GAPDH and MgPa are present. When neither GAPDH or MgPa are present in the reaction (determined from FIG. 19C), the need for calculation of the ratio is negated and indicated as N/A, as shown in FIG. 19D.

Overall, this example demonstrates that two targets can be detected in a single fluorescent channel at two different temperatures wherein one target can be quantified, and the other target detected at endpoint, using one TaqMan probe and one LOCS probe respectively. This simple method does not require melt curve analysis. The example also demonstrates that qualitative data can be obtained for multiple targets at a single wavelength by comparison of pre-PCR and post-PCR fluorescence values at multiple discrete temperatures and that several alternative methods can be applied for analysis of this data. The example demonstrates a further advantage, namely the ability to combine one or more LOCS probes with existing commercial kits using other technologies such as TaqMan probes and thus expand their multiplexing capacity.

### Example 7

Real-time detection and quantification of two targets at a single wavelength using a combination of Molecular Beacons and LOCS.

The following example demonstrates an approach where the combination of one non-cleavable molecular beacon and one LOCS reporter allows simultaneous detection and quantification of two targets in a single fluorescent channel by acquiring fluorescence readings at two temperatures in real time during PCR. This strategy eliminates the requirement for specialized post amplification analysis methods as demonstrated in Example 4. As illustrated in FIG. 5, both the Molecular Beacon and the LOCS probe are labelled with the same fluorophore and quencher moieties for simultaneous detection in the same fluorescence channel. The Molecular Beacon contains a stem region with Tm A and a Loop region which can specifically hybridize with target 1 (TVbtub) with Tm B; where Tm B is greater than Tm A (Tm B>Tm A). In this example, the intact LOCS probe has a stem region with Tm C (82° C.) and a Loop region, which when cleaved by an MNAzyme in the presence a second target 2 (MgPa), generates a Split LOCS with Tm D (62° C.); where Tm D is less than Tm C (Tm D<Tm C). The presence of target 1 (TVbtub) and/or target 2 (MgPa) can be discriminated by measuring the fluorescence at two temperatures (D₁ and D₂; 52° C. and 74° C.) either in real time after each PCR cycle; or using single measurements acquired either at, or near, the beginning of amplification and following amplification. In the following example, Tm A is ˜60° C., Tm B is ˜68° C., Tm C is ˜82° C. and Tm D is ˜62° C. which is consistent with Scenario 3 described in Example 5 wherein D₁<Tm A<Tm B<D₂ and D₁<Tm D<D₂<Tm C.

### Oligonucleotides

The oligonucleotides specific to this experiment include: Forward Primer 12 (SEQ ID: 63), Reverse Primer 12 (SEQ ID: 64) and Molecular Beacon 1 (SEQ ID: 68) for the amplification and quantification of Target 1 (TVbtub). Forward Primer 5 (SEQ ID: 20) and Reverse Primer 5 (SEQ ID: 21), Partzyme A5 (SEQ ID: 22), Partzyme B5 (SEQ ID: 23) and LOCS-2 (SEQ ID: 25) for the amplification and quantification of Target 2 (MgPa). The sequences are listed in the Sequence Listing.

### Reaction Conditions

Real-time detection of the target sequence was performed in a total reaction volume of 20 μL using a BioRad® CFX96 thermocycler. The cycling parameters were 95° C. for 2 minutes, 52° C. for 15 seconds, 74° C. for 15 seconds (data collected at 52° C. and 74° C. steps), followed by 10 touchdown cycles of 95° C. for 5 seconds and 61° C. for 30 seconds (0.5° C. decrement per cycle) and 40 cycles of 95° C. for 5 seconds, 52° C. for 40 seconds, and 74° C. for 5 sec (data collected at both the 52° C. and 74° C. steps). Each reaction contained 20 nM of forward primer 12, 40 nM of forward primer 5, 200 nM of each reverse primer, 200 nM of each Partzyme, 200 nM of Molecular Beacon 1, 200 nM of LOCS-1 reporter and 1× PlexMastermix (Bioline).

The reactions contained either no target (NF H₂O), synthetic G-Block containing a region of the TVbtub gene (25600, 6400, 1600, 400 or 100 copies), synthetic G-Block containing a region of the MgPa gene (25600, 6400, 1600, 400 or 100 copies), various concentrations of synthetic TVbtub G-Block (25600, 6400, 1600, 400 or 100 copies) in a background of synthetic MgPa G-Block (25600 copies) or various concentrations of synthetic TVbtub G-Block (25600, 6400, 1600, 400 or 100 copies) in a background of synthetic MgPa G-Block (25600 copies). Four reactions contained both targets in varying concentrations: 12800 copies of TVbtub with 200 copies of MgPa, 200 copies of TVbtub with 12800 copies of MgPa, 3200 copies of TVbtub with 800 copies of MgPa and 800 copies of TVbtub with 3200 copies of MgPa.

### Results

During PCR amplification, fluorescence was measured at two temperatures in real time to detect and quantify the presence of target 1 (TVbtub) and/or target 2 (MgPa). The Molecular Beacon was designed to detect sequences homologous to TVbtub for detection of Trichomonas vaginalis (TV). The MNAzyme was designed to cleave LOCS-2 in the presence of sequences homologous to MgPa for detection of Mycoplasma genitalium. The presence or absence of specific signal during PCR is summarized in Table 12 and the overall scheme is akin to that described in Scenario 3 (Table 1) of Example 5.

TABLE 12
Signal generated during PCR by Molecular Beacons and LOCS probes.

| | | Signal at D₁ | Signal at D₂ |
|---|---|---|---|
| | | (52° C.) | (74° C.) |
| Beacon | If TV | Beacons remain | Background (F) at D₂ |
| Stem | is | Quenched | Beacon is ALWAYS |
| Tm A | absent | during PCR | Fluorescing (pre-PCR and |
| ~60° C. | | with their stems | post-PCR) regardless of |
| | | hybridized | the presence or absence of |
| | | (Tm A > D1) | either target since the |
| Beacon | If TV | Beacon fluorescence | stem cannot internally |
| loop/ | is | increases | hybridized (Tm A < D2) |
| target | present | during PCR due to | and the loop cannot |
| Tm B | | hybridization of | hybridized to TV (if |
| ~68° C. | | its loop to TV | present) (Tm B < D2) |
| | | (Tm B < D1) | |
| Intact | If | Background (Q) at D₁ | Intact LOCS remains |
| LOCS | MgPa | LOCS are ALWAYS | Quenched during PCR as they |
| stem | is | Quenched | are not cleaved and their stems |
| Tm C | absent | (pre-PCR | are hybridized |
| ~82° C. | | and Post-PCR) | (Tm C > D2) |
| Split | If | regardless of | Split LOCS fluorescence |
| LOCS | MgPa | the presence or | increases during |
| stem | is | absence of either | PCR indicating MgPa- |
| Tm D | present | target since the Tms | specific cleavage by an |
| ~62° C. | | of the stems | MNAzyme and dissociation |
| | | of both Intact | of the stem (Tm D < D2) |
| | | LOCS and Split | |
| | | LOCS are above | |
| | | D1 and are | |
| | | hybridized | |
| | | (Tm C > D1; | |
| | | Tm D > D1) | |

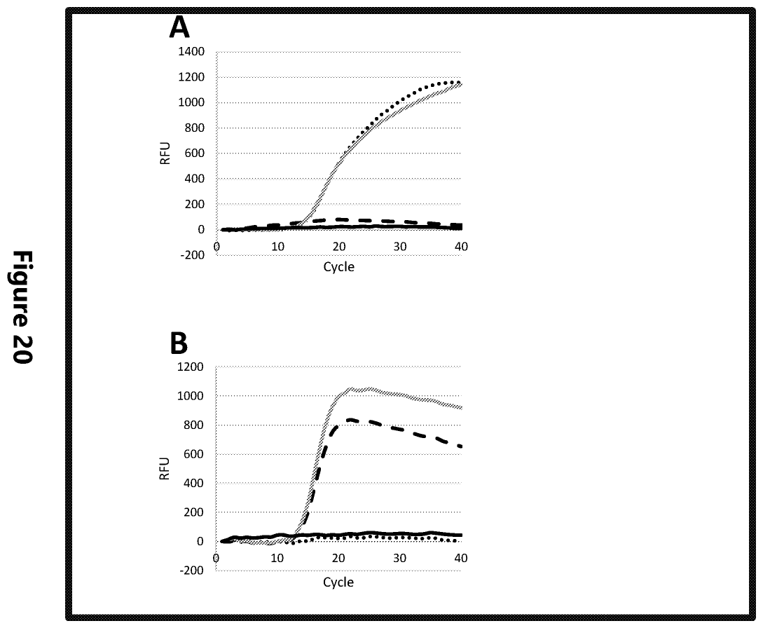

The results shown in FIG. 20 illustrate the comparative amplification curves of fluorescence at 52° C. (D₁, FIG. 20A) and 74° C. (D₂, FIG. 20B) measured in the FAM channel from reactions containing either 25600 copies of TVbtub (black dotted line) or MgPa (black dashed line), a mixture of 25600 copies of both gene targets (grey solid line), or no gene targets (NF H₂O; black solid line). The results are plotted as the averages from triplicate reactions.

FIG. 20A shows at 52° C. (D₁), there is an increase in fluorescence in the presence of TVbtub (black dotted line), or both TVbtub and MgPa (grey solid line) in the reaction, whereas there is no increase in fluorescence in the absence of TVbtub, including the reactions containing MgPa only (black dashed line). At this temperature, the Molecular Beacon fluoresces in response to hybridization to the TVbtub gene target when present or is quenched in the absence its target and maintains an internally hybridized stem. At the same temperature D₁, both intact and/or split LOCS species are quenched due to hybridization of their respective stems at this temperature, regardless of the presence or absence of either TVbtub or MgPa in the reaction. The cycle number at which fluorescence begins to increase exponentially is comparable in reactions containing TVbtub only, and TVbtub plus MgPa, indicating that the presence of the MgPa does not affect the quantification of TVbtub at D₁. Therefore, an increase in fluorescence at 52° C. (D₁) indicates the presence of TVbtub, and the Cq value obtained at D₁ can be used for direct quantification of TVbtub, regardless of the presence or absence of MgPa in the reaction.

FIG. 20B shows at 74° C. (D₂), there is an increase in fluorescence in the presence of MgPa (black dashed line), or both TVbtub and MgPa (grey solid line) in the reaction, whereas there is no increase in fluorescence in the absence of MgPa, including the reactions containing TVbtub only (black dotted line). At D₂, the Molecular Beacon is unable to hybridize to TVbtub, even if present, and is always fluorescing due to dissociation of its stem at this temperature, regardless of the presence or absence of either target in the reaction. As such, fluorescence of the Molecular Beacon provides the background fluorescence level at this temperature, before, during and following amplification. Additionally, at D₂ when MgPa is absent, the LOCS probe remains intact and quenched. When MgPa is present in the reaction, fluorescence increases above background levels during PCR since cleavage by MgPa-specific MNAzymes generates split LOCS with stem regions dissociated at this temperature. As such, an increase in fluorescence at D₂ during PCR indicates presence of MgPa. The cycle number where fluorescence begins to increase exponentially is comparable in the presence of MgPa only and in the presence of a coinfection containing both MgPa and TVbtub, indicating that the presence of TVbtub does not affect the quantification of MgPa at D₂. Therefore, an increase in fluorescence at 72° C. (D₂) indicates the presence of MgPa, and the Cq value obtained at D₂ can be used for direct quantification of MgPa, regardless of the presence of TVbtub in the reaction.

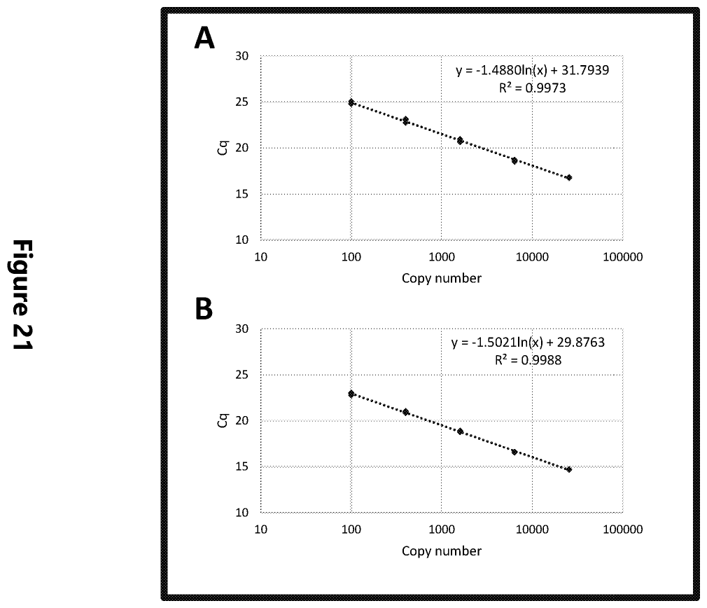

The standard reactions containing 25600, 6400, 1600, 400 and 100 copies of TVbtub were used to construct a standard curve (FIG. 21A) used to quantify the starting concentrations of TVbtub at 52° C. (D₁) in samples that contained TVbtub in the presence or absence of MgPa. Table 13 shows that the presence of 25600 copies of MgPa did not significantly affect the quantification of TVbtub detected at 52° C. (paired Student's t-test, p-value=0.418). Similarly, the standards containing 25600, 6400, 1600, 400 and 100 copies of MgPa were used to construct a standard curve (FIG. 21B) used to quantify the starting concentrations of MgPa at 74° C. (D₂) in samples that contained MgPa in the presence or absence of TVbtub. Table 13 shows the presence of 25600 copies of TVbtub did not significantly affect the quantification of MgPa detected at 74° C. (paired Student's t-test, p-value=0.150). Samples containing random concentrations of TVbtub and MgPa gene targets were also quantified, and the resultant estimated copy numbers were comparable to the known concentration of TVbtub added to the sample (Table 14). This indicates that TVbtub can be accurately quantified at D₁ (52° C.) regardless of the presence of MgPa and likewise, MgPa can be accurately quantified at D₂ (74° C.) regardless of the presence of TVbtub.

TABLE 13
Copy number for TVbtub and MgPa estimated from Standard
Curves generated at 52° C. (FIG. 21A, D₁ for
TVbtub) and at 74° C. (FIG. 21B, D2 for MgPa)

| | 25600 | 6400 | 1600 | 400 | 100 | 0 |
|---|---|---|---|---|---|---|
| | Copies of TVbtub (Standard Curve) | | | | | |
| Estimated copy number | 24134 | 6996 | 1650 | 374 | 102 | N/A |
| of TV (No MgPa in | | | | | | |
| background) | | | | | | |
| Estimated copy number | 25902 | 6972 | 1583 | 341 | 88 | N/A |
| of TV in background | | | | | | |
| of 25600 MgPa | | | | | | |
| p-value | 0.418 | | | | | |
| (paired Student's t-test) | | | | | | |
| | Copies of MgPa (Standard Curve) | | | | | |
| Estimated copy number | 24647 | 6989 | 1547 | 384 | 103 | N/A |
| of MgPa (No TV in | | | | | | |
| background) | | | | | | |
| Estimated copy number | 26159 | 7433 | 1864 | 485 | 122 | N/A |
| of MgPa in background | | | | | | |
| of 25600 TV | | | | | | |

| | |
|---|---|
| p-value | 0.150 |
| (paired Student's t-test) | |

TABLE 14
Copy number for TVbtub and MgPa estimated from Standard
Curves generated at 52° C. (FIG. 21A, D₁ for TVbtub)
and at 74° C. (FIG. 21B, D₂ for MgPa) for
samples containing varying copy numbers of both targets

| | Added copy number of templates | | | |
|---|---|---|---|---|
| TVbtub | 12800 | 200 | 3200 | 800 |
| MgPa | 200 | 12800 | 800 | 3200 |
| Estimated TVbtub | 13831 | 213 | 3614 | 899 |
| Estimated MgPa | 282 | 12305 | 929 | 3202 |

This method provides a major advantage over other methods known in the art which exploit measurements at multiple temperatures followed by analysis using a Fluorescence Adjustment Factor (FAF) (e.g. TOCE) to distinguish multiple targets at a single wavelength. The embodiment within this example requires no adjustment to account for temperature related differences in fluorescence output of the same molecules. Unlike other methods which detect one target at a first temperature and two targets at a second temperature, the example shown here combining a LOCS and a Molecular Beacon allows detection and quantification of one target at one temperature, and detection and quantification of the second target at a second temperature without the interference of the first target (and vice versa). Similarly, in the absence of real time monitoring, an observed increase in fluorescence measured at discrete time points (Post-PCR minus Pre-PCR) indicates the presence of target 1 only at a first temperature and target 2 only at the second temperature.

### Prophetic Example 8

Real-time detection and quantification of two targets at a single wavelength using a combination of Dual Hybridization Probes and LOCS.

The following example illustrates an approach whereby the combination of one pair of dual hybridization probes and one LOCS reporter could allow simultaneous detection and quantification of two targets in a single fluorescent channel by acquiring fluorescence readings at two temperatures in real-time during PCR. Alternatively, in the absence of real-time monitoring the approach could be applied to fluorescent data collected at discrete time points, for example near or at the beginning of amplification and following amplification. This strategy would eliminate the requirement for specialized post amplification analysis methods as outlined in Example 4.

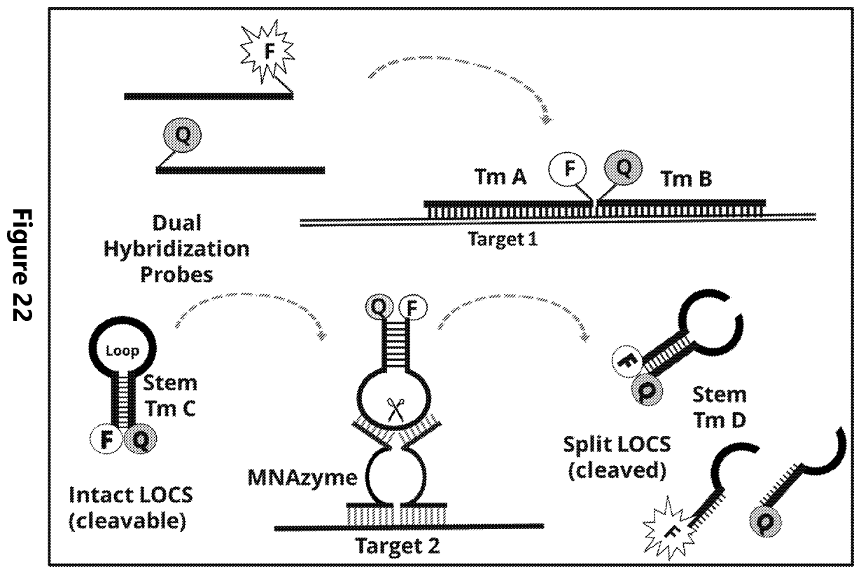

As illustrated in FIG. 22, both the Dual Hybridization probes and the LOCS probes may be labelled with the same fluorophore and quencher moieties for simultaneous detection within the same fluorescence channel. The Dual Hybridization Probe may contain a first probe with a Tm A and second probe with a Tm B, wherein Tm A and Tm B may be equal, or Tm A and Tm B can be different. One probe could be labelled at its 3′ terminus with a fluorophore and the other probe could be labelled at its 5′ terminus with a quencher. (Alternatively, one probe could be labelled at its 3′ terminus with a quencher and the other probe could be labelled at its 5′ terminus with a fluorophore). Dual hybridization probes could be designed to hybridize adjacently on target 1 with a Tm A of, for example, 60° C. and a Tm B of, for example, 60° C. In this scenario, the Dual hybridization probes would be fluorescent prior to amplification but would be quenched following amplification if target 1 were present and the temperature of detection was below Tm A and Tm B. As such, an observed decrease in fluorescence at a first temperature (D₁), for example at 50° C., would be indicative of the presence of Target 1.

Additionally, the reaction could contain an intact LOCS probe which has a stem region with a Tm C of, for example, 80° C. and a Loop region which when cleaved by an MNAzyme in the presence a second target 2, could generates a Split LOCS with a Tm D of, for example, 60° C. (Tm D<Tm C). In this scenario, the Intact LOCS would be quenched prior to amplification but would increase in fluorescence following amplification only if target 2 were present and the detection temperature were above Tm D but below Tm C, and above Tm A and Tm B. As such, an observed increase in fluorescence at a second temperature (D₂), for example at 70° C. would be indicative of the presence of Target 2.

As such, the combination would allow detection of target 1 only using Dual Hybridization probes, determined as a decrease in fluorescence at a first temperature (D₁); and detection of target 2 only using LOCS probes, determined by an increase in fluorescence at a second temperature (D₂).

TABLE 15
Signal generated during PCR in a single channel using a combination
of Dual Hybridization Probes and LOCS probes.

| | | Signal at D₁ | Signal at D₂ |
|---|---|---|---|
| | | (50° C.) | (70° C.) |
| Hybridization | Target | Hybridization | Background Signal at D₂ |
| Probes | 1 | Probes would | Hybridization Probes would |
| Tm A | absent | be Fluorescent | ALWAYS be Fluorescent |
| and Tm B | | before and | (pre-PCR and post-PCR) |
| ~60° C. | | during PCR | regardless of the presence |
| | Target | Hybridization | or absence of either target |
| | 1 | Probes would | since the probes would |
| | present | result in a | not hybridize to |
| | | decrease in | Target 1 (if present) at this |
| | | fluorescence | temperature |
| | | during PCR due | (Tm A < D₂; Tm B < D₂) |
| | | to hybridization | |
| | | to Target 1 | |
| | | (Tm A > D1; | |
| | | Tm B > D₁) | |
| Intact LOCS | Target | Background | Intact LOCS would remain |
| stem | 2 | (Q) at D1 | Quenched during |
| Tm C | absent | LOCS would | PCR as they would |
| ~80° C. | | ALWAYS | not be cleaved and their |
| | | be Quenched | stem would be hybridized |
| | | (Pre-PCR and | (Tm C > D₂) |
| Split LOCS | Target | Post-PCR) | Split LOCS fluorescence |
| stem | 2 | regardless of | would increase during |
| Tm D | present | the presence | PCR indicating |
| ~60° C. | | or absence of | Target 2-specific |
| | | either target | cleavage by an |
| | | since the | MNAzyme and |
| | | Tms of the stems | dissociation of |
| | | of both Intact | the stem (Tm D < D₂) |
| | | LOCS and | |
| | | Split LOCS | |
| | | are above D₁ | |
| | | and are | |
| | | hybridized | |
| | | (Tm C > D₁; | |
| | | Tm D > D₁) | |

### Prophetic Example 9

Real-time detection and quantification of two targets at a single wavelength using a combination of a Scorpion Probe and a LOCS probe.

The following example illustrates an approach whereby the combination of one Scorpion probe and one LOCS reporter could allow simultaneous detection and quantification of two targets in a single fluorescent channel by acquiring fluorescence readings at two temperatures in real time during PCR. Alternatively, in the absence of real time monitoring the approach could be applied to fluorescent data collected at discrete time points, for example near or at the beginning of amplification and following amplification. This strategy would eliminate the requirement for specialized post amplification analysis methods as described in Example 4.

In this embodiment both the Scorpion probe and the LOCS probe may be labelled with the same fluorophore and quencher moieties for simultaneous detection in the same fluorescence channel. Two types of Scorpions probes can be used in this strategy, namely Scorpion Uni-probes and Scorpion Bi-Probes. The Scorpion Uni-Probe may consist of a single-stranded dual-labeled fluorescent probe sequence held in a stem-loop conformation with a 5′ end reporter and an internal quencher directly linked to the 5′ end of a PCR primer via a blocker. During PCR, the primer portion could be designed to hybridize to and extend target 1, wherein the stem region of the stem loop could have a Tm A of, for example, 55° C. and the loop region of the stem loop could bind to target 1 amplicons with a Tm B of, for example, 60° C. During the PCR, the hairpin-loop could unfold and the loop-region of the uni-probe could hybridize intramolecularly to the newly synthesized target 1 sequence, thus separating the fluorophore and quencher. As such, during PCR an observed increase in fluorescence at a first temperature (D₁), for example at 50° C., would be indicative of the presence of Target 1.

Additionally, the reaction could contain an intact LOCS probe which has a stem region with a Tm C of, for example, 80° C. and a Loop region which when cleaved by an MNAzyme in the presence a second target 2, could generate a Split LOCS with a Tm D of, for example, 60° C. (Tm D<Tm C). In this scenario, the Intact LOCS would be quenched prior to amplification but would increase in fluorescence following amplification, only if target 2 were present and the detection temperature were above Tm D but below Tm C, and above Tm A and Tm B. As such, an observed increase in fluorescence at a second temperature (D₂), for example at 65° C. would be indicative of the presence of Target 2.

As such, the combination would allow detection of target 1 only using Scorpion uni-probes, as monitored by an increase in fluorescence at a first temperature (D₁); and detection of target 2 only using LOCS probes, as monitored by an increase in fluorescence at a second temperature (D₂).

TABLE 16
Signal generated during PCR by Scorpion Uni-Probes and LOCS probes.

| | | Signal at D₁ | Signal at D₂ |
|---|---|---|---|
| | | (50° C.) | (65° C.) |
| Scorpion | Target | Scorpion | Background (F) at D₂ |
| Uni-Probes | 1 | Uni-Probes | Scorpion Uni-Probes would |
| with | absent | would | ALWAYS be Fluorescent |
| stem with | | be Quenched | (pre-PCR and post-PCR) |
| a Tm A | | before and | regardless of the presence or |
| ~55° C. | | during PCR | absence of either target since |
| and a loop | Target | Scorpion | the stem would be open and |
| region which | 1 | Uni-Probes would | fluorescing and the loop |
| binds to | present | result in an | would be unable to bind to |
| amplicons of | | increase in | the amplicons of Target 1 (if |
| target 1 with | | fluorescence | present) at this temperature |
| a Tm B | | during PCR | (Tm A < D2 and |
| ~60° C. | | due to | Tm B < D2) |
| | | hybridization of | |
| | | the loop region | |
| | | to Target 1 | |
| | | amplicons | |
| | | (Tm A > D1 and | |
| | | Tm B > D1) | |
| Intact LOCS | Target | Background | Intact LOCS would remain |
| stem | 2 | (Q) at D1 | Quenched during PCR as |
| Tm C | absent | LOCS would | they would not |
| ~80° C. | | ALWAYS be | be cleaved and |
| | | Quenched | their stem would be |
| | | (pre-PCR and | hybridized |
| | | Post-PCR) | (Tm C > D2) |
| Split LOCS | Target | regardless of | Split LOCS fluorescence |
| stem | 2 | the presence or | would increase during PCR |
| Tm D | present | absence of either | indicating Target 2-specific |
| ~60° C. | | target since | cleavage by an MNAzyme |
| | | the Tms of the | and dissociation of the stem |
| | | stems of both | (Tm D < D2) |
| | | Intact LOCS | |
| | | and Split LOCS | |
| | | are above | |
| | | D1 and are | |
| | | hybridized | |
| | | (Tm C > D1; | |
| | | Tm D > D1) | |

Similarly, Scorpion Bi-probes can be combined with LOCS probes. The Scorpion Bi-Probe may consist of a single-stranded fluorescent probe sequence directly linked to the 5′ end of a PCR primer via a blocker. Additionally, a sequence which is complementary to the probe and which is labelled with a quencher can bind to the primer/probe molecule with a Tm A, forming a duplex which is quenched prior to PCR or in the absence of target. During PCR, the primer portion could be designed to hybridize to and extend target 1, wherein the probe region and complementary quencher sequence could have a Tm A of, for example, 55° C. and the probe region could bind to target 1 amplicons with a Tm B of, for example, 60° C. During the PCR, the complementary quencher sequence could dissociate and the probe of the bi-probe could hybridize intramolecularly to the newly synthesized target 1 sequence, therefore blocking binding of the complementary quencher sequence and thus generating fluoresence. As such, during PCR an observed increase in fluorescence at a first temperature (D₁), for example at 50° C., would be indicative of the presence of Target 1.

Additionally, the reaction could contain an intact LOCS probe which has a stem region with a Tm C of, for example, 80° C. and a Loop region which when cleaved by an MNAzyme in the presence a second target 2, could generate a Split LOCS with a Tm D of, for example, 60° C. (Tm D<Tm C). In this scenario, the Intact LOCS would be quenched prior to amplification but would increase in fluorescence following amplification, only if target 2 were present and the detection temperature were above Tm D but below Tm C, and above Tm A and Tm B. As such, an observed increase in fluorescence at a second temperature (D₂), for example at 65° C. would be indicative of the presence of Target 2.

As such, the combination would allow detection of target 1 only using Scorpion bi-probes, as monitored by an increase in fluorescence at a first temperature (D₁); and detection of target 2 only using LOCS probes, as monitored by an increase in fluorescence at a second temperature (D₂).

TABLE 17
Signal generated during PCR by a Scorpion Bi-Probe and a LOCS probe.

| | | Signal at D₁ | Signal at D₂ |
|---|---|---|---|
| | | (50° C.) | (65° C.) |
| Scorpion | Target | Scorpion | Background (F) at D₂ |
| Bi-Probes | 1 | Bi-Probes | Scorpion Bi-Probes would |
| with | absent | would | ALWAYS be Fluorescent |
| complementary | | be Quenched | (pre-PCR and post-PCR) |
| quencher | | before and | regardless of the presence or |
| sequence with | | during PCR | absence of either target since |
| a Tm A | Target | Scorpion | the complementary quencher |
| ~55° C. | 1 | Bi-Probes | sequence could |
| and a probe | present | would | not bind to the |
| region which | | result in | probe sequence and the |
| binds to | | an increase in | probe would be unable |
| amplicons of | | fluorescence | to bind to the |
| target 1 with | | during PCR | amplicons of Target 1 (if |
| a Tm B | | due to | present) at this temperature |
| ~60° C. | | hybridization of | (Tm A < D2 and |
| | | the probe region | Tm B < D2) |
| | | to Target 1 | |
| | | amplicons | |
| | | (Tm A > D1 and | |
| | | Tm B > D1) | |
| Intact LOCS | Target | Background | Intact LOCS would remain |
| stem | 2 | (Q) at D1 | Quenched during PCR as |
| Tm C | absent | LOCS would | they would not |
| ~80° C. | | ALWAYS be | be cleaved and |
| | | Quenched | their stem would be |
| | | (pre-PCR | hybridized |
| | | and Post-PCR) | (Tm C > D2) |
| Split LOCS | Target | regardless of | Split LOCS fluorescence |
| stem | 2 | the presence or | would increase during PCR |
| Tm D | present | absence of either | indicating Target 2-specific |
| ~60° C. | | target since | cleavage by an MNAzyme |
| | | the Tms of the | and dissociation of the stem |
| | | stems of both | (Tm D < D2) |
| | | Intact LOCS | |
| | | and Split LOCS | |
| | | are above | |
| | | D1 and | |
| | | are hybridized | |
| | | (Tm C > D1; | |
| | | Tm D > D1) | |

### Example 10

Methods for analysis of multiple targets at a single wavelength using one of Molecular Beacon and one LOCS reporter with and without internal fluorescence calibration.

The following example demonstrates a method for analysis of multiple targets at a single wavelength using one non-cleavable Molecular Beacon and one LOCS reporter by acquiring pre-amplification and post-amplification fluorescence readings at two temperatures. This strategy eliminates the requirement for the specialized endpoint analysis methods demonstrated in Example 1. Furthermore, this strategy negates the need for data acquisition every cycle required for real-time detection and therefore helps in reducing the overall time to result. As illustrated in FIG. 5, both the Molecular Beacon and LOCS probe are labelled with the same fluorophore and quencher moieties for simultaneous detection in the same fluorescence channel. The Molecular Beacon contains a stem region with a Tm A and a Loop region which can specifically hybridize with target 1 (TVbtub) with a Tm B; where Tm B is greater than Tm A (Tm B>Tm A). In this example, the intact LOCS probe has a stem region with a Tm C (82° C.) and a Loop region which when cleaved by an MNAzyme in the presence target 2 (MgPa), generates a Split LOCS with a Tm D (62° C.); where Tm D is less than Tm C (Tm D<Tm C). The presence of target 1 (TVbtub) and/or target 2 (MgPa) can be differentiated by measuring the increase in fluorescence at two temperatures (D₁ and D₂; 52° C. and 74° C.) using single measurements acquired either at, or near, the beginning of amplification and following amplification. In the following example, the Tm A is ˜60° C., Tm B is ˜68° C., Tm C is ˜82° C. and Tm D is ˜62° C. which is consistent with Scenario 3 described in Example 5 wherein D₁<Tm A<Tm B<D₂ and D₁<Tm D<D₂<Tm C.

### Oligonucleotides

The oligonucleotides specific to this experiment include: Forward Primer 12 (SEQ ID NO: 63), Reverse Primer 12 (SEQ ID: 64) and Molecular Beacon 1 (SEQ ID: 68) for the amplification and quantification of Target 1 (TVbtub); Forward primer 5 (SEQ ID NO: 20), Reverse primer 5 (SEQ ID NO: 21), Partzyme A5 (SEQ ID NO: 22), Partzyme B5 (SEQ ID NO: 23), Substrate 3 (SEQ ID NO: 24), LOCS-2 (SEQ ID NO: 25) for the amplification and quantification of Target 2 (MgPa). The sequences are listed in the Sequence Listing.

### Reaction Conditions

Real-time detection of the target sequence was performed in a total reaction volume of 20 μL using a BioRad® CFX96 thermocycler. The cycling parameters, and fluorescent data acquisition (DA) points, were: 1 cycle of 95° C. for 2 minutes, 52° C. for 15 seconds (DA) and 74° C. for 15 seconds (DA); 50 cycles of 95° C. for 1 seconds and 60° C. for 20 seconds; and 1 cycle of 52° C. for 5 minutes (DA) and 74° C. for 15 seconds (DA). All reactions were performed in triplicate except for the reactions containing 10 copies of templates, for which 6 replicates were used. Each reaction contained 40 nM of each forward primer, 200 nM of each reverse primer, 200 nM of each Partzyme, 200 nM of Molecular Beacon 1, 300 nM of LOCS-2 reporter and 1× PlexMastermix (Bioline). The reactions on the first plate contained either no target (NF H₂O), synthetic G-Block of TVbtub (10000 or 200 copies), synthetic G-Block of MgPa gene (10000 or 200 copies) or various combinations of synthetic G-Block of TVbtub gene (10000 or 200 copies) in a background of synthetic G-Block of MgPa gene (10000 or 200 copies). The reactions on the second plate contained either no target (NF H₂O), synthetic G-Block of TVbtub (200, 100, 50, 25 or 10 copies), synthetic G-Block of MgPa gene (200, 100, 50, 25 or 10 copies) or both of synthetic G-Block of TVbtub and MgPa genes (200, 100, 50, 25 or 10 copies each).

### Results

The increase in fluorescence was determined by calculating the difference between the pre-PCR and post-PCR measurements (ΔS) at two temperatures, (D₁ and D₂; 52° C. and 74° C.), to determine the presence of target 1 (TVbtub) and target 2 (MgPa), respectively. The Molecular Beacon was designed to detect sequences homologous to TVbtub for detection of Trichomonas vaginalis (TV). The MNAzyme was designed to cleave LOCS-1 in the presence of MgPa for detection of Mycoplasma genitalium. The presence or absence of specific signal during PCR is akin to that described in Scenario 3 of Example 5.

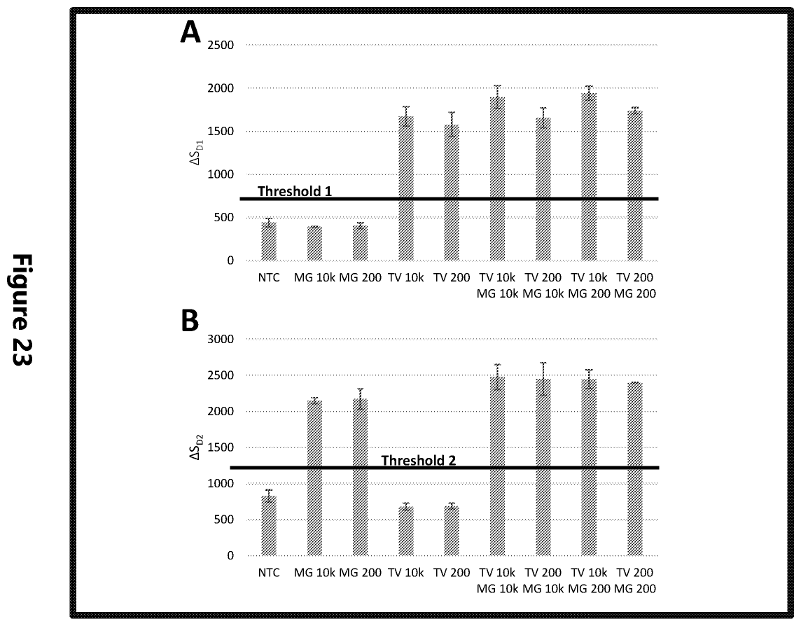

FIG. 23 illustrates the summary of endpoint detection of TVbtub and MgPa by determining the increase in fluorescence at 52° C. (ΔS_{D1}, FIG. 23A) and 74° C. (ΔS_{D2}, FIG. 23B), respectively, as an average of triplicate reactions measured using a first 96-well plate. Where ΔS_{D1} is above the specified threshold (Threshold 1) in FIG. 23A, it indicates the presence of TVbtub in the reaction. All reactions containing either 10,000 or 200 copies of TVbtub were correctly determined positive for TVbtub, regardless of the presence or absence of MgPa in the reaction. In all reactions where TVbtub is absent, ΔS_{D1} stays below the specified threshold and is similar to the no template control (NTC). Similarly, where ΔS_{D2} is above a specified threshold (Threshold 2) in FIG. 23B, it indicates the presence of MgPa in the reaction. All reactions containing either 10,000 or 200 copies of MgPa were correctly determined positive for MgPa, regardless of the presence or absence of TVbtub in the reaction. In all reactions where MgPa is absent, ΔS_{D2} stays below the specified threshold and is similar to the no template control (NTC).

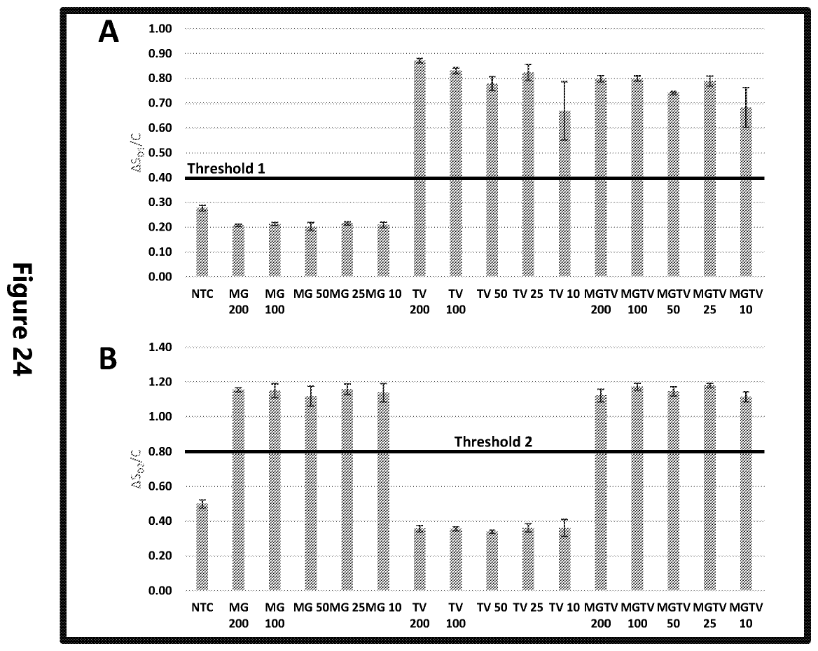

FIG. 24 illustrates a summary of endpoint detection of TVbtub and MgPa, measured on a second 96-well plate. Results are the average of replicates and show the increase in calibrated signal at 52° C. (ΔS_{D1}/C, FIG. 24A) and 74° C. (ΔS_{D2}/C, FIG. 24B). The calibration factor was determined as the difference in the pre-PCR fluorescence between 52° C. and 74° C. (C=S_{D2}-Pre-PCR−S_{D1-Pre-PCR}) which is indicative of the fluorescence arising from dissociation of the stem of the beacon in closed and open conformations. This signal arising from the molecular beacon at D₂ is present in all reactions in the same amount and is unaffected by the presence of either targets. Therefore, the Pre-PCR variances in signals at D₁ and D₂ (C) between the wells reflects the true well-to-well variances in the channel used. The measured signals ΔS_{D1} and ΔS_{D2} were calibrated by dividing each by the calibration factor (C) (ΔS_{D1}/C and ΔS_{D2}/C). FIG. 24A shows that the calibrated signal ΔS_{D1}/C is above the specified threshold (Threshold 1) in all reactions where TVbtub is present, or below if TVbtub is absent. In all reactions where TVbtub is absent, ΔS_{D1}/C stays below the specified threshold and is similar to the no template control (NTC). FIG. 24B shows that the calibrated signal ΔS_{D2}/C is above the specified threshold (Threshold 2) in all reactions where MgPa is present, or below if MgPa is absent. In all reactions where MgPa is absent, ΔS_{D2}/C stays below the specified threshold and is similar to the no template control (NTC). FIG. 24 demonstrates high sensitivity of the assay towards both targets as they were detectable with 10 copies per reaction.

This example demonstrates the use of one molecular beacon and one LOCS reporter for rapid qualitative endpoint detection of two targets in a single fluorescence channel by determining ΔS_{D1} (indicating target 1 only) and ΔS_{D2} (indicating target 2 only). This approach negates the need for specialised endpoint analysis methods. In this example it was also demonstrated that the signals ΔS_{D1} and ΔS_{D2} can be calibrated in the same channel without requiring an additional calibrator reagent using the formulas ΔS_{D1}/C and ΔS_{D2}/C. This approach is advantageous in that run-to-run and machine-to-machine variances can be normalised for more consistent fluorescence outputs. Furthermore, in this example real-time acquisition was not required, which reduces the run time, which in this example measured 54 minutes in total.

### Prophetic Example 11

Real-time detection and quantification of two targets at a single wavelength using a combination of Catcher and Pitcher and LOCS probes.

The following example illustrates an approach whereby the combination of one Catcher and Pitcher pair and one LOCS reporter could allow simultaneous detection and quantification of two targets in a single fluorescent channel by acquiring fluorescence readings at two temperatures in real-time during PCR. Alternatively, in absence of real-time monitoring the approach could be applied to fluorescent data collected at discrete time points, for example near or at the beginning of amplification and following amplification. This strategy would eliminate the requirement for specialized post-amplification analysis methods such as those demonstrated in Example 4.

In this embodiment both the Catcher and the LOCS probe may be labelled with the same fluorophore and quencher moieties for simultaneous detection in the same fluorescence channel. The Pitcher may consist of a single-stranded oligonucleotide that includes a 5′ tagging region which is complementary to the Catcher and a 3′ sensor region which is complementary to a first target 1. The Catcher may consist of a single-stranded oligonucleotide labelled with a quencher at the 5′ end and a fluorophore downstream to the quencher and may include a 3′ tagging region that is complementary to the pitcher. When the Catcher is in a single-stranded conformation, the fluorophore would be in close proximity to the quencher and would remain quenched. During PCR, the primers and the 3′ sensor region of the Pitcher may hybridize to the target. During primer extension, the Pitcher may be degraded by the exonuclease activity of the DNA polymerase and may release the tagging portion of the pitcher. The released tagging portion may then hybridize to the complementary 3′ tagging portion of the Catcher. Extension of the tagging portion by the DNA polymerase during PCR may generate a double-stranded Catcher duplex wherein the fluorophore and quencher are separated. The separation of the fluorophore and quencher may result in an increase in fluorescence which could indicate the presence of target 1. At a first detection temperature (D₁), of 50° C. for example, which is lower than the Tm of the double-stranded Catcher duplex (Tm A, 60° C.), an increase in fluorescence may be observed because the fluorophore and the quencher are separated when in the Catcher duplex conformation. At a second detection temperature (D₂), of 70° C. for example, which is higher than the Tm of the double stranded Catcher duplex (Tm A, 60° C.), the Catcher and Pitcher may dissociate so that they are both in the single-stranded conformation. This may result in a decrease in fluorescence as the fluorophore and quencher are no longer separated. Therefore, an increase in fluorescence during PCR at D₁ may be used to indicate the presence of the double-stranded Catcher duplex and therefore the presence of target 1. In the absence of target 1, the Catcher may remain single-stranded and quenched, and therefore would not contribute to an increase in fluorescence at D₁. The fluorescence at D₂ would not be affected by the presence or absence of target 1 because the Catcher would always remain single-stranded and quenched at a temperature above the Tm of the double stranded Catcher duplex (Tm A, 60° C.).

Additionally, the reaction could contain an intact LOCS probe which could have a stem region with a Tm B of, for example, 80° C. and a Loop region which when cleaved by an MNAzyme in the presence a second target 2, could generate a Split LOCS with a Tm C of, for example, 60° C. (Tm C<Tm B). In this scenario, the Intact LOCS would be quenched prior to amplification but would increase in fluorescence following amplification if target 2 were present and the detection temperature were above Tm C and Tm A but below Tm B. As such, an observed increase in fluorescence at a second temperature (D₂), for example at 70° C. would be indicative of the presence of Target 2. Both the Intact and Split LOCS would remain quenched at a first detection temperature (D₁), since their Tms are higher (D₁<Tm B and D₁<Tm C), and therefore the presence of Target 2 would not contribute to change in signal at a first temperature.

As such, the combination of LOCS and Catcher-Pitcher probes could allow detection of target 1 only using Catcher-Pitcher probes, as monitored by an increase in fluorescence at a first temperature; and detection of target 2 only using LOCS probes, as monitored by an increase in fluorescence at a second temperature.

TABLE 18
Signal generated during PCR by combining Catcher-Pitcher
and LOCS probes.

| | | Signal at D₁ | Signal at D₂ |
|---|---|---|---|
| | | (50° C.) | (70° C.) |
| Catcher- | Target | The Catcher | Background (Q) at D₂ |
| Duplex | 1 | oligonucleotide | Catcher would ALWAYS |
| with a | absent | would be quenched | be Quenched |
| Tm A | | before and during | (pre-PCR and post-PCR) |
| ~60° C. | | PCR as it would | both the Catcher and |
| | | remain single | Pitcher, or the Catcher |
| | | stranded | and extended |
| | Target | Catcher would | Pitcher, would be single- |
| | 1 | result in an | stranded of |
| | present | increase in | the absence and |
| | | fluorescence | presence of target |
| | | during PCR due | respectively |
| | | to extension of the | (Tm A < D₂) |
| | | Pitcher to produce a | |
| | | double-stranded | |
| | | duplex (Tm A > D₁) | |
| Intact | Target | Background (Q) | Intact LOCS |
| LOCS | 2 | at D1 | would remain |
| Tm C | absent | LOCS would | Quenched |
| ~80° C. | | ALWAYS be | during PCR as |
| | | Quenched | they would not |
| | | (pre-PCR and | be cleaved and |
| | | Post-PCR) | their stem would be |
| | | regardless of | hybridized |
| | | the presence or | (Tm C > D₂) |
| Split | Target | absence of either | Split LOCS fluorescence |
| LOCS | 2 | target since the Tms | would increase |
| Tm D | present | of the stems of both | during PCR indicating |
| ~60° C. | | Intact LOCS | Target 2-specific |
| | | and Split LOCS | cleavage by |
| | | are above D₁ and | an MNAzyme |
| | | are hybridized | and dissociation of the |
| | | (Tm C > D₁; | stem (Tm D < D₂) |
| | | Tm D > D1) | |

### Example 12

Method for endpoint analysis of multiple targets at a single wavelength using one linear MNAzyme substrate and one LOCS probe using the same LOCS probe as a calibrator to minimise machine-to machine variability.

The following example demonstrates an approach where one linear MNAzyme substrate and one LOCS reporter are used for detection and differentiation of two gene targets (CTcry and NGopa) in a single fluorescent channel without the need for melt curve analysis. Here, a calibration factor is determined from the pre-amplification signal from the LOCS reporter at two different temperatures and is used to normalise the data to minimise run-to-run and machine-to-machine variations.

During PCR, the linear MNAzyme substrate is cleaved in the presence of CTcry to separate a fluorophore and quencher to produce an increase in signal that can be detected across a broad range of temperatures. In this example, real-time detection and Cq determination of CTcry is achieved by acquiring fluorescence at each cycle during PCR at temperature 1 (52° C., D₁). Qualitative detection of CTcry is also achieved by measuring the fluorescence at D₁ both before and following amplification. The LOCS probe in both the intact and split configurations has a melting temperature (Tm) that is higher than D₁ (52° C.) and therefore the LOCS reporter does not contribute signal at D₁ regardless of the presence or absence of NGopa in the sample.

In the presence of NGopa, MNAzyme 2 can cleave the LOCS probe during PCR. Qualitative detection of NGopa is achieved by measuring the fluorescence at a higher temperature 2 (70° C., D₂) both before and following amplification. Since the Tm of the intact LOCS stem is higher than D₂ (Tm>70° C.) and the Tm of the stem of split LOCS is lower than D₂ (Tm<70° C.), then the increase in fluorescence is associated with the split and dissociated LOCS and is indicative of the presence of NGopa. This increase in fluorescence must be above any background fluorescence caused by cleavage of the linear MNAzyme substrate at this temperature to be considered indicative of the presence of NGopa target.

In this example, fluorescence signal (ΔS) is calibrated against the pre-amplification fluorescence signal at two different temperatures (S_{D1-pre-PCR} and S_{D3-pre-PCR}) which accounts for the differences in signal arising from the two different conformational states of an intact LOCS (i.e. intact, hybridised LOCS at D₁ and intact, dissociated LOCS at D₃). In this example, the dissociation of the intact LOCS occurs at a temperature 3, (D₃; 85° C.), which is higher than the Tm of the intact LOCS (Tm<85° C.). The calibration factor (C) is calculated as the difference between the pre-amplification signal at D₃ (S_{D3-pre-PCR}), where the stem of the intact LOCS is dissociated and fluorescing, and the signal at D₁ (S_{D1-pre-PCR}), where the stem of the intact LOCS is hybridised and quenched (C=S_{D3-pre-PCR}−S_{D1-pre-PCR}). The Calibration factor (C) represents the relative relationship between negative and positive signal (i.e. the dynamic range) for each reaction which should remain unaffected by the presence of any target. Any variation observed in the calibration factor (C), may then reflect any well-to-well variation in each specific channel. Therefore this calibration factor (C) can be used to minimise run-to-run and machine-to-machine variations. In this example, the fluorescence signal obtained from each reaction is calibrated by dividing the ΔS at each temperature (ΔS_{D1} and ΔS_{D2}) by the calibration factor (ΔS_{D1}/C and ΔS_{D2}/C).

This example demonstrates an additional function of LOCS which serves as a calibrator to minimise run-to-run and machine-to-machine variations. While using the pre-PCR measurements arising from LOCS reporter as a calibrator, this example demonstrates simultaneous qualitative analysis of two targets in a single channel by measurement of fluorescence at discrete temperatures taken before and after PCR and Cq determination of one target.

### Oligonucleotides

The oligonucleotides specific to this experiment include; Forward primer 1 (SEQ ID NO: 1), Reverse primer 1 (SEQ ID NO: 2), Partzyme A1 (SEQ ID NO: 3), Partzyme B1 (SEQ ID NO: 4), Forward primer 2 (SEQ ID NO: 5), Reverse primer 2 (SEQ ID NO: 6), Partzyme A2 (SEQ ID NO: 7), Partzyme B2 (SEQ ID NO: 8), Substrate 1 (SEQ ID NO: 13) and LOCS-1 (SEQ ID NO: 14). The sequences are listed in the Sequence Listing.

The oligonucleotides specific for target 1 (CTcry) amplification and detection are Substrate 1, Partzyme A1, Partzyme B1 (MNAzyme 1), Forward Primer 1 and Reverse Primer 1. The oligonucleotides specific for target 2 (NGopa) amplification and detection are LOCS-1, Partzyme A2, Partzyme B2 (MNAzyme 2), Forward Primer 2 and Reverse Primer 2.

### Reaction Conditions

Real-time PCR amplification and detection were performed in a total reaction volume of 20 μL using three different BioRad® CFX96 thermocycler devices on three plates (Machines 1-3). The cycling parameters, and fluorescent data acquisition (DA) points, were: 1 cycle of 95° C. for 2 minutes, 52° C. for 15 seconds (DA), 70° C. for 15 seconds (DA), 85° C. for 15 seconds (DA); 10 cycles of 95° C. for 5 second and 61° C. for 30 seconds (0.5° C. decrement per cycle); 40 cycles of 95° C. for 5 second and 52° C. for 40 seconds (DA at each cycle); and 1 cycle of 70° C. for 15 seconds (DA). All reactions on each plate were run in triplicate and contained 40 nM of each forward primer, 200 nM of each reverse primer, 200 nM of each partzyme A, 200 nM of each partzyme B, 200 nM of each substrate, 200 nM LOCS-1 and 1× SensiFast Buffer (Bioline). The reactions contained either no target (NF H₂O), or synthetic G-Block of CTcry (10,000 or 40 copies); or NGopa (10,000 or 40 copies); or both CTcry and NGopa genes (10,000 or 40 copies each). All reactions except for the no target control (NF H₂O) further contained a background of 34.5 ng (10,000 gene copies) of human genomic DNA.

### Results

In this example, one linear MNAzyme substrate cleavable by MNAzyme 1, and one LOCS probe cleavable by MNAzyme 2, were combined in a single PCR reaction to simultaneously detect and differentiate two targets CTcry and NGopa respectively using only a single fluorescent channel (HEX). The presence of CTcry gene was detected by monitoring the cleavage of linear Substrate-1 through analysis of fluorescence at discrete time points at 52° C. before and after PCR cycling (ΔS_{D1}) in the HEX channel. Also, Cq values obtained from real-time data acquisition at 52° C. is indicative of the CTcry quantity in the reaction (data not shown). The presence of NGopa was detected by monitoring the cleavage of LOCS-1 through analysis of fluorescence at discrete time points at 70° C. before and after PCR cycling in the same channel (ΔS_{D2}). The calibration factor (C) was determined as the difference between the pre-PCR fluorescence signals at 52° C. (D₁) and 85° C. (D₃).

The mean ΔS_{D1} and ΔS_{D2} from triplicate reactions containing varying amounts of CTcry and NGopa templates across three Bio-Rad CFX96 machines are shown in Table 19 and Table 20 respectively. Further, these values were normalised using the calibration factor as shown in Table 21 (ΔS_{D1}/C) and Table 22 (ΔS_{D2}/C) wherein the results are the mean of triplicate reactions. Table 19 and Table 20 show large variations in ΔS_{D1} and ΔS_{D2} values for reactions containing the same template across the three machines tested, as evident with the high coefficient of variation (CV) value (CV≥23.05%). These run-to-run and/or machine-to-machine variations make it difficult to place a single threshold value for determining the presence or absence of target for each of the three machines. However, after normalisation using the calibration factor, there is much less variations in the results obtained from the three different machines, as evidenced by a reduction in CV values (CV≤4.13%) as shown in Table 21 and Table 22. The results demonstrate that normalisation using C is an effective method to reduce run-to-run or machine-to-machine variations.

TABLE 19
Mean difference in fluorescence signals before and after PCR at 52° C.
(ΔS_{D1}) in three Bio-Rad CFX96 machines for reactions
containing varying amounts of CTcry and
NGopa templates

| | ΔS_{D1} in | ΔS_{D1} in | ΔS_{D1} in | Coefficient | CT |
|---|---|---|---|---|---|
| | Machine | Machine | Machine | of | detection |
| | 1 | 2 | 3 | variation | using a |
| | (RFU) | (RFU) | (RFU) | (%) | threshold |
| CTcry only | 2640 | 4136 | 4112 | 23.61 | YES (>2,000) |
| (10,000 copies) | | | | | |
| CTcry only | 2712 | 4308 | 4396 | 24.91 | YES (>2,000) |
| (40 copies) | | | | | |
| NGopa only | 415 | 650 | 656 | 23.94 | NO (<2,000) |
| (10,000 copies) | | | | | |
| NGopa only | 452 | 684 | 710 | 23.05 | NO (<2,000) |
| (40 copies) | | | | | |
| CTcry + NGopa | 2787 | 4329 | 4612 | 25.12 | YES (>2,000) |
| (10,000 copies | | | | | |
| each) | | | | | |
| CTcry + NGopa | 2855 | 4411 | 4722 | 25.03 | YES (>2,000) |
| (40 copies each) | | | | | |
| No target | 471 | 721 | 781 | 24.99 | NO (<2,000) |

TABLE 20
Mean difference in fluorescence signals before and after PCR
at 70° C. (ΔS_{D2}) in three Bio-Rad CFX96 machines for reactions
containing varying amounts of CTcry and
NGopa templates

| | ΔS_{D1} in | ΔS_{D1} in | ΔS_{D1} in | Coefficient | CT or NG |
|---|---|---|---|---|---|
| | Machine | Machine | Machine | of | detection |
| | 1 | 2 | 3 | variation | using |
| | (RFU) | (RFU) | (RFU) | (%) | a threshold |
| CTcry only | 2134 | 3683 | 3500 | 27.26 | Not possible |
| (10,000 copies) | | | | | with a single |
| | | | | | threshold |
| CTcry only | 2174 | 3767 | 3733 | 28.23 | Not possible |
| (40 copies) | | | | | with a single |
| | | | | | threshold |
| NGopa only | 1940 | 3293 | 3442 | 28.61 | Not possible |
| (10,000 copies) | | | | | with a single |
| | | | | | threshold |
| NGopa only | 2025 | 3349 | 3572 | 28.05 | Not possible |
| (40 copies) | | | | | with a single |
| | | | | | threshold |
| CTcry + NGopa | 3806 | 6469 | 6879 | 29.18 | Not possible |
| (10,000 copies | | | | | with a single |
| each) | | | | | threshold |
| CTcry + NGopa | 3922 | 6529 | 7049 | 28.73 | Not possible |
| (40 copies each) | | | | | with a single |
| | | | | | threshold |
| No target | 593 | 1024 | 1070 | 29.39 | Not possible |
| | | | | | with a single |
| | | | | | threshold |

TABLE 21
Mean difference in fluorescence signals before and after PCR at
52° C. after calibration with C (ΔS_{D1}/C) in three Bio-Rad
CFX96 machines for reactions containing
varying amounts of CTcry and NGopa templates

| | ΔS_{D1}/ | ΔS_{D1}/ | ΔS_{D1}/ | Coefficient | CT |
|---|---|---|---|---|---|
| | C in | C in | C in | of | detection |
| | Machine | Machine | Machine | variation | using a |
| | 1 | 2 | 3 | (%) | threshold |
| CTcry only | 0.843 | 0.812 | 0.807 | 2.41 | YES |
| (10,000 | | | | | (>0.5) |
| copies) | | | | | |
| CTcry only | 0.811 | 0.780 | 0.791 | 1.96 | YES |
| (40 copies) | | | | | (>0.5) |
| NGopa only | 0.132 | 0.126 | 0.121 | 4.13 | NO |
| (10,000 | | | | | (<0.5) |
| copies) | | | | | |
| NGopa only | 0.132 | 0.126 | 0.125 | 3.07 | NO |
| (40 copies) | | | | | (<0.5) |
| CTcry + | 0.864 | 0.813 | 0.807 | 3.81 | YES |
| NGopa | | | | | (>0.5) |
| (10,000 | | | | | |
| copies | | | | | |
| each) | | | | | |
| CTcry + | 0.835 | 0.801 | 0.784 | 3.23 | NO |
| NGopa | | | | | (<0.5) |
| (40 copies | | | | | |
| each) | | | | | |
| No target | 0.162 | 0.148 | 0.149 | 5.35 | NO |
| | | | | | (<0.5) |

TABLE 22
Mean difference in fluorescence signals before and after PCR
at 70° C. after calibration with C (ΔS_{D2}/C)
in three Bio-Rad CFX96 machines for reactions
containing varying amounts of CTcry and NGopa templates

| | ΔS_{D2}/ | ΔS_{D2}/ | ΔS_{D2}/ | Coefficient | CT or NG |
|---|---|---|---|---|---|
| | C in | C in | C in | of | detection |
| | Machine | Machine | Machine | variation | using |
| | 1 | 2 | 3 | (%) | a threshold |
| CTcry | 0.681 | 0.723 | 0.686 | 3.29 | CT only |
| only | | | | | or NG |
| (10,000 | | | | | only |
| copies) | | | | | (>0.4, <0.9) |
| CTcry | 0.650 | 0.683 | 0.672 | 2.51 | CT only |
| only | | | | | or NG |
| (40 | | | | | only |
| copies) | | | | | (>0.4, <0.9) |
| NGopa | 0.616 | 0.636 | 0.637 | 1.88 | CT only |
| only | | | | | or NG |
| (10,000 | | | | | only |
| copies) | | | | | (>0.4, <0.9) |
| NGopa | 0.591 | 0.618 | 0.627 | 3.04 | CT only |
| only | | | | | or NG |
| (40 | | | | | only |
| copies) | | | | | (>0.4, <0.9) |
| CTcry + | 1.180 | 1.214 | 1.203 | 1.46 | Both |
| NGopa | | | | | CT and |
| (10,000 | | | | | NG |
| copies | | | | | (>0.9) |
| each) | | | | | |
| CTcry + | 1.147 | 1.185 | 1.170 | 1.64 | Both |
| NGopa | | | | | CT and |
| (40 | | | | | NG |
| copies | | | | | (>0.9) |
| each) | | | | | |
| No target | 0.204 | 0.210 | 0.204 | 1.60 | None of |
| | | | | | CT or NG |
| | | | | | (<0.4) |

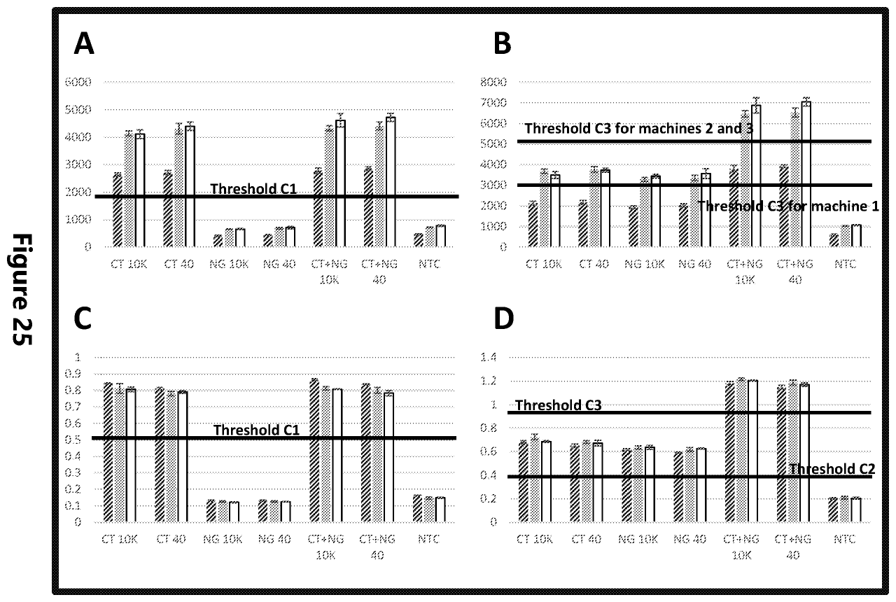

Normalisation using C allows for an alternative method for accurately determining the presence or absence of CTcry, NGopa or both CTcry and NGopa in a sample with fixed threshold values used across all machines. FIG. 25 illustrates the ΔS_{D1} (FIG. 21A), ΔS_{D2} (FIG. 25B), ΔS_{D1}/C (FIG. 25C) and ΔS_{D2}/C (FIG. 25D) across the three Bio-Rad CFX96 machines tested (mean of triplicates; Machine 1 in black stripes, Machine 2 in grey and Machine 3 in white). FIG. 25C shows ΔS_{D1}/C larger than 0.4 (Threshold C₁) indicates the presence of CTcry in the reaction in all three machines. FIG. 25D shows ΔS_{D2}/C values between 0.4 (Threshold C₂) and 0.9 (Threshold C₃) indicates the presence of only one of CTcry or NGopa present in the reaction in all three machines. If ΔS_{D2}/C is above 0.9 (Threshold C₃), it indicates both CTcry and NGopa are present in the reaction in all three machines. If ΔS_{D2}/C is below 0.4, it indicates none of CTcry or NGopa are present in the reaction in all three machines. These results are summarised in Table 21 and Table 22. The combined information from ΔS_{D1}/C and ΔS_{D2}/C can then be used to correctly determine the presence of CTcry and/or NGopa in the reaction. For the ΔS_{D1} values without normalisation, FIG. 25A shows it is possible to use the same Threshold C₁ (2000 RFU) for determination of the presence of CTcry despite the variations between the machines, as summarised in Table 19. However, due to observed run-to-run or machine-to-machine variations in ΔS_{D2}, FIG. 25B shows that different Threshold C3 values are required for different machines and further analysis is not possible using a fixed threshold value. Therefore, normalisation using C allows for the use of fixed threshold values for accurate determination of the presence or absence of targets, regardless of machine-to-machine or run-to-run variations.

This example demonstrates an additional function of LOCS which serves as a calibrator to minimise run-to-run and machine-to-machine variability, which in turn allows for an alternative analysis method that can accurately determine the presence or absence of two targets in a single channel by measurement of discrete fluorescence taken before and after PCR using fixed threshold values and Cq determination of one target.

The calibrator method demonstrated in this example has several advantages including that it does not require the use of additional reagents to be added to the reaction nor does it require the use of data obtained from other wells. This method functions to calibrate and correct for well-to-well variations that may be present. Furthermore, the calibration is processed using the data acquired in the same channel and therefore is not affected by any channel-to-channel variations that may be present between the instruments. Where multiple channels are utilised for a multiplex reaction, each channel can be independently calibrated against the LOCS calibration signal in each channel. This is favorable to a scenario where the signals are calibrated against signals in a different channel, such as that from the internal control or endogenous control, as the calibration is adversely affected where the ratio of the expected signal intensity between the channels differs significantly between the instruments, causing channel-to-channel variations.

### Prophetic Example 13

Method for simultaneous colorimetric detection of two targets using one linear MNAzyme substrate and one LOCS probe.

The following example demonstrates the use of gold nanoparticles for simultaneous colorimetric detection and differentiation of two targets in a single reaction which could be achieved using one linear MNAzyme substrate, detected at one temperature (D₁), and one LOCS reporter, detected at a second temperature (D₂).

Both linear MNAzyme substrates and LOCS reporters could be labelled at each end with gold nanoparticles (GNPs) wherein the GNPs would be in an aggregated state when the substrate and LOCS reporters remain intact and un-cleaved. In the aggregated state, GNPs would exhibit absorbance at a longer wavelength due to coupling of their individual localized plasmon and would be visualised as a purple colour. This purple colour could be observed both by the naked eye and also by spectroscopy in the UV/VIS absorbance region. Cleavage of linear substrates by their respective MNAzyme in the presence of a specific Target 1 would cause separation of the GNPs which would produce a colour change from purple to red that could be observed across a broad range of temperatures. In this example, the colour change from the linear MNAzyme substrate could be detected by measuring the UV/VIS spectroscopic shift of absorbance at Temperature 1 (D₁=52° C.), prior to, and following amplification (ΔS_{D1}), wherein absorbance at a shorter wavelength indicates the presence of target 1. At Temperature 1 (D₁), both the split and the intact LOCS reporter for a second target (Target 2) would not produce any colour change or detectable spectroscopic shift of absorbance, since the Tm of its stem region is higher than Temperature 1 (D₁). Therefore, any colour change or spectroscopic shift of absorbance obtained at Temperature 1 (D₁) following PCR would reflect the presence of Target 1 in the reaction, regardless of the presence or absence of Target 2.

Target-mediated cleavage of a LOCS reporter by its respective MNAzyme would also produce a colour change (purple to red) and/or a spectroscopic shift of absorbance, however, this would only occur within a specific range of temperatures of which are higher than the Tm of the LOCS stem region. In this example, a colour change and/or spectroscopic shift of absorbance could be measured at Temperature 2 (D₂=70° C.) prior to, and following amplification (ΔS_{D2}), which is higher than the Tm of the split LOCS reporter, but lower than the Tm of the intact LOCS reporters (Tm intact LOCS reporter>Temperature 2>Tm split LOCS reporter). Therefore, an intact LOCS reporter would not produce any colour change and/or spectroscopic shift of absorbance at Temperature 2 (D₂) and thus an increased shift of absorbance during PCR, above any that is related to cleaved linear substrate 1 at this temperature (if present), would be associated with split LOCS-1 and would be indicative of the presence of Target 2.

### Prophetic Example 14

Method for simultaneous Surface Plasmon Resonance (SPR) detection of two targets using one linear MNAzyme substrate and one LOCS probe.

The following example demonstrates the use of gold nanoparticles for simultaneous Surface Plasmon resonance (SPR) detection and differentiation of two targets in a single reaction which could be achieved using one linear MNAzyme substrate, detected at one temperature (D₁), and one LOCS reporter, detected at a second temperature (D₂).

Both linear MNAzyme substrates and LOCS reporters could be attached to a gold surface at one end and labelled at the other end with a gold nanoparticle (GNP). When the linear MNAzyme substrate and LOCS are un-cleaved and intact, the GNPs would remain in close proximity to the gold surface and would exhibit a measurable baseline SPR signal. Cleavage of linear substrates by their respective MNAzyme in the presence of a specific Target 1 would cause separation of the GNPs from the gold surface which would produce a measurable shift in SPR signal. In this example, the shift in SPR signal from the linear MNAzyme substrate could be detected at a first Temperature 1 (D₁=52° C.), prior to, and following amplification (ΔS_{D1}), wherein a measurable shift in SPR signal indicates the presence of Target 1. At Temperature 1 (D₁), both the split and the intact LOCS reporter for a second target (Target 2) would not produce any measurable shift in SPR signal, since the Tm of its stem region is higher than Temperature 1 (D₁) and the GNP would remain hybridised to the gold surface. Therefore, any measurable SPR shift obtained at Temperature 1 (D₁) following PCR would reflect the presence of Target 1 in the reaction, regardless of the presence or absence of Target 2.

Target-mediated cleavage of a LOCS reporter by, for example an MNAzyme, could also produce a measurable shift in SPR signal, however, this would only occur within a specific range of temperatures of which are higher than the Tm of the LOCS stem region. In this example, a further measurable shift in SPR signal could be measured at Temperature 2 (for example with D₂=70° C.) prior to, and following amplification (ΔS_{D2}), which is higher than the Tm of the split LOCS reporter, but lower than the Tm of the intact LOCS reporters (Tm intact LOCS reporter>Temperature 2>Tm split LOCS reporter). Therefore, an intact LOCS reporter would not produce any measurable shift in SPR signal at Temperature 2 (D₂) and thus an increased measurable shift in SPR signal during PCR, above any that is related to cleaved linear substrate 1 at this temperature (if present), would be associated with split LOCS-1 and would be indicative of the presence of Target 2.

### Prophetic Example 15

Method for simultaneous electrochemical detection of two targets using one linear MNAzyme substrate and one LOCS probe.

The following example demonstrates the use of redox active species, such as Methylene blue, for simultaneous electrochemical detection and differentiation of two targets in a single reaction which could be achieved using one linear MNAzyme substrate, detected at one temperature (D₁), and one LOCS reporter, detected at a second temperature (D₂).

Both linear MNAzyme substrates and LOCS reporters could be immobilised on an electrode surface, such as a gold electrode by Au—S bonds, at one end and labelled at the other end with Methylene blue which is an electrochemically active molecule. When the linear MNAzyme substrate and LOCS are un-cleaved and intact, the Methylene blue molecule would be restricted in close proximity to the electrode surface and would generate a large current that could be detected by an electrochemical reader.

Cleavage of linear substrates by their respective MNAzyme in the presence of a specific Target 1 would cause separation of the methylene blue molecules from the electrode surface which would cause a significant decrease in current. In this example, the decrease in current from the cleaved linear MNAzyme substrate could be detected at a first Temperature 1 (D₁=52° C.), prior to, and following amplification (ΔS_{D1}), wherein this decrease in current could be used to indicate the presence of Target 1. At Temperature 1 (D₁), both the split and the intact LOCS reporter for a second target (Target 2) would not produce any measurable decrease in current, since the Tm of its stem region is higher than Temperature 1 (D₁) and the Methylene blue molecule would remain hybridised and in close proximity to the electrode surface. Therefore, any measurable decrease in current obtained at Temperature 1 (D₁) following PCR would reflect the presence of Target 1 in the reaction, regardless of the presence or absence of Target 2.

Target-mediated cleavage of a LOCS reporter by its respective MNAzyme could also produce a measurable decrease in current, however, this would only occur within a specific range of temperatures of which are higher than the Tm of the LOCS stem region. In this example, a further measurable decrease in current could be measured at Temperature 2 (D₂=70° C.) prior to, and following amplification (ΔS_{D2}), which is higher than the Tm of the split LOCS reporter, but lower than the Tm of the intact LOCS reporters (Tm intact LOCS reporter>Temperature 2>Tm split LOCS reporter). Therefore, an intact LOCS reporter would not produce any measurable decrease in current at Temperature 2 (D₂) and thus a further decrease in current during PCR, above any that is related to cleaved linear substrate 1 (if present) at this temperature, would be associated with split LOCS-1 and would be indicative of the presence of Target 2.

### Example 16

Methods for simultaneous detection and quantification of multiple targets at a single wavelength using one Molecular Beacon and one LOCS reporter and a strand displacing polymerase lacking 5′ to 3′ exonuclease activity.

The following example demonstrates a method for simultaneous detection and quantification of two targets in a single fluorescent channel by acquiring fluorescence readings at two temperatures in real-time during PCR using one non-cleavable Molecular Beacon and one LOCS reporter. This experiment demonstrates the use of a strand displacing polymerase lacking in 5′ exonuclease activity to eliminate degradation of the Molecular Beacon in the presence of target. This strategy does not require the use of specialized analysis methods demonstrated in Example 4. As illustrated in FIG. 5, both the Molecular Beacon and LOCS probe are labelled with the same fluorophore and quencher moieties for simultaneous detection in the same fluorescence channel. The Molecular Beacon contains a stem region with a Tm A and a Loop region which can specifically hybridize with target 1 (TVbtub) with a Tm B; where Tm B is greater than Tm A (Tm B>Tm A). In this example, the intact LOCS probe has a stem region with a Tm C (82° C.) and a Loop region which when cleaved by an MNAzyme in the presence target 2 (MgPa), generates a Split LOCS with a Tm D (62° C.); where Tm D is less than Tm C (Tm D<Tm C). The presence of target 1 (TVbtub) and/or target 2 (MgPa) can be discriminated by measuring the fluorescence at two temperatures (D₁ and D₂; 50° C. and 72° C.) either in real time after each PCR cycle. In the following example, the Tm A is ˜60° C., Tm B is ˜68° C., Tm C is ˜82° C. and Tm D is ˜62° C. which is consistent with Scenario 3 described in Example 5 wherein D₁<Tm A<Tm B<D₂ and D₁<Tm D<D₂<Tm C.

### Oligonucleotides

The oligonucleotides specific to this experiment include: Forward Primer 12 (SEQ ID NO: 63), Reverse Primer 12 (SEQ ID: 64) and Molecular Beacon 1 (SEQ ID: 68) for the amplification and quantification of Target 1 (TVbtub); Forward primer 5 (SEQ ID NO: 20), Reverse primer 5 (SEQ ID NO: 21), Partzyme A5 (SEQ ID NO: 22), Partzyme B5 (SEQ ID NO: 23), LOCS-2 (SEQ ID NO: 25) for the amplification and quantification of Target 2 (MgPa). The sequences are listed in the Sequence Listing.

### Reaction Conditions

Real-time detection of the target sequence was performed in a total reaction volume of 20 μL using a BioRad® CFX96 thermocycler. The cycling parameters, and fluorescent data acquisition (DA) points, were: 1 cycle of 92° C. for 2 minutes, 50° C. for 15 seconds (DA) and 72° C. for 15 seconds (DA); 50 cycles of 92° C. for 5 seconds, 50° C. for 40 seconds (DA) and 72° C. for 5 seconds (DA). All reactions were performed in triplicates. Each reaction contained 40 nM of each forward primer, 200 nM of each reverse primer, 200 nM of each Partzyme, 200 nM of Molecular Beacon 1, 200 nM of LOCS-2 reporter, 2 units of SD polymerase Hotstart (Bioron), 800 μM dNTP Mix (Bioline), 8 mM MgCl2 (Bioron) and 1× NH4 buffer (Bioline). The reactions on the first plate contained either no target (NF H₂O), various concentrations of synthetic G-Block of TVbtub (25600, 6400, 1600, 400 or 100 copies) in a background of 0 or 25600 copies of MgPa gene or synthetic G-Block of MgPa gene (25600, 6400, 1600, 400 or 100 copies) in a background of 0 or 25600 copies of TVbtub gene.

### Results

During PCR amplification, fluorescence was measured at two temperatures in real time to detect and quantify the presence of target 1 (TVbtub) and/or target 2 (MgPa). The Molecular Beacon was designed to detect sequences homologous to TVbtub for detection of Trichomonas vaginalis (TV). The MNAzyme was designed to cleave LOCS-2 in the presence of MgPa for detection of Mycoplasma genitalium. The cycle number (Cq) at which fluorescence crosses a dynamic threshold (set at 20% of maximum fluorescence) was determined from real-time fluorescence acquisition at 50° C. and 72° C. for detecting and quantifying TVbtub and MgPa, respectively. The presence or absence of specific signal during PCR is akin to that described in Scenario 3 of Example 5.

The standard reactions containing 25600, 6400, 1600, 400 and 100 copies of TVbtub were used to construct a standard curve (R²=0.989; E=139.14%) to quantify the starting concentrations of TVbtub at 50° C. (D₁) in samples that contained TVbtub in the presence or absence of MgPa. Table 23 summarises the copy number of TVbtub determined from the standard curve obtained from the real-time data acquired at 50° C. The calculated copy numbers of TVbtub are unaffected by the presence of 25,600 copies of MgPa in the reaction, as the p-value of 0.297 confirms statistical insignificance (Student's paired t-test). Similarly, the standards containing 25600, 6400, 1600, 400 and 100 copies of MgPa were used to construct a standard curve (R²=0.990; E=116.49%) to quantify the starting concentrations of MgPa at 72° C. (D₂) in samples that contained MgPa in the presence or absence of TVbtub. Table 24 summarises the copy number of MgPa determined from the standard curve obtained from real-time data acquired at 72° C. The calculated copy numbers of MgPa are unaffected by the presence of 25,600 copies of TVbtub in the reaction, as the p-value of 0.319 confirms statistical insignificance (Student's paired t-test).

TABLE 23
Copy number of TVbtub determined from the real-time data acquired
at 50° C. from the samples containing various concentrations
TVbtub, in the presence or absence of 25600 copies of MgPa

| | | Copy number of TVbtub added | | | | | |
|---|---|---|---|---|---|---|---|
| | | 25600 | 6400 | 1600 | 400 | 100 | 0 |
| Calculated | with 0 copies | 31009 | 6142 | 1245 | 352 | 128 | N/A |
| copy | of MgPa | | | | | | |
| number of | with 25600 | 28501 | 5740 | 1235 | 364 | 118 | N/A |
| TVbtub | copies of | | | | | | |
| | MgPa | | | | | | |

TABLE 24
Copy number of MgPa determined from the real-time data acquired
at 72° C. from the samples containing various concentrations
MgPa, in the presence or absence of 25600 copies of TVbtub

| | | Copy number of MgPa added | | | | | |
|---|---|---|---|---|---|---|---|
| | | 25600 | 6400 | 1600 | 400 | 100 | 0 |
| Calculated | with 0 copies | 30996 | 5922 | 1330 | 346 | 125 | N/A |
| copy | of TVbtub | | | | | | |
| number of | with 25600 | 30735 | 6778 | 1665 | 466 | 141 | N/A |
| MgPa | copies of | | | | | | |
| | TVbtub | | | | | | |

### Example 17

Methods for determining background signal using one or more measurement(s) acquired prior to, or following amplification; where the background signal is measured either in the experimental reaction or in an equivalent control reaction lacking target.

The following example demonstrates various strategies allowing qualitative analysis of multiple targets at a single wavelength. The analysis method shown in this example is similar to the Endpoint Analysis Method 2 shown in Example 1, but this example demonstrates the background signal may be determined by different methods rather than using only the pre-amplification readings at D₁ and D₂ in the same reaction well.

The assay is designed such that target 1 (CTcry) can be detected and differentiated using a linear MNAzyme substrate, and target 2 (NGopa) can be detected and differentiated using a LOCS reporter which comprises a different MNAzyme substrate within its Loop. During PCR, MNAzyme 1 can cleave linear substrate 1 in the presence of CTcry to separate a fluorophore and quencher to produce an increase in signal that can be detected across a broad range of temperatures. In this example, endpoint detection of CTcry can be achieved by determining the normalised fluorescence signal (NS_{D1}), which serves a similar function to ΔS_{D1} in Example 1, and is determined as the difference between the post-PCR signal at temperature 1 (D₁; 52° C.) and background signal, which was determined using several different approaches as summarised in Table 25 below. The stem of LOCS-1 in both the intact and split configurations has a Tm above D₁, and therefore NS_{D1} remains unaffected by cleavage of LOCS-1, and hence unaffected by the presence or absence of NGopa in the sample. As such, when NS_{D1} is greater than threshold (X₁), this indicates the presence of the cleaved linear MNAzyme substrate 1 and hence target 1, CTcry.

In the presence of NGopa, the MNAzyme 2 can cleave LOCS-1 during PCR. The normalised fluorescence signal (NS_{D2}) at temperature 2 (D₂, 70° C.) can be calculated as the difference between the post-PCR signal at temperature 2 and the background signal, again measured according to Table 25. NS_{D2} has a similar function to ΔS_{D2} in Example 1. Since the cleavage of LOCS-1 during PCR contributes to increased normalised fluorescence signal at temperature 2 (NS_{D2}), but does not affect NS_{D1}, where the difference between NS_{D2} and NS_{D1} (NS_{D2}−NS_{D1}) crosses a second threshold (X₂); such that NS_{D2}−NS_{D1}=ΔNS_{D2}NS_{D1}>X₂, is an indicative of the presence of split LOCS-1. In contrast, the cleavage of substrate 1 alone contributes toward increase in both NS_{D2} and NS_{D1} values, wherein the difference between these two values (ΔNS_{D2}NS_{D1}) is less than a second threshold (X₂). Therefore when the difference between NS_{D2} and NS_{D1} (ΔNS_{D2}NS_{D1}=NS_{D2}−NS_{D1}) is greater than a second threshold (X₂) this indicates specific detection of the split LOCS-1 and hence target 2, NGopa.

TABLE 25
Methods of calculating normalised signals at D₁ (NS_{D1}) and D₂ (NS_{D2}) with various parameters
for measuring Background Signal measured at the first (D₁) and second (D₂) and at various
third temperatures (D₃ = D_{3A} or D_{3B} or D_{3C})

| Temperature | | | | | | |
|---|---|---|---|---|---|---|
| for measuring | | | | Positive NG | | |
| Background | | Time-point | Positive CT | ΔNS_{D1}NS_{D2} > | | |
| Signal | | and | NS_{D1} > X₁ | X₂ | | |
| for | for | Reaction | Determine | Determine | | |
| NS_{D1} | NS_{D2} | Well | NS_{D1} using | NS_{D2} using | Comment | Fig. |
| 40° C. | | Pre-PCR in | S_{D1-post-PCR} − | S_{D2-post-PCR -} | D_{3A} < D₁ < D₂ | 26 |
| (D_{3A}) | | same well | S_{D3A-pre-PCR} | S_{D3A-pre-PCR} | | A-B |
| 52° C. | | (Sample | S_{D1-post-PCR} − | S_{D2-post-PCR -} | D_{3B} = D₁ < D₂ | 26 |
| (D_{3B}/D₁) | | well) | S_{D3B-pre-PCR} | S_{D3B-pre-PCR} | | C-D |
| 62° C. | | | S_{D1-post-PCR} − | S_{D2-post-PCR -} | D₁ < D_{3C} < D₂ | 26 |
| (D_{3C}) | | | S_{D3C-pre-PCR} | S_{D3C-pre-PCR} | | E-F |
| 52° C. | | Pre-PCR in | S_{D1-post-PCR} − | S_{D2-post-PCR -} | D_{3B} = D₁ < D₂ | 27 |
| (D_{3B}/D₁) | | Negative | S_{D3B-pre-PCR (NTC)} | S_{D3B-pre-PCR (NTC)} | | A-B |
| 52° C. | 70° C. | Control | S_{D1-post-PCR} − | S_{D2-post-PCR -} | D₃ not used | 27 |
| (D₁) | (D₂) | well (NTC) | S_{D1-pre-PCR (NTC)} | S_{D2-pre-PCR (NTC)} | | C-D |
| | | Post-PCR in | S_{D1-post-PCR} − | S_{D2-post-PCR -} | D₃ not used | 27 |
| | | NTC | S_{D1-post-PCR (NTC)} | S_{D2-post-PCR (NTC)} | | E-F |

The following example demonstrates that background signals (S_{D3}) measured before PCR at a third temperature (D₃) within the same well can be used to calculate NS_{D1} and NS_{D2}. Three different Pre-PCR temperatures were tested namely D_{3A}=40° C.; D_{3B}/D₁=52° C. and D_{3C}=60° C. (Table 25). This strategy negates the need for multiple pre-PCR data acquisition points (i.e. once at D₃ instead of twice at D₁ and D₂) and reduces the time and complexity of experiments. Furthermore, the following example demonstrates that background signals can be measured in separate negative control reactions lacking template. In one case a single pre-PCR background measurement taken at D_{3B}=52° C.=D₁ was used to calculate both NS_{D1} and NS_{D2}; while in other cases two background reading were taken at D₁ (52° C.) and D₂ (70° C.) with acquisition either taken before, or following PCR from separate negative control reactions.

### Oligonucleotides

The oligonucleotides specific to this experiment include: Forward Primer 1 (SEQ ID NO: 1), Reverse Primer 1 (SEQ ID: 2), Partzyme A1 (SEQ ID: 3), Partzyme B1 (SEQ ID NO: 4) and linear MNAzyme Substrate 1 (SED ID: 13) for the amplification and detection of Target 1 (CTcry); Forward primer 2 (SEQ ID NO: 5), Reverse primer 2 (SEQ ID NO: 6), Partzyme A5 (SEQ ID NO: 7), Partzyme B5 (SEQ ID NO: 8) and LOCS-1 (SEQ ID NO: 14) for the amplification and detection of Target 2 (NGopa). The sequences are listed in the sequence listing.

### Reaction Conditions

Real-time detection of the target sequence was performed in a total reaction volume of 20 μL using a BioRad® CFX96 thermocycler. The cycling parameters, and fluorescent data acquisition (DA) points were: 1 cycle of 95° C. for 2 minutes, 40° C. for 15 seconds (DA), 52° C. for 15 seconds (DA), 62° C. for 15 seconds (DA) and 70° C. for 15 seconds (DA); 10 cycles of 95° C. for 5 second and 61° C. for 30 seconds (0.5° C. decrement per cycle); 40 cycles of 95° C. for 5 seconds and 52° C. for 40 seconds; and 1 cycle of 52° C. for 15 seconds (DA) and 70° C. for 15 seconds (DA). All reactions were performed in triplicate and each reaction contained 40 nM of each forward primer, 200 nM of each reverse primer, 200 nM of each Partzyme, 200 nM of linear MNAzyme Substrate 1, 200 nM of LOCS-1 reporter and 1× PlexMastermix (Bioline). The reactions contained either no target (NF H₂O), or synthetic G-Block of CTcry (10,000 or 40 copies); or NGopa gene (10,000 or 40 copies); or various concentrations of CTcry gene (10,000 or 40 copies) in a background of NGopa gene (10,000 and 40 copies). All reactions except for the negative (no target) control (NF H₂O) further contained a background of 34.5 ng (10,000 copies) of human genomic DNA.

### Results

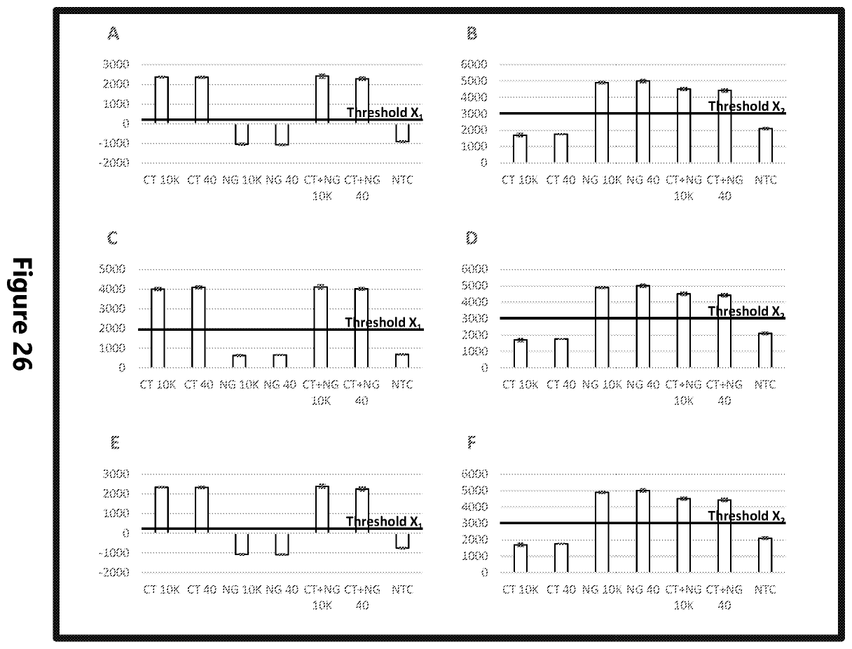

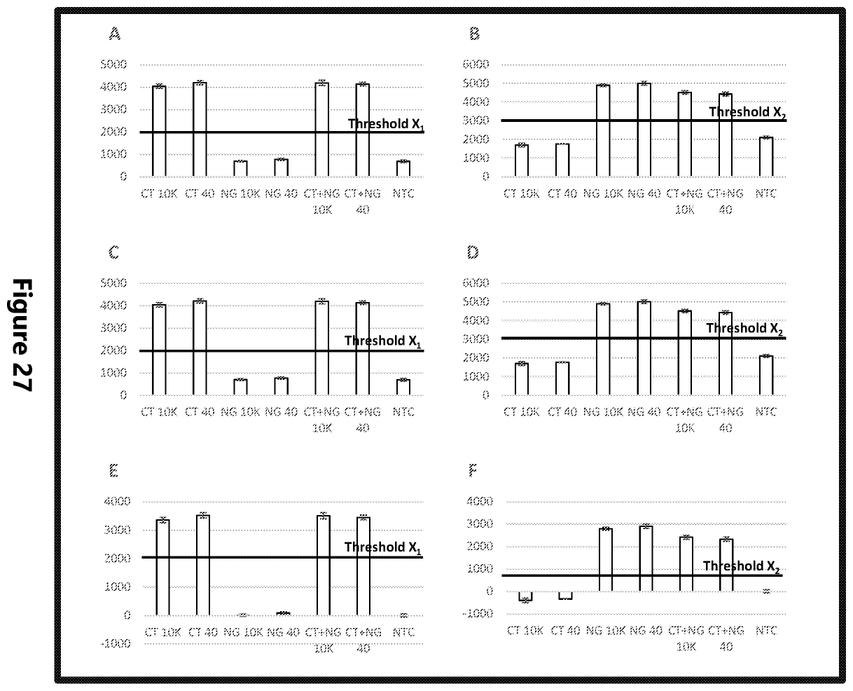

The results in FIG. 26 illustrate the calculated values for NS_{D1} (LHS) and ΔNS_{D2}NS_{D1} (RHS) for the detection of CTcry and NGopa, respectively, using background signal determined using pre-PCR fluorescence measurements (S_{D3}) from within the same reaction well at 40° C. (FIG. 26A-B); 52° C. (FIG. 26C-D) and 62° C. (FIG. 26E-F). The results in FIG. 27 illustrate the calculated values for NS_{D1} (LHS) and ΔNS_{D2} NS_{D1} (RHS) for the detection of CTcry and NGopa, respectively, using background signal values determined from separate negative control reactions. Background signals were determined as the mean of no template control signals measured at D_{3B}/D₁ prior to PCR (FIGS. 27A-B) and at D₁ and D₂ prior to PCR (FIG. 27C-D) and following PCR (FIG. 27E-F).

The results in FIG. 26A, FIG. 26C, FIG. 26E, FIG. 27A, FIG. 27C and FIG. 27E show that for all scenarios, the normalised signal at temperature 1 (NS_{D1}) is greater than threshold 1 (X₁) when CTcry is present within the sample regardless of whether NGopa is present or absent, but does not cross this threshold in the presence NGopa only and/or when CTcry is absent from the sample. Therefore, a normalised endpoint signal greater than threshold 1 at temperature 1 is indicative of the presence of CTcry. The results in FIG. 26B, FIG. 26D, FIG. 26F, FIG. 27B, FIG. 27D and FIG. 27F illustrate for all six scenarios, ΔNS_{D2}NS_{D1} is greater than threshold 2 (X₂) when NGopa is present within the sample, but does not cross this threshold when CTcry only is present within the sample and/or when NGopa is absent from the sample (NTC). Therefore, a difference in normalised endpoint fluorescent signals, NS_{D2} and NS_{D1}, greater than threshold 2 (ΔNS_{D2}NS_{D1}>X₂) is indicative of the presence of NGopa.

This example demonstrates the use of one linear MNAzyme substrate and one LOCS reporter for endpoint detection and differentiation of two targets in a single fluorescence channel with flexibility of determining a background signal to be measured at a third temperature and/or from data from separate negative control reactions.

TABLE 26
seqeunces used in Examples 1-17

| Seq | | | |
|---|---|---|---|
| ID | Designation | Target | Sequence |
| 1 | Forward primer 1 | CTcry | AATATCATCTTTGCGGTTGCGTGTCC |
| 2 | Reverse primer 1 | CTcry | GCTGTGACGGAGTACAAACGCC |
| 3 | Partzyme Al | CTcry | TCCTGTGACCTTCATTATGTCGACAACGAGAGGAAACCTT/3Phos/ |
| 4 | Partzyme B1 | CTcry | TGCCCAGGGAGGCTAGCTGAGTCTGAGCACCCTAGGC/3Phos/ |
| 5 | Forward primer 2 | NGopa | GTGTTGAAACACCGCCCGG |
| 6 | Reverse primer 2 | NGopa | GCTCCTTATTCGGTTTGACCGG |
| 7 | Partzyme A2 | NGopa | CCGGAACCCGATATAATCCGCACAACGAGAGGGTCGAG/3Phos/ |
| 8 | Partzyme B2 | NGopa | GGACGAGGGAGGCTAGCTCCTTCAACATCAGTGAAAATCTTTT/3Phos/ |
| 9 | Forward primer 3 | TFRC | GCTAAAACAATAACTCAGAACTTACG |
| 10 | Reverse primer 3 | TFRC | CAGCTTTCTGAGGTTACCATCCTA |
| 11 | Partzyme A3 | TFRC | GGAATATGGAAGGAGACTGTCACAACGAGGGGTCGAG/3Phos/ |
| 12 | Partzyme B3 | TFRC | GGAATATGGAAGGAGACTGTCACAACGAGGGGTCGAG/3Phos/ |
| 13 | Substrate 1 | Universal | /56-JOEN/AAGGTTTCCTCguCCCTGGGCA/3IABkFQ/ |
| 14 | LOCS-1 | Universal | /56JOEN/AACGACAATGGCCTTTTCTCGACCCTCguCCCTCGTCCTTTTGGCCATTGTCG |
| | | | TT/3IABkFQ/ |
| 15 | Substrate 2 | Universal | /5RHO101N/CTCGACCCCguCTCCACGCCA/3IAbRQSp/ |
| 16 | Forward primer 4 | TVK | GTTTGTGTCTCGTGCCATAGTCG |
| 17 | Reverse primer 4 | TVK | ATTTCATGGTCGCCCTCGGAGT |
| 18 | Partzyme A4 | TVK | TATATGAGTTTGAGACCAAGAATGACAACGAGAGGCGTGAT/3Phos/ |
| 19 | Partzyme B4 | TVK | CTGGGAGGAGAGGCTAGCTGTGTAACTCGACCTGTCCGATTCA/3Phos/ |
| 20 | Forward primer 5 | MgPa | GGCTCGCCATTTAAACCCCTTTGCACCG |
| 21 | Reverse primer 5 | MgPa | AATACCTTGATGGTCAGCAAAACTTTGCAAT |
| 22 | Partzyme A5 | MgPa | TGCTAAGTTAATATCATATAAAGCTCTAACAACGAGGGACGTCGA/3Phos/ |
| 23 | Partzyme B5 | MgPa | GCGGTAGAGGAGGCTAGCTCCGTTGTTATCATACCTTCTGATTG/3Phos/ |
| 24 | Substrate 3 | Universal | /56-FAM/ATCACGCCTCguCTCCTCCCAG/3IABkFQ/ |
| 25 | LOCS-2 | Universal | /56-FAM/GCGTGACCGGTCCAAAATCGACGTCCCrGrUCC |
| | | | TCTACCGCAAAAGGACCGGTCACGC/3IABkFQ/ |
| 26 | Partzyme A6 | TFRC | GGAATATGGAAGGAGACTGTCACAACGAGTGGTTGGC/3Phos/ |
| 27 | Partzyme B6 | TFRC | GTCGTGTTGGAGGCTAGCTCCTCTGACTGGAAAACAGACT/3Phos/ |
| 28 | Substrate 4 | Universal | GCCAACCACguCCAACACGAC |
| 29 | Forward primer 6 | TPApolA | AGGGCTAGTACACCGGAGG |
| 30 | Reverse Primer 6 | TPApolA | CCTAAGATCTCTATTTTCTATAGG |
| 31 | Partzyme A7 | TPApolA | TTGAAGTCGGAGTTGAAGACGAACAACGAGGGACGTCGA/3Phos/ |
| 32 | Partzyme B7 | TPApolA | CGGTAGAGGAGGCTAGCTGTGCTGTGTCTGGCGCCATA/3Phos/ |
| 33 | LOCS-3 | Universal | /5Atto680N/AACGACAATGGCCTTTTTCGACGTCCCguCCTCTACCGCTTTTGGCCATTG |
| | | | TCGTT/3IAbRQSp/ |
| 34 | LOCS-4 | Universal | /5Cy5/CACGGTCTAGAGCTCTTTTAAGGTTTCCTCguCCCTGGGCATTTTGAGCTCTAG |
| | | | ACCGTG/3IAbRQSp/ |
| 35 | Partzyme A8 | NGopa | GGAACCCGATATAATCCGCACAACGAGAGGGTCGAG/3Phos/ |
| 36 | Partzyme B8 | NGopa | GGACGAGGGAGGCTAGCTCCTTCAACATCAGTGAAAATC/3Phos/ |
| 37 | LOCS-5 | Universal | /56-FAM/ |
| | | | CGCACTGGCTTTTCTCGACCCTCguCCCTCGTCCTTTTGCCAGTGCG/3IABkFQ/ |
| 38 | Forward Primer 7 | LGV | TACAGAAAAAATAGACCCTTTCC |
| 39 | Reverse Primer 7 | LGV | GTATTCTCCTTTATCTACTGTGC |
| 40 | Partzyme A9 | LGV | CCGAGCATCACTAACTGTTGACAACGAGAGGCGTG/3Phos/ |
| 41 | Partzyme B9 | LGV | CTGGGAGGAGAGGCTAGCTGAGCAGGCGGAGTTGATGAT/3Phos/ |
| 42 | LOCS-6 | Universal | /5Cy5/CGCACTGGCTTTTATCACGCCTCguCTCCTCCCAGTTTTGCCAGTGCG/3IAbRQSp/ |
| 43 | Forward Primer 8 | NGporA | AGCATTCAATTTGTTCCGAGTC |
| 44 | Reverse Primer 8 | NGporA | CAACAGCCGGAACTGGTTTCAT |
| 45 | Partzyme A10 | NGporA | AAGTCCGCCTATACGCCTGACAACGAGAGGTGCGGT/3Phos/ |
| 46 | Partzyme B10 | NGporA | AGCTGGGGAGGCTAGCTCTACTTTCACGCTGGAAAGTA/3Phos/ |
| 47 | LOCS-7 | Universal | /56- |
| | | | FAM/AACGACAATGGCCTTTTACCGCACCTCguCCCCAGCTCTTTTGGCCATTGTCGTT/ |
| | | | 3IABkFQ/ |
| 48 | Forward Primer 9 | HSV-1 | CTAACAGCGCGAACGACCAACTAC |
| 49 | Reverse Primer 9 | HSV-1 | CAGCCCCCATACCGGAACGC |
| 50 | Partzyme A11 | HSV-1 | CCGATCATCAGTTATCCTTAAGACAACGAGGGGTCGAG/3Phos/ |
| 51 | Partzyme B11 | HSV-1 | TGGCGTGGAGAGGCTAGCTGTCTCTTTTGTGTGGTGCGTT/3Phos/ |
| 52 | LOCS- | Universal | /56JOEN/TCGGGTAGCTTTTTTCTCGACCCCguCTCCACGCCATTTTAAGCTACCCGA/ |
| | | | 3IABkFQ/ |
| 53 | Forward Primer 10 | HSV-2 | CTACCAAATACGCCTTAGCAGACC |
| 54 | Reverse Primer 10 | HSV-2 | CAGGCTGAATGTGGTAAACACGCTTC |
| 55 | Partzyme A12 | HSV-2 | AGACCCCTCGCTTAAGATGGACAACGAGAGGACTAGG/3Phos/ |
| 56 | Partzyme B12 | HSV-2 | GACGTGAGGAGGCTAGCTCCGATCCCAATCGATTTCGC/3Phos/ |
| 57 | LOCS-9 | Universal | /56JOEN/ACAGTGCATTTTCCTAGTCCTCguCCTCACGTCCTTTTTGCACTGT/3IABkFQ/ |
| 58 | Forward Primer 11 | MgPa | GTTGAGAAATACCTTGATGGTCAGCAAAAC |
| 59 | Reverse Primer 11 | MgPa | ACCCCTTTGCACCGTTGAGG |
| 60 | Partzyme A13 | MgPa | ATCAGAAGGTATGATAACAACGGACAACGAGAGGGCGGTT/3Phos/ |
| 61 | Partzyme B13 | MgPa | GGTTCACGGGAGGCTAGCTTAGAGCTTTATATGATATTAACTTAG/3Phos/ |
| 62 | LOCS-10 | Unniversal | /5RHO101N/TCTACGCCACCAGTTTTACCGCCCTCguCCCGTGAACTTTTCTGGTGGCGT |
| | | | AGA/3IAbRQSp/ |
| 63 | Forward Primer 12 | TVbtub | TGCATTGATAACGAAGCTCTTTATGATATTTGC |
| 64 | Reverse Primer 12 | TVbtub | AACATGTTGTTCCGGACATAACCAT |
| 65 | Partzyme A14 | TVbtub | CCGTACACTCAAGCTCACAACACACAACGAGGGGAGAGGA/3Phos/ |
| 66 | Partzyme B14 | TVbtub | TTGAAGGGGAGGCTAGCTCAACATACGGCGATCTTAACCAC/3Phos/ |
| 67 | LOCS-11 | Universal | /5RHO101N/TCAAGGACTTTTTCCTCTCCCCguCCCCTTCAACTTTTGTCCTTGA/ |
| | | | 3IAbRQSp/ |
| 68 | Molecular Beacon 1 | TVbtub | /56-FAM/ |
| | | | TATGCCGACTTTTCACCAACATACGGCGATCTTAACTTTTCTCGGCATA/3IABkFQ/ |

### Description of Oligo Sequences in Table 26

Oligonucleotide sequences are listed from 5′ to 3′. UPPERCASE bases represent DNA and lowercase bases represent RNA./56-FAM/ indicates the location of a FAM fluorophore,/56-JOEN/ indicates the location of a JOE fluorophore,/5RHO101N/indicates the location of an ATTO Rho101 fluorophore,/5Atto680N/ indicates the location of an ATTO 680 fluorophore and/5Cy5/indicates the location of a cy5.5 fluorophore./3IABkFQ/represents the location of an Iowa Black FQ quencher capable of absorbing fluorescence in the range of 420-620 nm and/3IAbRQSp/ represents the location of an Iowa Black RQ quencher used for absorbing fluorescence in the range of 500-700 nm./3Phos/ indicates a 3′ phosphate group.

1. A method for determining the presence or absence of first and second targets in a sample, the method comprising:
(a) preparing a mixture for a reaction by contacting the sample or a derivative thereof putatively comprising the first and second targets with:
a first oligonucleotide for detection of the first target, and comprising a first detection moiety capable of generating a first detectable signal;
an intact stem-loop oligonucleotide for detection of the second target, and comprising a double-stranded stem portion of hybridised nucleotides, opposing strands of which are linked by an unbroken single-stranded loop portion of unhybridised nucleotides, wherein the stem portion comprises a second detection moiety capable of generating a second detectable signal,
wherein the first and second detection moieties are capable of generating detectable signals that cannot be differentiated at a single temperature using a single type of detector; and
a first enzyme capable of digesting one or more of the unhybridised nucleotides of the intact stem-loop oligonucleotide only when the second target is present in the sample;
(b) treating the mixture under conditions suitable for:
the first target to induce a modification to the first oligonucleotide thereby enabling the first detection moiety to generate a first detectable signal,
digestion of one or more of the unhybridised nucleotides of the intact stem-loop oligonucleotide by the first enzyme, only when the second target is present in the sample, to thereby break the single-stranded loop portion and provide a split stem-loop oligonucleotide;
(c) measuring:
a background signal provided by the first and the second detection moieties in the mixture, or, in a control mixture;
(d) determining whether at one or more timepoints during or after said treating:
a first detectable signal arising from said modification is generated at a first temperature which differs from the background signal and is indicative of the presence of the first target in the sample;
a second detectable signal is generated at a second temperature which differs from the background signal and is indicative of the presence of the second target in the sample;
wherein:
at the first temperature the second detectable signal does not differ from the background signal, and
at the second temperature:
if present, strands of the double-stranded stem portion of the split stem-loop oligonucleotide are partially or completely dissociated enabling the second detection moiety to provide the second detectable signal; and
if present, strands of the double-stranded stem portion of the intact stem-loop oligonucleotide cannot dissociate thereby preventing the second detectable moiety from providing the second detectable signal.

2. The method of claim 1, wherein said determining in part (d) comprises:
using a predetermined threshold value to determine if the first detectable signal arising from said modification differs from any said background signal at the first temperature; and/or
using a predetermined threshold value to determine if the second detectable signal differs from any said background signal at the second temperature.

3. The method of claim 1 or claim 2, wherein the control mixture does not comprise:
the first target; or
the second target; or
the first and second targets,
but is otherwise equivalent to the mixture.

4. The method of any one of claims 1 to 3, wherein the control mixture comprises a predetermined amount of:
the first target; or
the second target; or
the first and second targets,
but is otherwise equivalent to the mixture.

5. The method of any one of claims 1 to 4, wherein:
the modification to the first oligonucleotide enables the first detection moiety to provide the first detectable signal at or below the first temperature; and
generation of the first detectable signal is reversible.

6. The method of claim 5, wherein:
part (c) comprises measuring:
a first background signal at or within 1° C., 2° C., 3° C., 4° C., or 5° C. of a first temperature, and a second background signal at or within 1° C., 2° C., 3° C., 4° C., or 5° C. of a second temperature;
provided by the first and the second detection moieties in the mixture, or, in the control mixture; and
part (d) comprises determining whether at one or more timepoints during or after said treating:
a first detectable signal arising from said modification is generated at the first temperature which differs from the first background signal and is indicative of the presence of the first target in the sample;
a second detectable signal is generated at the second temperature which differs from the second background signal and is indicative of the presence of the second target in the sample.

7. The method of claim 5 or claim 6, wherein:
the first target is a nucleic acid sequence;
the first oligonucleotide is a stem-loop oligonucleotide comprising a double-stranded stem portion of hybridised nucleotides on opposing strands of which are linked by an unbroken single-stranded loop portion of unhybridised nucleotides of which all or a portion is/are complementary to the first target; and
the modification of the first oligonucleotide is a conformational change arising from hybridisation of the target to the single-stranded loop portion of the first oligonucleotide by complementary base pairing.

8. The method of claim 7, wherein:
the conformational change is dissociation of strands in the double-stranded stem portion of the first oligonucleotide arising from said hybridisation of the target to the single-stranded loop portion of the first oligonucleotide by complementary base pairing.

9. The method of claim 7 or claim 8, wherein:
the stem portion of the first oligonucleotide has a melting temperature (Tm) that is: below the Tm of a double-stranded duplex formed from said hybridisation of the target to the single-stranded loop portion of the first oligonucleotide, and above the Tm of the stem portion of the split stem-loop oligonucleotide;
said double-stranded duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: said double-stranded duplex, the stem portion of the first oligonucleotide, the stem portion of the intact stem-loop oligonucleotide, and the stem portion of the split stem-loop oligonucleotide;
the second temperature is below the Tm of: said double-stranded duplex, the stem portion of the first oligonucleotide, and the stem portion of the intact stem-loop oligonucleotide; and is above the Tm of the stem portion of the split stem-loop oligonucleotide; and
the first temperature is below the second temperature.

10. The method of claim 7 or claim 8, wherein:
the stem portion of the first oligonucleotide has a melting temperature (Tm) that is: below the Tm of a double-stranded duplex formed from said hybridisation of the target to the single-stranded loop portion of the first oligonucleotide, below the Tm of the stem portion of the intact stem-loop oligonucleotide;
said double-stranded duplex has a Tm that is above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: said double-stranded duplex, the stem portion of the first oligonucleotide, the stem portion of the intact stem-loop oligonucleotide, and the stem portion of the split stem-loop oligonucleotide;
the second temperature is above the Tm of: the stem portion of the first oligonucleotide, and the stem portion of the split stem-loop oligonucleotide; and is below the Tm of: said double-stranded duplex, and the stem portion of the intact stem-loop oligonucleotide; and
the first temperature is below the second temperature.

11. The method of claim 7 or claim 8, wherein:
the stem portion of the first oligonucleotide has a melting temperature (Tm) that is: below the Tm of a double-stranded duplex formed from said hybridisation of the target to the single-stranded loop portion of the first oligonucleotide, below the Tm of the stem portion of the intact stem-loop oligonucleotide, and below the Tm of the stem portion of the split stem-loop oligonucleotide;
said double-stranded duplex has a Tm that is: below the Tm of the stem portion of the intact stem-loop oligonucleotide;
the stem portion of the intact stem-loop oligonucleotide has a Tm that is above the Tm of the stem portion of the split stem-loop oligonucleotide;
the first temperature is below the Tm of: said double-stranded duplex, the stem portion of the first oligonucleotide, the stem portion of the intact stem-loop oligonucleotide, and the stem portion of the split stem-loop oligonucleotide;
the second temperature is above the Tm of: said double-stranded duplex, the stem portion of the first oligonucleotide, and the stem portion of the split stem-loop oligonucleotide; and is below the Tm of: the stem portion of the intact stem-loop oligonucleotide; and
the first temperature is below the second temperature.

12. The method of claim 7 or claim 8, wherein:
the stem portion of the first oligonucleotide has a melting temperature (Tm) that is: below the Tm of a double-stranded duplex formed from said hybridisation of the target to the single-stranded loop portion of the first oligonucleotide, above the Tm of the stem portion of the intact stem-loop oligonucleotide, and above the Tm of the stem portion of the split stem-loop oligonucleotide;
said double-stranded duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the stem portion of the first oligonucleotide and said double-stranded duplex; and above the Tm of: the stem portion of the intact stem-loop oligonucleotide and the stem portion of the split stem-loop oligonucleotide;
the second temperature is below the Tm of: the stem portion of the first oligonucleotide, said double-stranded duplex, and the stem portion of the intact stem-loop oligonucleotide; and is above the Tm of the stem portion of the split stem-loop oligonucleotide; and
the first temperature is above the second temperature.

13. The method of any one of claims 7 to 12, wherein:
the Tm of the stem portion of the first oligonucleotide is between 1° C. and 10° C., 1° C. and 5° C., 5° C. and 10° C., or more than 10° C., below the Tm of said double-stranded duplex; and/or
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of: the stem portion of the first oligonucleotide, and/or said double-stranded duplex; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

14. The method of claim 5 or claim 6, wherein:
the first target is a nucleic acid sequence;
the first oligonucleotide is a stem-loop oligonucleotide comprising:
a double-stranded stem portion of hybridised nucleotides, opposing strands of which are linked by a single-stranded loop portion of unhybridised nucleotides, all or a portion of which is/are complementary to the first target, and a second single-stranded portion extending from one of said opposing strands in a 3′ direction and terminating with a sequence that is complementary to a portion of the first target, and
a blocker molecule preceding said sequence that is complementary to the portion of the first target;
the mixture further comprises a polymerase;
said treating the mixture comprises:
hybridising the second single-stranded portion to the first target by complementary base pairing;
extending the second single-stranded portion using the polymerase and the first target as a template sequence to provide a double-stranded nucleic acid, wherein said blocker molecule prevents the polymerase extending the first target using the stem portion of the first oligonucleotide as a template; and
denaturing the double-stranded nucleic acid and hybridising the second single-stranded portion extended by the polymerase to the single-stranded loop portion of the first oligonucleotide by complementary base pairing to produce a signaling duplex and thereby provide said modification to the first oligonucleotide enabling the first detection moiety to provide the first detectable signal.

15. The method of claim 14, wherein:
the stem portion of the first oligonucleotide has a melting temperature (Tm) that is: below the Tm of the signaling duplex and above the Tm of the stem portion of the split stem-loop oligonucleotide;
the signaling duplex has a Tm that is: above the Tm of the stem portion of the intact stem-loop oligonucleotide, and above the Tm of the stem portion of the split stem-loop oligo nucleotide;
the stem portion of the intact stem-loop oligonucleotide has a Tm that is above the Tm of the stem portion of the split stem-loop oligonucleotide;
the first temperature is below the Tm of: the signaling duplex, the stem portion of the first oligonucleotide, the stem portion of the intact stem-loop oligonucleotide, and the stem portion of the split stem-loop oligonucleotide;
the second temperature is below the Tm of: the signaling duplex, the stem portion of the first oligonucleotide, and the stem portion of the intact stem-loop oligonucleotide; and is above the Tm of the stem portion of the split stem-loop oligonucleotide; and
the first temperature is below the second temperature.

16. The method of claim 14, wherein:
the stem portion of the first oligonucleotide has a melting temperature (Tm) that is: below the Tm of the signaling duplex, below the Tm of the stem portion of the intact stem-loop oligonucleotide;
the signaling duplex has a Tm that is above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the signaling duplex, the stem portion of the first oligonucleotide, the stem portion of the intact stem-loop oligonucleotide, and the stem portion of the split stem-loop oligonucleotide;
the second temperature is above the Tm of: the stem portion of the first oligonucleotide, and the stem portion of the split stem-loop oligonucleotide; and is below the Tm of: the signaling duplex, and the stem portion of the intact stem-loop oligonucleotide; and
the first temperature is below the second temperature.

17. The method of claim 14, wherein:
the stem portion of the first oligonucleotide has a melting temperature (Tm) that is: below the Tm of the signaling duplex, below the Tm of the stem portion of the intact stem-loop oligonucleotide, and below the Tm of the stem portion of the split stem-loop oligonucleotide;
the signaling duplex has a Tm that is: below the Tm of the stem portion of the intact stem-loop oligonucleotide,
the stem portion of the intact stem-loop oligonucleotide has a Tm that is above the Tm of the stem portion of the split stem-loop oligonucleotide;
the first temperature is below the Tm of: the signaling duplex, the stem portion of the first oligonucleotide, the stem portion of the intact stem-loop oligonucleotide, and the stem portion of the split stem-loop oligonucleotide;
the second temperature is above the Tm of: the signaling duplex, the stem portion of the first oligonucleotide, and the stem portion of the split stem-loop oligonucleotide; and is below the Tm of: the stem portion of the intact stem-loop oligonucleotide; and
the first temperature is below the second temperature.

18. The method of claim 14, wherein:
the stem portion of the first oligonucleotide has a melting temperature (Tm) that is: below the Tm of the signaling duplex, above the Tm of the stem portion of the intact stem-loop oligonucleotide, and above the Tm of the stem portion of the split stem-loop oligonucleotide;
the signaling duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the stem portion of the first oligonucleotide and the signaling duplex; and above the Tm of: the stem portion of the intact stem-loop oligonucleotide and the stem portion of the split stem-loop oligonucleotide;
the second temperature is below the Tm of: the stem portion of the first oligonucleotide, the signaling duplex, and the stem portion of the intact stem-loop oligonucleotide; and is above the Tm of the stem portion of the split stem-loop oligonucleotide; and
the first temperature is above the second temperature.

19. The method of any one of claims 14 to 18, wherein:
the Tm of the stem portion of the first oligonucleotide is between 1° C. and 10° C., 1° C. and 5° C., 5° C. and 10° C., or more than 10° C., below the Tm of the signaling duplex; and/or
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of: the stem portion of the first oligonucleotide, and/or the signaling duplex; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

20. The method of any one of claims 5 to 19, wherein:
the first detection moiety is a fluorophore and the modification increases its distance from a quencher molecule.

21. The method of claim 20, wherein:
the first oligonucleotide comprises the quencher molecule.

22. The method of claim 21, wherein:
the fluorophore and the quencher molecule are located on opposing strands of the double-stranded stem portion of the first oligonucleotide.

23. The method of claim 5 or claim 6, wherein:
the first target is a nucleic acid sequence;
the first oligonucleotide comprises:
a first double-stranded portion of hybridised nucleotides, a first strand of which extends into a single-stranded portion terminating with a complementary sequence capable of hybridising to a portion of the first target, wherein the first strand comprises a blocker molecule preceding said complementary sequence;
the mixture further comprises a polymerase;
said treating the mixture comprises:
hybridising said complementary sequence of the single-stranded portion to a portion of the first target by complementary base pairing;
extending the complementary sequence using the polymerase and the first target as a template sequence to provide a second double-stranded portion, wherein said blocker molecule prevents the polymerase extending the first target using the first strand of the said first double-stranded portion as a template;
denaturing the first and second double-stranded portions; and
hybridising the complementary sequence extended by the polymerase to the first strand of the first double-stranded portion by complementary base pairing to produce a signaling duplex and thereby provide said modification to the first oligonucleotide enabling the first detection moiety to provide the first detectable signal.

24. The method of claim 23, wherein:
the first double-stranded portion has a melting temperature (Tm) that is: below the Tm of the signaling duplex, and above the Tm of the stem portion of the split stem-loop oligonucleotide;
the signaling duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the signaling duplex, the first double-stranded portion, the stem portion of the intact stem-loop oligonucleotide, and the stem portion of the split stem-loop oligonucleotide;
the second temperature is below the Tm of: the signaling duplex, the first double-stranded portion, and the stem portion of the intact stem-loop oligonucleotide; and is above the Tm of the stem portion of the split stem-loop oligonucleotide; and
the first temperature is below the second temperature.

25. The method of claim 23, wherein:
the first double-stranded portion has a melting temperature (Tm) that is: below the Tm of the signaling duplex, below the Tm of the stem portion of the intact stem-loop oligonucleotide;
the signaling duplex has a Tm that is above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the signaling duplex, the first double-stranded portion, the stem portion of the intact stem-loop oligonucleotide, and the stem portion of the split stem-loop oligonucleotide;
the second temperature is above the Tm of: the first double-stranded portion, and the stem portion of the split stem-loop oligonucleotide; and is below the Tm of: the signaling duplex, and the stem portion of the intact stem-loop oligonucleotide; and
the first temperature is below the second temperature.

26. The method of claim 23, wherein:
the first double-stranded portion has a melting temperature (Tm) that is: below the Tm of the signaling duplex, below the Tm of the stem portion of the intact stem-loop oligonucleotide, and below the Tm of the stem portion of the split stem-loop oligonucleotide;
the signaling duplex has a Tm that is below the Tm of the stem portion of the intact stem-loop oligonucleotide;
the stem portion of the intact stem-loop oligonucleotide has a Tm that is above the Tm of the stem portion of the split stem-loop oligonucleotide;
the first temperature is below the Tm of: the signaling duplex, the first double-stranded portion, the stem portion of the intact stem-loop oligonucleotide, and the stem portion of the split stem-loop oligonucleotide;
the second temperature is above the Tm of: the signaling duplex, the first double-stranded portion, and the stem portion of the split stem-loop oligonucleotide;
and is below the Tm of: the stem portion of the intact stem-loop oligonucleotide; and
the first temperature is below the second temperature.

27. The method of claim 23, wherein:
the first double-stranded portion has a melting temperature (Tm) that is: below the Tm of the signaling duplex, above the Tm of the stem portion of the intact stem-loop oligonucleotide, and above the Tm of the stem portion of the split stem-loop oligonucleotide;
the signaling duplex has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the first double-stranded portion and the signaling duplex; and above the Tm of: the stem portion of the intact stem-loop oligonucleotide and the stem portion of the split stem-loop oligonucleotide;
the second temperature is below the Tm of: the first double-stranded portion, the signaling duplex, and the stem portion of the intact stem-loop oligonucleotide; and is above the Tm of the stem portion of the split stem-loop oligonucleotide; and
the first temperature is above the second temperature.

28. The method of any one of claims 23 to 27, wherein:
the Tm of the first double-stranded portion is between 1° C. and 10° C., 1° C. and 5° C., 5° C. and 10° C., or more than 10° C., below the Tm of the signaling duplex; and/or
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of: the first double-stranded portion, and/or the signaling duplex; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

29. The method of any one of claims 23 to 28, wherein:
the first detection moiety is a fluorophore and the modification increases its distance from a quencher molecule.

30. The method of claim 29, wherein:
the first oligonucleotide comprises the quencher molecule.

31. The method of claim 30, wherein:
the fluorophore and the quencher molecule are located on opposing strands of the first double-stranded portion.

32. The method of claim 5 or claim 6, wherein:
the first target is a nucleic acid sequence;
the mixture further comprises:
a first primer complementary to a first sequence in the first target,
a second oligonucleotide comprising a component complementary to a second sequence in the first target that differs from the first sequence, and a tag portion that is not complementary to the first target,
a first polymerase comprising exonuclease activity, and
optionally a second polymerase, and
said treating the mixture comprises:
suitable conditions to hybridise the first primer and the second oligonucleotide to the first target,
extending the first primer using the first polymerase and the target as a template to thereby cleave off the tag portion,
hybridising the cleaved tag portion to the first oligonucleotide by complementary base pairing,
and extending the tag portion using the first or second polymerase and the first oligonucleotide as a template to generate a double-stranded sequence comprising the first oligonucleotide thereby providing said modification to the first oligonucleotide and enabling the first detection moiety to provide the first detectable signal.

33. The method of claim 32, wherein:
the double-stranded sequence has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the double-stranded sequence, the stem portion of the intact stem-loop oligonucleotide, and the stem portion of the split stem-loop oligonucleotide;
the second temperature is below the Tm of: the double-stranded sequence, and the stem portion of the intact stem-loop oligonucleotide; and is above the Tm of the stem portion of the split stem-loop oligonucleotide; and
the first temperature is below the second temperature.

34. The method of claim 32, wherein:
the double-stranded sequence has a Tm that is: above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the double-stranded sequence, the stem portion of the intact stem-loop oligonucleotide, and the stem portion of the split stem-loop oligonucleotide;
the second temperature is above the Tm of: and the stem portion of the split stem-loop oligonucleotide; and is below the Tm of: the double-stranded sequence, and the stem portion of the intact stem-loop oligonucleotide; and
the first temperature is below the second temperature.

35. The method of claim 32, wherein:
the double-stranded sequence has a Tm that is: below the Tm of the stem portion of the intact stem-loop oligonucleotide;
the stem portion of the intact stem-loop oligonucleotide has a Tm that is above the Tm of the stem portion of the split stem-loop oligonucleotide;
the first temperature is below the Tm of: the double-stranded sequence, the stem portion of the intact stem-loop oligonucleotide, and the stem portion of the split stem-loop oligonucleotide;
the second temperature is above the Tm of: the double-stranded sequence, and the stem portion of the split stem-loop oligonucleotide; and is below the Tm of: the stem portion of the intact stem-loop oligonucleotide; and
the first temperature is below the second temperature.

36. The method of claim 32, wherein:
the double-stranded sequence has a Tm that is: above the Tm of the stem portion of the intact stem-loop oligonucleotide, and above the Tm of the stem portion of the split stem-loop oligonucleotide;
the stem portion of the intact stem-loop oligonucleotide has a Tm that is above the Tm of the stem portion of the split stem-loop oligonucleotide;
the first temperature is below the Tm of: the double-stranded sequence;
and above the Tm of: the stem portion of the intact stem-loop oligonucleotide and the stem portion of the split stem-loop oligonucleotide;
the second temperature is below the Tm of: the double-stranded sequence and the stem portion of the intact stem-loop oligonucleotide; and is above the Tm of the stem portion of the split stem-loop oligonucleotide; and
the first temperature is above the second temperature.

37. The method of any one of claims 32 to 36, wherein:
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of the double-stranded sequence; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

38. The method of any one of claims 32 to 37, wherein:
the first oligonucleotide comprises a fluorophore and a quencher molecule, and
said extending the tag portion increases the distance between the fluorophore and the quencher molecule.

39. The method of claim 5 or claim 6, wherein:
the first target is a nucleic acid sequence;
the first oligonucleotide is complementary to a first portion of the target;
the mixture further comprises a further oligonucleotide complementary to a second portion the first target, wherein the first and second portions of the first target flank one another but do not overlap;
said treating the mixture comprises:
forming a duplex structure comprising:
(iii) a first double-stranded component by hybridising the first oligonucleotide to the target by complementary base pairing, and
(iv) a second double-stranded component by hybridising the further oligonucleotide to the target by complementary base pairing,
thereby bringing the first and further oligonucleotides into proximity, and providing said modification to the first oligonucleotide enabling the first detection moiety to provide the first detectable signal.

40. The method of claim 39, wherein:
the duplex structure has a Tm that is below the Tm of the stem portion of the intact stem-loop oligonucleotide;
the stem portion of the intact stem-loop oligonucleotide has a Tm that is above the Tm of the stem portion of the split stem-loop oligonucleotide;
the first temperature is below the Tm of: the duplex structure, the stem portion of the intact stem-loop oligonucleotide, and the stem portion of the split stem-loop oligonucleotide;
the second temperature is above the Tm of: the duplex structure, and the stem portion of the split stem-loop oligonucleotide; and is below the Tm of: the stem portion of the intact stem-loop oligonucleotide; and
the first temperature is below the second temperature.

41. The method of claim 39 of claim 40, wherein:
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the Tm of the duplex structure; and/or
the second temperature is between 1° C. and 10° C., 1° C. and 5° C., 5° C. and 10° C., or more than 10° C., below the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide.

42. The method of any one of claims 39 to 41, wherein:
the first detectable moiety is a fluorophore and the further oligonucleotide comprises a quencher;
said forming of the duplex structure further brings the fluorophore and quencher into proximity; and
said detectable signal is a decrease in fluorescence provided by the first detection moiety.

43. The method of claim 5 or claim 6, wherein:
the first target is a nucleic acid sequence;
the first detection moiety is: a nanoparticle, a metallic nanoparticle, a noble metal nanoparticle, an alkali metal nanoparticle, a gold nanoparticle, or a silver nanoparticle; to which the first oligonucleotide is bound;
said treating the mixture comprises:
hybridising the first target to the first oligonucleotide to thereby induce the modification to the first oligonucleotide enabling the first detection moiety to provide a first detectable signal indicative of the presence of the first target in the sample;
wherein the first detectable signal is:
(i) a change in refractive index,
(ii) a change in colour; and/or
(iii) a change in absorption spectrum,
arising from the first detection moiety following said modification of the first oligonucleotide.

44. The method of claim 5 or claim 6, wherein:
the first target is a nucleic acid sequence;
the first detection moiety is an electrochemical agent to which the first oligonucleotide is bound;
said treating the mixture comprises:
hybridising the first target to the first oligonucleotide to thereby induce or facilitate the modification to the first oligonucleotide enabling the first detection moiety to provide a first detectable signal indicative of the presence of the first target in the sample;
wherein the first detectable signal is a change in electrochemical signal arising from the first detection moiety following said modification of the first oligonucleotide.

45. The method of claim 44, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

46. The method of any one of claims 5 to 19, 23 to 28, 32 to 37, and 39 to 41 wherein:
the first detection moiety is a nanoparticle, a metallic nanoparticle, a noble metal nanoparticle, an alkali metal nanoparticle, a gold nanoparticle, or a silver nanoparticle; to which the first oligonucleotide is bound; and
the first detectable signal is:
(i) a change in refractive index,
(ii) a change in colour; and/or
(iii) a change in absorption spectrum,
arising from the first detection moiety following said modification of the first oligonucleotide.

47. The method of any one of claims 5 to 19, 23 to 28, 32 to 37, and 39 to 41 wherein:
the first detection moiety is an electrochemical agent to which the first oligonucleotide is bound; and
the first detectable signal is a change in electrochemical signal arising from the first detection moiety following said modification of the first oligonucleotide.

48. The method of claim 47, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

49. The method of any one of claims 43 to 48, wherein:
the second detection moiety is a nanoparticle, a metallic nanoparticle, a noble metal nanoparticle, an alkali metal nanoparticle, a gold nanoparticle, or a silver nanoparticle; to which the intact stem-loop oligonucleotide is bound; and
the second detectable signal is:
(i) a change in refractive index,
(ii) a change in colour; and/or
(iii) a change in absorption spectrum,
arising from said strands of the double-stranded stem portion of the split stem-loop oligonucleotide dissociating.

50. The method of any one of any one of claims 43 to 48, wherein:
the second detection moiety is an electrochemical agent to which the intact stem-loop oligonucleotide is bound; and
the second detectable signal is a change in electrochemical signal arising from said strands of the double-stranded stem portion of the split stem-loop oligonucleotide dissociating.

51. The method of claim 50, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

52. The method of any one of claims 20 to 22, 29 to 31, 38, and 42, wherein:
the second detection moiety is a fluorophore, and
the second detectable signal provided by said strands of the double-stranded stem portion of the split stem-loop oligonucleotide dissociating increases the distance of the fluorophore from a quencher molecule.

53. The method of claim 52, wherein:
the fluorophore and quencher molecule are located on opposing strands of the double-stranded stem portion of the split stem-loop oligonucleotide.

54. The method of any one of claims 1 to 4, wherein:
generation of the first detectable signal is not reversible;
the modification to the first oligonucleotide enables the first detection moiety to provide the first detectable signal at or below the first temperature; and
the first detectable signal provided at or below the first temperature remains detectable at the second temperature.

55. The method of claim 54, wherein:
part (c) comprises measuring:
(i) a first background signal at or within 1° C., 2° C., 3° C., 4° C., or 5° C. of a first temperature, and a second background signal at or within 1° C., 2° C., 3° C., 4° C., or 5° C. of a second temperature, and/or
(ii) a third background signal at a third temperature;
provided by the first and the second detection moieties in the mixture, or, in a control mixture; and
part (d) comprises determining whether at one or more timepoints during or after said treating:
(i) a first detectable signal arising from said modification is generated at the first temperature which differs from the first or third background signal, wherein:
at the first temperature the second detectable signal does not differ from the first or third background signal, and
detection of a difference between the first detectable signal and the first or third background signal is indicative of said modification of the first oligonucleotide and the presence of the first target in the sample; and
(ii) a second detectable signal is generated at the second temperature which differs from the second or third background signal and is indicative of the presence of the second target in the sample.

56. The method of claim 55, wherein:
when a first target is present in the sample, said determining whether a second detectable signal is generated at the second temperature comprises compensating for the first detectable signal present when measuring the second detectable signal.

57. The method of claim 55 or claim 56, wherein:
the first signal that differs from the first background signal is generated,
the second signal that differs from the second background signal is generated, and
the second detectable signal differs from the second background signal to a greater extent than the first detectable signal differs from the first background signal, thereby indicating that the second target is present in the sample.

58. The method of claim 57, wherein:
the first temperature is below: the second temperature, the Tm of the double-stranded stem portion of the intact stem-loop oligonucleotide, and the Tm of the stem portion of the split stem-loop oligonucleotide.

59. The method of claim 57, wherein:
the first temperature is higher than: the second temperature, the Tm of the stem portion of the intact stem-loop oligonucleotide, and the Tm of the stem portion of the split stem-loop oligonucleotide.

60. The method of claim 55, wherein:
the first signal that differs from the third background signal is generated,
the second signal that differs from the third background signal is generated, and
the second signal differs from the third background signal to a greater extent than the first signal differs from the third background signal,
thereby indicating that the second target is present in the sample.

61. The method of claim 55, wherein:
the second temperature is higher than the first temperature,
the third temperature is lower the Tm of the double-stranded stem portion of the intact stem-loop oligonucleotide,
the first detectable signal that differs from the third background signal is generated,
the second detectable signal that differs from the third background signal is generated, and
the second detectable signal differs from the third background signal to a greater extent than the first signal differs from the third background signal, thereby indicating that the second target is present in the sample.

62. The method of any one of claims 55 to 61 wherein:
the Tm of the stem portion of the intact stem-loop oligonucleotide is above the Tm of the stem portion of the split stem-loop oligonucleotide;
the first temperature is below the second temperature, and is below the Tm of the stem portion of the split stem-loop oligonucleotide; and
the second temperature is above the Tm of the stem portion of the split stem-loop oligonucleotide and below the Tm of the stem portion of the intact stem-loop oligonucleotide.

63. The method of claim 62, wherein:
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., below the second temperature; and/or
the first temperature is between 1° C. and 10° C., 1° C. and 5° C., 5° C. and 10° C., or more than 10° C., below the Tm of the stem portion of the split stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide; and/or
the second temperature is between 1° C. and 10° C., 1° C. and 5° C., 5° C. and 10° C., or more than 10° C., below the Tm of the stem portion of the intact stem-loop oligonucleotide.

64. The method of claim 62 or claim 63, comprising:
measuring said third background signal, wherein the third temperature is below the second temperature.

65. The method of claim 64, wherein:
the third temperature is between 1° C. and 10° C., 1° C. and 5° C., 5° C. and 10° C., or more than 10° C., below the second temperature.

66. The method of any one of claims 55 to 61 wherein:
the Tm of the stem portion of the intact stem-loop oligonucleotide is above the Tm of the stem portion of the split stem-loop oligonucleotide;
the first temperature is above the second temperature, is above the Tm of the stem portion of the split stem-loop oligonucleotide, and is above the Tm of the stem portion of the intact stem-loop oligonucleotide; and
the second temperature is above the Tm of the stem portion of the split stem-loop oligonucleotide and is below the Tm of the stem portion of the intact stem-loop oligonucleotide.

67. The method of claim 66, wherein:
the Tm of the stem portion of the intact stem-loop oligonucleotide is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., above the second temperature; and/or
the first temperature is between 1° C. and 10° C., 1° C. and 5° C., 5° C. and 10° C., or more than 10° C., above the Tm of the stem portion of the split stem-loop oligonucleotide; and/or
the first temperature is between 1° C. and 10° C., 1° C. and 5° C., 5° C. and 10° C., or more than 10° C., above the Tm of the stem portion of the intact stem-loop oligonucleotide; and/or
the second temperature is between 1° C. and 10° C., 1° C. and 5° C., 5° C. and 10° C., or more than 10° C., above the Tm of the stem portion of the split stem-loop oligonucleotide; and/or
the second temperature is between 1° C. and 10° C., 1° C. and 5° C., 5° C. and 10° C., or more than 10° C., below the Tm of the stem portion of the intact stem-loop oligonucleotide.

68. The method of any one of claims 54 to 67, wherein:
the first oligonucleotide is a substrate for a multi-component nucleic acid enzyme (MNAzyme);
the mixture further comprises:
an MNAzyme capable of cleaving the first oligonucleotide when the first target is present in the sample; and
said treating the mixture further comprises:
binding of the MNAzyme to the first target and hybridisation of the substrate arms of the MNAzyme to the first oligonucleotide by complementary base pairing to facilitate cleavage of the first oligonucleotide thereby providing said modification to the first oligonucleotide and enabling the first detection moiety to provide the first detectable signal.

69. The method of claim 68, wherein:
the first target is a nucleic acid sequence; and
said treating the reaction mixture further comprises:
hybridising the first target to the sensor arms of the MNAzyme by complementary base pairing to thereby facilitate assembly of the MNAzyme.

70. The method of any one of claims 54 to 67, wherein:
the first oligonucleotide is a substrate for an aptazyme;
the first target is an analyte, protein, compound or molecule;
the mixture further comprises an aptazyme comprising an aptamer capable of binding to the first target; and
said treating the mixture further comprises:
binding of the aptazyme to the first target and the first oligonucleotide to facilitate cleavage of the first oligonucleotide thereby providing said modification to the first oligonucleotide and enabling the first detection moiety to generate the first detectable signal.

71. The method of any one of claims 54 to 67, wherein:
the first target is a nucleic acid sequence;
the first oligonucleotide comprises a sequence that is complementary to the first target,
the mixture further comprises:
a primer complementary to a portion of the first target, and
a polymerase with exonuclease activity;
said treating the mixture comprises:
hybridising the primer to the first target by complementary base pairing,
hybridising the first oligonucleotide to the first target by complementary base pairing
extending the primer using the polymerase and the first target as a template sequence to thereby digest the first oligonucleotide and provide said modification to the first oligonucleotide enabling the first detection moiety to generate the first detectable signal.

72. The method any one of claims 54 to 67, wherein:
the first target is a nucleic acid sequence;
the mixture further comprises:
a restriction endonuclease capable of digesting a double-stranded duplex comprising the first target; and
said treating the mixture comprises:
hybridising the first oligonucleotide to the first target by complementary base pairing to thereby form a double-stranded duplex,
digesting the duplex using the restriction endonuclease to thereby provide said modification to the first oligonucleotide and enabling the first detection moiety to provide the first detectable signal.

73. The method of claim 72, wherein:
the restriction endonuclease is a nicking endonuclease capable of associating with and cleaving a strand of said double-stranded duplex, and said strand comprises all or a portion of the first oligonucleotide.

74. The method of any one of claims 54 to 67, wherein:
the mixture further comprises a DNAzyme or a ribozyme requiring a co-factor for catalytic activity;
said treating of the mixture comprises using conditions suitable for:
binding of the cofactor to the DNAzyme or ribozyme to render it catalytically active,
hybridisation of the DNAzyme or ribozyme to the first oligonucleotide by complementary base pairing, and
catalytic activity of the DNAzyme or ribozyme to thereby digest the first oligonucleotide and thereby provide said modification to the first oligonucleotide enabling the first detection moiety to provide the first detectable signal.
wherein
the first target is the co-factor.

75. The method of claim 74, wherein the co-factor is a metal ion, or a metal ion selected from: Mg²⁺, Mn²⁺, Ca²⁺, Pb²⁺.

76. The method of any one of claims 54 to 75, wherein:
the first detection moiety is a fluorophore and the modification to the first oligonucleotide increases the distance of the fluorophore from a quencher molecule.

77. The method of claim 76, wherein:
the first oligonucleotide comprises the quencher molecule.

78. The method of claim 76 or claim 77, wherein:
the second detection moiety is a fluorophore, and
the second detectable signal provided by said strands of the double-stranded stem portion of the split stem-loop oligonucleotide dissociating increases the distance of the fluorophore from a quencher molecule.

79. The method of claim 78, wherein:
the fluorophore and quencher molecule are located on opposing strands of the double-stranded stem portion of the split stem-loop oligonucleotide.

80. The method of any one of claims 54 to 79, wherein:
the first detection moiety is a nanoparticle, a metallic nanoparticle, a noble metal nanoparticle, an alkali metal nanoparticle, a gold nanoparticle, or a silver nanoparticle; to which the first oligonucleotide is bound; and
the first detectable signal is:
(i) a change in refractive index,
(ii) a change in colour; and/or
(iii) a change in absorption spectrum,
arising from the first detection moiety following said modification of the first oligonucleotide.

81. The method of any one of claims 54 to 79, wherein:
the first detection moiety is an electrochemical agent to which the first oligonucleotide is bound; and
the first detectable signal is a change in electrochemical signal arising from the first detection moiety following said modification of the first oligonucleotide.

82. The method of claim 81, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

83. The method of any one of claims 80 to 82, wherein:
the second detection moiety is a nanoparticle, a metallic nanoparticle, a noble metal nanoparticle, an alkali metal nanoparticle, a gold nanoparticle, or a silver nanoparticle; to which the intact stem-loop oligonucleotide is bound; and
the second detectable signal is:
(i) a change in refractive index,
(ii) a change in colour; and/or
(iii) a change in absorption spectrum,
arising from said strands of the double-stranded stem portion of the split stem-loop oligonucleotide dissociating.

84. The method of any one of any one of claims 80 to 82, wherein:
the second detection moiety is an electrochemical agent to which the intact stem-loop oligonucleotide is bound; and
the second detectable signal is a change in electrochemical signal arising from said strands of the double-stranded stem portion of the split stem-loop oligonucleotide dissociating.

85. The method of claim 84, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

86. The method of any one of claims 1 to 85, wherein the intact stem-loop oligonucleotide is not hybridised to the second target during said digestion of the one or more unhybridised nucleotides of the intact stem-loop oligonucleotide by the first enzyme.

87. The method of any one of claims 1 to 86, wherein:
the first enzyme is a first MNAzyme, and
said treating the mixture comprises:
binding of the first MNAzyme to the second target and hybridisation of substrate arms of said first MNAzyme to the loop portion of the intact stem-loop oligonucleotide, to thereby digest the one or more unhybridised nucleotides of the intact stem-loop oligonucleotide and provide the split stem-loop oligonucleotide.

88. The method of claim 87, wherein:
the second target is a nucleic acid sequence; and
said treating the mixture further comprises:
hybridising the second target to the sensor arms of the first MNAzyme by complementary base pairing to thereby facilitate assembly of the first MNAzyme.

89. The method of any one of claims 1 to 86, wherein:
the second target is an analyte, protein, compound or molecule;
the first enzyme is an aptazyme comprising an aptamer capable of binding to the second target; and
binding of the second target to the aptamer is capable of rendering the first enzyme catalytically active.

90. The method of claim 89, wherein:
the first enzyme is any one of an: apta-DNAzyme, apta-ribozyme, apta-MNAzyme.

91. The method of any one of claims 1 to 86, wherein:
the second target is an analyte, protein, compound or molecule;
the first oligonucleotide is a substrate for an aptazyme;
the first enzyme is an aptazyme comprising an aptamer portion capable of binding to the second target, and a nucleic acid enzyme portion capable of digesting the one or more unhybridised nucleotides of the intact stem-loop oligonucleotide
said treating the mixture further comprises:
binding the second target to the aptamer portion of the aptazyme to facilitate activation of catalytic activity of the nucleic acid enzyme portion, and hybridising the intact stem-loop oligonucleotide to the active nucleic acid enzyme portion to thereby digest the one or more unhybridised nucleotides of the intact stem-loop oligonucleotide.

92. The method of any one of claims 1 to 85, wherein:
the second target is a nucleic acid sequence; and
the first enzyme is a first restriction endonuclease, and said treating the mixture comprises:
using conditions suitable for hybridisation of the second target to the single-stranded loop portion of the intact stem-loop oligonucleotide by complementary base pairing to form a double-stranded sequence for the first restriction endonuclease to associate with and digest the one or more unhybridised nucleotides of the single-stranded loop portion thereby forming the split stem-loop oligonucleotide.

93. The method of claim 92, wherein:
the first restriction endonuclease is a first nicking endonuclease capable of associating with and cleaving a strand of said double-stranded sequence for the first restriction endonuclease, and said strand comprises all or a portion of the single-stranded loop portion of the intact stem-loop oligonucleotide.

94. The method of any one of claims 1 to 85, wherein:
the first enzyme comprises a polymerase with exonuclease activity,
said treating the mixture comprises using conditions suitable for:
hybridisation of the second target to the single-stranded loop portion of the intact stem-loop oligonucleotide by complementary base pairing to form a first double-stranded sequence comprising a portion of the second target,
hybridisation of a first primer oligonucleotide to the second target to form a second double-stranded sequence located upstream relative to the first double-stranded sequence comprising the portion of the second target,
extending the primer using the polymerase with exonuclease activity and using the second target as a template sequence,
wherein the first polymerase comprising exonuclease activity digests the single-stranded loop portion of the first double-stranded sequence and thereby forms the split stem-loop oligonucleotide.

95. The method of any one of claims 1 to 85, wherein:
the first enzyme is an exonuclease, and
said treating the mixture comprises using conditions suitable for:
hybridisation of the second target to the single-stranded loop portion of the intact stem-loop oligonucleotide by complementary base pairing to form a first double-stranded sequence comprising a portion of the second target,
association of the first enzyme comprising exonuclease activity with the double-stranded sequence comprising the second target, and
catalytic activity of the first enzyme comprising exonuclease activity allowing it to digest the single-stranded loop portion of the first double-stranded sequence comprising the second target and thereby form the split stem-loop oligonucleotide.

96. The method of any one of claims 1 to 85, wherein:
the first enzyme is a DNAzyme or a ribozyme requiring a co-factor for catalytic activity, and said treating the mixture comprises using conditions suitable for:
binding of the cofactor to the first enzyme to render it catalytically active,
hybridisation of the DNAzyme or ribozyme to the single-stranded loop portion of the intact stem-loop oligonucleotide by complementary base pairing,
catalytic activity of the DNAzyme or ribozyme to digest the one or more unhybridised nucleotides of the single-stranded loop portion of the intact stem-loop oligonucleotide and thereby form the split stem-loop oligonucleotide,
wherein:
the second target is the co-factor.

97. The method of claim 96, wherein the co-factor is a metal ion, or a metal ion selected from: Mg²⁺, Mn²⁺, Ca²⁺, Pb²⁺.

98. The method of any one of claims 1 to 97, wherein:
the first target differs from the second target; and/or
the first oligonucleotide comprises or consists of a sequence that is not within the single-stranded loop portion of the intact stem-loop oligonucleotide.

99. The method of any one of claims 1 to 98, wherein:
the first enzyme does not digest the second target.

100. The method of any one of claims 1 to 71, 74 to 91, or 94 to 99, wherein:
any said enzyme does not digest the first target and/or the second target.

101. The method of any one of claims 1 to 100, wherein:
the first temperature differs from the second temperature by more than: 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 25° C., 30° C., 35° C., 40° C., 45° C., 50° C., 55° C., or 60° C.

102. The method of any one of claims 1 to 101, wherein said determining comprises detection of the first detectable signal and/or any said background signal(s):
at one or more timepoints during said treating; or
at one or more timepoints during said treating and at one or more timepoints after said treating.

103. The method of any one of claims 1 to 101, wherein said determining comprises detection of the first detectable signal and/or any said background signal(s):
at one or more timepoints after said treating.

104. The method of any one of claims 1 to 101, wherein said determining comprises detection of the second detectable signal and/or any said background signal(s):
at one or more timepoints during said treating; or
at one or more timepoints during said treating and at one or more timepoints after said treating.

105. The method of any one of claims 1 to 101, wherein said determining comprises detection of the second detectable signal and/or any said background signal(s):
one or more timepoints after said treating.

106. The method of any one of claims 1 to 105, wherein:
said determining the presence or absence of the first and second targets comprises a melt curve analysis.

107. The method of claim 6, wherein:
said determining the presence or absence of the first and second targets comprises a melt curve analysis comprising the first and second detectable signals and the optionally the first and second background signals.

108. The method of claim 55, wherein:
said determining the presence or absence of the first and second targets comprises a melt curve analysis comprising the first and second detectable signals and the optionally the first and second background signals; or
the first and second detectable signals and optionally the third background signal.

109. The method of any one of claims 1 to 108, wherein:
the first target and/or the second target is an amplicon of a nucleic acid.

110. The method of any one of claims 1 to 109, wherein:
the first target is a nucleic acid and/or the second target is a nucleic acid, and
the mixture further comprises reagents for amplification of said first and/or second target,
said treating the mixture further comprises conditions suitable for conducting amplification of the first and/or second targets.

111. The method of claim 110, wherein:
the amplification is any one or more of polymerase chain reaction (PCR), strand displacement amplification (SDA), nicking enzyme amplification reaction (NEAR), helicase dependent amplification (HDA), Recombinase Polymerase Amplification (RPA), loop-mediated isothermal amplification (LAMP), rolling circle amplification (RCA), transcription-mediated amplification (TMA), self-sustained sequence replication (3SR), nucleic acid sequence based amplification (NASBA), Ligase Chain Reaction (LCR) or Ramification Amplification Method (RAM), and/or reverse transcription polymerase chain reaction (RT-PCR).

112. The method of claim 110 or claim 111, wherein said determining:
occurs prior to said amplification or within 1, 2, 3, 4, or 5 cycles of said amplification commencing; and/or
occurs after completion of said amplification.

113. The method of any one of claims 110 to 112, wherein said determining:
occurs prior to said amplification or within 1, 2, 3, 4, or 5 minutes of said amplification commencing; and/or
occurs after completion of said amplification.

114. The method of any one of claims 110 to 113, wherein said determining occurs:
at a first timepoint prior to said amplification; and
at a second timepoint after completion of said amplification.

115. The method of any one of claims 110 to 114, wherein:
the amplification method is polymerase chain reaction (PCR); and
said determining occurs at multiple cycles optionally at each cycle.

116. The method of claim 110 or claim 111, further comprising normalising:
the first detectable signal at the first temperature measured at a timepoint during or after said amplification using a positive control signal generated at the first temperature prior to said amplification and/or prior to said treating the reaction; and/or
the second detectable signal at the second temperature measured at a timepoint during or after said amplification using a positive control signal generated at the second temperature prior to said amplification and/or prior to said treating the reaction.

117. The method of claim 110 or claim 111, further comprising normalising:
the first detectable signal using a detectable signal generated by the intact stem-loop oligonucleotide at the first temperature prior to said amplification and/or prior to said treating the reaction; and/or
the second detectable signal using a detectable signal generated by the intact stem-loop oligonucleotide at an additional temperature prior to said amplification and/or prior to said treating the reaction;
wherein the additional temperature is above the Tm of the intact stem-loop oligonucleotide.

118. The method of any one of claims 1 to 117 further comprising:
generating a first target positive control signal using a known concentration of the first target and/or a known concentration of the first oligonucleotide after said modification.

119. The method of any one of claims 1 to 118:
further comprising generating a first target positive control signal by repeating the method on a separate control sample comprising said first target.

120. The method of claim 119, wherein:
the separate control sample comprising the first target comprises a known concentration of the first target.

121. The method of claim 119 or claim 120, wherein:
the separate control sample comprising the first target further comprises the second target.

122. The method of any one of claims 1 to 121, further comprising:
generating a second target positive control signal using a known concentration of the second target and/or a known concentration of the stem-loop oligonucleotide after said modification.

123. The method of any one of claims 1 to 122, further comprising:
generating a second target positive control signal by repeating said method on a separate control sample comprising the second target.

124. The method of claim 123, wherein:
the control sample comprising the second target comprises a known concentration of the second target.

125. The method of claim 123 or claim 124, wherein:
said control sample comprising the second target further comprises said first target.

126. The method of any one of claims 1 to 125, further comprising:
generating a combined positive control signal by repeating said method on a separate control sample comprising the first target and the second target.

127. The method of claim 126, wherein:
the combined control sample comprises a known concentration of the first target and/or a known concentration of the second target.

128. The method of any one of claims 116 to 127, further comprising:
normalising the first detectable signal and/or the second detectable signal using any said positive control signal.

129. The method of any one of claims 116 to 128, further comprising:
assessing levels of a negative control signal by repeating the method of any one of claims 1 to 115 on a separate negative control sample that does not contain:
(i) said first target; or
(ii) said second target; or
(iii) said first target or said second target.

130. The method of claim 129, further comprising:
normalising the first detectable signal and/or the second detectable signal using said negative control signal.

131. The method of any one of claims 116 to 130, wherein:
any said control signal is a fluorescent control signal.

132. The method of any one of claims 1 to 131, further comprising comparing the first and/or second detectable signals to a threshold value wherein:
the threshold value is generated using detectable signals derived from a series of samples or derivatives thereof tested according to the method of any one of claims 1 to 115, and comprising any one or more of:
(i) a no template control and the first target
(ii) a no template control and the second target
(iii) a no template control, the first target, and the second target to thereby determine said presence or absence of the first and second targets in the sample.

133. The method of claim 132, wherein:
the series of samples or derivatives thereof is tested using a known concentration of the first oligonucleotide and/or a known concentration of the intact stem-loop oligonucleotide.

134. The method of any one of claims 1 to 133, wherein:
the sample is a biological sample obtained from a subject.

135. The method of any one of claims 1 to 133:
wherein the method is performed in vitro.

136. The method of any one of claims 1 to 133:
wherein the method is performed ex vivo.

137. The method of any one of claims 1 to 136, wherein:
the first and second detectable moieties emit in the same colour region of the visible spectrum.

138. A composition comprising:
a first oligonucleotide for detection of a first target, wherein the first target is a nucleic acid and complementary to at least a portion of the first oligonucleotide, and
a first detection moiety, wherein:
the first detection moiety is capable of generating a first detectable signal upon modification of the first oligonucleotide, and
the modification is induced by hybridisation of the first target to the first oligonucleotide by complementary base pairing;
an intact stem-loop oligonucleotide for detection of the second target, and comprising a double-stranded stem portion of hybridised nucleotides opposing strands of which are linked by an unbroken single-stranded loop portion of unhybridised nucleotides, wherein at least one strand of the double-stranded stem portion comprises a second detection moiety; and
a first enzyme capable of digesting one or more of the unhybridised nucleotides of the intact stem-loop oligonucleotide only when the second target is present in the sample, to thereby break the single-stranded loop portion and provide a split stem-loop oligonucleotide;
wherein:
the second detection moiety is capable of generating a second detectable signal upon dissociation of the double-stranded stem portion of the split stem-loop oligonucleotide, and
the first and second detection moieties are capable of generating detectable signals that cannot be differentiated at a single temperature using a single type of detector.

139. The composition of claim 138, wherein:
the region of the first oligonucleotide which is complementary to the first target has a different melting temperature (Tm) to each strand of the double-stranded stem portion of the intact stem-loop oligonucleotide.

140. The composition of claim 138 or claim 139, wherein the first oligonucleotide differs in sequence from:
each strand of the double-stranded stem portion of the intact stem-loop oligonucleotide; and
the single-stranded loop portion of the intact stem-loop oligonucleotide.

141. The composition of any one of claims 138 to 140, wherein:
the first oligonucleotide is a stem-loop oligonucleotide comprising a double-stranded stem portion of hybridised nucleotides on opposing strands of which are linked by an unbroken single-stranded loop portion of unhybridised nucleotides of which all or a portion, is/are complementary to the first target.

142. The composition of claim 141, wherein:
the first target is hybridised to the first oligonucleotide by complementary base pairing causing dissociation of strands in the double-stranded stem portion of the first oligonucleotide thereby enabling the first detection moiety to provide the first detectable signal.

143. The composition of any one of claims 138 to 140, wherein:
the first oligonucleotide is a stem-loop oligonucleotide comprising:
a double-stranded stem portion of hybridised nucleotides, opposing strands of which are linked by a single-stranded loop portion of unhybridised nucleotides, all or a portion of which is/are complementary to the first target, and
a second single-stranded portion extending from one of said opposing strands in a 3′ direction and terminating with a sequence that is complementary to a portion of the first target, and
a blocker molecule preceding said sequence that is complementary to the portion of the first target.

144. The composition of claim 143, wherein:
the first target is hybridised to the second single-stranded portion thereof by complementary base pairing;
the composition further comprises a polymerase capable of extending the second single-stranded portion using the first target as a template sequence to provide a double-stranded nucleic acid, wherein said blocker molecule is capable of preventing the polymerase extending the first target using said one opposing strand as a template, and
upon denaturing the double-stranded nucleic acid, the second single-stranded portion extended by the polymerase is capable of hybridising to the single-stranded loop portion of the first oligonucleotide by complementary base pairing to produce a signaling duplex and thereby enable the first detection moiety to provide a first detectable signal.

145. The composition of any one of claims 141 to 144, wherein:
the first detection moiety is a fluorophore.

146. The composition of claim 145, wherein:
the first oligonucleotide comprises a quencher molecule, and the fluorophore and the quencher molecule are located on opposing strands of the double-stranded stem portion of the first oligonucleotide.

147. The composition of any one of claims 138 to 140, wherein:
the first oligonucleotide comprises:
a first double-stranded portion of hybridised nucleotides, a first strand of which extends into a single-stranded portion terminating with a complementary sequence capable of hybridising to a portion of the first target, wherein the first strand comprises a blocker molecule preceding said complementary sequence.
the composition further comprises a polymerase.

148. The composition of claim 147, wherein:
a portion of the first target is hybridised to said complementary sequence of the single-stranded portion by complementary base pairing; and
the composition further comprises a polymerase capable of extending the complementary sequence using the first target as a template sequence to provide a second double-stranded portion, wherein said blocker molecule prevents the polymerase extending the first target using the single-stranded portion as a template; and
when the first and second double-stranded portions are denatured, the complementary sequence extended by the polymerase is capable of hybridising to the first strand of the first double-stranded portion by complementary base pairing to produce a signaling duplex and thereby enable the first detection moiety to provide the first detectable signal.

149. The composition of claim 147 or claim 148, wherein:
the first detection moiety is a fluorophore and the modification increases its distance from a quencher molecule;

150. The composition of claim 149, wherein:
the first oligonucleotide comprises a quencher molecule, and the fluorophore and the quencher molecule are located on opposing strands of the first double-stranded portion.

151. The composition of any one of claims 138 to 140, wherein:
the first oligonucleotide is complementary to a first portion of the target;
the composition further comprises an additional oligonucleotide complementary to a second portion the first target, wherein the first and second portions of the first target flank one another but do not overlap, and are each capable of hybridising to the first target to form a duplex structure comprising:
(iii) a first double-stranded component by hybridising the first oligonucleotide to the target or by complementary base pairing, and
(iv) a second double-stranded component by hybridising the additional oligonucleotide to the target by complementary base pairing,
thereby bringing the first and additional oligonucleotides into proximity, and enabling the first detection moiety to provide the first detectable signal.

152. The composition of claim 151, wherein:
the first detectable moiety is a fluorophore and the additional oligonucleotide comprises a quencher;
said forming of the duplex structure further brings the fluorophore and quencher into proximity; and
said detectable signal is a decrease in fluorescence provided by the first detection moiety.

153. The method of any one of claims 138 to 140, wherein:
the first oligonucleotide is hybridised to the first target by complementary base pairing,
the composition further comprises:
a primer hybridised to a portion of the first target by complementary base pairing, and
a polymerase with exonuclease activity capable of extending the primer using the first target as a template sequence to thereby digest the first oligonucleotide and modify the first oligonucleotide enabling the first detection moiety to provide the first detectable signal.

154. The composition of any one of claims 138 to 140, wherein:
the first target is hybridised to the first oligonucleotide by complementary base pairing to thereby form a double-stranded duplex,
the composition further comprises a restriction endonuclease capable of digesting a double-stranded duplex comprising the first target thereby modifying the first oligonucleotide and enable the first detection moiety to provide the first detectable signal.

155. The composition of claim 154, wherein:
the restriction endonuclease is a nicking endonuclease capable of associating with and cleaving a strand of said double-stranded duplex, and said strand comprises the first oligonucleotide.

156. The composition of any one of claims 153 to 155, wherein:
the first detection moiety is a fluorophore and said modifying of the first oligonucleotide increases the distance of the fluorophore from a quencher molecule.

157. The composition of claim 156, wherein:
the first oligonucleotide comprises the quencher molecule.

158. A composition comprising:
a first oligonucleotide for detection of a first target comprising a first detection moiety, wherein:
the first detection moiety is capable of generating a first detectable signal upon modification of the first oligonucleotide, and
the modification is induced by the first target;
an intact stem-loop oligonucleotide for detection of the second target, and comprising a double-stranded stem portion of hybridised nucleotides opposing strands of which are linked by an unbroken single-stranded loop portion of unhybridised nucleotides, wherein at least one strand of the double-stranded stem portion comprises a second detection moiety; and
a first enzyme capable of digesting one or more of the unhybridised nucleotides of the intact stem-loop oligonucleotide only when the second target is present in the sample, to thereby break the single-stranded loop portion and provide a split stem-loop oligonucleotide;
wherein:
the second detection moiety is capable of generating a second detectable signal upon dissociation of the double-stranded stem portion of the split stem-loop oligonucleotide, and
the first and second detection moieties are capable of generating detectable signals that cannot be differentiated at a single temperature using a single type of detector.

159. The composition of claim 158, wherein the first oligonucleotide differs in sequence from:
each strand of the double-stranded stem portion of the intact stem-loop oligonucleotide; and
the single-stranded loop portion of the intact stem-loop oligonucleotide.

160. The composition of claim 158 or claim 159 wherein:
the first target is a nucleic acid sequence;
the composition further comprises:
a first primer complementary to a first sequence in the first target,
a second oligonucleotide comprising a component complementary to a second sequence in the first target that differs from the first sequence, and a tag portion that is not complementary to the first target,
a first polymerase comprising exonuclease activity, and
optionally a second polymerase.

161. The composition of claim 160, wherein:
the first primer and the second oligonucleotide are each hybridised to the first target by complementary base pairing,
the first polymerase is capable of extending the first primer using the target as a template to thereby cleave off the tag portion, allowing the cleaved tag portion to hybridise to the first oligonucleotide by complementary base pairing, and
the first polymerase or the optional second polymerase is/are capable of extending the tag portion using the first oligonucleotide as a template to generate a double-stranded sequence comprising the first oligonucleotide thereby modify the first oligonucleotide and enabling the first detection moiety to provide the first detectable signal.

162. The composition of claim 160 or claim 161, wherein:
the first oligonucleotide comprises a fluorophore and a quencher molecule.

163. The composition of claim 162, wherein:
the first oligonucleotide comprises a fluorophore and a quencher molecule, and
said extending the tag portion increases the distance between the fluorophore and the quencher molecule.

164. The composition of claim 158 or claim 159, wherein:
the first target is a co-factor for enzyme catalytic activity;
the composition further comprises a DNAzyme or a ribozyme requiring the co-factor for catalytic activity, and
DNAzyme or ribozyme is capable of binding to the first target and hybridising to the first oligonucleotide by complementary base pairing, thereby digesting and modifying the first oligonucleotide enabling the first detection moiety to generate the first detectable signal.

165. The composition of claim 164, wherein the co-factor is a metal ion, or a metal ion selected from: Mg²⁺, Mn²⁺, Ca²⁺, Pb²⁺.

166. The method of claim 158 or claim 159, wherein:
the first oligonucleotide is a substrate for a multi-component nucleic acid enzyme (MNAzyme);
the composition further comprises an MNAzyme capable of cleaving the first oligonucleotide when the first target is present in the sample; and
wherein the MNAzyme is capable of binding to the first target and hybridising to the first oligonucleotide by complementary base pairing via its substrate arms, and said hybridisation facilitates cleavage of the first oligonucleotide thereby modifying it and enabling the first detection moiety to provide the first detectable signal.

167. The composition of claim 166, wherein:
the first target is a nucleic acid sequence; and
the first target is hybridised to the sensor arms of the MNAzyme by complementary base pairing to thereby facilitate assembly of the MNAzyme.

168. The composition of claim 158 or claim 159, wherein:
the first target is an analyte, protein, compound or molecule;
the first oligonucleotide is a substrate for an aptazyme; and
the composition further comprises an aptazyme comprising an aptamer portion capable of binding to the first target, and a nucleic acid enzyme portion capable of digesting the first oligonucleotide and thereby modifying it enabling the first detection moiety to provide the first detectable signal.

169. The composition of claim 168, wherein:
the first target is bound to the aptamer portion of the aptazyme and the first oligonucleotide is hybridised to the active nucleic acid enzyme portion by complementary base pairing facilitating digestion of the first oligonucleotide and thereby modifying it enabling the first detection moiety to provide the first detectable signal.

170. The composition of any one of claims 166 to 169, wherein:
the first detection moiety is a fluorophore and said modifying the first oligonucleotide increases the distance of the fluorophore from a quencher molecule.

171. The composition of claim 170, wherein:
the first oligonucleotide comprises the quencher molecule.

172. The composition of any one of claims 138 to 144, 147, 148, 151, 153 to 155, 158 to 161, and 164 to 169, wherein:
the first detection moiety is: a nanoparticle, a metallic nanoparticle, a noble metal nanoparticle, an alkali metal nanoparticle, a gold nanoparticle, or a silver nanoparticle; to which the first oligonucleotide is bound; and
the first detectable signal is:
(i) a change in refractive index,
(ii) a change in colour; and/or
(iii) a change in absorption spectrum,
arising from the first detection moiety following said modification of the first oligonucleotide.

173. The composition of claim 172, wherein:
the first detection moiety is an electrochemical agent to which the first oligonucleotide is bound;
the first detectable signal is a change in electrochemical signal.

174. The composition of claim 173, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

175. The composition of any one of claims 172 to 174, wherein:
the second detection moiety is: a nanoparticle, a metallic nanoparticle, a noble metal nanoparticle, an alkali metal nanoparticle, a gold nanoparticle, or a silver nanoparticle; to which at least one strand of the double-stranded stem portion of the second oligonucleotide is bound and
the second detectable signal is:
(i) a change in refractive index,
(ii) a change in colour; and/or
(iii) a change in absorption spectrum,
arising upon said dissociation of the double-stranded stem portion of the split stem-loop oligonucleotide.

176. The composition any one of claims 172 to 174, wherein:
the second detection moiety is an electrochemical agent to which the second oligonucleotide is bound; and
the second detectable signal is a change in electrochemical signal arising upon said dissociation of the double-stranded stem portion of the split stem-loop oligonucleotide.

177. The composition of claim 176, wherein:
the electrochemical agent is selected from any one or more of a nanoparticle, Methylene blue, Toluene blue, Oracet Blue, Hoechst 33258, [Ru(phen)3]2+, ferrocene, and/or daunomycin.

178. The composition of any one of claims 145, 146, 149, 150, 152, 156, 157, 162, 163, 170, and 171 wherein:
the second detection moiety is a fluorophore, and
the second detectable signal provided by said second detection moiety upon dissociation of the double-stranded stem portion of the split stem-loop oligonucleotide increases the distance of the fluorophore from a quencher molecule.

179. The composition of claim 178, wherein:
the fluorophore and quencher molecule are located on opposing strands of the double-stranded stem portion of the stem-loop oligonucleotide.

180. The composition of any one of claims 138 to 179, wherein:
the first enzyme is a first MNAzyme,
the first MNAzyme is bound to the second target,
the substrate arms of said first MNAzyme are hybridised by complementary base pairing to the single loop portion of the intact stem-loop oligonucleotide, thereby facilitating digestion of the one or more unhybridised nucleotides of the intact stem-loop oligonucleotide and providing the split stem-loop oligonucleotide.

181. The composition of claim 180, wherein:
the second target or is a nucleic acid sequence; and
the second target is hybridised to the sensor arms of the first MNAzyme by complementary base pairing to thereby facilitate assembly of the first MNAzyme.

182. The composition of any one of any one of claims 138 to 179, wherein:
the second target is an analyte, protein, compound or molecule;
the first enzyme is an aptazyme comprising an aptamer capable of binding to the second target; and
the aptamer is bound to the second target thereby rendering the first enzyme catalytically active.

183. The composition of claim 182, wherein:
the first enzyme is any one of an: apta-DNAzyme, apta-ribozyme, apta-MNAzyme.

184. The composition of any one of claims 138 to 179, wherein:
the second target is an analyte, protein, compound or molecule;
the single-stranded loop portion of the intact stem-loop oligonucleotide is a substrate for an aptazyme; and
the composition further comprises an aptazyme comprising an aptamer portion capable of binding to the second target, and a nucleic acid enzyme portion capable of digesting the one or more unhybridised nucleotides of the intact stem-loop oligonucleotide to thereby form the split stem-loop oligonucleotide.

185. The composition of claim 184, wherein:
the second target is bound to the aptamer portion of the aptazyme and the single-stranded loop portion of the intact stem-loop oligonucleotide is hybridised to the active nucleic acid enzyme portion by complementary base pairing, facilitating digestion of the one or more unhybridised nucleotides of the intact stem-loop oligonucleotide to thereby form the split stem-loop oligonucleotide.

186. The composition of any one of claims 138 to 179, wherein:
the second target is a nucleic acid sequence; and
the first enzyme is a first restriction endonuclease, and
the second target is hybridised to the single-stranded loop portion of the intact stem-loop oligonucleotide by complementary base pairing to form a double-stranded sequence for the first restriction endonuclease to associate with and digest the one or more unhybridised nucleotides of the intact stem-loop oligonucleotide to thereby form the split stem-loop oligonucleotide.

187. The composition of claim 186, wherein:
the first restriction endonuclease is a first nicking endonuclease capable of associating with and cleaving a strand of said double-stranded sequence for the first restriction endonuclease, and said strand comprises the intact stem-loop oligonucleotide.

188. The composition of any one of claims 138 to 179, wherein:
the first enzyme comprises a polymerase with exonuclease activity,
the second target is hybridised to the single-stranded loop portion of the intact stem-loop oligonucleotide by complementary base pairing to form a first double-stranded sequence comprising a portion of the second target,
the composition further comprises a first primer oligonucleotide hybridised by complementary base pairing to the second target to form a second double-stranded sequence located upstream relative to the first double-stranded sequence comprising the portion of the second target, and
the primer can be extended using the polymerase with exonuclease activity and the second target as a template sequence, digesting the single-stranded loop portion of the first double stranded sequence and thereby forming a split stem-loop oligonucleotide.

189. The composition of any one of claims 138 to 179, wherein:
the first enzyme is an exonuclease, and
the second target is hybridised by complementary base pairing to the single-stranded loop portion of the intact stem-loop oligonucleotide forming a first double-stranded sequence comprising a portion of the second target, to which the first enzyme comprising exonuclease activity can associate and thereby digest the single-stranded loop portion of the first double stranded sequence comprising the second target to form the split stem-loop oligonucleotide.

190. The composition of any one of claims 138 to 179, wherein:
the first enzyme is a DNAzyme or a ribozyme requiring a co-factor for catalytic activity, and
the second target is the co-factor and is bound to the DNAzyme or ribozyme,
the DNAzyme or ribozyme is hybridised to the single-stranded loop portion of the intact stem-loop oligonucleotide by complementary base pairing, allowing it to digest the one or more unhybridised nucleotides of the single-stranded loop portion of the intact stem-loop oligonucleotide and thereby form the split stem-loop oligonucleotide.

191. The composition of claim 190, wherein the co-factor is a metal ion, or a metal ion selected from: Mg²⁺, Mn²⁺, Ca²⁺, Pb²⁺.

192. The composition of any one of claim 138 to 150, 153, 156 to 158, 166 or 167, wherein:
the first oligonucleotide is selected from any one or more of: a Molecular Beacon®, a Scorpions® primer, a TaqMan® primer, or an MNAzyme substrate.

193. The composition of any one of claims 138 to 192 wherein:
the first target and/or the second target is an amplicon of a nucleic acid.


SEQUENCE LISTING (68 sequences)

SEQ ID NO: 1 - DNA, 26 bp, Artificial Sequence
  Forward primer 1 targeting CTcry
aatatcatctttgcggttgcgtgtcc

SEQ ID NO: 2 - DNA, 22 bp, Artificial Sequence
  Reverse primer 1 targeting CTcry
gctgtgacggagtacaaacgcc

SEQ ID NO: 3 - DNA, 40 bp, Artificial Sequence
  Partzyme A1 targeting CTcry
  misc_feature (40)..(40): /3Phos/ indicates a 3′ phosphate group
tcctgtgaccttcattatgtcgacaacgagaggaaacctt

SEQ ID NO: 4 - DNA, 37 bp, Artificial Sequence
  Partzyme B1 targeting CTcry
  misc_feature (37)..(37): /3Phos/ indicates a 3′ phosphate group
tgcccagggaggctagctgagtctgagcaccctaggc

SEQ ID NO: 5 - DNA, 19 bp, Artificial Sequence
  Forward primer 2 targeting NGopa
gtgttgaaacaccgcccgg

SEQ ID NO: 6 - DNA, 22 bp, Artificial Sequence
  Reverse primer 2 targeting NGopa
gctccttattcggtttgaccgg

SEQ ID NO: 7 - DNA, 38 bp, Artificial Sequence
  Partzyme A2 targeting NGopa
  misc_feature (38)..(38): /3Phos/ indicates a 3′ phosphate group
ccggaacccgatataatccgcacaacgagagggtcgag

SEQ ID NO: 8 - DNA, 43 bp, Artificial Sequence
  Partzyme B2 targeting NGopa
  misc_feature (43)..(43): /3Phos/ indicates a 3′ phosphate group
ggacgagggaggctagctccttcaacatcagtgaaaatctttt

SEQ ID NO: 9 - DNA, 26 bp, Artificial Sequence
  Forward primer 3 targeting TFRC
gctaaaacaataactcagaacttacg

SEQ ID NO: 10 - DNA, 24 bp, Artificial Sequence
  Reverse primer 3 targeting TFRC
cagctttctgaggttaccatccta

SEQ ID NO: 11 - DNA, 37 bp, Artificial Sequence
  Partzyme A3 targeting TFRC
  misc_feature (37)..(37): /3Phos/ indicates a 3′ phosphate group
ggaatatggaaggagactgtcacaacgaggggtcgag

SEQ ID NO: 12 - DNA, 37 bp, Artificial Sequence; identical to SEQ ID NO: 11
  Partzyme B3 targeting TFRC
  misc_feature (37)..(37): /3Phos/ indicates a 3′ phosphate group

SEQ ID NO: 13 - DNA, 22 bp, Artificial Sequence
  Substrate 1 (Universal target)
  misc_feature (1)..(1): /56-JOEN/ indicates the location of a JOE fluorophore
  misc_feature (22)..(22): /3IABkFQ/represents the location of an Iowa Black FQ quencher capable of absorbing fluorescence in the range of 420 - 620 nm
aaggtttcctcguccctgggca

SEQ ID NO: 14 - DNA, 55 bp, Artificial Sequence
  LOCS-1 (Universal target)
  misc_feature (1)..(1): /56-JOEN/ indicates the location of a JOE fluorophore
  misc_feature (55)..(55): /3IABkFQ/represents the location of an Iowa Black FQ quencher capable of absorbing fluorescence in the range of 420 - 620 nm
aacgacaatggccttttctcgaccctcguccctcgtccttttggccattgtcgtt

SEQ ID NO: 15 - DNA, 21 bp, Artificial Sequence
  Substrate 2 (Universal target)
  misc_feature (1)..(1): /5RHO101N/ indicates the location of an ATTO Rho101 fluorophore
  misc_feature (21)..(21): /3IAbRQSp/ represents the location of an Iowa Black RQ quencher used for absorbing fluorescence in the range of 500 - 700 nm
ctcgaccccguctccacgcca

SEQ ID NO: 16 - DNA, 23 bp, Artificial Sequence
  Forward primer 4 targeting TVK
gtttgtgtctcgtgccatagtcg

SEQ ID NO: 17 - DNA, 22 bp, Artificial Sequence
  Reverse primer 4 targeting TVK
atttcatggtcgccctcggagt

SEQ ID NO: 18 - DNA, 41 bp, Artificial Sequence
  Partzyme A4 targeting TVK
  misc_feature (41)..(41): /3Phos/ indicates a 3′ phosphate group
tatatgagtttgagaccaagaatgacaacgagaggcgtgat

SEQ ID NO: 19 - DNA, 43 bp, Artificial Sequence
  Partzyme B4 targeting TVK
  misc_feature (43)..(43): /3Phos/ indicates a 3′ phosphate group
ctgggaggagaggctagctgtgtaactcgacctgtccgattca

SEQ ID NO: 20 - DNA, 28 bp, Artificial Sequence
  Forward primer 5 targeting MgPa
ggctcgccatttaaacccctttgcaccg

SEQ ID NO: 21 - DNA, 31 bp, Artificial Sequence
  Reverse primer 5 targeting MgPa
aataccttgatggtcagcaaaactttgcaat

SEQ ID NO: 22 - DNA, 45 bp, Artificial Sequence
  Partzyme A5 targeting MgPa
  misc_feature (45)..(45): /3Phos/ indicates a 3′ phosphate group
tgctaagttaatatcatataaagctctaacaacgagggacgtcga

SEQ ID NO: 23 - DNA, 44 bp, Artificial Sequence
  Partzyme B5 targeting MgPa
  misc_feature (44)..(44): /3Phos/ indicates a 3′ phosphate group
gcggtagaggaggctagctccgttgttatcataccttctgattg

SEQ ID NO: 24 - DNA, 22 bp, Artificial Sequence
  Substrate 3 (Universal target)
  misc_feature (1)..(1): /56-FAM/ indicates the location of a FAM fluorophore
  misc_feature (22)..(22): /3IABkFQ/represents the location of an Iowa Black FQ quencher capable of absorbing fluorescence in the range of 420 - 620 nm
atcacgcctcguctcctcccag

SEQ ID NO: 25 - DNA, 58 bp, Artificial Sequence
  LOCS-2 (Universal target)
  misc_feature (1)..(1): /56-FAM/ indicates the location of a FAM fluorophore
  misc_feature (58)..(58): /3IABkFQ/represents the location of an Iowa Black FQ quencher capable of absorbing fluorescence in the range of 420 - 620 nm
gcgtgaccggtccaaaatcgacgtcccrgrucctctaccgcaaaaggaccggtcacgc

SEQ ID NO: 26 - DNA, 37 bp, Artificial Sequence
  Partzyme A6 targeting TFRC
  misc_feature (37)..(37): /3Phos/ indicates a 3′ phosphate group
ggaatatggaaggagactgtcacaacgagtggttggc

SEQ ID NO: 27 - DNA, 40 bp, Artificial Sequence
  Partzyme B6 targeting TFRC
  misc_feature (40)..(40): /3Phos/ indicates a 3′ phosphate group
gtcgtgttggaggctagctcctctgactggaaaacagact

SEQ ID NO: 28 - DNA, 21 bp, Artificial Sequence
  Substrate 4 (Universal target)
gccaaccacguccaacacgac

SEQ ID NO: 29 - DNA, 19 bp, Artificial Sequence
  Forward primer 6 targeting TPApolA
agggctagtacaccggagg

SEQ ID NO: 30 - DNA, 24 bp, Artificial Sequence
  Reverse Primer 6 targeting TPApolA
cctaagatctctattttctatagg

SEQ ID NO: 31 - DNA, 39 bp, Artificial Sequence
  Partzyme A7 targeting TPApolA
  misc_feature (39)..(39): /3Phos/ indicates a 3′ phosphate group
ttgaagtcggagttgaagacgaacaacgagggacgtcga

SEQ ID NO: 32 - DNA, 38 bp, Artificial Sequence
  Partzyme B7 targeting TPApolA
  misc_feature (38)..(38): /3Phos/ indicates a 3′ phosphate group
cggtagaggaggctagctgtgctgtgtctggcgccata

SEQ ID NO: 33 - DNA, 56 bp, Artificial Sequence
  LOCS-3 (Universal target)
  misc_feature (1)..(1): /5Atto680N/ indicates the location of an ATTO 680 fluorophor
  misc_feature (56)..(56): /3IAbRQSp/ represents the location of an Iowa Black RQ quencher used for absorbing fluorescence in the range of 500 - 700 nm
aacgacaatggcctttttcgacgtcccgucctctaccgcttttggccattgtcgtt

SEQ ID NO: 34 - DNA, 60 bp, Artificial Sequence
  LOCS-4 (Universal target)
  misc_feature (1)..(1): /5Cy5/ indicates the location of a cy5.5 fluorophore
  misc_feature (60)..(60): /3IAbRQSp/ represents the location of an Iowa Black RQ quencher used for absorbing fluorescence in the range of 500 - 700 nm
cacggtctagagctcttttaaggtttcctcguccctgggcattttgagctctagaccgtg

SEQ ID NO: 35 - DNA, 36 bp, Artificial Sequence
  Partzyme A8 targeting NGopa
  misc_feature (36)..(36): /3Phos/ indicates a 3′ phosphate group
ggaacccgatataatccgcacaacgagagggtcgag

SEQ ID NO: 36 - DNA, 39 bp, Artificial Sequence
  Partzyme B8 targeting NGopa
  misc_feature (39)..(39): /3Phos/ indicates a 3′ phosphate group
ggacgagggaggctagctccttcaacatcagtgaaaatc

SEQ ID NO: 37 - DNA, 47 bp, Artificial Sequence
  LOCS-5 (Universal target)
  misc_feature (1)..(1): /56-FAM/ indicates the location of a FAM fluorophore
  misc_feature (47)..(47): /3IABkFQ/represents the location of an Iowa Black FQ quencher capable of absorbing fluorescence in the range of 420 - 620 nm
cgcactggcttttctcgaccctcguccctcgtccttttgccagtgcg

SEQ ID NO: 38 - DNA, 23 bp, Artificial Sequence
  Forward Primer 7 targeting LGV
tacagaaaaaatagaccctttcc

SEQ ID NO: 39 - DNA, 23 bp, Artificial Sequence
  Reverse Primer 7 targeting LGV
gtattctcctttatctactgtgc

SEQ ID NO: 40 - DNA, 35 bp, Artificial Sequence
  Partzyme A9 targeting LGV
  misc_feature (35)..(35): /3Phos/ indicates a 3′ phosphate group
ccgagcatcactaactgttgacaacgagaggcgtg

SEQ ID NO: 41 - DNA, 39 bp, Artificial Sequence
  Partzyme B9 targeting LGV
  misc_feature (39)..(39): /3Phos/ indicates a 3′ phosphate group
ctgggaggagaggctagctgagcaggcggagttgatgat

SEQ ID NO: 42 - DNA, 48 bp, Artificial Sequence
  LOCS-6 (Universal target)
  misc_feature (1)..(1): /5Cy5/ indicates the location of a cy5.5 fluorophore
  misc_feature (48)..(48): /3IAbRQSp/ represents the location of an Iowa Black RQ quencher used for absorbing fluorescence in the range of 500 - 700 nm
cgcactggcttttatcacgcctcguctcctcccagttttgccagtgcg

SEQ ID NO: 43 - DNA, 22 bp, Artificial Sequence
  Forward Primer 8 targeting NGporA
agcattcaatttgttccgagtc

SEQ ID NO: 44 - DNA, 22 bp, Artificial Sequence
  Reverse Primer 8 targeting NGporA
caacagccggaactggtttcat

SEQ ID NO: 45 - DNA, 36 bp, Artificial Sequence
  Partzyme A10 targeting NGporA
  misc_feature (36)..(36): /3Phos/ indicates a 3′ phosphate group
aagtccgcctatacgcctgacaacgagaggtgcggt

SEQ ID NO: 46 - DNA, 38 bp, Artificial Sequence
  Partzyme B10 targeting NGporA
  misc_feature (38)..(38): /3Phos/ indicates a 3′ phosphate group
agctggggaggctagctctactttcacgctggaaagta

SEQ ID NO: 47 - DNA, 55 bp, Artificial Sequence
  LOCS-7 (Universal target)
  misc_feature (1)..(1): /56-FAM/ indicates the location of a FAM fluorophore
  misc_feature (55)..(55): /3IABkFQ/represents the location of an Iowa Black FQ quencher capable of absorbing fluorescence in the range of 420 - 620 nm
aacgacaatggccttttaccgcacctcguccccagctcttttggccattgtcgtt

SEQ ID NO: 48 - DNA, 24 bp, Artificial Sequence
  Forward Primer 9 targeting HSV-1
ctaacagcgcgaacgaccaactac

SEQ ID NO: 49 - DNA, 20 bp, Artificial Sequence
  Reverse Primer 9 targeting HSV-1
cagcccccataccggaacgc

SEQ ID NO: 50 - DNA, 38 bp, Artificial Sequence
  Partzyme A11 targeting HSV-1
  misc_feature (38)..(38): /3Phos/ indicates a 3′ phosphate group
ccgatcatcagttatccttaagacaacgaggggtcgag

SEQ ID NO: 51 - DNA, 40 bp, Artificial Sequence
  Partzyme B11 targeting HSV-1
  misc_feature (40)..(40): /3Phos/ indicates a 3′ phosphate group
tggcgtggagaggctagctgtctcttttgtgtggtgcgtt

SEQ ID NO: 52 - DNA, 51 bp, Artificial Sequence
  LOCS- (Universal target)
  misc_feature (1)..(1): /56-JOEN/ indicates the location of a JOE fluorophore
  misc_feature (51)..(51): /3IABkFQ/represents the location of an Iowa Black FQ quencher capable of absorbing fluorescence in the range of 420 - 620 nm
tcgggtagcttttttctcgaccccguctccacgccattttaagctacccga

SEQ ID NO: 53 - DNA, 24 bp, Artificial Sequence
  Forward Primer 10 targeting HSV-2
ctaccaaatacgccttagcagacc

SEQ ID NO: 54 - DNA, 26 bp, Artificial Sequence
  Reverse Primer 10 targeting HSV-2
caggctgaatgtggtaaacacgcttc

SEQ ID NO: 55 - DNA, 37 bp, Artificial Sequence
  Partzyme A12 targeting HSV-2
  misc_feature (37)..(37): /3Phos/ indicates a 3′ phosphate group
agacccctcgcttaagatggacaacgagaggactagg

SEQ ID NO: 56 - DNA, 38 bp, Artificial Sequence
  Partzyme B12 targeting HSV-2
  misc_feature (38)..(38): /3Phos/ indicates a 3′ phosphate group
gacgtgaggaggctagctccgatcccaatcgatttcgc

SEQ ID NO: 57 - DNA, 46 bp, Artificial Sequence
  LOCS-9 (Universal target)
  misc_feature (1)..(1): /56-JOEN/ indicates the location of a JOE fluorophore
  misc_feature (46)..(46): /3IABkFQ/represents the location of an Iowa Black FQ quencher capable of absorbing fluorescence in the range of 420 - 620 nm
acagtgcattttcctagtcctcgucctcacgtcctttttgcactgt

SEQ ID NO: 58 - DNA, 30 bp, Artificial Sequence
  Forward Primer 11 targeting MgPa
gttgagaaataccttgatggtcagcaaaac

SEQ ID NO: 59 - DNA, 20 bp, Artificial Sequence
  Reverse Primer 11 targeting MgPa
acccctttgcaccgttgagg

SEQ ID NO: 60 - DNA, 40 bp, Artificial Sequence
  Partzyme A13 targeting MgPa
  misc_feature (40)..(40): /3Phos/ indicates a 3′ phosphate group
atcagaaggtatgataacaacggacaacgagagggcggtt

SEQ ID NO: 61 - DNA, 45 bp, Artificial Sequence
  Partzyme B13 targeting MgPa
  misc_feature (45)..(45): /3Phos/ indicates a 3′ phosphate group
ggttcacgggaggctagcttagagctttatatgatattaacttag

SEQ ID NO: 62 - DNA, 54 bp, Artificial Sequence
  LOCS-10 (Universal target)
  misc_feature (1)..(1): /5RHO101N/ indicates the location of an ATTO Rho101 fluorophore
  misc_feature (54)..(54): /3IAbRQSp/ represents the location of an Iowa Black RQ quencher used for absorbing fluorescence in the range of 500 - 700 nm
tctacgccaccagttttaccgccctcgucccgtgaacttttctggtggcgtaga

SEQ ID NO: 63 - DNA, 33 bp, Artificial Sequence
  Forward Primer 12 targeting TVbtub
tgcattgataacgaagctctttatgatatttgc

SEQ ID NO: 64 - DNA, 25 bp, Artificial Sequence
  Reverse Primer 12 targeting TVbtub
aacatgttgttccggacataaccat

SEQ ID NO: 65 - DNA, 40 bp, Artificial Sequence
  Partzyme A14 targeting TVbtub
  misc_feature (40)..(40): /3Phos/ indicates a 3′ phosphate group
ccgtacactcaagctcacaacacacaacgaggggagagga

SEQ ID NO: 66 - DNA, 41 bp, Artificial Sequence
  Partzyme B14 targeting TVbtub
  misc_feature (41)..(41): /3Phos/ indicates a 3′ phosphate group
ttgaaggggaggctagctcaacatacggcgatcttaaccac

SEQ ID NO: 67 - DNA, 46 bp, Artificial Sequence
  LOCS-11 (Universal target)
  misc_feature (1)..(1): /5RHO101N/ indicates the location of an ATTO Rho101 fluorophore
  misc_feature (46)..(46): /3IAbRQSp/ represents the location of an Iowa Black RQ quencher used for absorbing fluorescence in the range of 500 - 700 nm
tcaaggactttttcctctccccguccccttcaacttttgtccttga

SEQ ID NO: 68 - DNA, 49 bp, Artificial Sequence
  Molecular Beacon 1 targeting TVbtub
  misc_feature (1)..(1): /56-FAM/ indicates the location of a FAM fluorophore
  misc_feature (49)..(49): /3IABkFQ/represents the location of an Iowa Black FQ quencher capable of absorbing fluorescence in the range of 420 - 620 nm
tatgccgacttttcaccaacatacggcgatcttaacttttctcggcata